(19) United States
(12) Patent Application Publication
(10) Pub. No.: US 2006/0167268 A1
(43) Pub. Date: Jul. 27, 2006

(54) Growth hormone secretagogues

(75) Inventors: Britta Evers, Hamburg (DE); Ruehter Gerd, Hamburg (DE); Eva Maria Martin De La Nava, Madrid (ES); Mark Joseph Tebbe, Hamburg (DE)

Correspondence Address: ELI LILLY & COMPANY, PATENT DIVISION, P.O. BOX 6288, INDIANAPOLIS, IN, 46206-6288, US

(73) Assignee: Eli Lilly and Company, Patent Division,, Indianapolis, IN (US)

(21) Appl. No.: 10/510,305

(22) PCT Filed: Mar. 31, 2003
(86) PCT No.: PCT/US03/08680

§ 371 Date: Oct. 5, 2004

Related U.S. Application Data
(60) Provisional application No. 60/371,271, filed on Apr. 9, 2002.
(60) Provisional application No. 60/371,270, filed on Apr. 9, 2002.
(60) Provisional application No. 60/371,278, filed on Apr. 9, 2002.
(60) Provisional application No. 60/371,275, filed on Apr. 9, 2002.
(60) Provisional application No. 60/371,277, filed on Apr. 9, 2002.

(30) Foreign Application Priority Data
Apr. 9, 2002 (GB) 0208116.4
Apr. 9, 2002 (GB) 0208117.2
Apr. 9, 2002 (GB) 0208118.0
Apr. 9, 2002 (GB) 0208119.8
Apr. 9, 2002 (GB) 0208120.6

(51) Int. Cl. C07D 275/02; C07D 207/24

(52) U.S. Cl. 548/214; 548/550

(57) ABSTRACT

This invention relates to novel compounds which are useful in the modulation of endogenous growth hormone levels in a mammal. The invention further relates to novel intermediates for use in the synthesis of said compounds, as well as novel processes employed in these syntheses. Also included are methods of treating a mammal which include the administration of said compounds.

Growth hormone, which is secreted by the pituitary gland, has wide-ranging developmental effects on the organism. Artificial manipulation of growth hormone levels has been demonstrated to have significant therapeutic utility. Human growth hormone supplementation has been shown to be an effective treatment for growth hormone deficiencies and their related disease states in humans. Apart from this application, studies have uncovered new and significant properties of growth hormone which lend further importance to the ability to control growth hormone levels. For example, clinical studies have indicated that growth hormone supplementation may be useful in combating the maladies of ageing in humans. Elevated growth hormone levels in animals have been shown to result in increased lean muscle mass. One application of this latter observation could result in higher production of leaner meat products or in the production of larger and/or stronger animals.

While growth hormone is naturally produced by the pituitary gland, the secretion of growth hormone into the bloodstream is controlled by a second protein, Growth

Hormone Releasing Factor (GRF). This hormone is also commonly known in the art as somatocrinin, Growth Hormone Releasing Hormone (GHRH), and Growth Releasing Hormone (GRH).

There are two ways to approach the problem of increasing circulating levels of growth hormone: (1) increase the level of human growth hormone in the organism directly or (2) increase the organism's natural tendency to produce growth hormone. The latter strategy may be achieved via supplementation with GRF. GRF has been demonstrated to increase the circulatory levels of growth hormone in vivo. (Rivier, et al., Nature (London), 300:276 (1982). The effect of GRF, including structural analogs thereof, on growth hormone production has been-widely studied. A primary obstacle to the use of GRF as a direct supplement is its short lifespan in vivo. L. A. Frohman, et al., Journal of Clinical Investigation, 78:906 (1986). More potent and/or longer lasting GRF molecules are therefore desirable for the development of effective human therapeutic or animal husbandry agents.

The structure of GRF has been modified in numerous ways resulting in longer lasting and/or more potent GRF analogs. It has been demonstrated that the first 29 amino acids from the N-terminus are sufficient to retain full GRF activity. Speiss, et al., Biochemistry, 21:6037 (1982). One strategy has been the incorporation of novel D-amino acid residues in various regions of the GRF molecule. V. A. Lance; et al., Biochemical and Biophysical Research Communications, 119:265 (1984); D. H. Coy, et al., Peptides, 8(suppl. 1):49 (1986). Another strategy has modified the peptide backbone of GRF by the incorporation of peptide bond isosteres in the N-terminal region. D. Tourwe, Janssen. Chim. Acta, 3:3 (1985); S. J. Hocart, et al., Journal of Medicinal Chemistry, 33:1954-58 (1990). A series of very active analogs of GHRH is described in European Patent Publication 511,003, published Oct. 28, 1992.

In addition to the actions of GHRH there are various ways known to release growth hormone. For example, chemicals such as arginine, L-3,4-dihydroxyphenylalanine (L-DOPA), glucagon, vasopressin, and insulin-induced hypoglycemia, as well as activities such as sleep and exercise, indirectly cause growth hormone to be released from the pituitary by acting in some fashion on the hypothalamus, perhaps either to decrease somatostatin secretion or to increase the secretion of GHRH.

In cases where increased levels of growth hormone are desired, the problem has generally been solved by providing exogenous growth hormone or by administering GHRH, or a related peptidyl compound which stimulates growth hormone production or release. In either instance the peptidyl nature of the compound has necessitated that it be administered by injection.

Other compounds have been developed which stimulate the release of endogenous growth hormone, such as analogous peptidyl compounds related to GHRH. These peptides, while considerably smaller than growth hormones are still susceptible to metabolic instability.

Administration of the hexapeptide growth hormone releasing peptide-6 (GHRP-6) results in the secretion of growth hormone in many species, including humans. This peptide is one of a series of synthetic peptides, the structures of which were based on the pentapeptide Met-enkephalin. It has been shown that GHRP binds specifically to the pituitary, although the binding does not involve the opioid, GHRH, or the somatostatin receptors.

In recent years significant efforts have been taken to develop nonpeptidyl analogs of this series of compounds. Such compounds, termed growth hormone secretagogues, should be orally bioavailable, induce the production or release of growth hormone, and act in concert, or synergistically with GHRH. These compounds are non-peptidyl in nature and are, therefore, more metabolically stable than growth hormone, growth hormone releasing hormone, or analogs of either of these proteins.

The compounds of this invention are especially desired due to the enhanced in vivo pharmaceutical activity of the compounds.

The present invention relates to compounds of Formula I 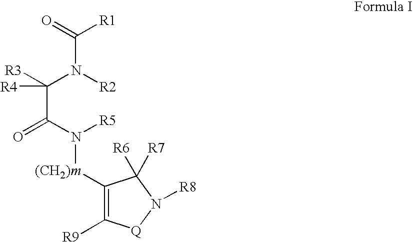
wherein:

R1 is NHR10, (substituted or unsubstituted C₁-C₆alkyl)NHR10 or (unsubstituted or substituted C₃-C₈ cycloalkyl)NHR10;

R10 is hydrogen, C₁-C₆alkyl, C₁-C₆alkyl(OH), C₁-C₆alkylidenyl(OH)R11, or an amino protecting group;

R11 is C₁-C₆alkyl, C₂-C₆alkenyl, C₁-C₆alkyl(O)C₁-C₆alkyl, C(O)O—C₃-C₆alkyl, aryl, or C₁-C₆alkylaryl;

R2 is hydrogen, C₁-C₆alkyl, aryl, or C₁-C₆alkylaryl;

R3 is unsubstituted or substituted aryl, unsubstituted or substituted C₁-C₆alkylaryl, unsubstituted or substituted C₁-C₆alkyl(O)—C₁-C₆alkylaryl, unsubstituted or substituted C₃-C₈cycloalkyl, unsubstituted or substituted (C₁-C₆alkyl) C₃-C₈cycloalkyl, indolinyl;

R4 is hydrogen, C₁-C₆alkyl, aryl, C₁-C₆alkylaryl, or C₂-C₆alkenyl;

R5 is hydrogen, aryl, C₁-C₆alkylaryl, hydroxy, C₁-C₆alkoxy, unsubstituted or substituted C₁-C₆alkyl;

R6 and R7 are independently hydrogen, unsubstituted or substituted C₁-C₆alkyl, unsubstituted or substituted C₂-C₆alkenyl, or R6 and R7 together with the carbon atom to which they are attached form a carbocyclic ring of up to 8 atoms which is optionally partly unsaturated or a substituted C₃-C₈cycloalkyl group which is optionally partly unsaturated;

R8 is hydrogen, unsubstituted or substituted C₁-C₆alkyl, unsubstituted or substituted aryl, unsubstituted or substituted (C₁-C₆alkyl)C₃-C₈cycloalkyl, or unsubstituted or substituted C₁-C₆alkylaryl;

R9 is hydrogen, C₁-C₆alkyl, C₂-C₆alkenyl, C₂-C₆alkynyl, C₃-C₈cycloalkyl, C₃-C₈cycloalkenyl, cyano, unsubstituted or substituted aryl, unsubstituted or substituted —O-aryl, unsubstituted or substituted —N-aryl, unsubstituted or substituted —S-aryl, -aryl-aryl(K1)(K2), —O-aryl-aryl (K1)(K2), —N-aryl-aryl (K1)(K2), —S-aryl-aryl(K1) (K2), —O—C₁-C₆alkyl, or C₁-C₆alkylaryl, wherein K1 is halo or —CF₃, and K2 is hydrogen, halo or —CF₃ or K1 and K2 together form a methylenedioxy group;

Q is —S(O)₂— or —C(O)—; and

m is a number selected from 1 or 2;

provided that R1 is (substituted C₁-C₆alkyl)NHR10 or (unsubstituted or substituted C₃-C₈cycloalkyl)NHR10; or

R5 is hydroxy, C₁-C₆alkoxy, or substituted C₁-C₆alkyl; or
R6 and R7 are independently unsubstituted or substituted C₁-C₆alkyl or unsubstituted or substituted C₂-C₆alkenyl with the proviso that at least one group is substituted; or

R6 is hydrogen and R7 is substituted C₁-C₆alkyl or substituted C₂-C₆alkenyl; or

R6 and R7 together with the carbon atom to which they are attached may form a substituted C₃-C₈cycloalkyl group which is optionally partly unsaturated; or

R8 is substituted C₁-C₆alkyl, substituted aryl, unsubstituted or substituted (C₁-C₆alkyl)C₃-C₈cycloalkyl, or substituted C₁-C₆alkylaryl;

or a pharmaceutically acceptable salt or solvate thereof.

In particular the present invention relates to a group of compounds of Formula I wherein:
R1 is NHR10 or C₁-C₆alkylNHR10;
R10 is hydrogen, C₁-C₆alkyl, C₁-C₆alkyl(OH), C₁-C₆alkylidenyl(OH)R11, or an amino protecting group;

R11 is C₁-C₆alkyl, C₂-C₆alkenyl, C₁-C₆alkyl(O)C₁-C₆alkyl, C(O)O—C₁-C₆alkyl, aryl, or C₁-C₆alkylaryl;

R2 is hydrogen, C₁-C₆alkyl, aryl, or C₁-C₆alkylaryl;

R3 is unsubstituted or substituted aryl, unsubstituted or substituted C₃-C₆alkylaryl, unsubstituted or substituted C₁-C₆alkyl(O)-C₁-C₆alkylaryl, unsubstituted or substituted C₃-C₈cycloalkyl, unsubstituted or substituted (C₁-C₆ alkyl) C₃-C₈cycloalkyl, indolinyl;

R4 is hydrogen, C₁-C₆alkyl, aryl, C₁-C₆alkylaryl, or C₂-C₆alkenyl;

R5 is hydroxy, C₁-C₆alkoxy, or substituted C₁-C₆alkyl;

R6 and R7 are independently hydrogen, C₁-C₆alkyl, C₂-C₆alkenyl, or R6 and R7 together with the carbon atom to which they are attached form a carbocyclic ring of up to 8 atoms which is optionally partly unsaturated;

R8 is hydrogen, C₁-C₆alkyl, aryl, or C₁-C₆alkylaryl;

R9 is hydrogen, C₁-C₆alkyl, C₂-C₆alkenyl, C₂-C₆alkynyl, C₃-C₈cycloalkyl, C₃-C₈cycloalkenyl, cyano, unsubstituted or substituted aryl, unsubstituted or substituted —O-aryl, unsubstituted or substituted —N-aryl, unsubstituted or substituted —S-aryl, -aryl-aryl(K1)(K2), —O-aryl-aryl(K1)(K2), —N-aryl-aryl(K1)(K2), —S-aryl-aryl(K1)(K2), —O—C₁-C₆alkyl, or C₁-C₆alkylaryl, wherein K1 is halo or —CF₃, and K2 is hydrogen, halo or —CF₃ or K1 and K2 together form a methylenedioxy group;

Q is —S(O)₂— or —C(O)—;

m is a number selected from 1 or 2;

or a pharmaceutically acceptable salt or solvate thereof.

In particular the present invention also relates to a group of compounds of Formula I wherein:

R1 is NHR10 or C₁-C₆alkylNHR10;

R10 is hydrogen, C₁-C₆alkyl, C₁-C₆alkyl(OH), C₃-C₆alkylidenyl(OH)R11, or an amino protecting group;

R11 is C₁-C₆alkyl, C₂-C₆alkenyl, C₃-C₆alkyl(O)C₁-C₆alkyl, C(O)O—C₁-C₆alkyl, aryl, or C₁-C₆alkylaryl;

R2 is hydrogen, C₁-C₆alkyl, aryl, or C₃-C₆alkylaryl;

R3 is unsubstituted or substituted aryl, unsubstituted or substituted C₁-C₆alkylaryl, unsubstituted or substituted C₁-C₆alkyl (O)—C₁-C₆alkylaryl, unsubstituted or substituted C₃-C₈cycloalkyl, unsubstituted or substituted (C₁-C₆ alkyl) C₃-C₈cycloalkyl, indolinyl;

R4 is hydrogen, C₁-C₆alkyl, aryl, C₃-C₆alkylaryl, or C₂-C₆alkenyl;

R5 is hydrogen, aryl, C₁-C₆alkylaryl, hydroxy, C₃-C₆alkoxy, unsubstituted or substituted C₁-C₆alkyl;

R6 and R7 are independently hydrogen, C₁-C₆alkyl, C₂-C₆alkenyl, or R6 and R7 together with the carbon atom to which they are attached form a carbocyclic ring of up to 8 atoms which is optionally partly unsaturated;

R8 is substituted C₁-C₆alkyl, substituted aryl, or substituted C₁-C₆alkylaryl;

R9 is hydrogen, C₁-C₆alkyl, C₂-C₆alkenyl, C₂-C₆alkynyl, C₃-C₈cycloalkyl, C₃-C₈cycloalkenyl, cyano, unsubstituted or substituted aryl, unsubstituted or substituted —O-aryl, unsubstituted or substituted —N-aryl, unsubstituted or substituted —S-aryl, -aryl-aryl(K1)(K2), —O-aryl-aryl(K1)(K2), —N-aryl-aryl(K1)(K2), —S-aryl-aryl(K1)(K2), —O—C₁-C₆alkyl, or C₁-C₆alkylaryl, wherein K1 is halo or —CF₃, and K2 is hydrogen, halo or —CF₃ or K1 and K2 together form a methylenedioxy group;

Q is —S(O)₂— or —C(O)—;

m is a number selected from 1 or 2;

or a pharmaceutically acceptable salt or solvate thereof.

In particular the present invention also relates to a group of compounds of Formula I wherein:

R1 is NHR10 or C₁-C₆alkylNHR10;

R10 is hydrogen, C₁-C₆alkyl, C₁-C₆alkyl(OH), C₃-C₆alkylidenyl(OH)R11, or an amino protecting group;

R11 is C₁-C₆alkyl, C₂-C₆alkenyl, C₃-C₆alkyl (O) C₁-C₆alkyl, C(O)O—C₁-C₆alkyl, aryl, or C₃-C₆alkylaryl;

R2 is hydrogen, C₁-C₆alkyl, aryl, or C₁-C₆alkylaryl;

R3 is unsubstituted or substituted aryl, unsubstituted or substituted C₁-C₆alkylaryl, unsubstituted or substituted C₁-C₆alkyl (O)—C₁-C₆alkylaryl, unsubstituted or substituted C₃-C₈ cycloalkyl, unsubstituted or substituted (C₁-C₆ alkyl) C₃-C₈ cycloalkyl, indolinyl;

R4 is hydrogen, C₁-C₆alkyl, aryl, C₃-C₆alkylaryl, or C₂-C₆alkenyl;

R5 is hydrogen, aryl, C₁-C₆alkylaryl, hydroxy, C₁-C₆alkoxy, unsubstituted or substituted C₁-C₆alkyl;

R6 and R7 are independently unsubstituted or substituted C₃-C₆alkyl or unsubstituted or substituted C₂-C₆alkenyl with the proviso that at least one group is substituted; or

R6 is hydrogen and R7 is substituted C₁-C₆alkyl or substituted C₂-C₆alkenyl; or

or R6 and R7 together with the carbon atom to which they are attached may form a substituted C₃-C₈cycloalkyl group which is optionally partly unsaturated;

R8 is hydrogen, unsubstituted or substituted C₁-C₆alkyl, unsubstituted or substituted aryl, unsubstituted or substituted (C₁-C₆alkyl)C₃-C₈cycloalkyl, or unsubstituted or substituted C₁-C₆alkylaryl;

R9 is hydrogen, C₁-C₆alkyl, C₂-C₆alkenyl, C₂-C₆alkynyl, C₃-C₈cycloalkyl, C₃-C₈cycloalkenyl, cyano, unsubstituted or substituted aryl, unsubstituted or substituted —O-aryl, unsubstituted or substituted —N-aryl, unsubstituted or substituted —S-aryl, -aryl-aryl(K1)(K2), —O-aryl-aryl(K1)(K2), —N-aryl-aryl(K1)(K2), —S-aryl-aryl(K1)(K2), —O-C₁-C₆alkyl, or C₁-C₆alkylaryl, wherein K1 is halo or —CF₃, and K2 is hydrogen, halo or —CF₃ or K1 and K2 together form a methylenedioxy group;

Q is —S(O)₂— or —C(O)—;

m is a number selected from 1 or 2;

or a pharmaceutically acceptable salt or solvate thereof.

In particular the present invention also relates to a group of compounds of Formula I wherein:

R1 is (substituted C₃-C₆alkyl)NHR10 or (unsubstituted or substituted C₃-C₈cycloalkyl)NHR10;

R10 is hydrogen, C₁-C₆alkyl, C₁-C₆alkyl(OH), C₁-C₆alkylidenyl(OH)R11, or an amino protecting group;

R11 is C₁-C₆alkyl, C₂-C₆alkenyl, C₁-C₆alkyl (O)C₁-C₆alkyl, C(O)O—C₁-C₆alkyl, aryl, or C₁-C₆alkylaryl;

R2 is hydrogen, C₃-C₆alkyl, aryl, or C₁-C₆alkylaryl;

R3 is unsubstituted or substituted aryl, unsubstituted or substituted C₁-C₆alkylaryl, unsubstituted or substituted C₁-C₆alkyl (O)—C₁-C₆alkylaryl, unsubstituted or substituted C₃-C₈cycloalkyl, unsubstituted or substituted (C₁-C₆alkyl) C₃-C₈cycloalkyl, indolinyl;

R4 is hydrogen, C₁-C₆alkyl, aryl, C₁-C₆alkylaryl, or C₂-C₆alkenyl;

R5 is hydrogen, aryl, C₁-C₆alkylaryl, hydroxy, C₁-C₆alkoxy, unsubstituted or substituted C₁-C₆alkyl;

R6 and R7 are independently hydrogen, unsubstituted or substituted C₁-C₆alkyl, unsubstituted or substituted C₂-C₆alkenyl, or R6 and R7 together with the carbon atom to which they are attached form a carbocyclic ring of up to 8 atoms which is optionally partly unsaturated or a substituted C₃-C₈cycloalkyl group which is optionally partly unsaturated;

R8 is hydrogen, unsubstituted or substituted C₁-C₆alkyl, unsubstituted or substituted aryl, or unsubstituted or substituted C₁-C₆alkylaryl;

R9 is hydrogen, C₁-C₆alkyl, C₂-C₆alkenyl, C₂-C₆alkynyl, C₃-C₈cycloalkyl, C₃-C₈cycloalkenyl, cyano, unsubstituted or substituted aryl, unsubstituted or substituted —O-aryl, unsubstituted or substituted —N-aryl, unsubstituted or substituted —S-aryl, -aryl-aryl(K1)(K2), —O-aryl-aryl(K1)(K2), —N-aryl-aryl(K1)(K2), —S-aryl-aryl(K1)(K2), —O-C₁-C₆alkyl, or C₁-C₆alkylaryl, wherein K1 is halo or —CF₃, and K2 is hydrogen, halo or —CF₃ or K1 and K2 together form a methylenedioxy group;

Q is —S(O) 2- or —C(O)—;

m is a number selected from 1 or 2;

or a pharmaceutically acceptable salt or solvate thereof.

The present invention further relates to pharmaceutical formulations containing compounds of formula I, alone or in combination with other growth hormone secretagogue compounds, and/or in combination with suitable bone-antiresorptive agents, and the use of said compounds and/or formulations at least for the increase in endogenous levels of growth hormone in a mammal.

The present invention yet further relates to methods for the treatment or prevention of a physiological condition which may be modulated by an increase in endogenous growth hormone, which method comprises administering to an animal in need of said treatment an effective amount of a compound of formula I.

A preferred embodiment of the invention is a compound of Formula II 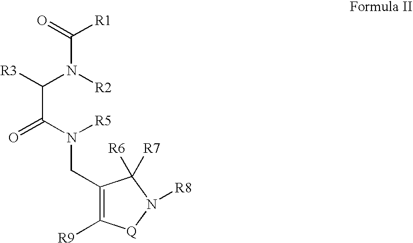
wherein

R1, R2, R3, R5, R6, R7, R8, R9 and Q are as defined for formula I above or a pharmaceutically acceptable salt or solvate thereof.

A further preferred embodiment of the invention is a compound of Formula III 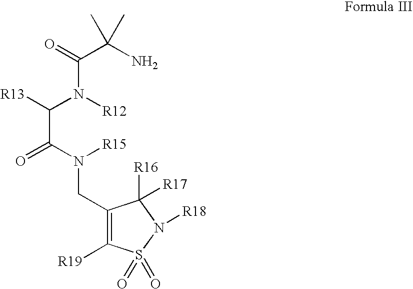
or a pharmaceutically acceptable salt or solvate thereof, wherein:

R12 is hydrogen, methyl or ethyl;

R13 is unsubstituted or substituted aryl, unsubstituted or substituted 3-arylpropyl, unsubstituted or substituted 2-arylethyl, unsubstituted or substituted arylmethoxymethyl, unsubstituted or substituted 3-indolylmethyl, or unsubstituted or substituted cyclohexylmethyl;

R19 is thienyl, naphthyl, thiazolyl, oxazolyl, pyridyl, O-phenyl, or phenyl, which are unsubstituted or substituted with one or more substituents independently selected from the group consisting of C₁-C₆alkyl, C₁-C₆alkoxy, CONH₂, CONH(C₁-C₆alkyl), NHCO(C₁-C₆alkyl), SO₂NH₂, SO₂NH(C₁-C₆alkyl), NHSO₂(C₁-C₆alkyl), COOH, COO(C1-C6alkyl), hydroxy, nitro, halo, SO₂(C₁₋₆ alkyl), SO₂CF₃, OCF₃, CF₃ and cyano; and either

R15 is hydroxy, methoxy, 2-hydroxyethyl, 2-fluoroethyl, or 2,2,2-trifluoroethyl;

R16 and R17 both are methyl or ethyl, or together with the carbon atom to which they are attached form a cyclopentane or cyclohexane ring; and

R18 is hydrogen, methyl, ethyl or arylmethyl;

or

R15 is hydrogen, methyl, ethyl, n-propyl, isopropyl, hydroxy, methoxy, 2-hydroxyethyl, 2-fluoroethyl, or 2,2,2-trifluoroethyl;

R16 and R17 both are methyl or ethyl, or together with the carbon atom to which they are attached form a cyclopentane or cyclohexane ring; and

R18 is hydrogen, methyl, ethyl, propyl, isopropyl, butyl, isobutyl, cyclopropylmethyl, pentyl, arylmethyl, 1-arylethyl, 2-methoxyethyl, 2-hydroxyethyl, 2-fluoroethyl, 4,4,4-trifluorobutyl, 3,3,3-trifluoropropyl, 2,2-difluoroethyl, 3-fluoropropyl, 4-fluorobutyl, 2,2,2-trifluoroethyl, —CH₂CONH₂, or —CH₂CON(CH₃)₂;

or

R15 is hydrogen, methyl, ethyl, n-propyl, isopropyl, hydroxy, methoxy, 2-hydroxyethyl, 2-fluoroethyl, or 2,2,2-trifluoroethyl;

R16 and R17 both are fluoromethyl, trifluoromethyl, 2-fluoroethyl, or 2,2,2-trifluoroethyl; or R16 is hydrogen and R17 is trifluoromethyl, 2,2,2-trifluoroethyl, or 3,3,3-trifluoropropyl; or R16 and R17 together with the carbon atom to which they are attached form a fluorocyclohexane or difluorocyclohexane ring;

R18 is hydrogen, methyl, ethyl, propyl, isopropyl, butyl, isobutyl, cyclopropylmethyl, pentyl, arylmethyl, 1-arylethyl, 2-methoxyethyl, 2-hydroxyethyl, 2-fluoroethyl, 4,4,4-trifluorobutyl, 3,3,3-trifluoropropyl, 2,2-difluoroethyl, 3-fluoropropyl, 4-fluorobutyl, 2,2,2-trifluoroethyl, —CH₂CONH₂, or —CH₂CON(CH₃)₂;

An also preferred embodiment of the invention is a compound of Formula IIIA or Formula IIIB 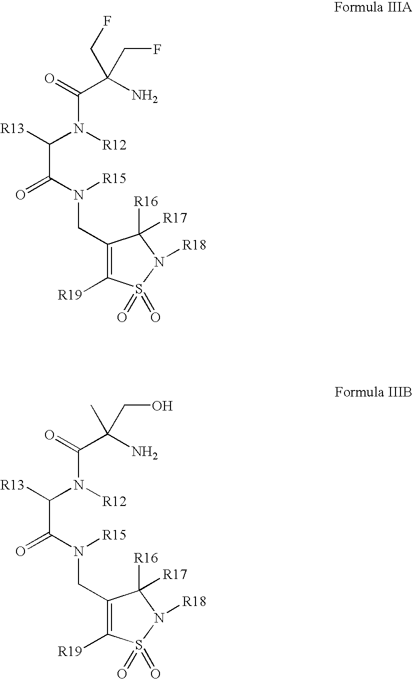
or a pharmaceutically acceptable salt or solvate thereof, wherein:

R12, R13 and R19 are as defined for Formula III and

R15 is hydrogen, methyl, ethyl, n-propyl, isopropyl, hydroxy, methoxy, 2-hydroxyethyl, 2-fluoroethyl, or 2,2,2-trifluoroethyl;

R16 and R17 are hydrogen, methyl, ethyl, fluoromethyl, trifluoromethyl, 2-fluoroethyl, or 2,2,2-trifluoroethyl or together with the carbon atom to which they are attached form a cyclopentane, cyclohexane, fluorocyclohexane or difluorocyclohexane ring; and

R18 is hydrogen, methyl, ethyl, propyl, isopropyl, butyl, isobutyl, cyclopropylmethyl, pentyl, arylmethyl, 1-arylethyl, 2-methoxyethyl, 2-hydroxyethyl, 2-fluoroethyl, 4,4,4-trifluorobutyl, 3,3,3-trifluoropropyl, 2,2-difluoroethyl, 3-fluoropropyl, 4-fluorobutyl, 2,2,2-trifluoroethyl, —CH₂CONH₂, or —CH₂CON(CH₃)₂;

The present invention additionally relates to compounds of formula IV and pharmaceutically acceptable salts or solvates thereof in which R12 to R19 have the same definition as in Formula III:

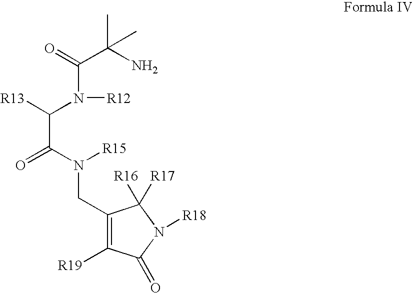

The present invention also relates to compounds of Formula IVA and Formula IVB and pharmaceutically acceptable salts or solvates thereof in which R12 to R19 have the same definition as in Formula IIIA and Formula IIIB:

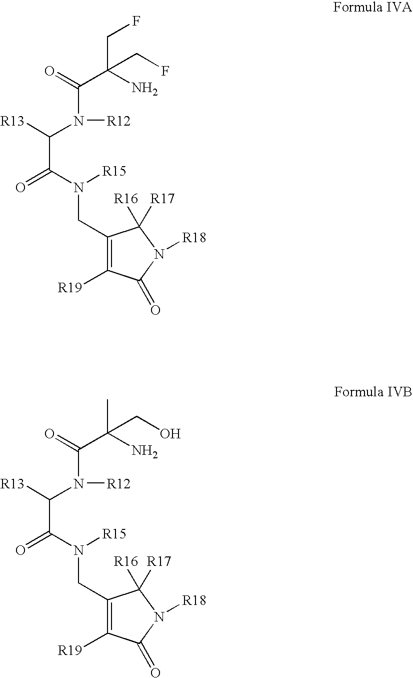

The present invention still further relates to processes for the preparation of compounds of formula I.

The terms and abbreviations used herein have their normal meanings unless otherwise designated. For example “° C.” refers to degrees Celsius; “N” refers to normal or normality; “mmol” refers to millimole or millimoles; “g” refers to gram or grams; “ml” means milliliter or milliliters; “M” refers to molar or molarity; “MS” refers to mass spectrometry; “FDMS” refers to field desorption mass spectrometry; “IS” refers to ion spray ionisation; “EI” refers to electron impact ionisation; “UV” refers to ultraviolet spectroscopy; “IR” refers to infrared spectroscopy; and “NMR” refers to nuclear magnetic resonance spectroscopy.

“TBTU” refers to O-(1H-benzotriazol-1-yl)-N,N,N′,N′-pentamethylene-uronium tetrafluoroborate.

As used herein, the term “C1-C6alkyl” refers to straight or branched, monovalent, saturated aliphatic chains of 1 to 6 carbon atoms and includes, but is not limited to, methyl, ethyl, propyl, isopropyl, butyl, isobutyl, t-butyl, pentyl, isopentyl, and hexyl. The term “C₁-C₆alkyl” includes within its definition the term “C₁-C₄alkyl”.

The term “substituted C₁-C₆ alkyl” means a C₁-C₆alkyl group as defined above which has been substituted by one or more, preferably from one to three groups selected from halo (preferably chloro or fluoro), hydroxy, —OC₁-C₆ alkyl, cyano, SO₂(C₁-C₆ alkyl), OCF₃, CF₃, CONH₂, CON(CH₃)₂, or NO₂.

As used herein, the term “C₂-C₆ alkenyl” refers to straight or branched, monovalent, unsaturated aliphatic chains of 2 to 6 carbon atoms including at least one carbon-carbon double bond and includes, but is not limited to, ethenyl, propenyl, isopropenyl, butenyl, isobutenyl, pentenyl, isopentenyl, and hexenyl. The term “C₂-C₆ alkenyl” includes within its definition the term “C₂-C₄ alkenyl”.

As used herein, the term “C₂-C₆ alkynyl” refers to straight or branched, monovalent, unsaturated aliphatic chains of 2 to 6 carbon atoms including at least one carbon-carbon triple bond and includes, but is not limited to, ethynyl, propynyl, butynyl, isobutynyl, pentynyl, isopentynyl, and hexynyl. The term “C₂-C₆ alkynyl” includes within its definition the term “C₂-C₄ alkynyl”.

The term “substituted C₂-C₆ alkenyl” means a C₂-C₆ alkenyl group as defined above which has been substituted by one or more, preferably from one to three groups selected from halo (preferably chloro or fluoro), hydroxy, —OC₁-C₆ alkyl, cyano, SO₂(C₁-C₆ alkyl), OCF₃, CF₃, CONH₂, CON(CH₃)₂, or NO₂.

As used herein, the term “cycloalkyl” refers to cyclized chains of 3 to 8 carbon atoms and includes, but is not limited to, cyclopropyl, cyclobutyl, cyclopentyl, and cyclohexyl.

The term “substituted C₃-C₈ cycloalkyl” means a C₃-C₈ cycloalkyl group as defined above which has been substituted by one or more, preferably from one to three groups selected from halo (preferably chloro or fluoro), —OC₁-C₆ alkyl, cyano, SO₂(C₁-C₆ alkyl), OCF₃, CF₃, CONH₂, CON(CH₃)₂, or NO₂.

The term “halo” means chloro, fluoro, bromo or iodo. Halo may most preferably be fluoro or chloro.

“C₁-C₆ alkoxy” represents a straight or branched alkyl chain having from one to six carbon atoms attached to an oxygen atom. Typical C₁-C₆ alkoxy groups include methoxy, ethoxy, propoxy, isopropoxy, butoxy, t-butoxy, pentoxy and the like. The term “C₁-C₆ alkoxy” includes within its definition the term “C₁-C₄ alkoxy”.

“C₂-C₆ alkanoyl” represents a straight or branched alkyl chain having from one to five carbon atoms attached through a carbonyl moiety. Typical C₂-C₆ alkanoyl groups include ethanoyl (also referred to as acetyl), propanoyl, isopropanoyl, butanoyl, t-butanoyl, pentanoyl, hexanoyl, and the like.

“C₁-C₆ alkylidenyl” refers to a straight or branched, divalent, saturated aliphatic chain of one to six carbon atoms and includes, but is not limited to, methylenyl, ethylenyl, propylenyl, isopropylenyl, butylenyl, isobutylenyl, t-butylenyl, pentylenyl, isopentylenyl, hexylenyl, and the like.

The term “aryl” represents an aromatic ring or rings and aromatic residues of 5 to 7-membered mono- or bicyclic rings with 1 to 4 heteroatoms (a “heteroaryl”) including but not limited to such groups as phenyl, naphthyl, biphenyl, thiophenyl (also known as thienyl), benzothiophenyl, furanyl, benzofuranyl, oxazolyl, indolyl, pyridyl, thiazolyl, isoxazolyl, isothiazolyl and the like.

The term “substituted aryl”, “substituted N-aryl”, and “substituted S-aryl” means that each of the respective aryl groups (which aryl group may contain heteroatoms as described above), is substituted, at any available position, with from one to four substituents, independently selected from the group consisting of C₁-C₆ alkyl, —OC₁-C₆ alkyl, —OCF₃, amide, aryl, aryloxy, SO₂(C₁₋₆ alkyl), SO₂CF₃, NHamide, carboxamide, sulfonamide, NHsulfonamide, imide, hydroxy, carboxy, nitro, halo, tri(chloro or fluoro)methyl, and cyano. The aromatic ring may be attached at any carbon atom or heteroatom which affords a stable structure. The group, 3,4-methylenedioxyphenyl is embraced by this definition.

The term “unsubstituted C₁-C₆ alkylaryl” means an unsubstituted C₁-C₆ alkyl group, as defined above, bonded to an unsubstituted aryl group as defined above. In preferred unsubstituted C₁-C₆ alkylaryl groups the unsubstituted C₁-C₆ alkyl moiety has from 1 to 3 carbon atoms. Also, and independently, in preferred unsubstituted C₁-C₆ alkylaryl groups the aryl group is selected from phenyl, thiazolyl, pyridyl, naphthyl, thienyl, isoxazolyl, oxazolyl and indolyl.

The term “substituted C₁-C₆ alkylaryl” means either an unsubstituted or substituted C₁-C₆ alkyl group, as defined above, bonded to a substituted aryl group as defined above or a substituted C₁-C₆ alkyl group as defined above bonded to an unsubstituted aryl group as defined above. In preferred compounds of the invention substituted C₁-C₆ alkylaryl denotes an C₁-C₆ alkyl group as defined above, bonded to a substituted aryl group as defined above. In more preferred substituted C₁-C₆ alkylaryl groups the unsubstituted C₁-C₆ alkyl moiety has from 1 to 3 carbon atoms. Also, and independently, in more preferred substituted C₁-C₆ alkylaryl groups the substituted aryl group is a selected from phenyl, thiazolyl, pyridyl, naphthyl, thienyl, oxazolyl, isoxazolyl or indolyl substituted, at any available position, by from one to four, preferably one, two or three, substituents independently selected from halo (preferably chloro or fluoro), C₁-C₆ alkyl, —OC₁-C₆ alkyl, cyano, SO₂(C₁-C₆ alkyl), OCF₃, CF₃, CONH₂, NO₂, phenyl, phenoxy, thienyl, pyridyl, thiazolyl, oxazolyl, furanyl, benzothiophenyl, benzofuranyl.

The term “unsubstituted C₁-C₆ alkyl(O)—C₁-C₆ alkyl aryl” means an unsubstituted C₁-C₆ alkyl(O)—C₁-C₆ alkyl group, as defined above, bonded to an unsubstituted aryl group as defined above. In preferred unsubstituted C₁-C₆ alkyl(O)—C₁-C₆ alkylaryl groups the unsubstituted C₁-C₆ alkyl(O)—C₁-C₆ alkyl moiety is —CH₂—O—CH₂—, —CH₂—O—CH₂CH₂—, or —CH₂CH₂—O—CH₂—, most preferably —CH₂—O—CH₂—. Also, and independently, in preferred unsubstituted C₁-C₆ alkyl(O)—C₁-C₆ alkylaryl groups the aryl group is a selected from phenyl, thiazolyl, pyridyl, naphthyl, thienyl, oxazolyl, isoxazolyl and indolyl.

The term “substituted C₁-C₆ alkyl(O)—C₁-C₆ alkyl aryl” means either an unsubstituted or substituted C₃-C₆ alkyl(O)—C₁-C₆ alkyl group, as defined above, bonded to a substituted aryl group as defined above or a substituted C₁-C₆ alkyl(O)—C₁-C₆ alkyl group as defined above bonded to an unsubstituted aryl group as defined above. In preferred compounds of the invention substituted C₁-C₆ alkyl(O)—C₁-C₆ alkylaryl denotes an C₁-C₆ alkyl(O)—C₁-C₆ alkyl group as defined above, bonded to a substituted aryl group as defined above. In more preferred substituted C₁-C₆ alkyl(O)—C₁-C₆ alkylaryl groups the unsubstituted C₁-C₆ alkyl(O)—C₁-C₆ alkyl moiety is —CH₂—O—CH₂—, —CH₂—O—CH₂CH₂—, or —CH₂CH₂—O—CH₂—, most preferably —CH₂—O—CH₂—. Also, and independently, in more preferred substituted C₁-C₆ alkyl(O)—C₁-C₆ alkylaryl groups the substituted aryl group is selected from phenyl, thiazolyl, pyrifyl, naphthyl, thienyl, oxazolyl, isoxazolyl and indolyl substituted, at any available position, by from one to four, preferably one, two or three, substituents independently selected from halo (preferably chloro or fluoro), C₁-C₆ alkyl, —OC₁-C₆ alkyl, cyano, SO₂(C₁-C₆ alkyl), OCF₃, CF₃, CONH₂, NO₂, phenyl, phenoxy, thienyl, pyridyl, thiazolyl, oxazolyl, furanyl, benzothiophenyl, benzofuranyl.

The term “unsubstituted (C₁-C₆ alkyl) C₃-C₈ cycloalkyl” means an unsubstituted C₁-C₆ alkyl group, as defined above, bonded to an unsubstituted C₃-C₈ cycloalkyl group as defined above. In preferred unsubstituted (C₁-C₆ alkyl) C₃-C₈ cycloalkyl groups the unsubstituted C₁-C₆ alkyl moiety has from 1 to 3 carbon atoms. Also, and independently, in more preferred unsubstituted (C₁-C₆ alkyl) C₃-C₈ cycloalkyl groups the C₃-C₈ cycloalkyl group is cyclopropyl, cyclopentyl or cyclohexyl.

The term “substituted (C₁-C₆ alkyl) C₃-C₈ cycloalkyl” means either an unsubstituted or substituted C₁-C₆ alkyl group, as defined above, bonded to a substituted C₃-C₈ cycloalkyl group as defined above or a substituted C₁-C₆ alkyl group as defined above bonded to an unsubstituted C₃-C₈ cycloalkyl group as defined above. In preferred compounds of the invention substituted (C₁-C₆ alkyl) C₃-C₈ cycloalkyl denotes an C₁-C₆ alkyl group as defined above, bonded to a substituted C₃-C₈ cycloalkyl group as defined above. In more preferred substituted (C₁-C₆ alkyl) C₃-C₈ cycloalkyl groups the unsubstituted C₁-C₆ alkyl moiety has from 1 to 3 carbon atoms. Also, and independently, in more preferred substituted (C₁-C₆ alkyl) C₃-C₈ cycloalkyl groups the substituted C₃-C₈ cycloalkyl group is cyclopropyl, cyclopentyl or cyclohexyl substituted, at any available position, by at least one and preferably from one to four substituents independently selected from halo (preferably chloro or fluoro), C₁-C₆ alkyl, —OC₁-C₆ alkyl, cyano, SO₂(C₁-C₆ alkyl), OCF₃, CF₃, CONH₂, CON(CH₃)₂, NO₂, phenyl, phenoxy, thienyl, pyridyl, thiazolyl, oxazolyl, furanyl, benzothiophenyl, benzofuranyl.

The term “—O-aryl” means an aryloxy substituent which is bonded to the parent molecule through the 0 group. The term “unsubstituted or substituted —O-aryl” means that the aryl group of the —O-aryl substituent is unsubstituted or substituted with from one to four substituents independently selected from the group consisting of C₁-C₆ alkyl, —OC₁-C₆ alkyl, —OCF₃, amide, aryl, aryloxy, SO₂(C₁₋₆ alkyl), NHamide, CF₃SO₂, carboxamide, sulfonamide, NHsulfonamide, imide, hydroxy, carboxy, nitro, halo, tri(chloro or fluoro)methyl, and cyano.

The term “-aryl-aryl(K1)(K2)” refers to an aryl group substituted with an additional aryl group said additional aryl group being disubstituted with K1 and K2. K1 is defined to include halo and —CF₃, and K2 is defined to include hydrogen, halo, and —CF₃. Alternatively K1 and K2 together may form a methylenedioxy group. Similarly, the terms “—O-aryl-aryl(K1)(K2)”, “—N-aryl-aryl(K1)(K2)”, and “-S-aryl-aryl(K1)(K2)” are likewise defined. For example, the term “—O-aryl-aryl(K1)(K2)” means an aryloxy substituent as defined above which is substituted with an additional aryl group, said additional aryl group being disubstituted with K1 and K2. K1 and K2 are as defined immediately above.

The term “carboxy-protecting group” as used herein refers to substituents of the carboxy group commonly employed to block or protect the carboxy functionality while reacting other functional groups on the compound. Examples of such protecting groups include methyl, ethyl, p-nitrobenzyl, p-methylbenzyl, p-methoxybenzyl, 3,4-dimethoxybenzyl, 2,4-dimethoxybenzyl, 2,4,6-trimethoxybenzyl, 2,4,6-trimethylbenzyl, pentamethylbenzyl, 3,4-methylene-dioxybenzyl, benzhydryl, 4,4′-dimethoxy-benzhydryl, 2,2′,4,4′-tetramethoxybenzhydryl, t-butyl, t-amyl, trityl, 4-methoxytrityl, 4,4′-dimethoxytrityl, 4,4′,4″-trimethoxytrityl, 2-phenylprop-2-yl, trimethylsilyl, t-butyldimethylsilyl, phenacyl, 2,2,2-trichloroethyl, 2-(di(n-butyl)methylsilyl)ethyl, p-toluenesulfonylethyl, 4-nitrobenzylsulfonylethyl, allyl, cinnamyl, 1-(trimethylsilylmethyl)prop-1-en-3-yl, and the like. A preferred carboxy-protecting group for the practice of the present invention is methyl or ethyl. Further examples of these groups may be found in E. Haslam, supra, at Chapter 5, and T. W. Greene, et al., supra, at Chapter 5.

The term “amino-protecting group” as used herein refers to substituents of the amino group commonly employed to block or protect the amino functionality while reacting other functional groups on the compound. Examples of such amino-protecting groups can be found at T. W. Greene, et al., supra.

Examples of such amino-protecting groups include, but are not limited to, formyl, trityl, phthalimido, trichloroacetyl, chloroacetyl, bromoacetyl, iodoacetyl, and urethane-type blocking groups such as benzyloxycarbonyl, 4-phenylbenzyloxycarbonyl, 2-methylbenzyloxycarbonyl, 4-methoxybenzyloxycarbonyl, 4-fluorobenzyloxycarbonyl, 4-chlorobenzyloxycarbonyl, 3-chlorobenzyloxycarbonyl, 2-chlorobenzyloxycarbonyl, 2,4-dichlorobenzyloxycarbonyl, 4-bromobenzyloxycarbonyl, 3-bromobenzyloxycarbonyl, 4-nitrobenzyloxycarbonyl, 4-cyanobenzyloxycarbonyl, n-butoxycarbonyl, (NBoc) t-butoxycarbonyl, 1,1-diphenyleth-1-yloxycarbonyl, 1,1-diphenylprop-1-yloxycarbonyl, 2-phenylprop-2-yloxycarbonyl, 2-(p-toluyl)-prop-2-yloxycarbonyl, cyclopentanyloxycarbonyl, 1-methylcyclopentanyloxycarbonyl, cyclohexanyloxycarbonyl, 1-methylcyclohexanyloxycarbonyl, 2-methylcyclohexanyloxycarbonyl, 2-(4-toluylsulfonyl)-ethoxycarbonyl, 2-(methylsulfonyl)ethoxycarbonyl, 2-(triphenylphosphino)-ethoxycarbonyl, fluorenylmethoxy-carbonyl (FMOC), 2-(trimethylsilyl)ethoxycarbonyl, allyloxycarbonyl, 1-(trimethylsilylmethyl)prop-1-enyloxycarbonyl, 5-benzisoxalylmethoxycarbonyl, 4-acetoxybenzyloxycarbonyl, 2,2,2-trichloroethoxycarbonyl, 2-ethynyl-2-propoxycarbonyl, cyclopropylmethoxycarbonyl, 4-(decyloxy)benzyloxycarbonyl, isobornyloxycarbonyl, 1-piperidyloxycarbonyl, and the like; benzoylmethylsulfonyl group, 2-nitrophenylsulfenyl, diphenylphosphine oxide and like amino-protecting groups.

The amino-protecting group employed is usually not critical so long as the derivatized amino group is stable to the condition of subsequent reactions on other positions of the intermediate molecule, and may be selectively removed at the appropriate point without disrupting the remainder of the molecule including any other amino-protecting groups. A preferred amino-protecting group for the practice of the present invention is t-butoxycarbonyl (NBoc). Further examples of groups referred to by the above terms are described by E. Haslam, Protective Groups in Organic Chemistry, (J. G. W. McOmie, ed., 1973), at Chapter 2; and T. W. Greene and P. G. M. Wuts, Protective Groups in Organic Synthesis (1991), at Chapter 7.

The term “activating group” as used herein refers a leaving group which, when taken with the carbonyl (—C═O) group to which it is attached, is more likely to take part in an acylation reaction than would be the case if the group were not present, as in the free acid. Such activating groups are well-known to those skilled in the art and may be, for example, succinimidoxy, phthalimidoxy, benzotriazolyloxy, azido, chloro, bromo, fluoro or —O—CO—(C₄-C₇ alkyl).

In one group of more preferred compounds of formula I, R1 is C₁-C₆alkylNHR10 where in R10 is selected from hydrogen and C₁-C₆ alkyl. In the most preferred compounds of the invention R1 is a group of formula —C(CH₃)₂NH₂.

In another group of more preferred compounds of formula I wherein R1 is (substituted C₁-C₆alkyl)NHR10, R10 is selected from hydrogen and C₁-C₆ alkyl and the substituted C₁-C₆ alkyl group is a C₁-C₅ alkyl group, which is more preferably branched, and which is substituted by from 1 to 3 halo atoms, most preferably fluoro atoms. Examples of more preferred R1 groups include —C(CH₂F)₂NH₂, —C(CH₂F) (CH₂CH₂F)NH₂, —C(CF₃) (CH₃)NH₂, —C(CH₂CH₂F)₂NH₂ and —C(CH₂CH₃)(CH₂CF₃)NH₂. In the most preferred compounds of the invention R1 is a group of formula —C(CH₂F)₂NH₂.

In another alternative group of more preferred compounds of formula I wherein R1 is (substituted C₁-C₆alkyl)NHR10, R1 is —C(CH₃) (CH₂OH)NH₂.

In a further group of more preferred compounds of formula I wherein R1 is (unsubstituted or substituted C₃-C₈ cycloalkyl)NHR10, R10 is selected from hydrogen and C1-C6 alkyl and the C₃-C₈ cycloalkyl group is unsubstituted. Still more preferably the C₃-C₈ cycloalkyl group is such that the carbonyl and the —NHR10 groups are connected at the same carbon atom. Examples of more preferred R1 groups of this type include

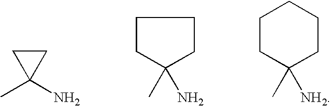

In the more preferred compounds of formula I, R2 is hydrogen or C₁-C₆ alkyl, preferably methyl. In the most preferred compounds of the invention R2 is hydrogen.

In the more preferred compounds of formula I, R3 is an unsubstituted or substituted aryl group, an unsubstituted or substituted C₁-C₆ alkylaryl group or an unsubstituted or substituted C₁-C₆alkyl(O)—C₁-C₆alkyl aryl group wherein:

the C₁-C₆alkyl moiety within the unsubstituted or substituted C₁-C₆ alkylaryl group is methyl, ethyl or propyl;

the C₁-C₆alkyl(O)—C₁-C₆alkyl moiety within the unsubstituted or substituted C₁-C₆alkyl(O)—C₁-C₆alkyl aryl group is a moiety of formula —CH₂OCH₂—;

the aryl moiety within said groups is selected from phenyl, thiazolyl, pyridyl, naphthyl, thienyl, oxazolyl, isoxazolyl and indolyl which is unsubstituted or substituted by from one to three groups independently selected from halo (preferably chloro or fluoro), methyl, methoxy, cyano, SO₂Me, trifluoromethyl, and trifluoromethoxy. Most preferably the unsubstituted aryl moiety is phenyl, naphthyl, thiazolyl or indolyl and the substituted aryl moiety in said groups is 2-fluorophenyl, 3-fluorophenyl, 4-fluorophenyl, 2,3-difluorophenyl, 2,4-difluorophenyl, 2,5-difluorophenyl, 2,6-difluorophenyl, 3,4-difluorophenyl, 3,5-difluorophenyl, 2,4,6-trifluorophenyl, 2,3,4-trifluorophenyl, 2,4,5-trifluorophenyl, 2,3,6-trifluorophenyl, 2,3,5-trifluorophenyl, 2-chlorophenyl, 3-chlorophenyl, 4-chlorophenyl, 2,6-dichlorophenyl, 2,3-dichlorophenyl, 2,4-dichlorophenyl, 2,5-dichlorophenyl, 2-chloro-4-fluorophenyl, 2-methylphenyl, 2,6-difluoro-3-methylphenyl, 3,6-difluoro-2-chlorophenyl, 2-fluoro-6-chlorophenyl, 2-fluoro-3-chlorophenyl, 2-fluoro-4-chlorophenyl, 2,6-difluoro-3-chlorophenyl, 4-trifluoromethylphenyl, 3-trifluoromethylphenyl, 2-trifluoromethylphenyl, 2-fluoro-5-trifluoromethylphenyl, 2-fluoro-3-trifluoromethylphenyl, 2-fluoro-6-trifluoromethylphenyl, 2-chloro-3-trifluoromethylphenyl, 4-trifluoromethoxyphenyl, 3-trifluoromethoxyphenyl, 2-trifluoromethoxyphenyl, 2-cyanophenyl, 3-cyanophenyl, 4-cyanophenyl, 4-methanesulphonylphenyl, and 2-methyl thiazolyl.

In the more preferred compounds of formula I R4 is hydrogen or C₁-C₆ alkyl. In the most preferred compounds of the invention R4 is hydrogen or methyl.

In the more preferred compounds of formula I R5 is hydrogen, C₁-C₆ alkyl, hydroxy, C₁-C₆alkoxy, C₁-C₆alkyl which is substituted by hydroxy or C₁-C₆alkyl which is substituted by one, two, or three halo atoms, preferably fluoro or chloro. In the most preferred compounds of the invention R5 is hydrogen, methyl, ethyl, i-propyl, n-propyl, 2-fluoroethyl, 2-hydroxyethyl, 2,2,2-trifluoroethyl, hydroxy or methoxy.

In the more preferred compounds of formula I R6 and R7 are independently C₁-C₆ alkyl groups; or R6 and R7 are independently C₁-C₆alkyl or C₂-C₆alkenyl, in which one or both groups are substituted by one, two, or three halo atoms; or R6 is hydrogen and R7 is C₁-C₆alkyl or C₂-C₆alkenyl which is substituted by one, two, or three halo atoms; or R6 and R7 together form a carbocyclic ring of up to 8 atoms or R6 and R7 together with the carbon atom to which they are attached may form a C₃-C₈cycloalkyl group which is optionally partly unsaturated and which is substituted by one, two, or three halo atoms. In the most preferred compounds of the invention R6 and R7 are both methyl, ethyl, fluoromethyl, trifluoromethyl, 2-fluoroethyl, or 2,2,2-trifluoroethyl; or R6 is hydrogen and R7 is trifluoromethyl, 2,2,2-trifluoroethyl, or 3,3,3-trifluoropropyl; or R6 and R7 together form a cyclohexane, cyclopentane, fluorocyclohexane or difluorocyclohexane ring.

In the more preferred compounds of formula I, R8 is hydrogen, C₁-C₆alkyl, (C₁-C₆alkyl)C₃-C₈cycloalkyl, benzyl, 1-phenylethyl, C₁-C₆alkyl which is substituted by hydroxy, methoxy, CONH₂, or CON(CH₃)₂, or C₁-C₆alkyl which is substituted by one, two, or three halo atoms, phenyl substituted by one, two, or three halo atoms or benzyl substituted by one, two, or three halo atoms. The Halo atoms are preferably fluoro or chloro. In the most preferred compounds of the invention R8 is hydrogen, methyl, ethyl, propyl, isopropyl, isobutyl, neopentyl, cyclopropylmethyl, 1-phenylethyl, benzyl, 2-hydroxyethyl, 2-methoxyethyl, 2-fluoroethyl, 2,2,2-trifluoroethyl, 4,4,4-trifluorobutyl, 3,3,3-trifluoropropyl, CH₂CONH₂, CH₂CON(CH₃)₂.

In the more preferred compounds of formula I, R9 is C₁-C₆ alkyl, C₃-C₈ cycloalkyl, unsubstituted or substituted aryl, unsubstituted or substituted-O-aryl, or -aryl-aryl(K1) (K2) wherein K1 is halo or —CF₃ and K2 is hydrogen, halo or CF₃ or K1 and K2 together form a methylenedioxy group.

In preferred compounds of the invention wherein R9 is a C₁-C₆ alkyl group, R9 is most preferably methyl or isopropyl.

In preferred compounds of the invention wherein R9 is a C₃-C₈ cycloalkyl group, R9 is most preferably cyclohexyl. In preferred compounds of the invention wherein R9 is an -aryl-aryl(K1)(K2) group, R9 is a -phenyl-phenyl(K1) (K2), or -phenyl-thienyl(K1)(K2) group, and most preferably is -phenyl-fluorophenyl, -phenyl-chlorophenyl, -phenyl-trifluoromethylphenyl, -phenyl-(3,4-methylenedioxyphenyl) or -phenyl-chlorothienyl.

In preferred compounds of the invention wherein R9 is an unsubstituted or substituted aryl or unsubstituted or substituted-O-aryl group, said unsubstituted or substituted aryl moiety is phenyl, naphthyl, pyridyl, thienyl, thiazolyl or oxazolyl, most preferably phenyl. Preferred optional substituents are halo (preferably chloro, fluoro or bromo), methyl, ethyl, propyl, t-butyl, trifluoromethyl, trifluoromethoxy, methoxy, ethoxy, cyano, methylsulphonyl, phenyl, phenoxy, thienyl, pyridyl, thiazolyl, oxazolyl, nitro, CONH₂, furanyl, benzothiophenyl and benzofuranyl. In the most preferred compounds of the invention wherein R9 is an unsubstituted or substituted aryl or unsubstituted or substituted-O-aryl group, R9 is selected from phenyl, 4-methylsulphonylphenyl, 3-methylsulphonylphenyl, 4-fluorophenyl, 2-fluorophenyl, 3-fluorophenyl, 3-chlorophenyl, 2-chlorophenyl, 4-chlorophenyl, 4-t-butylphenyl, 4-trifluoromethylphenyl, 3-trifluoromethylphenyl, 4-nitrophenyl, 3-nitrophenyl, 4-bromophenyl, 3-bromophenyl, 2-bromophenyl, 4-methylphenyl, 3-methylphenyl, 4-phenylphenyl, 3-phenylphenyl, 4-phenoxyphenyl, 3-phenoxyphenyl, 4-cyanophenyl, 3-cyanophenyl, 4-carbamoylphenyl, 4-methoxyphenyl, 3-methoxyphenyl, thienyl, thiazolyl, pyridyl, phenoxy, 4chlorophenoxy, 2,3-dichlorophenyl, 3,4-dichlorophenyl, naphthyl, oxazolyl, 2,4-difluorophenyl, 3,4-difluorophenyl, 3,5-difluorophenyl, 2,3-difluorophenyl, 2,6-difluorophenyl, 2,5-difluorophenyl, 2-fluoro-3-chlorophenyl, 4-ethylphenyl, 4-ethoxyphenyl, 3,4,5-trifluoromethyl, 3-fluoro-4-chlorophenyl and 4-carbamoylphenyl.

It will be understood that the preferred definitions given above in respect of R2, R3, R5, R6, R7, R8 and R9 in formula I and II apply to the substituents within the definitions at the corresponding positions in formulae III, IIIA, IV and IVA i.e. positions R12, R13, R15, R16, R17, R18 and R19 respectively.

Particularly preferred compounds of the invention are those set out in the following tables I to XIX and the pharmaceutically acceptable salts and solvates thereof:

TABLE I

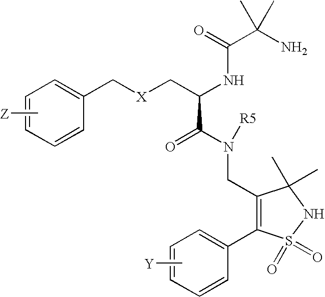

| X | Y | Z | R5 |
|---|---|---|---|
| O | 4-Cl | H | CH₂CH₂F |
| O | 4-Cl | 2-F | CH₂CH₂F |
| O | 4-Cl | 3-F | CH₂CH₂F |
| O | 4-Cl | 4-F | CH₂CH₂F |
| O | 4-Cl | 4-Cl | CH₂CH₂F |
| O | 4-Cl | 2,5-F₂ | CH₂CH₂F |
| O | 4-Cl | 2,4-F₂ | CH₂CH₂F |
| O | 4-Cl | 2-Cl | CH₂CH₂F |
| O | 4-Cl | 2,6-F₂ | CH₂CH₂F |
| O | 4-Cl | 3,5-F₂ | CH₂CH₂F |
| O | 4-Cl | 2,3-F₂ | CH₂CH₂F |
| O | 4-Cl | 3,4-F₂ | CH₂CH₂F |
| O | 4-Cl | 2,3,5-F₃ | CH₂CH₂F |
| O | 4-Cl | 2,3,6-F₂ | CH₂CH₂F |
| O | 4-Cl | 2,4,5-F₃ | CH₂CH₂F |
| O | 4-Cl | 2,6-Cl₂ | CH₂CH₂F |
| O | 4-Cl | 2-F-6-Cl | CH₂CH₂F |
| O | 4-Cl | 2-F-3-Cl | CH₂CH₂F |
| O | 4-Cl | 4-F-2-Cl | CH₂CH₂F |
| O | 4-Cl | 2-Cl-3,6-F₂ | CH₂CH₂F |
| O | 4-Cl | 3-Cl-2,6-F₂ | CH₂CH₂F |
| O | 4-Cl | 3-F-4-Cl | CH₂CH₂F |
| O | 4-Cl | 2-F-4-Cl | CH₂CH₂F |
| O | 4-Cl | 2,3-Cl₂ | CH₂CH₂F |
| O | 4-Cl | 2-CN | CH₂CH₂F |
| O | 4-F | H | CH₂CH₂F |
| O | 4-F | 2-F | CH₂CH₂F |
| O | 4-F | 3-F | CH₂CH₂F |
| O | 4-F | 4-F | CH₂CH₂F |
| O | 4-F | 4-Cl | CH₂CH₂F |
| O | 4-F | 2,5-F₂ | CH₂CH₂F |
| O | 4-F | 2,4-F₂ | CH₂CH₂F |
| O | 4-F | 2-Cl | CH₂CH₂F |
| O | 4-F | 2,6-F₂ | CH₂CH₂F |
| O | 4-F | 3,5-F₂ | CH₂CH₂F |
| O | 4-F | 2,3-F₂ | CH₂CH₂F |
| O | 4-F | 3,4-F₂ | CH₂CH₂F |
| O | 4-F | 2,3,5-F₃ | CH₂CH₂F |
| O | 4-F | 2,3,6-F₃ | CH₂CH₂F |
| O | 4-F | 2,4,5-F₃ | CH₂CH₂F |
| O | 4-F | 2,6-Cl₂ | CH₂CH₂F |
| O | 4-F | 2-F-6-Cl | CH₂CH₂F |
| O | 4-F | 2-F-3-Cl | CH₂CH₂F |
| O | 4-F | 4-F-2-Cl | CH₂CH₂F |
| O | 4-F | 2-Cl-3,6-F₂ | CH₂CH₂F |
| O | 4-F | 3-Cl-2,6-F₂ | CH₂CH₂F |
| O | 4-F | 3-F-4-Cl | CH₂CH₂F |
| O | 4-F | 2-F-4-Cl | CH₂CH₂F |
| O | 4-F | 2,3-Cl₂ | CH₂CH₂F |
| O | 4-F | 2-CN | CH₂CH₂F |
| CH₂ | 4-Cl | H | CH₂CH₂F |
| CH₂ | 4-Cl | 3,5-F₂ | CH₂CH₂F |
| CH₂ | 4-Cl | 4-F | CH₂CH₂F |
| CH₂ | 4-Cl | 2,6-F₂ | CH₂CH₂F |
| CH₂ | 4-F | H | CH₂CH₂F |
| CH₂ | 4-F | 3,5-F₂ | CH₂CH₂F |
| CH₂ | 4-F | 4-F | CH₂CH₂F |
| CH₂ | 4-F | 2,6-F₂ | CH₂CH₂F |
| O | 3-Cl | H | CH₂CH₂F |
| O | 3-Cl | 3,5-F₂ | CH₂CH₂F |
| O | 3-Cl | 4-F | CH₂CH₂F |
| O | 3-Cl | 2,6-F₂ | CH₂CH₂F |
| O | 3-Cl | 2,5-F₂ | CH₂CH₂F |
| O | 3-F | H | CH₂CH₂F |
| O | 3-F | 3,5-F₂ | CH₂CH₂F |
| O | 3-F | 4-F | CH₂CH₂F |
| O | 3-F | 2,6-F₂ | CH₂CH₂F |
| O | 3-F | 2,5-F₂ | CH₂CH₂F |
| O | 4-CN | H | CH₂CH₂F |
| O | 4-CN | 3,5-F₂ | CH₂CH₂F |
| O | 4-CN | 4-F | CH₂CH₂F |
| O | 4-CN | 2,6-F₂ | CH₂CH₂F |
| O | 4-CN | 2,5-F₂ | CH₂CH₂F |
| O | 2,5-F₂ | H | CH₂CH₂F |
| O | 2,5-F₂ | 3,5-F₂ | CH₂CH₂F |
| O | 2,5-F₂ | 4-F | CH₂CH₂F |
| O | 2,5-F₂ | 2,6-F₂ | CH₂CH₂F |
| O | 2,5-F₂ | 2,5-F₂ | CH₂CH₂F |
| O | 3,5-F₂ | H | CH₂CH₂F |
| O | 3,5-F₂ | 3,5-F₂ | CH₂CH₂F |
| O | 3,5-F₂ | 4-F | CH₂CH₂F |
| O | 3,5-F₂ | 2,6-F₂ | CH₂CH₂F |
| O | 3,5-F₂ | 2,5-F₂ | CH₂CH₂F |
| O | 3,4-F₂ | H | CH₂CH₂F |
| O | 3,4-F₂ | 3,5-F₂ | CH₂CH₂F |
| O | 3,4-F₂ | 4-F | CH₂CH₂F |
| O | 3,4-F₂ | 2,6-F₂ | CH₂CH₂F |
| O | 3,4-F₂ | 2,5-F₂ | CH₂CH₂F |
| O | 4-CF₃ | H | CH₂CH₂F |
| O | 4-CF₃ | 3,5-F₂ | CH₂CH₂F |
| O | 4-CF₃ | 4-F | CH₂CH₂F |
| O | 4-CF₃ | 2,6-F₂ | CH₂CH₂F |
| O | 4-CF₃ | 2,5-F₂ | CH₂CH₂F |
| O | 4-Cl | H | CH₂CH₂OH |
| O | 4-Cl | 2-F | CH₂CH₂OH |
| O | 4-Cl | 3-F | CH₂CH₂OH |
| O | 4-Cl | 4-F | CH₂CH₂OH |
| O | 4-Cl | 4-Cl | CH₂CH₂OH |
| O | 4-Cl | 2,5-F₂ | CH₂CH₂OH |
| O | 4-Cl | 2,4-F₂ | CH₂CH₂OH |
| O | 4-Cl | 2-Cl | CH₂CH₂OH |
| O | 4-Cl | 2,6-F₂ | CH₂CH₂OH |
| O | 4-Cl | 3,5-F₂ | CH₂CH₂OH |
| O | 4-Cl | 2,3-F₂ | CH₂CH₂OH |
| O | 4-Cl | 3,4-F₂ | CH₂CH₂OH |
| O | 4-Cl | 2,3,5-F₃ | CH₂CH₂OH |
| O | 4-Cl | 2,3,6-F₃ | CH₂CH₂OH |
| O | 4-Cl | 2,4,5-F₃ | CH₂CH₂OH |
| O | 4-Cl | 2,6-Cl₂ | CH₂CH₂OH |
| O | 4-Cl | 2-F-6-Cl | CH₂CH₂OH |
| O | 4-Cl | 2-F-3-Cl | CH₂CH₂OH |
| O | 4-Cl | 4-F-2-Cl | CH₂CH₂OH |
| O | 4-Cl | 2-Cl-3,6-F₂ | CH₂CH₂OH |
| O | 4-Cl | 3-Cl-2,6-F₂ | CH₂CH₂OH |
| O | 4-Cl | 3-F-4-Cl | CH₂CH₂OH |
| O | 4-Cl | 2-F-4-Cl | CH₂CH₂OH |
| O | 4-Cl | 2,3-Cl₂ | CH₂CH₂OH |
| O | 4-Cl | 2-CN | CH₂CH₂OH |
| O | 4-F | H | CH₂CH₂OH |
| O | 4-F | 2-F | CH₂CH₂OH |
| O | 4-F | 3-F | CH₂CH₂OH |
| O | 4-F | 4-F | CH₂CH₂OH |
| O | 4-F | 4-Cl | CH₂CH₂OH |
| O | 4-F | 2,5-F₂ | CH₂CH₂OH |
| O | 4-F | 2,4-F₂ | CH₂CH₂OH |
| O | 4-F | 2-Cl | CH₂CH₂OH |
| O | 4-F | 2,6-F₂ | CH₂CH₂OH |
| O | 4-F | 3,5-F₂ | CH₂CH₂OH |
| O | 4-F | 2,3-F₂ | CH₂CH₂OH |
| O | 4-F | 3,4-F₂ | CH₂CH₂OH |
| O | 4-F | 2,3,5-F₃ | CH₂CH₂OH |
| O | 4-F | 2,3,6-F₃ | CH₂CH₂OH |
| O | 4-F | 2,4,5-F₃ | CH₂CH₂OH |
| O | 4-F | 2,6-Cl₂ | CH₂CH₂OH |
| O | 4-F | 2-F-6-Cl | CH₂CH₂OH |
| O | 4-F | 2-F-3-Cl | CH₂CH₂OH |
| O | 4-F | 4-F-2-Cl | CH₂CH₂OH |
| O | 4-F | 2-Cl-3,6-F₂ | CH₂CH₂OH |
| O | 4-F | 3-Cl-2,6-F₂ | CH₂CH₂OH |
| O | 4-F | 3-F-4-Cl | CH₂CH₂OH |
| O | 4-F | 2-F-4-Cl | CH₂CH₂OH |
| O | 4-F | 2,3-Cl₂ | CH₂CH₂OH |
| O | 4-F | 2-CN | CH₂CH₂OH |
| CH₂ | 4-Cl | H | CH₂CH₂OH |
| CH₂ | 4-Cl | 3,5-F₂ | CH₂CH₂OH |
| CH₂ | 4-Cl | 4-F | CH₂CH₂OH |
| CH₂ | 4-Cl | 2,6-F₂ | CH₂CH₂OH |
| CH₂ | 4-F | H | CH₂CH₂OH |
| CH₂ | 4-F | 3,5-F₂ | CH₂CH₂OH |
| CH₂ | 4-F | 4-F | CH₂CH₂OH |
| CH₂ | 4-F | 2,6-F₂ | CH₂CH₂OH |
| O | 3-F | H | CH₂CH₂OH |
| O | 3-F | 3,5-F₂ | CH₂CH₂OH |
| O | 3-F | 4-F | CH₂CH₂OH |
| O | 3-F | 2,6-F₂ | CH₂CH₂OH |
| O | 3-F | 2,5-F₂ | CH₂CH₂OH |
| O | 2,5-F₂ | H | CH₂CH₂OH |
| O | 2,5-F₂ | 3,5-F₂ | CH₂CH₂OH |
| O | 2,5-F₂ | 4-F | CH₂CH₂OH |
| O | 2,5-F₂ | 2,6-F₂ | CH₂CH₂OH |
| O | 2,5-F₂ | 2,5-F₂ | CH₂CH₂OH |
| O | 3,5-F₂ | H | CH₂CH₂OH |
| O | 3,5-F₂ | 3,5-F₂ | CH₂CH₂OH |
| O | 3,5-F₂ | 4-F | CH₂CH₂OH |
| O | 3,5-F₂ | 2,6-F₂ | CH₂CH₂OH |
| O | 3,5-F₂ | 2,5-F₂ | CH₂CH₂OH |
| O | 3,4-F₂ | H | CH₂CH₂OH |
| O | 3,4-F₂ | 3,5-F₂ | CH₂CH₂OH |
| O | 3,4-F₂ | 4-F | CH₂CH₂OH |
| O | 3,4-F₂ | 2,6-F₂ | CH₂CH₂OH |
| O | 3,4-F₂ | 2,5-F₂ | CH₂CH₂OH |
| O | 4-CF₃ | H | CH₂CH₂OH |
| O | 4-CF₃ | 3,5-F₂ | CH₂CH₂OH |
| O | 4-CF₃ | 4-F | CH₂CH₂OH |
| O | 4-CF₃ | 2,6-F₂ | CH₂CH₂OH |
| O | 4-CF₃ | 2,5-F₂ | CH₂CH₂OH |
| O | 4-Cl | H | CH₂CF₃ |
| O | 4-F | H | CH₂CF₃ |
| CH₂ | 4-Cl | H | CH₂CF₃ |
| CH₂ | 4-F | H | CH₂CF₃ |
| O | 4-Cl | H | OH |
| O | 4-F | H | OH |
| CH₂ | 4-Cl | H | OH |
| CH₂ | 4-F | H | OH |
| O | 4-Cl | H | OCH₃ |
| O | 4-F | H | OCH₃ |
| CH₂ | 4-Cl | H | OCH₃ |
| CH₂ | 4-F | H | OCH₃ |

TABLE II

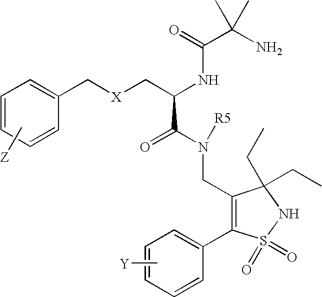

| X | Y | Z | R5 |
|---|---|---|---|
| O | 4-Cl | H | CH₂CH₂F |
| O | 4-Cl | 2-F | CH₂CH₂F |
| O | 4-Cl | 3-F | CH₂CH₂F |
| O | 4-Cl | 4-F | CH₂CH₂F |
| O | 4-Cl | 4-Cl | CH₂CH₂F |
| O | 4-Cl | 2,5-F₂ | CH₂CH₂F |
| O | 4-Cl | 2,4-F₂ | CH₂CH₂F |
| O | 4-Cl | 2-Cl | CH₂CH₂F |
| O | 4-Cl | 2,6-F₂ | CH₂CH₂F |
| O | 4-Cl | 3,5-F₂ | CH₂CH₂F |
| O | 4-Cl | 2,3-F₂ | CH₂CH₂F |
| O | 4-Cl | 3,4-F₂ | CH₂CH₂F |
| O | 4-Cl | 2,3,5-F₃ | CH₂CH₂F |
| O | 4-Cl | 2,3,6-F₃ | CH₂CH₂F |
| O | 4-Cl | 2,4,5-F₃ | CH₂CH₂F |
| O | 4-Cl | 2,6-Cl₂ | CH₂CH₂F |
| O | 4-Cl | 2-F-6-Cl | CH₂CH₂F |
| O | 4-Cl | 2-F-3-Cl | CH₂CH₂F |
| O | 4-Cl | 4-F-2-Cl | CH₂CH₂F |
| O | 4-Cl | 2-Cl-3,6-F₂ | CH₂CH₂F |
| O | 4-Cl | 3-Cl-2,6-F₂ | CH₂CH₂F |
| O | 4-Cl | 3-F-4-Cl | CH₂CH₂F |
| O | 4-Cl | 2-F-4-Cl | CH₂CH₂F |
| O | 4-Cl | 2,3-Cl₂ | CH₂CH₂F |
| O | 4-Cl | 2-CN | CH₂CH₂F |
| O | 4-F | H | CH₂CH₂F |
| O | 4-F | 2-F | CH₂CH₂F |
| O | 4-F | 3-F | CH₂CH₂F |
| O | 4-F | 4-F | CH₂CH₂F |
| O | 4-F | 4-Cl | CH₂CH₂F |
| O | 4-F | 2,5-F₂ | CH₂CH₂F |
| O | 4-F | 2,4-F₂ | CH₂CH₂F |
| O | 4-F | 2-Cl | CH₂CH₂F |
| O | 4-F | 2,6-F₂ | CH₂CH₂F |
| O | 4-F | 3,5-F₂ | CH₂CH₂F |
| O | 4-F | 2,3-F₂ | CH₂CH₂F |
| O | 4-F | 3,4-F₂ | CH₂CH₂F |
| O | 4-F | 2,3,5-F₃ | CH₂CH₂F |
| O | 4-F | 2,3,6-F₃ | CH₂CH₂F |
| O | 4-F | 2,4,5-F₃ | CH₂CH₂F |
| O | 4-F | 2,6-Cl₂ | CH₂CH₂F |
| O | 4-F | 2-F-6-Cl | CH₂CH₂F |
| O | 4-F | 2-F-3-Cl | CH₂CH₂F |
| O | 4-F | 4-F-2-Cl | CH₂CH₂F |
| O | 4-F | 2-Cl-3,6-F₂ | CH₂CH₂F |
| O | 4-F | 3-Cl-2,6-F₂ | CH₂CH₂F |
| O | 4-F | 3-F-4-Cl | CH₂CH₂F |
| O | 4-F | 2-F-4-Cl | CH₂CH₂F |
| O | 4-F | 2,3-Cl₂ | CH₂CH₂F |
| O | 4-F | 2-CN | CH₂CH₂F |
| CH₂ | 4-Cl | H | CH₂CH₂F |
| CH₂ | 4-Cl | 3,5-F₂ | CH₂CH₂F |
| CH₂ | 4-Cl | 4-F | CH₂CH₂F |
| CH₂ | 4-Cl | 2,6-F₂ | CH₂CH₂F |
| CH₂ | 4-F | H | CH₂CH₂F |
| CH₂ | 4-F | 3,5-F₂ | CH₂CH₂F |
| CH₂ | 4-F | 4-F | CH₂CH₂F |
| CH₂ | 4-F | 2,6-F₂ | CH₂CH₂F |
| O | 3-Cl | H | CH₂CH₂F |
| O | 3-Cl | 3,5-F₂ | CH₂CH₂F |
| O | 3-Cl | 4-F | CH₂CH₂F |
| O | 3-Cl | 2,6-F₂ | CH₂CH₂F |
| O | 3-Cl | 2,5-F₂ | CH₂CH₂F |
| O | 3-F | H | CH₂CH₂F |
| O | 3-F | 3,5-F₂ | CH₂CH₂F |
| O | 3-F | 4-F | CH₂CH₂F |
| O | 3-F | 2,6-F₂ | CH₂CH₂F |
| O | 3-F | 2,5-F₂ | CH₂CH₂F |
| O | 4-CN | H | CH₂CH₂F |
| O | 4-CN | 3,5-F₂ | CH₂CH₂F |
| O | 4-CN | 4-F | CH₂CH₂F |
| O | 4-CN | 2,6-F₂ | CH₂CH₂F |
| O | 4-CN | 2,5-F₂ | CH₂CH₂F |
| O | 2,5-F₂ | H | CH₂CH₂F |
| O | 2,5-F₂ | 3,5-F₂ | CH₂CH₂F |
| O | 2,5-F₂ | 4-F | CH₂CH₂F |
| O | 2,5-F₂ | 2,6-F₂ | CH₂CH₂F |
| O | 2,5-F₂ | 2,5-F₂ | CH₂CH₂F |
| O | 3,5-F₂ | H | CH₂CH₂F |
| O | 3,5-F₂ | 3,5-F₂ | CH₂CH₂F |
| O | 3,5-F₂ | 4-F | CH₂CH₂F |
| O | 3,5-F₂ | 2,6-F₂ | CH₂CH₂F |
| O | 3,5-F₂ | 2,5-F₂ | CH₂CH₂F |
| O | 3,4-F₂ | H | CH₂CH₂F |
| O | 3,4-F₂ | 3,5-F₂ | CH₂CH₂F |
| O | 3,4-F₂ | 4-F | CH₂CH₂F |
| O | 3,4-F₂ | 2,6-F₂ | CH₂CH₂F |
| O | 3,4-F₂ | 2,5-F₂ | CH₂CH₂F |
| O | 4-CF₃ | H | CH₂CH₂F |
| O | 4-CF₃ | 3,5-F₂ | CH₂CH₂F |
| O | 4-CF₃ | 4-F | CH₂CH₂F |
| O | 4-CF₃ | 2,6-F₂ | CH₂CH₂F |
| O | 4-CF₃ | 2,5-F₂ | CH₂CH₂F |
| O | 4-Cl | H | CH₂CH₂OH |
| O | 4-Cl | 2-F | CH₂CH₂OH |
| O | 4-Cl | 3-F | CH₂CH₂OH |
| O | 4-Cl | 4-F | CH₂CH₂OH |
| O | 4-Cl | 4-Cl | CH₂CH₂OH |
| O | 4-Cl | 2,5-F₂ | CH₂CH₂OH |
| O | 4-Cl | 2,4-F₂ | CH₂CH₂OH |
| O | 4-Cl | 2-Cl | CH₂CH₂OH |
| O | 4-Cl | 2,6-F₂ | CH₂CH₂OH |
| O | 4-Cl | 3,5-F₂ | CH₂CH₂OH |
| O | 4-Cl | 2,3-F₂ | CH₂CH₂OH |
| O | 4-Cl | 3,4-F₂ | CH₂CH₂OH |
| O | 4-Cl | 2,3,5-F₃ | CH₂CH₂OH |
| O | 4-Cl | 2,3,6-F₃ | CH₂CH₂OH |
| O | 4-Cl | 2,4,5-F₂ | CH₂CH₂OH |
| O | 4-Cl | 2,6-Cl₂ | CH₂CH₂OH |
| O | 4-Cl | 2-F-6-Cl | CH₂CH₂OH |
| O | 4-Cl | 2-F-3-Cl | CH₂CH₂OH |
| O | 4-Cl | 4-F-2-Cl | CH₂CH₂OH |
| O | 4-Cl | 2-Cl-3,6-F₂ | CH₂CH₂OH |
| O | 4-Cl | 3-Cl-2,6-F₂ | CH₂CH₂OH |
| O | 4-Cl | 3-F-4-Cl | CH₂CH₂OH |
| O | 4-Cl | 2-F-4-Cl | CH₂CH₂OH |
| O | 4-Cl | 2,3-Cl₂ | CH₂CH₂OH |
| O | 4-Cl | 2-CN | CH₂CH₂OH |
| O | 4-F | H | CH₂CH₂OH |
| O | 4-F | 2-F | CH₂CH₂OH |
| O | 4-F | 3-F | CH₂CH₂OH |
| O | 4-F | 4-F | CH₂CH₂OH |
| O | 4-F | 4-Cl | CH₂CH₂OH |
| O | 4-F | 2,5-F₂ | CH₂CH₂OH |
| O | 4-F | 2,4-F₂ | CH₂CH₂OH |
| O | 4-F | 2-Cl | CH₂CH₂OH |
| O | 4-F | 2,6-F₂ | CH₂CH₂OH |
| O | 4-F | 3,5-F₂ | CH₂CH₂OH |
| O | 4-F | 2,3-F₂ | CH₂CH₂OH |
| O | 4-F | 3,4-F₂ | CH₂CH₂OH |
| O | 4-F | 2,3,5-F₂ | CH₂CH₂OH |
| O | 4-F | 2,3,6-F₂ | CH₂CH₂OH |
| O | 4-F | 2,4,5-F₂ | CH₂CH₂OH |
| O | 4-F | 2,6-Cl₂ | CH₂CH₂OH |
| O | 4-F | 2-F-6-Cl | CH₂CH₂OH |
| O | 4-F | 2-F-3-Cl | CH₂CH₂OH |
| O | 4-F | 4-F-2-Cl | CH₂CH₂OH |
| O | 4-F | 2-Cl-3,6-F₂ | CH₂CH₂OH |
| O | 4-F | 3-Cl-2,6-F₂ | CH₂CH₂OH |
| O | 4-F | 3-F-4-Cl | CH₂CH₂OH |
| O | 4-F | 2-F-4-Cl | CH₂CH₂OH |
| O | 4-F | 2,3-Cl₂ | CH₂CH₂OH |
| O | 4-F | 2-CN | CH₂CH₂OH |
| CH₂ | 4-Cl | H | CH₂CH₂OH |
| CH₂ | 4-Cl | 3,5-F₂ | CH₂CH₂OH |
| CH₂ | 4-Cl | 4-F | CH₂CH₂OH |
| CH₂ | 4-Cl | 2,6-F₂ | CH₂CH₂OH |
| CH₂ | 4-F | H | CH₂CH₂OH |
| CH₂ | 4-F | 3,5-F₂ | CH₂CH₂OH |
| CH₂ | 4-F | 4-F | CH₂CH₂OH |
| CH₂ | 4-F | 2,6-F₂ | CH₂CH₂OH |
| O | 3-F | H | CH₂CH₂OH |
| O | 3-F | 3,5-F₂ | CH₂CH₂OH |
| O | 3-F | 4-F | CH₂CH₂OH |
| O | 3-F | 2,6-F₂ | CH₂CH₂OH |
| O | 3-F | 2,5-F₂ | CH₂CH₂OH |
| O | 2,5-F₂ | H | CH₂CH₂OH |
| O | 2,5-F₂ | 3,5-F₂ | CH₂CH₂OH |
| O | 2,5-F₂ | 4-F | CH₂CH₂OH |
| O | 2,5-F₂ | 2,6-F₂ | CH₂CH₂OH |
| O | 2,5-F₂ | 2,5-F₂ | CH₂CH₂OH |
| O | 3,5-F₂ | H | CH₂CH₂OH |
| O | 3,5-F₂ | 3,5-F₂ | CH₂CH₂OH |
| O | 3,5-F₂ | 4-F | CH₂CH₂OH |
| O | 3,5-F₂ | 2,6-F₂ | CH₂CH₂OH |
| O | 3,5-F₂ | 2,5-F₂ | CH₂CH₂OH |
| O | 3,4-F₂ | H | CH₂CH₂OH |
| O | 3,4-F₂ | 3,5-F₂ | CH₂CH₂OH |
| O | 3,4-F₂ | 4-F | CH₂CH₂OH |
| O | 3,4-F₂ | 2,6-F₂ | CH₂CH₂OH |
| O | 3,4-F₂ | 2,5-F₂ | CH₂CH₂OH |
| O | 4-CF₃ | H | CH₂CH₂OH |
| O | 4-CF₃ | 3,5-F₂ | CH₂CH₂OH |
| O | 4-CF₃ | 4-F | CH₂CH₂OH |
| O | 4-CF₃ | 2,6-F₂ | CH₂CH₂OH |
| O | 4-CF₃ | 2,5-F₂ | CH₂CH₂OH |
| O | 4-Cl | H | CH₂CF₃ |
| O | 4-F | H | CH₂CF₃ |
| CH₂ | 4-Cl | H | CH₂CF₃ |
| CH₂ | 4-F | H | CH₂CF₃ |
| O | 4-Cl | H | OH |
| O | 4-F | H | OH |
| CH₂ | 4-Cl | H | OH |
| CH₂ | 4-F | H | OH |
| O | 4-Cl | H | OCH₃ |
| O | 4-F | H | OCH₃ |
| CH₂ | 4-Cl | H | OCH₃ |
| CH₂ | 4-F | H | OCH₃ |

TABLE III

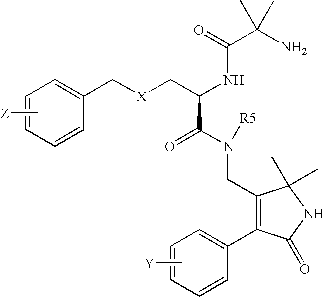

| X | Y | Z | R5 |
|---|---|---|---|
| O | 4-CXl | H | CH₂CH₂F |
| O | 4-Cl | 2-F | CH₂CH₂F |
| O | 4-Cl | 3-F | CH₂CH₂F |
| O | 4-Cl | 4-F | CH₂CH₂F |
| O | 4-Cl | 4-Cl | CH₂CH₂F |
| O | 4-Cl | 2,5-F₂ | CH₂CH₂F |
| O | 4-Cl | 2,4-F₂ | CH₂CH₂F |
| O | 4-Cl | 2-Cl | CH₂CH₂F |
| O | 4-Cl | 2,6-F₂ | CH₂CH₂F |
| O | 4-Cl | 3,5-F₂ | CH₂CH₂F |
| O | 4-Cl | 2,3-F₂ | CH₂CH₂F |
| O | 4-Cl | 3,4-F₂ | CH₂CH₂F |
| O | 4-Cl | 2,3,5-F₃ | CH₂CH₂F |
| O | 4-Cl | 2,3,6-F₃ | CH₂CH₂F |
| O | 4-Cl | 2,4,5-F₃ | CH₂CH₂F |
| O | 4-Cl | 2,6-Cl₂ | CH₂CH₂F |
| O | 4-Cl | 2-F-6-Cl | CH₂CH₂F |
| O | 4-Cl | 2-F-3-Cl | CH₂CH₂F |
| O | 4-Cl | 4-F-2-Cl | CH₂CH₂F |
| O | 4-Cl | 2-Cl-3,6-F₂ | CH₂CH₂F |
| O | 4-Cl | 3-Cl-2,6-F₂ | CH₂CH₂F |
| O | 4-Cl | 3-F-4-Cl | CH₂CH₂F |
| O | 4-Cl | 2-F-4-Cl | CH₂CH₂F |
| O | 4-Cl | 2,3-Cl₂ | CH₂CH₂F |
| O | 4-Cl | 2-CN | CH₂CH₂F |
| O | 4-F | H | CH₂CH₂F |
| O | 4-F | 2-F | CH₂CH₂F |
| O | 4-F | 3-F | CH₂CH₂F |
| O | 4-F | 4-F | CH₂CH₂F |
| O | 4-F | 4-Cl | CH₂CH₂F |
| O | 4-F | 2,5-F₂ | CH₂CH₂F |
| O | 4-F | 2,4-F₂ | CH₂CH₂F |
| O | 4-F | 2-Cl | CH₂CH₂F |
| O | 4-F | 2,6-F₂ | CH₂CH₂F |
| O | 4-F | 3,5-F₂ | CH₂CH₂F |
| O | 4-F | 2,3-F₂ | CH₂CH₂F |
| O | 4-F | 3,4-F₂ | CH₂CH₂F |
| O | 4-F | 2,3,5-F₃ | CH₂CH₂F |
| O | 4-F | 2,3,6-F₃ | CH₂CH₂F |
| O | 4-F | 2,4,5-F₃ | CH₂CH₂F |
| O | 4-F | 2,6-Cl₂ | CH₂CH₂F |
| O | 4-F | 2-F-6-Cl | CH₂CH₂F |
| O | 4-F | 2-F-3-Cl | CH₂CH₂F |
| O | 4-F | 4-F-2-Cl | CH₂CH₂F |
| O | 4-F | 2-Cl-3,6-F₂ | CH₂CH₂F |
| O | 4-F | 3-Cl-2,6-F₂ | CH₂CH₂F |
| O | 4-F | 3-F-4-Cl | CH₂CH₂F |
| O | 4-F | 2-F-4-Cl | CH₂CH₂F |
| O | 4-F | 2,3-Cl₂ | CH₂CH₂F |
| O | 4-F | 2-CN | CH₂CH₂F |
| CH₂ | 4-Cl | H | CH₂CH₂F |
| CH₂ | 4-Cl | 3,5-F₂ | CH₂CH₂F |
| CH₂ | 4-Cl | 4-F | CH₂CH₂F |
| CH₂ | 4-Cl | 2,6-F₂ | CH₂CH₂F |
| CH₂ | 4-F | H | CH₂CH₂F |
| CH₂ | 4-F | 3,5-F₂ | CH₂CH₂F |
| CH₂ | 4-F | 4-F | CH₂CH₂F |
| CH₂ | 4-F | 2,6-F₂ | CH₂CH₂F |
| O | 3-Cl | H | CH₂CH₂F |
| O | 3-Cl | 3,5-F₂ | CH₂CH₂F |
| O | 3-Cl | 4-F | CH₂CH₂F |
| O | 3-Cl | 2,6-F₂ | CH₂CH₂F |
| O | 3-Cl | 2,5-F₂ | CH₂CH₂F |
| O | 3-F | H | CH₂CH₂F |
| O | 3-F | 3,5-F₂ | CH₂CH₂F |
| O | 3-F | 4-F | CH₂CH₂F |
| O | 3-F | 2,6-F₂ | CH₂CH₂F |
| O | 3-F | 2,5-F₂ | CH₂CH₂F |
| O | 4-CN | H | CH₂CH₂F |
| O | 4-CN | 3,5-F₂ | CH₂CH₂F |
| O | 4-CN | 4-F | CH₂CH₂F |
| O | 4-CN | 2,6-F₂ | CH₂CH₂F |
| O | 4-CN | 2,5-F₂ | CH₂CH₂F |
| O | 2,5-F₂ | H | CH₂CH₂F |
| O | 2,5-F₂ | 3,5-F₂ | CH₂CH₂F |
| O | 2,5-F₂ | 4-F | CH₂CH₂F |
| O | 2,5-F₂ | 2,6-F₂ | CH₂CH₂F |
| O | 2,5-F₂ | 2,5-F₂ | CH₂CH₂F |
| O | 3,5-F₂ | H | CH₂CH₂F |
| O | 3,5-F₂ | 3,5-F₂ | CH₂CH₂F |
| O | 3,5-F₂ | 4-F | CH₂CH₂F |
| O | 3,5-F₂ | 2,6-F₂ | CH₂CH₂F |
| O | 3,5-F₂ | 2,5-F₂ | CH₂CH₂F |
| O | 3,4-F₂ | H | CH₂CH₂F |
| O | 3,4-F₂ | 3,5-F₂ | CH₂CH₂F |
| O | 3,4-F₂ | 4-F | CH₂CH₂F |
| O | 3,4-F₂ | 2,6-F₂ | CH₂CH₂F |
| O | 3,4-F₂ | 2,5-F₂ | CH₂CH₂F |
| O | 4-CF₃ | H | CH₂CH₂F |
| O | 4-CF₃ | 3,5-F₂ | CH₂CH₂F |
| O | 4-CF₃ | 4-F | CH₂CH₂F |
| O | 4-CF₃ | 2,6-F₂ | CH₂CH₂F |
| O | 4-CF₃ | 2,5-F₂ | CH₂CH₂F |
| O | 4-Cl | H | CH₂CH₂OH |
| O | 4-Cl | 2-F | CH₂CH₂OH |
| O | 4-Cl | 3-F | CH₂CH₂OH |
| O | 4-Cl | 4-F | CH₂CH₂OH |
| O | 4-Cl | 4-Cl | CH₂CH₂OH |
| O | 4-Cl | 2,5-F₂ | CH₂CH₂OH |
| O | 4-Cl | 2,4-F₂ | CH₂CH₂OH |
| O | 4-Cl | 2-Cl | CH₂CH₂OH |
| O | 4-Cl | 2,6-F₂ | CH₂CH₂OH |
| O | 4-Cl | 3,5-F₂ | CH₂CH₂OH |
| O | 4-Cl | 2,3-F₂ | CH₂CH₂OH |
| O | 4-Cl | 3,4-F₂ | CH₂CH₂OH |
| O | 4-Cl | 2,3,5-F₂ | CH₂CH₂OH |
| O | 4-Cl | 2,3,6-F₃ | CH₂CH₂OH |
| O | 4-Cl | 2,4,5-F₃ | CH₂CH₂OH |
| O | 4-Cl | 2,6-Cl₂ | CH₂CH₂OH |
| O | 4-Cl | 2-F-6-Cl | CH₂CH₂OH |
| O | 4-Cl | 2-F-3-Cl | CH₂CH₂OH |
| O | 4-Cl | 4-F-2-Cl | CH₂CH₂OH |
| O | 4-Cl | 2-Cl-3,6-F₂ | CH₂CH₂OH |
| O | 4-Cl | 3-Cl-2,6-F₂ | CH₂CH₂OH |
| O | 4-Cl | 3-F-4-Cl | CH₂CH₂OH |
| O | 4-Cl | 2-F-4-Cl | CH₂CH₂OH |
| O | 4-Cl | 2,3-Cl₂ | CH₂CH₂OH |
| O | 4-Cl | 2-CN | CH₂CH₂OH |
| O | 4-F | H | CH₂CH₂OH |
| O | 4-F | 2-F | CH₂CH₂OH |
| O | 4-F | 3-F | CH₂CH₂OH |
| O | 4-F | 4-F | CH₂CH₂OH |
| O | 4-F | 4-Cl | CH₂CH₂OH |
| O | 4-F | 2,5-F₂ | CH₂CH₂OH |
| O | 4-F | 2,4-F₂ | CH₂CH₂OH |
| O | 4-F | 2-Cl | CH₂CH₂OH |
| O | 4-F | 2,6-F₂ | CH₂CH₂OH |
| O | 4-F | 3,5-F₂ | CH₂CH₂OH |
| O | 4-F | 2,3-F₂ | CH₂CH₂OH |
| O | 4-F | 3,4-F₂ | CH₂CH₂OH |
| O | 4-F | 2,3,5-F₃ | CH₂CH₂OH |
| O | 4-F | 2,3,6-F₃ | CH₂CH₂OH |
| O | 4-F | 2,4,5-F₃ | CH₂CH₂OH |
| O | 4-F | 2,6-Cl₂ | CH₂CH₂OH |
| O | 4-F | 2-F-6-Cl | CH₂CH₂OH |
| O | 4-F | 2-F-3-Cl | CH₂CH₂OH |
| O | 4-F | 4-F-2-Cl | CH₂CH₂OH |
| O | 4-F | 2-Cl-3,6-F₂ | CH₂CH₂OH |
| O | 4-F | 3-Cl-2,6-F₂ | CH₂CH₂OH |
| O | 4-F | 3-F-4-Cl | CH₂CH₂OH |
| O | 4-F | 2-F-4-Cl | CH₂CH₂OH |
| O | 4-F | 2,3-Cl₂ | CH₂CH₂OH |
| O | 4-F | 2-CN | CH₂CH₂OH |
| CH₂ | 4-Cl | H | CH₂CH₂OH |
| CH₂ | 4-Cl | 3,5-F₂ | CH₂CH₂OH |
| CH₂ | 4-Cl | 4-F | CH₂CH₂OH |
| CH₂ | 4-Cl | 2,6-F₂ | CH₂CH₂OH |
| CH₂ | 4-F | H | CH₂CH₂OH |
| CH₂ | 4-F | 3,5-F₂ | CH₂CH₂OH |
| CH₂ | 4-F | 4-F | CH₂CH₂OH |
| CH₂ | 4-F | 2,6-F₂ | CH₂CH₂OH |
| O | 3-F | H | CH₂CH₂OH |
| O | 3-F | 3,5-F₂ | CH₂CH₂OH |
| O | 3-F | 4-F | CH₂CH₂OH |
| O | 3-F | 2,6-F₂ | CH₂CH₂OH |
| O | 3-F | 2,5-F₂ | CH₂CH₂OH |
| O | 2,5-F₂ | H | CH₂CH₂OH |
| O | 2,5-F₂ | 3,5-F₂ | CH₂CH₂OH |
| O | 2,5-F₂ | 4-F | CH₂CH₂OH |
| O | 2,5-F₂ | 2,6-F₂ | CH₂CH₂OH |
| O | 2,5-F₂ | 2,5-F₂ | CH₂CH₂OH |
| O | 3,5-F₂ | H | CH₂CH₂OH |
| O | 3,5-F₂ | 3,5-F₂ | CH₂CH₂OH |
| O | 3,,5-F₂ | 4-F | CH₂CH₂OH |
| O | 3,5-F₂ | 2,6-F₂ | CH₂CH₂OH |
| O | 3,5-F₂ | 2,5-F₂ | CH₂CH₂OH |
| O | 3,4-F₂ | H | CH₂CH₂OH |
| O | 3,4-F₂ | 3,5-F₂ | CH₂CH₂OH |
| O | 3,4-F₂ | 4-F | CH₂CH₂OH |
| O | 3,4-F₂ | 2,6-F₂ | CH₂CH₂OH |
| O | 3,4-F₂ | 2,5-F₂ | CH₂CH₂OH |
| O | 4-CF₃ | H | CH₂CH₂OH |
| O | 4-CF₃ | 3,5-F₂ | CH₂CH₂OH |
| O | 4-CF₃ | 4-F | CH₂CH₂OH |
| O | 4-CF₃ | 2,6-F₂ | CH₂CH₂OH |
| O | 4-CF₃ | 2,5-F₂ | CH₂CH₂OH |
| O | 4-Cl | H | CH₂CF₃ |
| O | 4-F | H | CH₂CF₃ |
| CH₂ | 4-Cl | H | CH₂CF₃ |
| CH₂ | 4-F | H | CH₂CF₃ |
| O | 4-Cl | H | OH |
| O | 4-F | H | OH |
| CH₂ | 4-Cl | H | OH |
| CH₂ | 4-F | H | OH |
| O | 4-Cl | H | OCH₃ |
| O | 4-F | H | OCH₃ |
| CH₂ | 4-Cl | H | OCH₃ |
| CH₂ | 4-F | H | OCH₃ |

TABLE IV

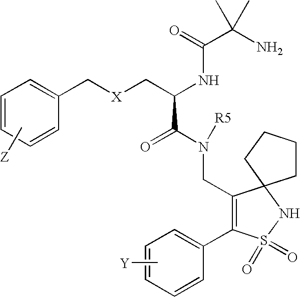

| X | Y | Z | R5 |
|---|---|---|---|
| O | 4-Cl | H | CH₂CH₂F |
| O | 4-Cl | 2-F | CH₂CH₂F |
| O | 4-Cl | 3-F | CH₂CH₂F |
| O | 4-Cl | 4-F | CH₂CH₂F |
| O | 4-Cl | 4-Cl | CH₂CH₂F |
| O | 4-Cl | 2,5-F₂ | CH₂CH₂F |
| O | 4-Cl | 2,4-F₂ | CH₂CH₂F |
| O | 4-Cl | 2-Cl | CH₂CH₂F |
| O | 4-Cl | 2,6-F₂ | CH₂CH₂F |
| O | 4-Cl | 3,5-F₂ | CH₂CH₂F |
| O | 4-Cl | 2,3-F₂ | CH₂CH₂F |
| O | 4-Cl | 3,4-F₂ | CH₂CH₂F |
| O | 4-Cl | 2,3,5-F₃ | CH₂CH₂F |
| O | 4-Cl | 2,3,6-F₃ | CH₂CH₂F |
| O | 4-Cl | 2,4,5-F₃ | CH₂CH₂F |
| O | 4-Cl | 2,6-Cl₂ | CH₂CH₂F |
| O | 4-Cl | 2-F-6-Cl | CH₂CH₂F |
| O | 4-Cl | 2-F-3-Cl | CH₂CH₂F |
| O | 4-Cl | 4-F-2-Cl | CH₂CH₂F |
| O | 4-Cl | 2-Cl-3,6-F₂ | CH₂CH₂F |
| O | 4-Cl | 3-Cl-2,6-F₂ | CH₂CH₂F |
| O | 4-Cl | 3-F-4-Cl | CH₂CH₂F |
| O | 4-Cl | 2-F-4-Cl | CH₂CH₂F |
| O | 4-Cl | 2,3-Cl₂ | CH₂CH₂F |
| O | 4-Cl | 2-CN | CH₂CH₂F |
| O | 4-F | H | CH₂CH₂F |
| O | 4-F | 2-F | CH₂CH₂F |
| O | 4-F | 3-F | CH₂CH₂F |
| O | 4-F | 4-F | CH₂CH₂F |
| O | 4-F | 4-Cl | CH₂CH₂F |
| O | 4-F | 2,5-F₂ | CH₂CH₂F |
| O | 4-F | 2,4-F₂ | CH₂CH₂F |
| O | 4-F | 2-Cl | CH₂CH₂F |
| O | 4-F | 2,6-F₂ | CH₂CH₂F |
| O | 4-F | 3,5-F₂ | CH₂CH₂F |
| O | 4-F | 2,3-F₂ | CH₂CH₂F |
| O | 4-F | 3,4-F₂ | CH₂CH₂F |
| O | 4-F | 2,3,5-F₃ | CH₂CH₂F |
| O | 4-F | 2,3,6-F₃ | CH₂CH₂F |
| O | 4-F | 2,4,5-F₃ | CH₂CH₂F |
| O | 4-F | 2,6-Cl₂ | CH₂CH₂F |
| O | 4-F | 2-F-6-Cl | CH₂CH₂F |
| O | 4-F | 2-F-3-Cl | CH₂CH₂F |
| O | 4-F | 4-F-2-Cl | CH₂CH₂F |
| O | 4-F | 2-Cl-3,6-F₂ | CH₂CH₂F |
| O | 4-F | 3-Cl-2,6-F₂ | CH₂CH₂F |
| O | 4-F | 3-F-4-Cl | CH₂CH₂F |
| O | 4-F | 2-F-4-Cl | CH₂CH₂F |
| O | 4-F | 2,3-Cl₂ | CH₂CH₂F |
| O | 4-F | 2-CN | CH₂CH₂F |
| CH₂ | 4-Cl | H | CH₂CH₂F |
| CH₂ | 4-Cl | 3,5-F₂ | CH₂CH₂F |
| CH₂ | 4-Cl | 4-F | CH₂CH₂F |
| CH₂ | 4-Cl | 2,6-F₂ | CH₂CH₂F |
| CH₂ | 4-F | H | CH₂CH₂F |
| CH₂ | 4-F | 3,5-F₂ | CH₂CH₂F |
| CH₂ | 4-F | 4-F | CH₂CH₂F |
| CH₂ | 4-F | 2,6-F₂ | CH₂CH₂F |
| O | 3-Cl | H | CH₂CH₂F |
| O | 3-Cl | 3,5-F₂ | CH₂CH₂F |
| O | 3-Cl | 4-F | CH₂CH₂F |
| O | 3-Cl | 2,6-F₂ | CH₂CH₂F |
| O | 3-Cl | 2,5-F₂ | CH₂CH₂F |
| O | 3-F | H | CH₂CH₂F |
| O | 3-F | 3,5-F₂ | CH₂CH₂F |
| O | 3-F | 4-F | CH₂CH₂F |
| O | 3-F | 2,6-F₂ | CH₂CH₂F |
| O | 3-F | 2,5-F₂ | CH₂CH₂F |
| O | 4-CN | H | CH₂CH₂F |
| O | 4-CN | 3,5-F₂ | CH₂CH₂F |
| O | 4-CN | 4-F | CH₂CH₂F |
| O | 4-CN | 2,6-F₂ | CH₂CH₂F |
| O | 4-CN | 2,5-F₂ | CH₂CH₂F |
| O | 2,5-F₂ | H | CH₂CH₂F |
| O | 2,5-F₂ | 3,5-F₂ | CH₂CH₂F |
| O | 2,5-F₂ | 4-F | CH₂CH₂F |
| O | 2,5-F₂ | 2,6-F₂ | CH₂CH₂F |
| O | 2,5-F₂ | 2,5-F₂ | CH₂CH₂F |
| O | 3,5-F₂ | H | CH₂CH₂F |
| O | 3,5-F₂ | 3,5-F₂ | CH₂CH₂F |
| O | 3,5-F₂ | 4-F | CH₂CH₂F |
| O | 3,5-F₂ | 2,6-F₂ | CH₂CH₂F |
| O | 3,5-F₂ | 2,5-F₂ | CH₂CH₂F |
| O | 3,4-F₂ | H | CH₂CH₂F |
| O | 3,4-F₂ | 3,5-F₂ | CH₂CH₂F |
| O | 3,4-F₂ | 4-F | CH₂CH₂F |
| O | 3,4-F₂ | 2,6-F₂ | CH₂CH₂F |
| O | 3,4-F₂ | 2,5-F₂ | CH₂CH₂F |
| O | 4-CF₃ | H | CH₂CH₂F |
| O | 4-CF₃ | 3,5-F₂ | CH₂CH₂F |
| O | 4-CF₃ | 4-F | CH₂CH₂F |
| O | 4-CF₃ | 2,6-F₂ | CH₂CH₂F |
| O | 4-CF₃ | 2,5-F₂ | CH₂CH₂F |
| O | 4-Cl | H | CH₂CH₂OH |
| O | 4-Cl | 2-F | CH₂CH₂OH |
| O | 4-Cl | 3-F | CH₂CH₂OH |
| O | 4-Cl | 4-F | CH₂CH₂OH |
| O | 4-Cl | 4-Cl | CH₂CH₂OH |
| O | 4-Cl | 2,5-F₂ | CH₂CH₂OH |
| O | 4-Cl | 2,4-F₂ | CH₂CH₂OH |
| O | 4-Cl | 2-Cl | CH₂CH₂OH |
| O | 4-Cl | 2,6-F₂ | CH₂CH₂OH |
| O | 4-Cl | 3,5-F₂ | CH₂CH₂OH |
| O | 4-Cl | 2,3-F₂ | CH₂CH₂OH |
| O | 4-Cl | 3,4-F₂ | CH₂CH₂OH |
| O | 4-Cl | 2,3,5-F₃ | CH₂CH₂OH |
| O | 4-Cl | 2,3,6-F₃ | CH₂CH₂OH |
| O | 4-Cl | 2,4,5-F₃ | CH₂CH₂OH |
| O | 4-Cl | 2,6-Cl₂ | CH₂CH₂OH |
| O | 4-Cl | 2-F-6-Cl | CH₂CH₂OH |
| O | 4-Cl | 2-F-3-Cl | CH₂CH₂OH |
| O | 4-Cl | 4-F-2-Cl | CH₂CH₂OH |
| O | 4-Cl | 2-Cl-3,6-F₂ | CH₂CH₂OH |
| O | 4-Cl | 3-Cl-2,6-F₂ | CH₂CH₂OH |
| O | 4-Cl | 3-F-4-Cl | CH₂CH₂OH |
| O | 4-Cl | 2-F-4-Cl | CH₂CH₂OH |
| O | 4-Cl | 2,3-Cl₂ | CH₂CH₂OH |
| O | 4-Cl | 2-CN | CH₂CH₂OH |
| O | 4-F | H | CH₂CH₂OH |
| O | 4-F | 2-F | CH₂CH₂OH |
| O | 4-F | 3-F | CH₂CH₂OH |
| O | 4-F | 4-F | CH₂CH₂OH |
| O | 4-F | 4-Cl | CH₂CH₂OH |
| O | 4-F | 2,5-F₂ | CH₂CH₂OH |
| O | 4-F | 2,4-F₂ | CH₂CH₂OH |
| O | 4-F | 2-Cl | CH₂CH₂OH |
| O | 4-F | 2,6-F₂ | CH₂CH₂OH |
| O | 4-F | 3,5-F₂ | CH₂CH₂OH |
| O | 4-F | 2,3-F₂ | CH₂CH₂OH |
| O | 4-F | 3,4-F₂ | CH₂CH₂OH |
| O | 4-F | 2,3,5-F₃ | CH₂CH₂OH |
| O | 4-F | 2,3,6-F₃ | CH₂CH₂OH |
| O | 4-F | 2,4,5-F₃ | CH₂CH₂OH |
| O | 4-F | 2,6-Cl₂ | CH₂CH₂OH |
| O | 4-F | 2-F-6-Cl | CH₂CH₂OH |
| O | 4-F | 2-F-3-Cl | CH₂CH₂OH |
| O | 4-F | 4-F-2-Cl | CH₂CH₂OH |
| O | 4-F | 2-Cl-3,6-F₂ | CH₂CH₂OH |
| O | 4-F | 3-Cl-2,6-F₂ | CH₂CH₂OH |
| O | 4-F | 3-F-4-Cl | CH₂CH₂OH |
| O | 4-F | 2-F-4-Cl | CH₂CH₂OH |
| O | 4-F | 2,3-Cl₂ | CH₂CH₂OH |
| O | 4-F | 2-CN | CH₂CH₂OH |
| CH₂ | 4-Cl | H | CH₂CH₂OH |
| CH₂ | 4-Cl | 3,5-F₂ | CH₂CH₂OH |
| CH₂ | 4-Cl | 4-F | CH₂CH₂OH |
| CH₂ | 4-Cl | 2,6-F₂ | CH₂CH₂OH |
| CH₂ | 4-F | H | CH₂CH₂OH |
| CH₂ | 4-F | 3,5-F₂ | CH₂CH₂OH |
| CH₂ | 4-F | 4-F | CH₂CH₂OH |
| CH₂ | 4-F | 2,6-F₂ | CH₂CH₂OH |
| O | 3-F | H | CH₂CH₂OH |
| O | 3-F | 3,5-F₂ | CH₂CH₂OH |
| O | 3-F | 4-F | CH₂CH₂OH |
| O | 3-F | 2,6-F₂ | CH₂CH₂OH |
| O | 3-F | 2,5-F₂ | CH₂CH₂OH |
| O | 2,5-F₂ | H | CH₂CH₂OH |
| O | 2,5-F₂ | 3,5-F₂ | CH₂CH₂OH |
| O | 2,5-F₂ | 4-F | CH₂CH₂OH |
| O | 2,5-F₂ | 2,6-F₂ | CH₂CH₂OH |
| O | 2,5-F₂ | 2,5-F₂ | CH₂CH₂OH |
| O | 3,5-F₂ | H | CH₂CH₂OH |
| O | 3,5-F₂ | 3,5-F₂ | CH₂CH₂OH |
| O | 3,5-F₂ | 4-F | CH₂CH₂OH |
| O | 3,5-F₂ | 2,6-F₂ | CH₂CH₂OH |
| O | 3,5-F₂ | 2,5-F₂ | CH₂CH₂OH |
| O | 3,4-F₂ | H | CH₂CH₂OH |
| O | 3,4-F₂ | 3,5-F₂ | CH₂CH₂OH |
| O | 3,4-F₂ | 4-F | CH₂CH₂OH |
| O | 3,4-F₂ | 2,6-F₂ | CH₂CH₂OH |
| O | 3,4-F₂ | 2,5-F₂ | CH₂CH₂OH |
| O | 4-CF₃ | H | CH₂CH₂OH |
| O | 4-CF₃ | 3,5-F₂ | CH₂CH₂OH |
| O | 4-CF₃ | 4-F | CH₂CH₂OH |
| O | 4-CF₃ | 2,6-F₂ | CH₂CH₂OH |
| O | 4-CF₃ | 2,5-F₂ | CH₂CH₂OH |
| O | 4-Cl | H | CH₂CF₃ |
| O | 4-F | H | CH₂CF₃ |
| CH₂ | 4-Cl | H | CH₂CF₃ |
| CH₂ | 4-F | H | CH₂CF₃ |
| O | 4-Cl | H | OH |
| O | 4-F | H | OH |
| CH₂ | 4-Cl | H | OH |
| CH₂ | 4-F | H | OH |
| O | 4-Cl | H | OCH₃ |
| O | 4-F | H | OCH₃ |
| CH₂ | 4-Cl | H | OCH₃ |
| CH₂ | 4-F | H | OCH₃ |

TABLE V

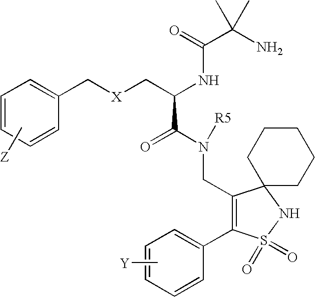

| X | Y | Z | R5 |
|---|---|---|---|
| O | 4-Cl | H | CH₂CH₂F |
| O | 4-Cl | 2-F | CH₂CH₂F |
| O | 4-Cl | 3-F | CH₂CH₂F |
| O | 4-Cl | 4-F | CH₂CH₂F |
| O | 4-Cl | 4-Cl | CH₂CH₂F |
| O | 4-Cl | 2,5-F₂ | CH₂CH₂F |
| O | 4-Cl | 2,4-F₂ | CH₂CH₂F |
| O | 4-Cl | 2-Cl | CH₂CH₂F |
| O | 4-Cl | 2,6-F₂ | CH₂CH₂F |
| O | 4-Cl | 3,5-F₂ | CH₂CH₂F |
| O | 4-Cl | 2,3-F₂ | CH₂CH₂F |
| O | 4-Cl | 3,4-F₂ | CH₂CH₂F |
| O | 4-Cl | 2,3,5-F₃ | CH₂CH₂F |
| O | 4-Cl | 2,3,6-F₃ | CH₂CH₂F |
| O | 4-CL | 2,4,5-F₃ | CH₂CH₂F |
| O | 4-Cl | 2,6-Cl₂ | CH₂CH₂F |
| O | 4-Cl | 2-F-6-Cl | CH₂CH₂F |
| O | 4-Cl | 2-F-3-Cl | CH₂CH₂F |
| O | 4-Cl | 4-F-2-Cl | CH₂CH₂F |
| O | 4-Cl | 2-Cl-3,6-F₂ | CH₂CH₂F |
| O | 4-Cl | 3-Cl-2,6-F₂ | CH₂CH₂F |
| O | 4-Cl | 3-F-4-Cl | CH₂CH₂F |
| O | 4-Cl | 2-F-4-Cl | CH₂CH₂F |
| O | 4-Cl | 2,3-Cl₂ | CH₂CH₂F |
| O | 4-Cl | 2-CN | CH₂CH₂F |
| O | 4-F | H | CH₂CH₂F |
| O | 4-F | 2-F | CH₂CH₂F |
| O | 4-F | 3-F | CH₂CH₂F |
| O | 4-F | 4-F | CH₂CH₂F |
| O | 4-F | 4-Cl | CH₂CH₂F |
| O | 4-F | 2,5-F₂ | CH₂CH₂F |
| O | 4-F | 2,4-F₂ | CH₂CH₂F |
| O | 4-F | 2-Cl | CH₂CH₂F |
| O | 4-F | 2,6-F₂ | CH₂CH₂F |
| O | 4-F | 3,5-F₂ | CH₂CH₂F |
| O | 4-F | 2,3-F₂ | CH₂CH₂F |
| O | 4-F | 3,4-F₂ | CH₂CH₂F |
| O | 4-F | 2,3,5-F₃ | CH₂CH₂F |
| O | 4-F | 2,3,6-F₃ | CH₂CH₂F |
| O | 4-F | 2,4,5-F₃ | CH₂CH₂F |
| O | 4-F | 2,6-Cl₂ | CH₂CH₂F |
| O | 4-F | 2-F-6-Cl | CH₂CH₂F |
| O | 4-F | 2-F-3-Cl | CH₂CH₂F |
| O | 4-F | 4-F-2-Cl | CH₂CH₂F |
| O | 4-F | 2-Cl-3,6-F₂ | CH₂CH₂F |
| O | 4-F | 3-Cl-2,6-F₂ | CH₂CH₂F |
| O | 4-F | 3-F-4-Cl | CH₂CH₂F |
| O | 4-F | 2-F-4-Cl | CH₂CH₂F |
| O | 4-F | 2,3-Cl₂ | CH₂CH₂F |
| O | 4-F | 2-CNN | CH₂CH₂F |
| CH₂ | 4-Cl | H | CH₂CH₂F |
| CH₂ | 4-Cl | 3,5-F₂ | CH₂CH₂F |
| CH₂ | 4-Cl | 4-F | CH₂CH₂F |
| CH₂ | 4-Cl | 2,6-F₂ | CH₂CH₂F |
| CH₂ | 4-F | H | CH₂CH₂F |
| CH₂ | 4-F | 3,5-F₂ | CH₂CH₂F |
| CH₂ | 4-F | 4-F | CH₂CH₂F |
| CH₂ | 4-F | 2,6-F₂ | CH₂CH₂F |
| O | 3-Cl | H | CH₂CH₂F |
| O | 3-Cl | 3,5-F₂ | CH₂CH₂F |
| O | 3-Cl | 4-F | CH₂CH₂F |
| O | 3-Cl | 2,6-F₂ | CH₂CH₂F |
| O | 3-Cl | 2,5-F₂ | CH₂CH₂F |
| O | 3-F | H | CH₂CH₂F |
| O | 3-F | 3,5-F₂ | CH₂CH₂F |
| O | 3-F | 4-F | CH₂CH₂F |
| O | 3-F | 2,6-F₂ | CH₂CH₂F |
| O | 3-F | 2,5-F₂ | CH₂CH₂F |
| O | 4-CN | H | CH₂CH₂F |
| O | 4-CN | 3,5-F₂ | CH₂CH₂F |
| O | 4-CN | 4-F | CH₂CH₂F |
| O | 4-CN | 2,6-F₂ | CH₂CH₂F |
| O | 4-CN | 2,5-F₂ | CH₂CH₂F |
| O | 2,5-F₂ | H | CH₂CH₂F |
| O | 2,5-F₂ | 3,5-F₂ | CH₂CH₂F |
| O | 2,5-F₂ | 4-F | CH₂CH₂F |
| O | 2,5-F₂ | 2,6-F₂ | CH₂CH₂F |
| O | 2,5-F₂ | 2,5-F₂ | CH₂CH₂F |
| O | 3,5-F₂ | H | CH₂CH₂F |
| O | 3,5-F₂ | 3,5-F₂ | CH₂CH₂F |
| O | 3,5-F₂ | 4-F | CH₂CH₂F |
| O | 3,5-F₂ | 2,6-F₂ | CH₂CH₂F |
| O | 3,5-F₂ | 2,5-F₂ | CH₂CH₂F |
| O | 3,4-F₂ | H | CH₂CH₂F |
| O | 3,4-F₂ | 3,5-F₂ | CH₂CH₂F |
| O | 3,4-F₂ | 4-F | CH₂CH₂F |
| O | 3,4-F₂ | 2,6-F₂ | CH₂CH₂F |
| O | 3,4-F₂ | 2,5-F₂ | CH₂CH₂F |
| O | 4-CF₃ | H | CH₂CH₂F |
| O | 4-CF₃ | 3,5-F₂ | CH₂CH₂F |
| O | 4-CF₃ | 4-F | CH₂CH₂F |
| O | 4-CF₃ | 2,6-F₂ | CH₂CH₂F |
| O | 4-CF₃ | 2,5-F₂ | CH₂CH₂F |
| O | 4-Cl | H | CH₂CH₂OH |
| O | 4-Cl | 2-F | CH₂CH₂OH |
| O | 4-Cl | 3-F | CH₂CH₂OH |
| O | 4-Cl | 4-F | CH₂CH₂OH |
| O | 4-Cl | 4-Cl | CH₂CH₂OH |
| O | 4-Cl | 2,5-F₂ | CH₂CH₂OH |
| O | 4-Cl | 2,4-F₂ | CH₂CH₂OH |
| O | 4-Cl | 2-Cl | CH₂CH₂OH |
| O | 4-Cl | 2,6-F₂ | CH₂CH₂OH |
| O | 4-Cl | 3,5-F₂ | CH₂CH₂OH |
| O | 4-Cl | 2,3-F₂ | CH₂CH₂OH |
| O | 4-Cl | 3,4-F₂ | CH₂CH₂OH |
| O | 4-Cl | 2,3,5-F₃ | CH₂CH₂OH |
| O | 4-Cl | 2,3,6-F₃ | CH₂CH₂OH |
| O | 4-Cl | 2,4,5-F₃ | CH₂CH₂OH |
| O | 4-Cl | 2,6-Cl₂ | CH₂CH₂OH |
| O | 4-Cl | 2-F-6-Cl | CH₂CH₂OH |
| O | 4-Cl | 2-F-3-Cl | CH₂CH₂OH |
| O | 4-Cl | 4-F-2-Cl | CH₂CH₂OH |
| O | 4-Cl | 2-Cl-3,6-F₂ | CH₂CH₂OH |
| O | 4-Cl | 3-Cl-2,6-F₂ | CH₂CH₂OH |
| O | 4-Cl | 3-F-4-Cl | CH₂CH₂OH |
| O | 4-Cl | 2-F-4-Cl | CH₂CH₂OH |
| O | 4-Cl | 2,3-Cl₂ | CH₂CH₂OH |
| O | 4-Cl | 2-CN | CH₂CH₂OH |
| O | 4-F | H | CH₂CH₂OH |
| O | 4-F | 2-F | CH₂CH₂OH |
| O | 4-F | 3-F | CH₂CH₂OH |
| O | 4-F | 4-F | CH₂CH₂OH |
| O | 4-F | 4-Cl | CH₂CH₂OH |
| O | 4-F | 2,5-F₂ | CH₂CH₂OH |
| O | 4-F | 2,4-F₂ | CH₂CH₂OH |
| O | 4-F | 2-Cl | CH₂CH₂OH |
| O | 4-F | 2,6-F₂ | CH₂CH₂OH |
| O | 4-F | 3,5-F₂ | CH₂CH₂OH |
| O | 4-F | 2,3-F₂ | CH₂CH₂OH |
| O | 4-F | 3,4-F₂ | CH₂CH₂OH |
| O | 4-F | 2,3,5-F₃ | CH₂CH₂OH |
| O | 4-F | 2,3,6-F₃ | CH₂CH₂OH |
| O | 4-F | 2,4,5-F₃ | CH₂CH₂OH |
| O | 4-F | 2,6-Cl₂ | CH₂CH₂OH |
| O | 4-F | 2-F-6-Cl | CH₂CH₂OH |
| O | 4-F | 2-F-3-Cl | CH₂CH₂OH |
| O | 4-F | 4-F-2-Cl | CH₂CH₂OH |
| O | 4-F | 2-Cl-3,6-F₂ | CH₂CH₂OH |
| O | 4-F | 3-Cl-2,6-F₂ | CH₂CH₂OH |
| O | 4-F | 3-F-4-Cl | CH₂CH₂OH |
| O | 4-F | 2-F-4-Cl | CH₂CH₂OH |
| O | 4-F | 2,3-Cl₂ | CH₂CH₂OH |
| O | 4-F | 2-CN | CH₂CH₂OH |
| CH₂ | 4-Cl | H | CH₂CH₂OH |
| CH₂ | 4-Cl | 3,5-F₂ | CH₂CH₂OH |
| CH₂ | 4-Cl | 4-F | CH₂CH₂OH |
| CH₂ | 4-Cl | 2,6-F₂ | CH₂CH₂OH |
| CH₂ | 4-F | H | CH₂CH₂OH |
| CH₂ | 4-F | 3,5-F₂ | CH₂CH₂OH |
| CH₂ | 4-F | 4-F | CH₂CH₂OH |
| CH₂ | 4-F | 2,6-F₂ | CH₂CH₂OH |
| O | 3-F | H | CH₂CH₂OH |
| O | 3-F | 3,5-F₂ | CH₂CH₂OH |
| O | 3-F | 4-F | CH₂CH₂OH |
| O | 3-F | 2,6-F₂ | CH₂CH₂OH |
| O | 3-F | 2,5-F₂ | CH₂CH₂OH |
| O | 2,5-F₂ | H | CH₂CH₂OH |
| O | 2,5-F₂ | 3,5-F₂ | CH₂CH₂OH |
| O | 2,5-F₂ | 4-F | CH₂CH₂OH |
| O | 2,5-F₂ | 2,6-F₂ | CH₂CH₂OH |
| O | 2,5-F₂ | 2,5-F₂ | CH₂CH₂OH |
| O | 3,5-F₂ | H | CH₂CH₂OH |
| O | 3,5-F₂ | 3,5-F₂ | CH₂CH₂OH |
| O | 3,5-F₂ | 4-F | CH₂CH₂OH |
| O | 3,5-F₂ | 2,6-F₂ | CH₂CH₂OH |
| O | 3,5-F₂ | 2,5-F₂ | CH₂CH₂OH |
| O | 3,4-F₂ | H | CH₂CH₂OH |
| O | 3,4-F₂ | 3,5-F₂ | CH₂CH₂OH |
| O | 3,4-F₂ | 4-F | CH₂CH₂OH |
| O | 3,4-F₂ | 2,6-F₂ | CH₂CH₂OH |
| O | 3,4-F₂ | 2,5-F₂ | CH₂CH₂OH |
| O | 4-CF₃ | H | CH₂CH₂OH |
| O | 4-CF₃ | 3,5-F₂ | CH₂CH₂OH |
| O | 4-CF₃ | 4-F | CH₂CH₂OH |
| O | 4-CF₃ | 2,6-F₂ | CH₂CH₂OH |
| O | 4-CF₃ | 2,5-F₂ | CH₂CH₂OH |
| O | 4-Cl | H | CH₂CF₃ |
| O | 4-F | H | CH₂CF₃ |
| CH₂ | 4-Cl | H | CH₂CF₃ |
| CH₂ | 4-F | H | CH₂CF₃ |
| O | 4-Cl | H | OH |
| O | 4-F | H | OH |
| CH₂ | 4-Cl | H | OH |
| CH₂ | 4-F | H | OH |
| O | 4-Cl | H | OCH₃ |
| O | 4-F | H | OCH₃ |
| CH₂ | 4-Cl | H | OCH₃ |
| CH₂ | 4-F | H | OCH₃ |

TABLE VI

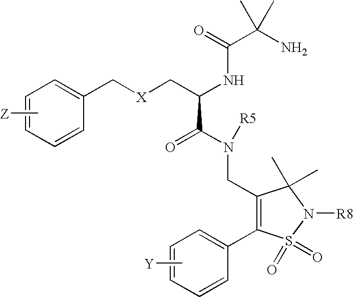

| X | Y | Z | R5 | R8 |
|---|---|---|---|---|
| O | 4-Cl | H | CH₃ | CH₂CH₂F |
| O | 4-F | H | CH₃ | CH₂CH₂F |
| O | 4-Cl | H | CH₂CH₃ | CH₂CH₂F |
| O | 4-F | H | CH₂CH₃ | CH₂CH₂F |
| CH₂ | 4-Cl | H | CH₃ | CH₂CH₂F |
| CH₂ | 4-F | H | CH₃ | CH₂CH₂F |
| CH₂ | 4-Cl | H | CH₂CH₃ | CH₂CH₂F |
| CH₂ | 4-F | H | CH₂CH₃ | CH₂CH₂F |
| O | 4-Cl | H | CH₃ | CH₂CH₂OH |
| O | 4-F | H | CH₃ | CH₂CH₂OH |
| O | 4-Cl | H | CH₂CH₃ | CH₂CH₂OH |
| O | 4-F | H | CH₂CH₃ | CH₂CH₂OH |
| CH₂ | 4-Cl | H | CH₃ | CH₂CH₂OH |
| CH₂ | 4-F | H | CH₃ | CH₂CH₂OH |
| CH₂ | 4-Cl | H | CH₂CH₃ | CH₂CH₂OH |
| CH₂ | 4-F | H | CH₂CH₃ | CH₂CH₂OH |
| O | 4-Cl | H | CH₃ | CH₂CF₃ |
| O | 4-F | H | CH₃ | CH₂CF₃ |
| O | 4-Cl | H | CH₂CH₃ | CH₂CF₃ |
| O | 4-F | H | CH₂CH₃ | CH₂CF₃ |
| CH₂ | 4-Cl | H | CH₃ | CH₂CF₃ |
| CH₂ | 4-F | H | CH₃ | CH₂CF₃ |
| CH₂ | 4-Cl | H | CH₂CH₃ | CH₂CF₃ |
| CH₂ | 4-F | H | CH₂CH₃ | CH₂CF₃ |
| O | 4-Cl | H | CH₃ | CH₂CH₂OCH₃ |
| O | 4-F | H | CH₃ | CH₂CH₂OCH₃ |
| O | 4-Cl | H | CH₂CH₃ | CH₂CH₂OCH₃ |
| O | 4-F | H | CH₂CH₃ | CH₂CH₂OCH₃ |
| CH₂ | 4-Cl | H | CH₃ | CH₂CH₂OCH₃ |
| CH₂ | 4-F | H | CH₃ | CH₂CH₂OCH₃ |
| CH₂ | 4-Cl | H | CH₂CH₃ | CH₂CH₂OCH₃ |
| CH₂ | 4-F | H | CH₂CH₃ | CH₂CH₂OCH₃ |
| O | 4-Cl | H | CH₃ | cyclopropylmethyl |
| O | 4-F | H | CH₃ | cyclopropylmethyl |
| O | 4-Cl | H | CH₂CH₃ | cyclopropylmethyl |
| O | 4-F | H | CH₂CH₃ | cyclopropylmethyl |
| CH₂ | 4-Cl | H | CH₃ | cyclopropylmethyl |
| CH₂ | 4-F | H | CH₃ | cyclopropylmethyl |
| CH₂ | 4-Cl | H | CH₂CH₃ | cyclopropylmethyl |
| CH₂ | 4-F | H | CH₂CH₃ | cyclopropylmethyl |
| O | 4-Cl | H | CH₃ | CH₂CH₂CH₂CF₃ |
| O | 4-F | H | CH₃ | CH₂CH₂CH₂CF₃ |
| O | 4-Cl | H | CH₂CH₃ | CH₂CH₂CH₂CF₃ |
| O | 4-F | H | CH₂CH₃ | CH₂CH₂CH₂CF₃ |
| CH₂ | 4-Cl | H | CH₃ | CH₂CH₂CH₂CF₃ |
| CH₂ | 4-F | H | CH₃ | CH₂CH₂CH₂CF₃ |
| CH₂ | 4-Cl | H | CH₂CH₃ | CH₂CH₂CH₂CF₃ |
| CH₂ | 4-F | H | CH₂CH₃ | CH₂CH₂CH₂CF₃ |
| O | 4-Cl | H | CH₃ | CH₂CH₂CF₃ |
| O | 4-F | H | CH₃ | CH₂CH₂CF₃ |
| O | 4-Cl | H | CH₂CH₃ | CH₂CH₂CF₃ |
| O | 4-F | H | CH₂CH₃ | CH₂CH₂CF₃ |
| CH₂ | 4-Cl | H | CH₃ | CH₂CH₂CF₃ |
| CH₂ | 4-F | H | CH₃ | CH₂CH₂CF₃ |
| CH₂ | 4-Cl | H | CH₂CH₃ | CH₂CH₂CF₃ |
| CH₂ | 4-F | H | CH₂CH₃ | CH₂CH₂CF₃ |
| O | 4-Cl | H | CH₃ | CH₂CONH₂ |
| O | 4-F | H | CH₃ | CH₂CONH₂ |
| O | 4-Cl | H | CH₂CH₃ | CH₂CONH₂ |
| O | 4-F | H | CH₂CH₃ | CH₂CONH₂ |
| CH₂ | 4-Cl | H | CH₃ | CH₂CONH₂ |
| CH₂ | 4-F | H | CH₃ | CH₂CONH₂ |
| CH₂ | 4-Cl | H | CH₂CH₃ | CH₂CONH₂ |
| CH₂ | 4-F | H | CH₂CH₃ | CH₂CONH₂ |
| O | 4-Cl | H | CH₃ | CH₂CON(CH₃)₂ |
| O | 4-F | H | CH₃ | CH₂CON(CH₃)₂ |
| O | 4-Cl | H | CH₂CH₃ | CH₂CON(CH₃)₂ |
| O | 4-F | H | CH₂CH₃ | CH₂CON(CH₃)₂ |
| CH₂ | 4-Cl | H | CH₃ | CH₂CON(CH₃)₂ |
| CH₂ | 4-F | H | CH₃ | CH₂CON(CH₃)₂ |
| CH₂ | 4-Cl | H | CH₂CH₃ | CH₂CON(CH₃)₂ |
| CH₂ | 4-F | H | CH₂CH₃ | CH₂CON(CH₃)₂ |

TABLE VII

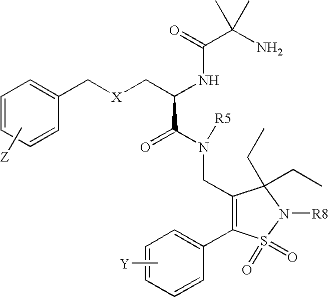

| X | Y | Z | R5 | R8 |
|---|---|---|---|---|
| O | 4-Cl | H | CH₃ | CH₂CH₂F |
| O | 4-F | H | CH₃ | CH₂CH₂F |
| O | 4-Cl | H | CH₂CH₃ | CH₂CH₂F |
| O | 4-F | H | CH₂CH₃ | CH₂CH₂F |
| CH₂ | 4-Cl | H | CH₃ | CH₂CH₂F |
| CH₂ | 4-F | H | CH₃ | CH₂CH₂F |
| CH₂ | 4-Cl | H | CH₂CH₃ | CH₂CH₂F |
| CH₂ | 4-F | H | CH₂CH₃ | CH₂CH₂F |
| O | 4-Cl | H | CH₃ | CH₂CH₂OH |
| O | 4-F | H | CH₃ | CH₂CH₂OH |
| O | 4-Cl | H | CH₂CH₃ | CH₂CH₂OH |
| O | 4-F | H | CH₂CH₃ | CH₂CH₂OH |
| CH₂ | 4-Cl | H | CH₃ | CH₂CH₂OH |
| CH₂ | 4-F | H | CH₃ | CH₂CH₂OH |
| CH₂ | 4-Cl | H | CH₂CH₃ | CH₂CH₂OH |
| CH₂ | 4-F | H | CH₂CH₃ | CH₂CH₂OH |
| O | 4-Cl | H | CH₃ | CH₂CF₃ |
| O | 4-F | H | CH₃ | CH₂CF₃ |
| O | 4-Cl | H | CH₂CH₃ | CH₂CF₃ |
| O | 4-F | H | CH₂CH₃ | CH₂CF₃ |
| CH₂ | 4-Cl | H | CH₃ | CH₂CF₃ |
| CH₂ | 4-F | H | CH₃ | CH₂CF₃ |
| CH₂ | 4-Cl | H | CH₂CH₃ | CH₂CF₃ |
| CH₂ | 4-F | H | CH₂CH₃ | CH₂CF₃ |
| O | 4-Cl | H | CH₃ | CH₂CH₂OCH₃ |
| O | 4-F | H | CH₃ | CH₂CH₂OCH₃ |
| O | 4-Cl | H | CH₂CH₃ | CH₂CH₂OCH₃ |
| O | 4-F | H | CH₂CH₃ | CH₂CH₂OCH₃ |
| CH₂ | 4-Cl | H | CH₃ | CH₂CH₂OCH₃ |
| CH₂ | 4-F | H | CH₃ | CH₂CH₂OCH₃ |
| CH₂ | 4-Cl | H | CH₂CH₃ | CH₂CH₂OCH₃ |
| CH₂ | 4-F | H | CH₂CH₃ | CH₂CH₂OCH₃ |
| O | 4-Cl | H | CH₃ | cyclopropylmethyl |
| O | 4-F | H | CH₃ | cyclopropylmethyl |
| O | 4-Cl | H | CH₂CH₃ | cyclopropylmethyl |
| O | 4-F | H | CH₂CH₃ | cyclopropylmethyl |
| CH₂ | 4-Cl | H | CH₃ | cyclopropylmethyl |
| CH₂ | 4-F | H | CH₃ | cyclopropylmethyl |
| CH₂ | 4-Cl | H | CH₂CH₃ | cyclopropylmethyl |
| CH₂ | 4-F | H | CH₂CH₃ | cyclopropylmethyl |
| O | 4-Cl | H | CH₃ | CH₂CH₂CH₂CF₃ |
| O | 4-F | H | CH₃ | CH₂CH₂CH₂CF₃ |
| O | 4-Cl | H | CH₂CH₃ | CH₂CH₂CH₂CF₃ |
| O | 4-F | H | CH₂CH₃ | CH₂CH₂CH₂CF₃ |
| CH₂ | 4-Cl | H | CH₃ | CH₂CH₂CH₂CF₃ |
| CH₂ | 4-F | H | CH₃ | CH₂CH₂CH₂CF₃ |
| CH₂ | 4-Cl | H | CH₂CH₃ | CH₂CH₂CH₂CF₃ |
| CH₂ | 4-F | H | CH₂CH₃ | CH₂CH₂CH₂CF₃ |
| O | 4-Cl | H | CH₃ | CH₂CH₂CF₃ |
| O | 4-F | H | CH₃ | CH₂CH₂CF₃ |
| O | 4-Cl | H | CH₂CH₃ | CH₂CH₂CF₃ |
| O | 4-F | H | CH₂CH₃ | CH₂CH₂CF₃ |
| CH₂ | 4-Cl | H | CH₃ | CH₂CH₂CF₃ |
| CH₂ | 4-F | H | CH₃ | CH₂CH₂CF₃ |
| CH₂ | 4-Cl | H | CH₂CH₃ | CH₂CH₂CF₃ |
| CH₂ | 4-F | H | CH₂CH₃ | CH₂CH₂CF₃ |
| O | 4-Cl | H | CH₃ | CH₂CONH₂ |
| O | 4-F | H | CH₃ | CH₂CONH₂ |
| O | 4-Cl | H | CH₂CH₃ | CH₂CONH₂ |
| O | 4-F | H | CH₂CH₃ | CH₂CONH₂ |
| CH₂ | 4-Cl | H | CH₃ | CH₂CONH₂ |
| CH₂ | 4-F | H | CH₃ | CH₂CONH₂ |
| CH₂ | 4-Cl | H | CH₂CH₃ | CH₂CONH₂ |
| CH₂ | 4-F | H | CH₂CH₃ | CH₂CONH₂ |
| O | 4-Cl | H | CH₃ | CH₂CON(CH₃)₂ |
| O | 4-F | H | CH₃ | CH₂CON(CH₃)₂ |
| O | 4-Cl | H | CH₂CH₃ | CH₂CON(CH₃)₂ |
| O | 4-F | H | CH₂CH₃ | CH₂CON(CH₃)₂ |
| CH₂ | 4-Cl | H | CH₃ | CH₂CON(CH₃)₂ |
| CH₂ | 4-F | H | CH₃ | CH₂CON(CH₃)₂ |
| CH₂ | 4-Cl | H | CH₂CH₃ | CH₂CON(CH₃)₂ |
| CH₂ | 4-F | H | CH₂CH₃ | CH₂CON(CH₃)₂ |

TABLE VIII

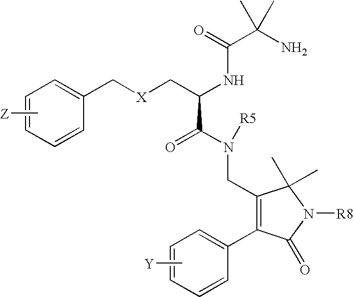

| X | Y | Z | R5 | R8 |
|---|---|---|---|---|
| O | 4-Cl | H | CH₃ | CH₂CH₂F |
| O | 4-F | H | CH₃ | CH₂CH₂F |
| O | 4-Cl | H | CH₂CH₃ | CH₂CH₂F |
| O | 4-F | H | CH₂CH₃ | CH₂CH₂F |
| CH₂ | 4-Cl | H | CH₃ | CH₂CH₂F |
| CH₂ | 4-F | H | CH₃ | CH₂CH₂F |
| CH₂ | 4-Cl | H | CH₂CH₃ | CH₂CH₂F |
| CH₂ | 4-F | H | CH₂CH₃ | CH₂CH₂F |
| O | 4-Cl | H | CH₃ | CH₂CH₂OH |
| O | 4-F | H | CH₃ | CH₂CH₂OH |
| O | 4-Cl | H | CH₂CH₃ | CH₂CH₂OH |
| O | 4-F | H | CH₂CH₃ | CH₂CH₂OH |
| CH₂ | 4-Cl | H | CH₃ | CH₂CH₂OH |
| CH₂ | 4-F | H | CH₃ | CH₂CH₂OH |
| CH₂ | 4-Cl | H | CH₂CH₃ | CH₂CH₂OH |
| CH₂ | 4-F | H | CH₂CH₃ | CH₂CH₂OH |
| O | 4-Cl | H | CH₃ | CH₂CF₃ |
| O | 4-F | H | CH₃ | CH₂CF₃ |
| O | 4-Cl | H | CH₂CH₃ | CH₂CF₃ |
| O | 4-F | H | CH₂CH₃ | CH₂CF₃ |
| CH₂ | 4-Cl | H | CH₃ | CH₂CF₃ |
| CH₂ | 4-F | H | CH₃ | CH₂CF₃ |
| CH₂ | 4-Cl | H | CH₂CH₃ | CH₂CF₃ |
| CH₂ | 4-F | H | CH₂CH₃ | CH₂CF₃ |
| O | 4-Cl | H | CH₃ | CH₂CH₂OCH₃ |
| O | 4-F | H | CH₃ | CH₂CH₂OCH₃ |
| O | 4-Cl | H | CH₂CH₃ | CH₂CH₂OCH₃ |
| O | 4-F | H | CH₂CH₃ | CH₂CH₂OCH₃ |
| CH₂ | 4-Cl | H | CH₃ | CH₂CH₂OCH₃ |
| CH₂ | 4-F | H | CH₃ | CH₂CH₂OCH₃ |
| CH₂ | 4-Cl | H | CH₂CH₃ | CH₂CH₂OCH₃ |
| CH₂ | 4-F | H | CH₂CH₃ | CH₂CH₂OCH₃ |
| O | 4-Cl | H | CH₃ | cyclopropylmethyl |
| O | 4-F | H | CH₃ | cyclopropylmethyl |
| O | 4-Cl | H | CH₂CH₃ | cyclopropylmethyl |
| O | 4-F | H | CH₂CH₃ | cyclopropylmeethyl |
| CH₂ | 4-Cl | H | CH₃ | cyclopropylmethyl |
| CH₂ | 4-F | H | CH₃ | cyclopropylmethyl |
| CH₂ | 4-Cl | H | CH₂CH₃ | cyclopropylmethyl |
| CH₂ | 4-F | H | CH₂CH₃ | cyclopropylmethyl |
| O | 4-Cl | H | CH₃ | CH₂CH₂CH₂CF₃ |
| O | 4-F | H | CH₃ | CH₂CH₂CH₂CF₃ |
| O | 4-Cl | H | CH₂CH₃ | CH₂CH₂CH₂CF₃ |
| O | 4-F | H | CH₂CH₃ | CH₂CH₂CH₂CF₃ |
| CH₂ | 4-Cl | H | CH₃ | CH₂CH₂CH₂CF₃ |
| CH₂ | 4-F | H | CH₃ | CH₂CH₂CH₂CF₃ |
| CH₂ | 4-Cl | H | CH₂CH₃ | CH₂CH₂CH₂CF₃ |
| CH₂ | 4-F | H | CH₂CH₃ | CH₂CH₂CH₂CF₃ |
| O | 4-Cl | H | CH₃ | CH₂CH₂CF₃ |
| O | 4-F | H | CH₃ | CH₂CH₂CF₃ |
| O | 4-Cl | H | CH₂CH₃ | CH₂CH₂CF₃ |
| O | 4-F | H | CH₂CH₃ | CH₂CH₂CF₃ |
| CH₂ | 4-Cl | H | CH₃ | CH₂CH₂CF₃ |
| CH₂ | 4-F | H | CH₃ | CH₂CH₂CF₃ |
| CH₂ | 4-Cl | H | CH₂CH₃ | CH₂CH₂CF₃ |
| CH₂ | 4-F | H | CH₂CH₃ | CH₂CH₂CF₃ |
| O | 4-Cl | H | CH₃ | CH₂CONH₂ |
| O | 4-F | H | CH₃ | CH₂CONH₂ |
| O | 4-Cl | H | CH₂CH₃ | CH₂CONH₂ |
| O | 4-F | H | CH₂CH₃ | CH₂CONH₂ |
| CH₂ | 4-Cl | H | CH₃ | CH₂CONH₂ |
| CH₂ | 4-F | H | CH₃ | CH₂CONH₂ |
| CH₂ | 4-Cl | H | CH₂CH₃ | CH₂CONH₂ |
| CH₂ | 4-F | H | CH₂CH₃ | CH₂CONH₂ |
| O | 4-Cl | H | CH₃ | CH₂CON(CH₃)₂ |
| O | 4-F | H | CH₃ | CH₂CON(CH₃)₂ |
| O | 4-Cl | H | CH₂CH₃ | CH₂CON(CH₃)₂ |
| O | 4-F | H | CH₂CH₃ | CH₂CON(CH₃)₂ |
| CH₂ | 4-Cl | H | CH₃ | CH₂CON(CH₃)₂ |
| CH₂ | 4-F | N | CH₃ | CH₂CON(CH₃)₂ |
| CH₂ | 4-Cl | H | CH₂CH₃ | CH₂CON(CH₃)₂ |
| CH₂ | 4-F | H | CH₂CH₃ | CH₂CON(CH₃)₂ |

TABLE IX

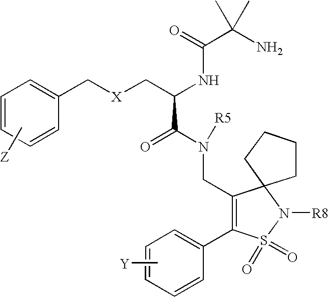

| X | Y | Z | R5 | R8 |
|---|---|---|---|---|
| O | 4-Cl | H | CH₃ | CH₂CH₂F |
| O | 4-F | H | CH₃ | CH₂CH₂F |
| O | 4-Cl | H | CH₂CH₃ | CH₂CH₂F |
| O | 4-F | H | CH₂CH₃ | CH₂CH₂F |
| CH₂ | 4-Cl | H | CH₃ | CH₂CH₂F |
| CH₂ | 4-F | H | CH₃ | CH₂CH₂F |
| CH₂ | 4-Cl | H | CH₂CH₃ | CH₂CH₂F |
| CH₂ | 4-F | H | CH₂CH₃ | CH₂CH₂F |
| O | 4-Cl | H | CH₃ | CH₂CH₂OH |
| O | 4-F | H | CH₃ | CH₂CH₂OH |
| O | 4-Cl | H | CH₂CH₃ | CH₂CH₂OH |
| O | 4-F | H | CH₂CH₃ | CH₂CH₂OH |
| CH₂ | 4-Cl | H | CH₃ | CH₂CH₂OH |
| CH₂ | 4-F | H | CH₃ | CH₂CH₂OH |
| CH₂ | 4-Cl | H | CH₂CH₃ | CH₂CH₂OH |
| CH₂ | 4-F | H | CH₂CH₃ | CH₂CH₂OH |
| O | 4-Cl | H | CH₃ | CH₂CF₃ |
| O | 4-F | H | CH₃ | CH₂CF₃ |
| O | 4-Cl | H | CH₂CH₃ | CH₂CF₃ |
| O | 4-F | H | CH₂CH₃ | CH₂CF₃ |
| CH₂ | 4-Cl | H | CH₃ | CH₂CF₃ |
| CH₂ | 4-F | H | CH₃ | CH₂CF₃ |
| CH₂ | 4-Cl | H | CH₂CH₃ | CH₂CF₃ |
| CH₂ | 4-F | H | CH₂CH₃ | CH₂CF₃ |
| O | 4-Cl | H | CH₃ | CH₂CH₂OCH₃ |
| O | 4-F | H | CH₃ | CH₂CH₂OCH₃ |
| O | 4-Cl | H | CH₂CH₃ | CH₂CH₂OCH₃ |
| O | 4-F | H | CH₂CH₃ | CH₂CH₂OCH₃ |
| CH₂ | 4-Cl | H | CH₃ | CH₂CH₂OCH₃ |
| CH₂ | 4-F | H | CH₃ | CH₂CH₂OCH₃ |
| CH₂ | 4-Cl | H | CH₂CH₃ | CH₂CH₂OCH₃ |
| CH₂ | 4-F | H | CH₂CH₃ | CH₂CH₂OCH₃ |
| O | 4-Cl | H | CH₃ | cyclopropylmethyl |
| O | 4-F | H | CH₃ | cyclopropylmethyl |
| O | 4-Cl | H | CH₂CH₃ | cyclopropylmethyl |
| O | 4-F | H | CH₂CH₃ | cyclopropylmethyl |
| CH₂ | 4-Cl | H | CH₃ | cyclopropylmethyl |
| CH₂ | 4-F | H | CH₃ | cyclopropylmethyl |
| CH₂ | 4-Cl | H | CH₂CH₃ | cyclopropylmethyl |
| CH₂ | 4-F | H | CH₂CH₃ | cyclopropylmethyl |
| O | 4-Cl | H | CH₃ | CH₂CH₂CH₂CF₃ |
| O | 4-F | H | CH₃ | CH₂CH₂CH₂CF₃ |
| O | 4-Cl | H | CH₂CH₃ | CH₂CH₂CH₂CF₃ |
| O | 4-F | H | CH₂CH₃ | CH₂CH₂CH₂CF₃ |
| CH₂ | 4-Cl | H | CH₃ | CH₂CH₂CH₂CF₃ |
| CH₂ | 4-F | H | CH₂ | CH₂CH₂CH₂CF₃ |
| CH₂ | 4-Cl | H | CH₂CH₃ | CH₂CH₂CH₂CF₃ |
| CH₂ | 4-F | H | CH₂CH₃ | CH₂CH₂CH₂CF₃ |
| O | 4-Cl | H | CH₃ | CH₂CH₂CF₃ |
| O | 4-F | H | CH₃ | CH₂CH₂CF₃ |
| O | 4-Cl | H | CH₂CH₃ | CH₂CH₂CF₃ |
| O | 4-F | H | CH₂CH₃ | CH₂CH₂CF₃ |
| CH₂ | 4-Cl | H | CH₃ | CH₂CH₂CF₃ |
| CH₂ | 4-F | H | CH₃ | CH₂CH₂CF₃ |
| CH₂ | 4-Cl | H | CH₂CH₃ | CH₂CH₂CF₃ |
| CH₂ | 4-F | H | CH₂CH₃ | CH₂CH₂CF₃ |
| O | 4-Cl | H | CH₃ | CH₂CONH₂ |
| O | 4-F | H | CH₃ | CH₂CONH₂ |
| O | 4-Cl | H | CH₂CH₃ | CH₂CONH₂ |
| O | 4-F | H | CH₂CH₃ | CH₂CONH₂ |
| CH₂ | 4-Cl | H | CH₃ | CH₂CONH₂ |
| CH₂ | 4-F | H | CH₃ | CH₂CONH₂ |
| CH₂ | 4-Cl | H | CH₂CH₃ | CH₂CONH₂ |
| CH₂ | 4-F | H | CH₂CH₃ | CH₂CONH₂ |
| O | 4-Cl | H | CH₃ | CH₂CON(CH₃)₂ |
| O | 4-F | H | CH₃ | CH₂CON(CH₃)₂ |
| O | 4-Cl | H | CH₂CH₃ | CH₂CON(CH₃)₂ |
| O | 4-F | H | CH₂CH₃ | CH₂CON(CH₃)₂ |
| CH₂ | 4-Cl | H | CH₃ | CH₂CON(CH₃)₂ |
| CH₂ | 4-F | H | CH₃ | CH₂CON(CH₃)₂ |
| CH₂ | 4-Cl | H | CH₂CH₃ | CH₂CON(CH₃)₂ |
| CH₂ | 4-F | H | CH₂CH₃ | CH₂CON(CH₃)₂ |

TABLE X

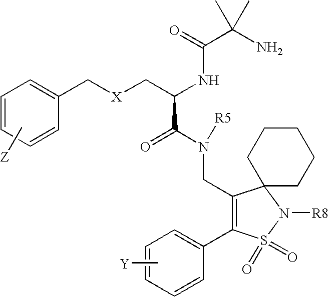

| X | Y | Z | R5 | R8 |
|---|---|---|---|---|
| O | 4-Cl | H | CH₃ | CH₂CH₂F |
| O | 4-F | H | CH₃ | CH₂CH₂F |
| O | 4-Cl | H | CH₂CH₃ | CH₂CH₂F |
| O | 4-F | H | CH₂CH₃ | CH₂CH₂F |
| CH₂ | 4-Cl | H | CH₃ | CH₂CH₂F |
| CH₂ | 4-F | H | CH₃ | CH₂CH₂F |
| CH₂ | 4-Cl | H | CH₂CH₃ | CH₂CH₂F |
| CH₂ | 4-F | H | CH₂CH₃ | CH₂CH₂F |
| O | 4-Cl | H | CH₃ | CH₂CH₂OH |
| O | 4-F | H | CH₃ | CH₂CH₂OH |
| O | 4-Cl | H | CH₂CH₃ | CH₂CH₂OH |
| O | 4-F | H | CH₂CH₃ | CH₂CH₂OH |
| CH₂ | 4-Cl | H | CH₃ | CH₂CH₂OH |
| CH₂ | 4-F | H | CH₃ | CH₂CH₂OH |
| CH₂ | 4-Cl | H | CH₂CH₃ | CH₂CH₂OH |
| CH₂ | 4-F | H | CH₂CH₃ | CH₂CH₂OH |
| O | 4-Cl | H | CH₃ | CH₂CF₃ |
| O | 4-F | H | CH₃ | CH₂CF₃ |
| O | 4-Cl | H | CH₂CH₃ | CH₂CF₃ |
| O | 4-F | H | CH₂CH₃ | CH₂CF₃ |
| CH₂ | 4-Cl | H | CH₃ | CH₂CF₃ |
| CH₂ | 4-F | H | CH₃ | CH₂CF₃ |
| CH₂ | 4-Cl | H | CH₂CH₃ | CH₂CF₃ |
| CH₂ | 4-F | H | CH₂CH₃ | CH₂CF₃ |
| O | 4-Cl | H | CH₃ | CH₂CH₂OCH₃ |
| O | 4-F | H | CH₃ | CH₂CH₂OCH₃ |
| O | 4-Cl | H | CH₂CH₃ | CH₂CH₂OCH₃ |
| O | 4-F | H | CH₂CH₃ | CH₂CH₂OCH₃ |
| CH₂ | 4-Cl | H | CH₃ | CH₂CH₂OCH₃ |
| CH₂ | 4-F | H | CH₃ | CH₂CH₂OCH₃ |
| CH₂ | 4-Cl | H | CH₂CH₃ | CH₂CH₂OCH₃ |
| CH₂ | 4-F | H | CH₂CH₃ | CH₂CH₂OCH₃ |
| O | 4-Cl | H | CH₃ | cyclopropylmethyl |
| O | 4-F | H | CH₃ | cyclopropylmethyl |
| O | 4-Cl | H | CH₂CH₃ | cyclopropylmethyl |
| O | 4-F | H | CH₂CH₃ | cyclopropylmethyl |
| CH₂ | 4-Cl | H | CH₃ | cyclopropylmethyl |
| CH₂ | 4-F | H | CH₃ | cyclopropylmethyl |
| CH₂ | 4-Cl | H | CH₂CH₃ | cyclopropylmethyl |
| CH₂ | 4-F | H | CH₂CH₃ | cyclopropylmethyl |
| O | 4-Cl | H | CH₃ | CH₂CH₂CH₂CF₃ |
| O | 4-F | H | CH₃ | CH₂CH₂CH₂CF₃ |
| O | 4-Cl | H | CH₂CH₃ | CH₂CH₂CH₂CF₃ |
| O | 4-F | H | CH₂CH₃ | CH₂CH₂CH₂CF₃ |
| CH₂ | 4-Cl | H | CH₃ | CH₂CH₂CH₂CF₃ |
| CH₂ | 4-F | H | CH₃ | CH₂CH₂CH₂CF₃ |
| CH₂ | 4-Cl | H | CH₂CH₃ | CH₂CH₂CH₂CF₃ |
| CH₂ | 4-F | H | CH₂CH₃ | CH₂CH₂CH₂CF₃ |
| O | 4-Cl | H | CH₃ | CH₂CH₂CF₃ |
| O | 4-F | H | CH₃ | CH₂CH₂CF₃ |
| O | 4-Cl | H | CH₂CH₃ | CH₂CH₂CF₃ |
| O | 4-F | H | CH₂CH₃ | CH₂CH₂CF₃ |
| CH₂ | 4-Cl | H | CH₃ | CH₂CH₂CF₃ |
| CH₂ | 4-F | H | CH₃ | CH₂CH₂CF₃ |
| CH₂ | 4-Cl | H | CH₂CH₃ | CH₂CH₂CF₃ |
| CH₂ | 4-F | H | CH₂CH₃ | CH₂CH₂CF₃ |
| O | 4-Cl | H | CH₃ | CH₂CONH₂ |
| O | 4-F | H | CH₃ | CH₂CONH₂ |
| O | 4-Cl | H | CH₂CH₃ | CH₂CONH₂ |
| O | 4-F | H | CH₂CH₃ | CH₂CONH₂ |
| CH₂ | 4-Cl | H | CH₃ | CH₂CONH₂ |
| CH₂ | 4-F | H | CH₃ | CH₂CONH₂ |
| CH₂ | 4-Cl | H | CH₂CH₃ | CH₂CONH₂ |
| CH₂ | 4-F | H | CH₂CH₃ | CH₂CONH₂ |
| O | 4-Cl | H | CH₃ | CH₂CON(CH₃)₂ |
| O | 4-F | H | CH₃ | CH₂CON(CH₃)₂ |
| O | 4-Cl | H | CH₂CH₃ | CH₂CON(CH₃)₂ |
| O | 4-F | H | CH₂CH₃ | CH₂CON(CH₃)₂ |
| CH₂ | 4-Cl | H | CH₃ | CH₂CON(CH₃)₂ |
| CH₂ | 4-F | H | CH₃ | CH₂CON(CH₃)₂ |
| CH₂ | 4-Cl | H | CH₂CH₃ | CH₂CON(CH₃)₂ |
| CH₂ | 4-F | H | CH₂CH₃ | CH₂CON(CH₃)₂ |

TABLE XI

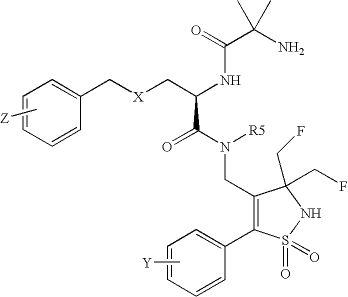

| X | Y | Z | R5 |
|---|---|---|---|
| O | 4-Cl | H | CH₂CH₃ |
| O | 4-Cl | 2-F | CH₂CH₃ |
| O | 4-Cl | 3-F | CH₂CH₃ |
| O | 4-Cl | 4-F | CH₂CH₃ |
| O | 4-Cl | 4-Cl | CH₂CH₃ |
| O | 4-Cl | 2,5-F₂ | CH₂CH₃ |
| O | 4-Cl | 2,4-F₂ | CH₂CH₃ |
| O | 4-Cl | 2-Cl | CH₂CH₃ |
| O | 4-Cl | 2,6-F₂ | CH₂CH₃ |
| O | 4-Cl | 3,5-F₂ | CH₂CH₃ |
| O | 4-Cl | 2,3-F₂ | CH₂CH₃ |
| O | 4-Cl | 3,4-F₂ | CH₂CH₃ |
| O | 4-Cl | 2,3,5-F₃ | CH₂CH₃ |
| O | 4-Cl | 2,3,6-F₃ | CH₂CH₃ |
| O | 4-Cl | 2,4,5-F₃ | CH₂CH₃ |
| O | 4-Cl | 2,6-Cl₂ | CH₂CH₃ |
| O | 4-Cl | 2-F-6-Cl | CH₂CH₃ |
| O | 4-Cl | 2-F-3-Cl | CH₂CH₃ |
| O | 4-Cl | 4-F-2-Cl | CH₂CH₃ |
| O | 4-Cl | 2-Cl-3,6-F₂ | CH₂CH₃ |
| O | 4-Cl | 3-Cl-2,6-F₂ | CH₂CH₃ |
| O | 4-Cl | 3-F-4-Cl | CH₂CH₃ |
| O | 4-Cl | 2-F-4-Cl | CH₂CH₃ |
| O | 4-Cl | 2,3-Cl₂ | CH₂CH₃ |
| O | 4-Cl | 2-CN | CH₂CH₃ |
| O | 4-F | H | CH₂CH₃ |
| O | 4-F | 2-F | CH₂CH₃ |
| O | 4-F | 3-F | CH₂CH₃ |
| O | 4-F | 4-F | CH₂CH₃ |
| O | 4-F | 4-Cl | CH₂CH₃ |
| O | 4-F | 2,5-F₂ | CH₂CH₃ |
| O | 4-F | 2,4-F₂ | CH₂CH₃ |
| O | 4-F | 2-Cl | CH₂CH₃ |
| O | 4-F | 2,6-F₂ | CH₂CH₃ |
| O | 4-F | 3,5-F₂ | CH₂CH₃ |
| O | 4-F | 2,3-F₂ | CH₂CH₃ |
| O | 4-F | 3,4-F₂ | CH₂CH₃ |
| O | 4-F | 2,3,5-F₃ | CH₂CH₃ |
| O | 4-F | 2,3,6-F₃ | CH₂CH₃ |
| O | 4-F | 2,4,5-F₃ | CH₂CH₃ |
| O | 4-F | 2,6-Cl₂ | CH₂CH₃ |
| O | 4-F | 2-F-6-Cl | CH₂CH₃ |
| O | 4-F | 2-F-3-Cl | CH₂CH₃ |
| O | 4-F | 4-F-2-Cl | CH₂CH₃ |
| O | 4-F | 2-Cl-3,6-F₂ | CH₂CH₃ |
| O | 4-F | 3-Cl-2,6-F₂ | CH₂CH₃ |
| O | 4-F | 3-F-4-Cl | CH₂CH₃ |
| O | 4-F | 2-F-4-Cl | CH₂CH₃ |
| O | 4-F | 2,3-Cl₂ | CH₂CH₃ |
| O | 4-F | 2-CN | CH₂CH₃ |
| CH₂ | 4-Cl | H | CH₂CH₃ |
| CH₂ | 4-Cl | 3,5-F₂ | CH₂CH₃ |
| CH₂ | 4-Cl | 4-F | CH₂CH₃ |
| CH₂ | 4-Cl | 2,6-F₂ | CH₂CH₃ |
| CH₂ | 4-F | H | CH₂CH₃ |
| CH₂ | 4-F | 3,5-F₂ | CH₂CH₃ |
| CH₂ | 4-F | 4-F | CH₂CH₃ |
| CH₂ | 4-F | 2,6-F₂ | CH₂CH₃ |
| O | 3-Cl | H | CH₂CH₃ |
| O | 3-Cl | 3,5-F₂ | CH₂CH₃ |
| O | 3-Cl | 4-F | CH₂CH₃ |
| O | 3-Cl | 2,6-F₂ | CH₂CH₃ |
| O | 3-Cl | 2,5-F₂ | CH₂CH₃ |
| O | 3-F | H | CH₂CH₃ |
| O | 3-F | 3,5-F₂ | CH₂CH₃ |
| O | 3-F | 4-F | CH₂CH₃ |
| O | 3-F | 2,6-F₂ | CH₂CH₃ |
| O | 3-F | 2,5-F₂ | CH₂CH₃ |
| O | 4-CN | H | CH₂CH₃ |
| O | 4-CN | 3,5-F₂ | CH₂CH₃ |
| O | 4-CN | 4-F | CH₂CH₃ |
| O | 4-CN | 2,6-F₂ | CH₂CH₃ |
| O | 4-CN | 2,5-F₂ | CH₂CH₃ |
| O | 2,5-F₂ | H | CH₂CH₃ |
| O | 2,5-F₂ | 3,5-F₂ | CH₂CH₃ |
| O | 2,5-F₂ | 4-F | CH₂CH₃ |
| O | 2,5-F₂ | 2,6-F₂ | CH₂CH₃ |
| O | 2,5-F₂ | 2,5-F₂ | CH₂CH₃ |
| O | 3,5-F₂ | H | CH₂CH₃ |
| O | 3,5-F₂ | 3,5-F₂ | CH₂CH₃ |
| O | 3,5-F₂ | 4-F | CH₂CH₃ |
| O | 3,5-F₂ | 2,6-F₂ | CH₂CH₃ |
| O | 3,5-F₂ | 2,5-F₂ | CH₂CH₃ |
| O | 3,4-F₂ | H | CH₂CH₃ |
| O | 3,4-F₂ | 3,5-F₂ | CH₂CH₃ |
| O | 3,4-F₂ | 4-F | CH₂CH₃ |
| O | 3,4-F₂ | 2,6-F₂ | CH₂CH₃ |
| O | 3,4-F₂ | 2,5-F₂ | CH₂CH₃ |
| O | 4-CF₃ | H | CH₂CH₃ |
| O | 4-CF₃ | 3,5-F₂ | CH₂CH₃ |
| O | 4-CF₃ | 4-F | CH₂CH₃ |
| O | 4-CF₃ | 2,6-F₂ | CH₂CH₃ |
| O | 4-CF₃ | 2,5-F₂ | CH₂CH₃ |
| O | 4-Cl | H | CH₃ |
| O | 4-Cl | 2-F | CH₃ |
| O | 4-Cl | 3-F | CH₃ |
| O | 4-Cl | 4-F | CH₃ |
| O | 4-Cl | 4-Cl | CH₃ |
| O | 4-Cl | 2,5-F₂ | CH₃ |
| O | 4-Cl | 2,4-F₂ | CH₃ |
| O | 4-Cl | 2-Cl | CH₃ |
| O | 4-Cl | 2,6-F₂ | CH₃ |
| O | 4-Cl | 3,5-F₂ | CH₃ |
| O | 4-Cl | 2,3-F₂ | CH₃ |
| O | 4-Cl | 3,4-F₂ | CH₃ |
| O | 4-Cl | 2,3,5-F₃ | CH₃ |
| O | 4-Cl | 2,3,6-F₃ | CH₃ |
| O | 4-Cl | 2,4,5-F₃ | CH₃ |
| O | 4-Cl | 2,6-Cl₂ | CH₃ |
| O | 4-Cl | 2-F-6-Cl | CH₃ |
| O | 4-Cl | 2-F-3-Cl | CH₃ |
| O | 4-Cl | 4-F-2-Cl | CH₃ |
| O | 4-Cl | 2-Cl-3,6-F₂ | CH₃ |
| O | 4-Cl | 3-Cl-2,6-F₂ | CH₃ |
| O | 4-Cl | 3-F-4-Cl | CH₃ |
| O | 4-Cl | 2-F-4-Cl | CH₃ |
| O | 4-Cl | 2,3-Cl₂ | CH₃ |
| O | 4-Cl | 2-CN | CH₃ |
| O | 4-F | H | CH₃ |
| O | 4-F | 2-F | CH₃ |
| O | 4-F | 3-F | CH₃ |
| O | 4-F | 4-F | CH₃ |
| O | 4-F | 4-Cl | CH₃ |
| O | 4-F | 2,5-F₂ | CH₃ |
| O | 4-F | 2,4-F₂ | CH₃ |
| O | 4-F | 2-Cl | CH₃ |
| O | 4-F | 2,6-F₂ | CH₃ |
| O | 4-F | 3,5-F₂ | CH₃ |
| O | 4-F | 2,3-F₂ | CH₃ |
| O | 4-F | 3,4-F₂ | CH₃ |
| O | 4-F | 2,3,5-F₃ | CH₃ |
| O | 4-F | 2,3,6-F₃ | CH₃ |
| O | 4-F | 2,4,5-F₃ | CH₃ |
| O | 4-F | 2,6-Cl₂ | CH₃ |
| O | 4-F | 2-F-6-Cl | CH₃ |
| O | 4-F | 2-F-3-Cl | CH₃ |
| O | 4-F | 4-F-2-Cl | CH₃ |
| O | 4-F | 2-Cl-3,6-F₂ | CH₃ |
| O | 4-F | 3-Cl-2,6-F₂ | CH₃ |
| O | 4-F | 3-F-4-Cl | CH₃ |
| O | 4-F | 2-F-4-Cl | CH₃ |
| O | 4-F | 2,3-Cl₂ | CH₃ |
| O | 4-F | 2-CN | CH₃ |
| CH₂ | 4-Cl | H | CH₃ |
| CH₂ | 4-Cl | 3,5-F₂ | CH₃ |
| CH₂ | 4-Cl | 4-F | CH₃ |
| CH₂ | 4-Cl | 2,6-F₂ | CH₃ |
| CH₂ | 4-F | H | CH₃ |
| CH₂ | 4-F | 3,5-F₂ | CH₃ |
| CH₂ | 4-F | 4-F | CH₃ |
| CH₂ | 4-F | 2,6-F₂ | CH₃ |
| O | 3-F | H | CH₃ |
| O | 3-F | 3,5-F₂ | CH₃ |
| O | 3-F | 4-F | CH₃ |
| O | 3-F | 2,6-F₂ | CH₃ |
| O | 3-F | 2,5-F₂ | CH₃ |
| O | 2,5-F₂ | H | CH₃ |
| O | 2,5-F₂ | 3,5-F₂ | CH₃ |
| O | 2,5-F₂ | 4-F | CH₃ |
| O | 2,5-F₂ | 2,6-F₂ | CH₃ |
| O | 2,5-F₂ | 2,5-F₂ | CH₃ |
| O | 3,5-F₂ | H | CH₃ |
| O | 3,5-F₂ | 3,5-F₂ | CH₃ |
| O | 3,5-F₂ | 4-F | CH₃ |
| O | 3,5-F₂ | 2,6-F₂ | CH₃ |
| O | 3,5-F₂ | 2,5-F₂ | CH₃ |
| O | 3,4-F₂ | H | CH₃ |
| O | 3,4-F₂ | 3,5-F₂ | CH₃ |
| O | 3,4-F₂ | 4-F | CH₃ |
| O | 3,4-F₂ | 2,6-F₂ | CH₃ |
| O | 3,4-F₂ | 2,5-F₂ | CH₃ |
| O | 4-CF₃ | H | CH₃ |
| O | 4-CF₃ | 3,5-F₂ | CH₃ |
| O | 4-CF₃ | 4-F | CH₃ |
| O | 4-CF₃ | 2,6-F₂ | CH₃ |
| O | 4-CF₃ | 2,5-F₂ | CH₃ |

TABLE XII

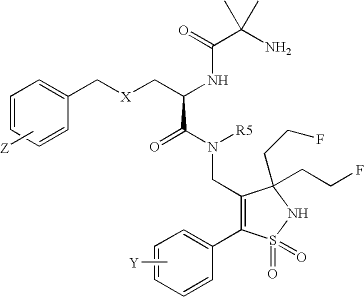

| X | Y | Z | R5 |
|---|---|---|---|
| O | 4-Cl | H | CH₂CH₃ |
| O | 4-Cl | 2-F | CH₂CH₃ |
| O | 4-Cl | 3-F | CH₂CH₃ |
| O | 4-Cl | 4-F | CH₂CH₃ |
| O | 4-Cl | 4-Cl | CH₂CH₃ |
| O | 4-Cl | 2,5-F₂ | CH₂CH₃ |
| O | 4-Cl | 2,4-F₂ | CH₂CH₃ |
| O | 4-Cl | 2-Cl | CH₂CH₃ |
| O | 4-Cl | 2,6-F₂ | CH₂CH₃ |
| O | 4-Cl | 3,5-F₂ | CH₂CH₃ |
| O | 4-Cl | 2,3-F₂ | CH₂CH₃ |
| O | 4-Cl | 3,4-F₂ | CH₂CH₃ |
| O | 4-Cl | 2,3,5-F₃ | CH₂CH₃ |
| O | 4-Cl | 2,3,6-F₃ | CH₂CH₃ |
| O | 4-Cl | 2,4,5-F₃ | CH₂CH₃ |
| O | 4-F | 2,5-F₂ | CH₂CH₃ |
| O | 4-F | 2,4-F₂ | CH₂CH₃ |
| O | 4-F | 2-Cl | CH₂CH₃ |
| O | 4-F | 2,6-F₂ | CH₂CH₃ |
| O | 4-F | 3,5-F₂ | CH₂CH₃ |
| O | 4-F | 2,3-F₂ | CH₂CH₃ |
| O | 4-F | 3,4-F₂ | CH₂CH₃ |
| O | 4-F | 2,3,5-F₃ | CH₂CH₃ |
| O | 4-F | 2,3,6-F₃ | CH₂CH₃ |
| O | 4-F | 2,4,5-F₃ | CH₂CH₃ |
| O | 4-F | 2,6-Cl₂ | CH₂CH₃ |
| O | 4-F | 2-F-6-Cl | CH₂CH₃ |
| O | 4-F | 2-F-3-Cl | CH₂CH₃ |
| O | 4-F | 4-F-2-Cl | CH₂CH₃ |
| O | 4-F | 2-Cl-3,6-F₂ | CH₂CH₃ |
| O | 4-F | 3-Cl-2,6-F₂ | CH₂CH₃ |
| O | 4-F | 3-F-4-Cl | CH₂CH₃ |
| O | 4-F | 2-F-4-Cl | CH₂CH₃ |
| O | 4-F | 2,3-Cl₂ | CH₂CH₃ |
| O | 4-F | 2-CN | CH₂CH₃ |
| CH₂ | 4-Cl | H | CH₂CH₃ |
| CH₂ | 4-Cl | 3,5-F₂ | CH₂CH₃ |
| CH₂ | 4-Cl | 4-F | CH₂CH₃ |
| CH₂ | 4-Cl | 2,6-F₂ | CH₂CH₃ |
| CH₂ | 4-F | H | CH₂CH₃ |
| CH₂ | 4-F | 3,5-F₂ | CH₂CH₃ |
| CH₂ | 4-F | 4-F | CH₂CH₃ |
| CH₂ | 4-F | 2,6-F₂ | CH₂CH₃ |
| O | 3-Cl | H | CH₂CH₃ |
| O | 3-Cl | 3,5-F₂ | CH₂CH₃ |
| O | 3-Cl | 4-F | CH₂CH₃ |
| O | 3-Cl | 2,6-F₂ | CH₂CH₃ |
| O | 3-Cl | 2,5-F₂ | CH₂CH₃ |
| O | 3-F | H | CH₂CH₃ |
| O | 3-F | 3,5-F₂ | CH₂CH₃ |
| O | 3-F | 4-F | CH₂CH₃ |
| O | 3-F | 2,6-F₂ | CH₂CH₃ |
| O | 3-F | 2,5-F₂ | CH₂CH₃ |
| O | 4-CN | H | CH₂CH₃ |
| O | 4-CN | 3,5-F₂ | CH₂CH₃ |
| O | 4-CN | 4-F | CH₂CH₃ |
| O | 4-CN | 2,6-F₂ | CH₂CH₃ |
| O | 4-CN | 2,5-F₂ | CH₂CH₃ |
| O | 4-Cl | 2,6-Cl₂ | CH₂CH₃ |
| O | 4-Cl | 2-F-6-Cl | CH₂CH₃ |
| O | 4-Cl | 2-F-3-Cl | CH₂CH₃ |
| O | 4-Cl | 4-F-2-Cl | CH₂CH₃ |
| O | 4-Cl | 2-Cl-3,6-F₂ | CH₂CH₃ |
| O | 4-Cl | 3-Cl-2,6-F₂ | CH₂CH₃ |
| O | 4-Cl | 3-F-4-Cl | CH₂CH₃ |
| O | 4-Cl | 2-F-4-Cl | CH₂CH₃ |
| O | 4-Cl | 2,3-Cl₂ | CH₂CH₃ |
| O | 4-Cl | 2-CN | CH₂CH₃ |
| O | 4-F | H | CH₂CH₃ |
| O | 4-F | 2-F | CH₂CH₃ |
| O | 4-F | 3-F | CH₂CH₃ |
| O | 4-F | 4-F | CH₂CH₃ |
| O | 4-F | 4-Cl | CH₂CH₃ |
| O | 2,5-F₂ | H | CH₂CH₃ |
| O | 2,5-F₂ | 3,5-F₂ | CH₂CH₃ |
| O | 2,5-F₂ | 4-F | CH₂CH₃ |
| O | 2,5-F₂ | 2,6-F₂ | CH₂CH₃ |
| O | 2,5-F₂ | 2,5-F₂ | CH₂CH₃ |
| O | 3,5-F₂ | H | CH₂CH₃ |
| O | 3,5-F₂ | 3,5-F₂ | CH₂CH₃ |
| O | 3,5-F₂ | 4-F | CH₂CH₃ |
| O | 3,5-F₂ | 2,6-F₂ | CH₂CH₃ |
| O | 3,5-F₂ | 2,5-F₂ | CH₂CH₃ |
| O | 3,4-F₂ | H | CH₂CH₃ |
| O | 3,4-F₂ | 3,5-F₂ | CH₂CH₃ |
| O | 3,4-F₂ | 4-F | CH₂CH₃ |
| O | 3,4-F₂ | 2,6-F₂ | CH₂CH₃ |
| O | 3,4-F₂ | 2,5-F₂ | CH₂CH₃ |
| O | 4-CF₃ | H | CH₂CH₃ |
| O | 4-CF₃ | 3,5-F₂ | CH₂CH₃ |
| O | 4-CF₃ | 4-F | CH₂CH₃ |
| O | 4-CF₃ | 2,6-F₂ | CH₂CH₃ |
| O | 4-CF₃ | 2,5-F₂ | CH₂CH₃ |
| O | 4-Cl | H | CH₃ |
| O | 4-Cl | 2-F | CH₃ |
| O | 4-Cl | 3-F | CH₃ |
| O | 4-Cl | 4-F | CH₃ |
| O | 4-Cl | 4-Cl | CH₃ |
| O | 4-Cl | 2,5-F₂ | CH₃ |
| O | 4-Cl | 2,4-F₂ | CH₃ |
| O | 4-Cl | 2-Cl | CH₃ |
| O | 4-Cl | 2,6-F₂ | CH₃ |
| O | 4-Cl | 3,5-F₂ | CH₃ |
| O | 4-Cl | 2,3-F₂ | CH₃ |
| O | 4-Cl | 3,4-F₂ | CH₃ |
| O | 4-Cl | 2,3,5-F₃ | CH₃ |
| O | 4-Cl | 2,3,6-F₃ | CH₃ |
| O | 4-Cl | 2,4,5-F₃ | CH₃ |
| O | 4-Cl | 2,6-Cl₂ | CH₃ |
| O | 4-Cl | 2-F-6-Cl | CH₃ |
| O | 4-Cl | 2-F-3-Cl | CH₃ |
| O | 4-Cl | 4-F-2-Cl | CH₃ |
| O | 4-Cl | 2-Cl-3,6-F₂ | CH₃ |
| O | 4-Cl | 3-Cl-2,6-F₂ | CH₃ |
| O | 4-Cl | 3-F-4-Cl | CH₃ |
| O | 4-Cl | 2-F-4-Cl | CH₃ |
| O | 4-Cl | 2,3-Cl₂ | CH₃ |
| O | 4-Cl | 2-CN | CH₃ |
| O | 4-F | H | CH₃ |
| O | 4-F | 2-F | CH₃ |
| O | 4-F | 3-F | CH₃ |
| O | 4-F | 4-F | CH₃ |
| O | 4-F | 4-Cl | CH₃ |
| O | 4-F | 2,5-F₂ | CH₃ |
| O | 4-F | 2,4-F₂ | CH₃ |
| O | 4-F | 2-Cl | CH₃ |
| O | 4-F | 2,6-F₂ | CH₃ |
| O | 4-F | 3,5-F₂ | CH₃ |
| O | 4-F | 2,3-F₂ | CH₃ |
| O | 4-F | 3,4-F₂ | CH₃ |
| O | 4-F | 2,3,5-F₃ | CH₃ |
| O | 4-F | 2,3,6-F₃ | CH₃ |
| O | 4-F | 2,4,5-F₃ | CH₃ |
| O | 4-F | 2,6-Cl₂ | CH₃ |
| O | 4-F | 2-F-6-Cl | CH₃ |
| O | 4-F | 2-F-3-Cl | CH₃ |
| O | 4-F | 4-F-2-Cl | CH₃ |
| O | 4-F | 2-Cl-3,6-F₂ | CH₃ |
| O | 4-F | 3-Cl-2,6-F₂ | CH₃ |
| O | 4-F | 3-F-4-Cl | CH₃ |
| O | 4-F | 2-F-4-Cl | CH₃ |
| O | 4-F | 2,3-Cl₂ | CH₃ |
| O | 4-F | 2-CN | CH₃ |
| CH₂ | 4-Cl | H | CH₃ |
| CH₂ | 4-Cl | 3,5-F₂ | CH₃ |
| CH₂ | 4-Cl | 4-F | CH₃ |
| CH₂ | 4-Cl | 2,6-F₂ | CH₃ |
| CH₂ | 4-F | H | CH₃ |
| CH₂ | 4-F | 3,5-F₂ | CH₃ |
| CH₂ | 4-F | 4-F | CH₃ |
| CH₂ | 4-F | 2,6-F₂ | CH₃ |
| O | 3-F | H | CH₃ |
| O | 3-F | 3,5-F₂ | CH₃ |
| O | 3-F | 4-F | CH₃ |
| O | 3-F | 2,6-F₂ | CH₃ |
| O | 3-F | 2,5-F₂ | CH₃ |
| O | 2,5-F₂ | H | CH₃ |
| O | 2,5-F₂ | 3,5-F₂ | CH₃ |
| O | 2,5-F₂ | 4-F | CH₃ |
| O | 2,5-F₂ | 2,6-F₂ | CH₃ |
| O | 2,5-F₂ | 2,5-F₂ | CH₃ |
| O | 3,5-F₂ | H | CH₃ |
| O | 3,5-F₂ | 3,5-F₂ | CH₃ |
| O | 3,5-F₂ | 4-F | CH₃ |
| O | 3,5-F₂ | 2,6-F₂ | CH₃ |
| O | 3,5-F₂ | 2,5-F₂ | CH₃ |
| O | 3,4-F₂ | H | CH₃ |
| O | 3,4-F₂ | 3,5-F₂ | CH₃ |
| O | 3,4-F₂ | 4-F | CH₃ |
| O | 3,4-F₂ | 2,6-F₂ | CH₃ |
| O | 3,4-F₂ | 2,5-F₂ | CH₃ |
| O | 4-CF₃ | H | CH₃ |
| O | 4-CF₃ | 3,5-F₂ | CH₃ |
| O | 4-CF₃ | 4-F | CH₃ |
| O | 4-CF₃ | 2,6-F₂ | CH₃ |
| O | 4-CF₃ | 2,5-F₂ | CH₃ |

TABLE XIII

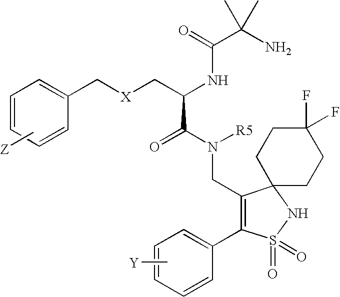

| X | Y | Z | R5 |
|---|---|---|---|
| O | 4-Cl | H | CH₂CH₃ |
| O | 4-Cl | 2-F | CH₂CH₃ |
| O | 4-Cl | 3-F | CH₂CH₃ |
| O | 4-Cl | 4-F | CH₂CH₃ |
| O | 4-Cl | 4-Cl | CH₂CH₃ |
| O | 4-Cl | 2,5-F₂ | CH₂CH₃ |
| O | 4-Cl | 2,4-F₂ | CH₂CH₃ |
| O | 4-Cl | 2-Cl | CH₂CH₃ |
| O | 4-Cl | 2,6-F₂ | CH₂CH₃ |
| O | 4-Cl | 3,5-F₂ | CH₂CH₃ |
| O | 4-Cl | 2,3-F₂ | CH₂CH₃ |
| O | 4-Cl | 3,4-F₂ | CH₂CH₃ |
| O | 4-Cl | 2,3,5-F₃ | CH₂CH₃ |
| O | 4-Cl | 2,3,6-F₃ | CH₂CH₃ |
| O | 4-Cl | 2,4,5-F₃ | CH₂CH₃ |
| O | 4-Cl | 2,6-Cl₂ | CH₂CH₃ |
| O | 4-Cl | 2-F-6-Cl | CH₂CH₃ |
| O | 4-Cl | 2-F-3-Cl | CH₂CH₃ |
| O | 4-Cl | 4-F-2-Cl | CH₂CH₃ |
| O | 4-Cl | 2-Cl-3,6-F₂ | CH₂CH₃ |
| O | 4-Cl | 3-Cl-2,6-F₂ | CH₂CH₃ |
| O | 4-Cl | 3-F-4-Cl | CH₂CH₃ |
| O | 4-Cl | 2-F-4-Cl | CH₂CH₃ |
| O | 4-Cl | 2,3-Cl₂ | CH₂CH₃ |
| O | 4-Cl | 2-CN | CH₂CH₃ |
| O | 4-F | H | CH₂CH₃ |
| O | 4-F | 2-F | CH₂CH₃ |
| O | 4-F | 3-F | CH₂CH₃ |
| O | 4-F | 4-F | CH₂CH₃ |
| O | 4-F | 4-Cl | CH₂CH₃ |
| O | 4-F | 2,5-F₂ | CH₂CH₃ |
| O | 4-F | 2,4-F₂ | CH₂CH₃ |
| O | 4-F | 2-Cl | CH₂CH₃ |
| O | 4-F | 2,6-F₂ | CH₂CH₃ |
| O | 4-F | 3,5-F₂ | CH₂CH₃ |
| O | 4-F | 2,3-F₂ | CH₂CH₃ |
| O | 4-F | 3,4-F₂ | CH₂CH₃ |
| O | 4-F | 2,3,5-F₃ | CH₂CH₃ |
| O | 4-F | 2,3,6-F₃ | CH₂CH₃ |
| O | 4-F | 2,4,5-F₃ | CH₂CH₃ |
| O | 4-F | 2,6-Cl₂ | CH₂CH₃ |
| O | 4-F | 2-F-6-Cl | CH₂CH₃ |
| O | 4-F | 2-F-3-Cl | CH₂CH₃ |
| O | 4-F | 4-F-2-Cl | CH₂CH₃ |
| O | 4-F | 2-Cl-3,6-F₂ | CH₂CH₃ |
| O | 4-F | 3-Cl-2,6-F₂ | CH₂CH₃ |
| O | 4-F | 3-F-4-Cl | CH₂CH₃ |
| O | 4-F | 2-F-4-Cl | CH₂CH₃ |
| O | 4-F | 2,3-Cl₂ | CH₂CH₃ |
| O | 4-F | 2-CN | CH₂CH₃ |
| CH₂ | 4-Cl | H | CH₂CH₃ |
| CH₂ | 4-Cl | 3,5-F₂ | CH₂CH₃ |
| CH₂ | 4-Cl | 4-F | CH₂CH₃ |
| CH₂ | 4-Cl | 2,6-F₂ | CH₂CH₃ |
| CH₂ | 4-F | H | CH₂CH₃ |
| CH₂ | 4-F | 3,5-F₂ | CH₂CH₃ |
| CH₂ | 4-F | 4-F | CH₂CH₃ |
| CH₂ | 4-F | 2,6-F₂ | CH₂CH₃ |
| O | 3-Cl | H | CH₂CH₃ |
| O | 3-Cl | 3,5-F₂ | CH₂CH₃ |
| O | 3-Cl | 4-F | CH₂CH₃ |
| O | 3-Cl | 2,6-F₂ | CH₂CH₃ |
| O | 3-Cl | 2,5-F₂ | CH₂CH₃ |
| O | 3-F | H | CH₂CH₃ |
| O | 3-F | 3,5-F₂ | CH₂CH₃ |
| O | 3-F | 4-F | CH₂CH₃ |
| O | 3-F | 2,6-F₂ | CH₂CH₃ |
| O | 3-F | 2,5-F₂ | CH₂CH₃ |
| O | 4-CN | H | CH₂CH₃ |
| O | 4-CN | 3,5-F₂ | CH₂CH₃ |
| O | 4-CN | 4-F | CH₂CH₃ |
| O | 4-CN | 2,6-F₂ | CH₂CH₃ |
| O | 4-CN | 2,5-F₂ | CH₂CH₃ |
| O | 2,5-F₂ | H | CH₂CH₃ |
| O | 2,5-F₂ | 3,5-F₂ | CH₂CH₃ |
| O | 2,5-F₂ | 4-F | CH₂CH₃ |
| O | 2,5-F₂ | 2,6-F₂ | CH₂CH₃ |
| O | 2,5-F₂ | 2,5-F₂ | CH₂CH₃ |
| O | 3,5-F₂ | H | CH₂CH₃ |
| O | 3,5-F₂ | 3,5-F₂ | CH₂CH₃ |
| O | 3,5-F₂ | 4-F | CH₂CH₃ |
| O | 3,5-F₂ | 2,6-F₂ | CH₂CH₃ |
| O | 3,5-F₂ | 2,5-F₂ | CH₂CH₃ |
| O | 3,4-F₂ | H | CH₂CH₃ |
| O | 3,4-F₂ | 3,5-F₂ | CH₂CH₃ |
| O | 3,4-F₂ | 4-F | CH₂CH₃ |
| O | 3,4-F₂ | 2,6-F₂ | CH₂CH₃ |
| O | 3,4-F₂ | 2,5-F₂ | CH₂CH₃ |
| O | 4-CF₃ | H | CH₂CH₃ |
| O | 4-CF₃ | 3,5-F₂ | CH₂CH₃ |
| O | 4-CF₃ | 4-F | CH₂CH₃ |
| O | 4-CF₃ | 2,6-F₂ | CH₂CH₃ |
| O | 4-CF₃ | 2,5-F₂ | CH₂CH₃ |
| O | 4-Cl | H | CH₃ |
| O | 4-Cl | 2-F | CH₃ |
| O | 4-Cl | 3-F | CH₃ |
| O | 4-Cl | 4-F | CH₃ |
| O | 4-Cl | 4-Cl | CH₃ |
| O | 4-Cl | 2,5-F₂ | CH₃ |
| O | 4-Cl | 2,4-F₂ | CH₃ |
| O | 4-Cl | 2-Cl | CH₃ |
| O | 4-Cl | 2,6-F₂ | CH₃ |
| O | 4-Cl | 3,5-F₂ | CH₃ |
| O | 4-Cl | 2,3-F₂ | CH₃ |
| O | 4-Cl | 3,4-F₂ | CH₃ |
| O | 4-Cl | 2,3,5-F₃ | CH₃ |
| O | 4-Cl | 2,3,6-F₃ | CH₃ |
| O | 4-Cl | 2,4,5-F₃ | CH₃ |
| O | 4-Cl | 2,6-Cl₂ | CH₃ |
| O | 4-Cl | 2-F-6-Cl | CH₃ |
| O | 4-Cl | 2-F-3-Cl | CH₃ |
| O | 4-Cl | 4-F-2-Cl | CH₃ |
| O | 4-Cl | 2-Cl-3,6-F₂ | CH₃ |
| O | 4-Cl | 3-Cl-2,6-F₂ | CH₃ |
| O | 4-Cl | 3-F-4-Cl | CH₃ |
| O | 4-Cl | 2-F-4-Cl | CH₃ |
| O | 4-Cl | 2,3-Cl₂ | CH₃ |
| O | 4-Cl | 2-CN | CH₃ |
| O | 4-F | H | CH₃ |
| O | 4-F | 2-F | CH₃ |
| O | 4-F | 3-F | CH₃ |
| O | 4-F | 4-F | CH₃ |
| O | 4-F | 4-Cl | CH₃ |
| O | 4-F | 2,5-F₂ | CH₃ |
| O | 4-F | 2,4-F₂ | CH₃ |
| O | 4-F | 2-Cl | CH₃ |
| O | 4-F | 2,6-F₂ | CH₃ |
| O | 4-F | 3,5-F₂ | CH₃ |
| O | 4-F | 2,3-F₂ | CH₃ |
| O | 4-F | 3,4-F₂ | CH₃ |
| O | 4-F | 2,3,5-F₃ | CH₃ |
| O | 4-F | 2,3,6-F₃ | CH₃ |
| O | 4-F | 2,4,5-F₃ | CH₃ |
| O | 4-F | 2,6-Cl₂ | CH₃ |
| O | 4-F | 2-F-6-Cl | CH₃ |
| O | 4-F | 2-F-3-Cl | CH₃ |
| O | 4-F | 4-F-2-Cl | CH₃ |
| O | 4-F | 2-Cl-3,6-F₂ | CH₃ |
| O | 4-F | 3-Cl-2,6-F₂ | CH₃ |
| O | 4-F | 3-F-4-Cl | CH₃ |
| O | 4-F | 2-F-4-Cl | CH₃ |
| O | 4-F | 2,3-Cl₂ | CH₃ |
| O | 4-F | 2-CN | CH₃ |
| CH₂ | 4-Cl | H | CH₃ |
| CH₂ | 4-Cl | 3,5-F₂ | CH₃ |
| CH₂ | 4-Cl | 4-F | CH₃ |
| CH₂ | 4-Cl | 2,6-F₂ | CH₃ |
| CH₂ | 4-F | H | CH₃ |
| CH₂ | 4-F | 3,5-F₂ | CH₃ |
| CH₂ | 4-F | 4-F | CH₃ |
| CH₂ | 4-F | 2,6-F₂ | CH₃ |
| O | 3-F | H | CH₃ |
| O | 3-F | 3,5-F₂ | CH₃ |
| O | 3-F | 4-F | CH₃ |
| O | 3-F | 2,6-F₂ | CH₃ |
| O | 3-F | 2,5-F₂ | CH₃ |
| O | 2,5-F₂ | H | CH₃ |
| O | 2,5-F₂ | 3,5-F₂ | CH₃ |
| O | 2,5-F₂ | 4-F | CH₃ |
| O | 2,5-F₂ | 2,6-F₂ | CH₃ |
| O | 2,5-F₂ | 2,5-F₂ | CH₃ |
| O | 3,5-F₂ | H | CH₃ |
| O | 3,5-F₂ | 3,5-F₂ | CH₃ |
| O | 3,5-F₂ | 4-F | CH₃ |
| O | 3,5-F₂ | 2,6-F₂ | CH₃ |
| O | 3,5-F₂ | 2,5-F₂ | CH₃ |
| O | 3,4-F₂ | H | CH₃ |
| O | 3,4-F₂ | 3,5-F₂ | CH₃ |
| O | 3,4-F₂ | 4-F | CH₃ |
| O | 3,4-F₂ | 2,6-F₂ | CH₃ |
| O | 3,4-F₂ | 2,5-F₂ | CH₃ |
| O | 4-CF₃ | H | CH₃ |
| O | 4-CF₃ | 3,5-F₂ | CH₃ |
| O | 4-CF₃ | 4-F | CH₃ |
| O | 4-CF₃ | 2,6-F₂ | CH₃ |
| O | 4-CF₃ | 2,5-F₂ | CH₃ |

TABLE XIV

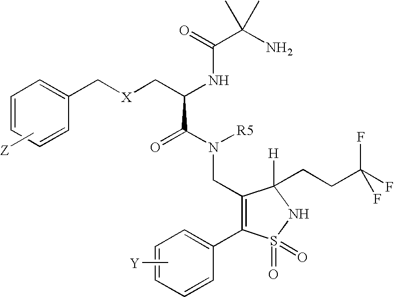

| X | Y | Z | R5 |
|---|---|---|---|
| O | 4-Cl | H | CH₂CH₃ |
| O | 4-Cl | 2-F | CH₂CH₃ |
| O | 4-Cl | 3-F | CH₂CH₃ |
| O | 4-Cl | 4-F | CH₂CH₃ |
| O | 4-Cl | 4-Cl | CH₂CH₃ |
| O | 4-Cl | 2,5-F₂ | CH₂CH₃ |
| O | 4-Cl | 2,4-F₂ | CH₂CH₃ |
| O | 4-Cl | 2-Cl | CH₂CH₃ |
| O | 4-Cl | 2,6-F₂ | CH₂CH₃ |
| O | 4-Cl | 3,5-F₂ | CH₂CH₃ |
| O | 4-Cl | 2,3-F₂ | CH₂CH₃ |
| O | 4-Cl | 3,4-F₂ | CH₂CH₃ |
| O | 4-Cl | 2,3,5-F₃ | CH₂CH₃ |
| O | 4-Cl | 2,3,6-F₃ | CH₂CH₃ |
| O | 4-Cl | 2,4,5-F₃ | CH₂CH₃ |
| O | 4-Cl | 2,6-Cl₂ | CH₂CH₃ |
| O | 4-Cl | 2-F-6-Cl | CH₂CH₃ |
| O | 4-Cl | 2-F-3-Cl | CH₂CH₃ |
| O | 4-Cl | 4-F-2-Cl | CH₂CH₃ |
| O | 4-Cl | 2-Cl-3,6-F₂ | CH₂CH₃ |
| O | 4-Cl | 3-Cl-2,6-F₂ | CH₂CH₃ |
| O | 4-Cl | 3-F-4-Cl | CH₂CH₃ |
| O | 4-Cl | 2-F-4-Cl | CH₂CH₃ |
| O | 4-Cl | 2,3-Cl₂ | CH₂CH₃ |
| O | 4-Cl | 2-CN | CH₂CH₃ |
| O | 4-F | H | CH₂CH₃ |
| O | 4-F | 2-F | CH₂CH₃ |
| O | 4-F | 3-F | CH₂CH₃ |
| O | 4-F | 4-F | CH₂CH₃ |
| O | 4-F | 4-Cl | CH₂CH₃ |
| O | 4-F | 2,5-F₂ | CH₂CH₃ |
| O | 4-F | 2,4-F₂ | CH₂CH₃ |
| O | 4-F | 2-Cl | CH₂CH₃ |
| O | 4-F | 2,6-F₂ | CH₂CH₃ |
| O | 4-F | 3,5-F₂ | CH₂CH₃ |
| O | 4-F | 2,3-F₂ | CH₂CH₃ |
| O | 4-F | 3,4-F₂ | CH₂CH₃ |
| O | 4-F | 2,3,5-F₃ | CH₂CH₃ |
| O | 4-F | 2,3,6-F₃ | CH₂CH₃ |
| O | 4-F | 2,4,5-F₃ | CH₂CH₃ |
| O | 4-F | 2,6-Cl₂ | CH₂CH₃ |
| O | 4-F | 2-F-6-Cl | CH₂CH₃ |
| O | 4-F | 2-F-3-Cl | CH₂CH₃ |
| O | 4-F | 4-F-2-Cl | CH₂CH₃ |
| O | 4-F | 2-Cl-3,6-F₂ | CH₂CH₃ |
| O | 4-F | 3-Cl-2,6-F₂ | CH₂CH₃ |
| O | 4-F | 3-F-4-Cl | CH₂CH₃ |
| O | 4-F | 2-F-4-Cl | CH₂CH₃ |
| O | 4-F | 2,3-Cl₂ | CH₂CH₃ |
| O | 4-F | 2-CN | CH₂CH₃ |
| CH₂ | 4-Cl | H | CH₂CH₃ |
| CH₂ | 4-Cl | 3,5-F₂ | CH₂CH₃ |
| CH₂ | 4-Cl | 4-F | CH₂CH₃ |
| CH₂ | 4-Cl | 2,6-F₂ | CH₂CH₃ |
| CH₂ | 4-F | H | CH₂CH₃ |
| CH₂ | 4-F | 3,5-F₂ | CH₂CH₃ |
| CH₂ | 4-F | 4-F | CH₂CH₃ |
| CH₂ | 4-F | 2,6-F₂ | CH₂CH₃ |
| O | 3-Cl | H | CH₂CH₃ |
| O | 3-Cl | 3,5-F₂ | CH₂CH₃ |
| O | 3-Cl | 4-F | CH₂CH₃ |
| O | 3-Cl | 2,6-F₂ | CH₂CH₃ |
| O | 3-Cl | 2,5-F₂ | CH₂CH₃ |
| O | 3-F | H | CH₂CH₃ |
| O | 3-F | 3,5-F₂ | CH₂CH₃ |
| O | 3-F | 4-F | CH₂CH₃ |
| O | 3-F | 2,6-F₂ | CH₂CH₃ |
| O | 3-F | 2,5-F₂ | CH₂CH₃ |
| O | 4-CN | H | CH₂CH₃ |
| O | 4-CN | 3,5-F₂ | CH₂CH₃ |
| O | 4-CN | 4-F | CH₂CH₃ |
| O | 4-CN | 2,6-F₂ | CH₂CH₃ |
| O | 4-CN | 2,5-F₂ | CH₂CH₃ |
| O | 2,5-F₂ | H | CH₂CH₃ |
| O | 2,5-F₂ | 3,5-F₂ | CH₂CH₃ |
| O | 2,5-F₂ | 4-F | CH₂CH₃ |
| O | 2,5-F₂ | 2,6-F₂ | CH₂CH₃ |
| O | 2,5-F₂ | 2,5-F₂ | CH₂CH₃ |
| O | 3,5-F₂ | H | CH₂CH₃ |
| O | 3,5-F₂ | 3,5-F₂ | CH₂CH₃ |
| O | 3,5-F₂ | 4-F | CH₂CH₃ |
| O | 3,5-F₂ | 2,6-F₂ | CH₂CH₃ |
| O | 3,5-F₂ | 2,5-F₂ | CH₂CH₃ |
| O | 3,4-F₂ | H | CH₂CH₃ |
| O | 3,4-F₂ | 3,5-F₂ | CH₂CH₃ |
| O | 3,4-F₂ | 4-F | CH₂CH₃ |
| O | 3,4-F₂ | 2,6-F₂ | CH₂CH₃ |
| O | 3,4-F₂ | 2,5-F₂ | CH₂CH₃ |
| O | 4-CF₃ | H | CH₂CH₃ |
| O | 4-CF₃ | 3,5-F₂ | CH₂CH₃ |
| O | 4-CF₃ | 4-F | CH₂CH₃ |
| O | 4-CF₃ | 2,6-F₂ | CH₂CH₃ |
| O | 4-CF₃ | 2,5-F₂ | CH₂CH₃ |
| O | 4-Cl | H | CH₃ |
| O | 4-Cl | 2-F | CH₃ |
| O | 4-Cl | 3-F | CH₃ |
| O | 4-Cl | 4-F | CH₃ |
| O | 4-Cl | 4-Cl | CH₃ |
| O | 4-Cl | 2,5-F₂ | CH₃ |
| O | 4-Cl | 2,4-F₂ | CH₃ |
| O | 4-Cl | 2-Cl | CH₃ |
| O | 4-Cl | 2,6-F₂ | CH₃ |
| O | 4-Cl | 3,5-F₂ | CH₃ |
| O | 4-Cl | 2,3-F₂ | CH₃ |
| O | 4-Cl | 3,4-F₂ | CH₃ |
| O | 4-Cl | 2,3,5-F₃ | CH₃ |
| O | 4-Cl | 2,3,6-F₃ | CH₃ |
| O | 4-Cl | 2,4,5-F₃ | CH₃ |
| O | 4-Cl | 2,6-Cl₂ | CH₃ |
| O | 4-Cl | 2-F-6-Cl | CH₃ |
| O | 4-Cl | 2-F-3-Cl | CH₃ |
| O | 4-Cl | 4-F-2-Cl | CH₃ |
| O | 4-Cl | 2-Cl-3,6-F₂ | CH₃ |
| O | 4-Cl | 3-Cl-2,6-F₂ | CH₃ |
| O | 4-Cl | 3-F-4-Cl | CH₃ |
| O | 4-Cl | 2-F-4-Cl | CH₃ |
| O | 4-Cl | 2,3-Cl₂ | CH₃ |
| O | 4-Cl | 2-CN | CH₃ |
| O | 4-F | H | CH₃ |
| O | 4-F | 2-F | CH₃ |
| O | 4-F | 3-F | CH₃ |
| O | 4-F | 4-F | CH₃ |
| O | 4-F | 4-Cl | CH₃ |
| O | 4-F | 2,5-F₂ | CH₃ |
| O | 4-F | 2,4-F₂ | CH₃ |
| O | 4-F | 2-Cl | CH₃ |
| O | 4-F | 2,6-F₂ | CH₃ |
| O | 4-F | 3,5-F₂ | CH₃ |
| O | 4-F | 2,3-F₂ | CH₃ |
| O | 4-F | 3,4-F₂ | CH₃ |
| O | 4-F | 2,3,5-F₃ | CH₃ |
| O | 4-F | 2,3,6-F₃ | CH₃ |
| O | 4-F | 2,4,5-F₃ | CH₃ |
| O | 4-F | 2,6-Cl₂ | CH₃ |
| O | 4-F | 2-F-6-Cl | CH₃ |
| O | 4-F | 2-F-3-Cl | CH₃ |
| O | 4-F | 4-F-2-Cl | CH₃ |
| O | 4-F | 2-Cl-3,6-F₂ | CH₃ |
| O | 4-F | 3-Cl-2,6-F₂ | CH₃ |
| O | 4-F | 3-F-4-Cl | CH₃ |
| O | 4-F | 2-F-4-Cl | CH₃ |
| O | 4-F | 2,3-Cl₂ | CH₃ |
| O | 4-F | 2-CN | CH₃ |
| CH₂ | 4-Cl | H | CH₃ |
| CH₂ | 4-Cl | 3,5-F₂ | CH₃ |
| CH₂ | 4-Cl | 4-F | CH₃ |
| CH₂ | 4-Cl | 2,6-F₂ | CH₃ |
| CH₂ | 4-F | H | CH₃ |
| CH₂ | 4-F | 3,5-F₂ | CH₃ |
| CH₂ | 4-F | 4-F | CH₃ |
| CH₂ | 4-F | 2,6-F₂ | CH₃ |
| O | 3-F | H | CH₃ |
| O | 3-F | 3,5-F₂ | CH₃ |
| O | 3-F | 4-F | CH₃ |
| O | 3-F | 2,6-F₂ | CH₃ |
| O | 3-F | 2,5-F₂ | CH₃ |
| O | 2,5-F₂ | H | CH₃ |
| O | 2,5-F₂ | 3,5-F₂ | CH₃ |
| O | 2,5-F₂ | 4-F | CH₃ |
| O | 2,5-F₂ | 2,6-F₂ | CH₃ |
| O | 2,5-F₂ | 2,5-F₂ | CH₃ |
| O | 3,5-F₂ | H | CH₃ |
| O | 3,5-F₂ | 3,5-F₂ | CH₃ |
| O | 3,5-F₂ | 4-F | CH₃ |
| O | 3,5-F₂ | 2,6-F₂ | CH₃ |
| O | 3,5-F₂ | 2,5-F₂ | CH₃ |
| O | 3,4-F₂ | H | CH₃ |
| O | 3,4-F₂ | 3,5-F₂ | CH₃ |
| O | 3,4-F₂ | 4-F | CH₃ |
| O | 3,4-F₂ | 2,6-F₂ | CH₃ |
| O | 3,4-F₂ | 2,5-F₂ | CH₃ |
| O | 4-CF₃ | H | CH₃ |
| O | 4-CF₃ | 3,5-F₂ | CH₃ |
| O | 4-CF₃ | 4-F | CH₃ |
| O | 4-CF₃ | 2,6-F₂ | CH₃ |
| O | 4-CF₃ | 2,5-F₂ | CH₃ |

TABLE XV

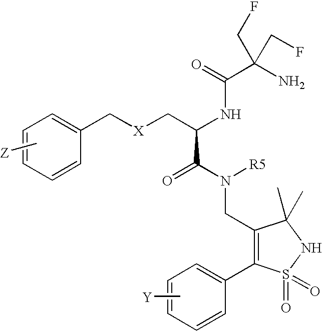

| X | Y | Z | R5 |
|---|---|---|---|
| O | 4-Cl | H | CH₂CH₃ |
| O | 4-Cl | 2-F | CH₂CH₃ |
| O | 4-Cl | 3-F | CH₂CH₃ |
| O | 4-Cl | 4-F | CH₂CH₃ |
| O | 4-Cl | 4-Cl | CH₂CH₃ |
| O | 4-Cl | 2,5-F₂ | CH₂CH₃ |
| O | 4-Cl | 2,4-F₂ | CH₂CH₃ |
| O | 4-Cl | 2-Cl | CH₂CH₃ |
| O | 4-Cl | 2,6-F₂ | CH₂CH₃ |
| O | 4-Cl | 3,5-F₂ | CH₂CH₃ |
| O | 4-Cl | 2,3-F₂ | CH₂CH₃ |
| O | 4-Cl | 3,4-F₂ | CH₂CH₃ |
| O | 4-Cl | 2,3,5-F₃ | CH₂CH₃ |
| O | 4-Cl | 2,3,6-F₃ | CH₂CH₃ |
| O | 4-Cl | 2,4,5-F₃ | CH₂CH₃ |
| O | 4-Cl | 2,6-Cl₂ | CH₂CH₃ |
| O | 4-Cl | 2-F-6-Cl | CH₂CH₃ |
| O | 4-Cl | 2-F-3-Cl | CH₂CH₃ |
| O | 4-Cl | 4-F-2-Cl | CH₂CH₃ |
| O | 4-Cl | 2-CI-3,6-F₂ | CH₂CH₃ |
| O | 4-Cl | 3-CI-2,6-F₂ | CH₂CH₃ |
| O | 4-Cl | 3-F-4-Cl | CH₂CH₃ |
| O | 4-Cl | 2-F-4-Cl | CH₂CH₃ |
| O | 4-Cl | 2,3-Cl₂ | CH₂CH₃ |
| O | 4-Cl | 2-CN | CH₂CH₃ |
| O | 4-F | H | CH₂CH₃ |
| O | 4-F | 2-F | CH₂CH₃ |
| O | 4-F | 3-F | CH₂CH₃ |
| O | 4-F | 4-F | CH₂CH₃ |
| O | 4-F | 4-Cl | CH₂CH₃ |
| O | 4-F | 2,5-F₂ | CH₂CH₃ |
| O | 4-F | 2,4-F₂ | CH₂CH₃ |
| O | 4-F | 2-Cl | CH₂CH₃ |
| O | 4-F | 2,6-F₂ | CH₂CH₃ |
| O | 4-F | 3,5-F₂ | CH₂CH₃ |
| O | 4-F | 2,3-F₂ | CH₂CH₃ |
| O | 4-F | 3,4-F₂ | CH₂CH₃ |
| O | 4-F | 2,3,5-F₃ | CH₂CH₃ |
| O | 4-F | 2,3,6-F₃ | CH₂CH₃ |
| O | 4-F | 2,4,5-F₃ | CH₂CH₃ |
| O | 4-F | 2,6-Cl₂ | CH₂CH₃ |
| O | 4-F | 2-F-6-Cl | CH₂CH₃ |
| O | 4-F | 2-F-3-Cl | CH₂CH₃ |
| O | 4-F | 4-F-2-Cl | CH₂CH₃ |
| O | 4-F | 2-Cl-3,6-F₂ | CH₂CH₃ |
| O | 4-F | 3-Cl-2,6-F₂ | CH₂CH₃ |
| O | 4-F | 3-F-4-Cl | CH₂CH₃ |
| O | 4-F | 2-F-4-Cl | CH₂CH₃ |
| O | 4-F | 2,3-Cl₂ | CH₂CH₃ |
| O | 4-F | 2-CN | CH₂CH₃ |
| CH₂ | 4-Cl | H | CH₂CH₃ |
| CH₂ | 4-Cl | 3,5-F₂ | CH₂CH₃ |
| CH₂ | 4-Cl | 4-F | CH₂CH₃ |
| CH₂ | 4-Cl | 2,6-F₂ | CH₂CH₃ |
| CH₂ | 4-F | H | CH₂CH₃ |
| CH₂ | 4-F | 3,5-F₂ | CH₂CH₃ |
| CH₂ | 4-F | 4-F | CH₂CH₃ |
| CH₂ | 4-F | 2,6-F₂ | CH₂CH₃ |
| O | 3-Cl | H | CH₂CH₃ |
| O | 3-Cl | 3,5-F₂ | CH₂CH₃ |
| O | 3-Cl | 4-F | CH₂CH₃ |
| O | 3-Cl | 2,6-F₂ | CH₂CH₃ |
| O | 3-Cl | 2,5-F₂ | CH₂CH₃ |
| O | 3-F | H | CH₂CH₃ |
| O | 3-F | 3,5-F₂ | CH₂CH₃ |
| O | 3-F | 4-F | CH₂CH₃ |
| O | 3-F | 2,6-F₂ | CH₂CH₃ |
| O | 3-F | 2,5-F₂ | CH₂CH₃ |
| O | 4-CN | H | CH₂CH₃ |
| O | 4-CN | 3,5-F₂ | CH₂CH₃ |
| O | 4-CN | 4-F | CH₂CH₃ |
| O | 4-CN | 2,6-F₂ | CH₂CH₃ |
| O | 4-CN | 2,5-F₂ | CH₂CH₃ |
| O | 2,5-F₂ | H | CH₂CH₃ |
| O | 2,5-F₂ | 3,5-F₂ | CH₂CH₃ |
| O | 2,5-F₂ | 4-F | CH₂CH₃ |
| O | 2,5-F₂ | 2,6-F₂ | CH₂CH₃ |
| O | 2,5-F₂ | 2,5-F₂ | CH₂CH₃ |
| O | 3,5-F₂ | H | CH₂CH₃ |
| O | 3,5-F₂ | 3,5-F₂ | CH₂CH₃ |
| O | 3,5-F₂ | 4-F | CH₂CH₃ |
| O | 3,5-F₂ | 2,6-F₂ | CH₂CH₃ |
| O | 3,5-F₂ | 2,5-F₂ | CH₂CH₃ |
| O | 3,4-F₂ | H | CH₂CH₃ |
| O | 3,4-F₂ | 3,5-F₂ | CH₂CH₃ |
| O | 3,4-F₂ | 4-F | CH₂CH₃ |
| O | 3,4-F₂ | 2,6-F₂ | CH₂CH₃ |
| O | 3,4-F₂ | 2,5-F₂ | CH₂CH₃ |
| O | 4-CF₃ | H | CH₂CH₃ |
| O | 4-CF₃ | 3,5-F₂ | CH₂CH₃ |
| O | 4-CF₃ | 4-F | CH₂CH₃ |
| O | 4-CF₃ | 2,6-F₂ | CH₂CH₃ |
| O | 4-CF₃ | 2,5-F₂ | CH₂CH₃ |
| O | 4-Cl | H | CH₃ |
| O | 4-Cl | 2-F | CH₃ |
| O | 4-Cl | 3-F | CH₃ |
| O | 4-Cl | 4-F | CH₃ |
| O | 4-Cl | 4-Cl | CH₃ |
| O | 4-Cl | 2,5-F₂ | CH₃ |
| O | 4-Cl | 2,4-F₂ | CH₃ |
| O | 4-Cl | 2-Cl | CH₃ |
| O | 4-Cl | 2,6-F₂ | CH₃ |
| O | 4-Cl | 3,5-F₂ | CH₃ |
| O | 4-Cl | 2,3-F₂ | CH₃ |
| O | 4-Cl | 3,4-F₂ | CH₃ |
| O | 4-Cl | 2,3,5-F₃ | CH₃ |
| O | 4-Cl | 2,3,6-F₃ | CH₃ |
| O | 4-Cl | 2,4,5-F₃ | CH₃ |
| O | 4-Cl | 2,6-Cl₂ | CH₃ |
| O | 4-Cl | 2-F-6-Cl | CH₃ |
| O | 4-Cl | 2-F-3-Cl | CH₃ |
| O | 4-Cl | 4-F-2-Cl | CH₃ |
| O | 4-Cl | 2-Cl-3,6-F₂ | CH₃ |
| O | 4-Cl | 3-Cl-2,6-F₂ | CH₃ |
| O | 4-Cl | 3-F-4-Cl | CH₃ |
| O | 4-Cl | 2-F-4-Cl | CH₃ |
| O | 4-Cl | 2,3-Cl₂ | CH₃ |
| O | 4-Cl | 2-CN | CH₃ |
| O | 4-F | H | CH₃ |
| O | 4-F | 2-F | CH₃ |
| O | 4-F | 3-F | CH₃ |
| O | 4-F | 4-F | CH₃ |
| O | 4-F | 4-Cl | CH₃ |
| O | 4-F | 2,5-F₂ | CH₃ |
| O | 4-F | 2,4-F₂ | CH₃ |
| O | 4-F | 2-Cl | CH₃ |
| O | 4-F | 2,6-F₂ | CH₃ |
| O | 4-F | 3,5-F₂ | CH₃ |
| O | 4-F | 2,3-F₂ | CH₃ |
| O | 4-F | 3,4-F₂ | CH₃ |
| O | 4-F | 2,3,5-F₃ | CH₃ |
| O | 4-F | 2,3,6-F₃ | CH₃ |
| O | 4-F | 2,4,5-F₃ | CH₃ |
| O | 4-F | 2,6-Cl₂ | CH₃ |
| O | 4-F | 2-F-6-Cl | CH₃ |
| O | 4-F | 2-F-3-Cl | CH₃ |
| O | 4-F | 4-F-2-Cl | CH₃ |
| O | 4-F | 2-Cl-3,6-F₂ | CH₃ |
| O | 4-F | 3-Cl-2,6-F₂ | CH₃ |
| O | 4-F | 3-F-4-Cl | CH₃ |
| O | 4-F | 2-F-4-Cl | CH₃ |
| O | 4-F | 2,3-Cl₂ | CH₃ |
| O | 4-F | 2-CN | CH₃ |
| CH₂ | 4-Cl | H | CH₃ |
| CH₂ | 4-Cl | 3,5-F₂ | CH₃ |
| CH₂ | 4-Cl | 4-F | CH₃ |
| CH₂ | 4-Cl | 2,6-F₂ | CH₃ |
| CH₂ | 4-F | H | CH₃ |
| CH₂ | 4-F | 3,5-F₂ | CH₃ |
| CH₂ | 4-F | 4-F | CH₃ |
| CH₂ | 4-F | 2,6-F₂ | CH₃ |
| O | 3-F | H | CH₃ |
| O | 3-F | 3,5-F₂ | CH₃ |
| O | 3-F | 4-F | CH₃ |
| O | 3-F | 2,6-F₂ | CH₃ |
| O | 3-F | 2,5-F₂ | CH₃ |
| O | 2,5-F₂ | H | CH₃ |
| O | 2,5-F₂ | 3,5-F₂ | CH₃ |
| O | 2,5-F₂ | 4-F | CH₃ |
| O | 2,5-F₂ | 2,6-F₂ | CH₃ |
| O | 2,5-F₂ | 2,5-F₂ | CH₃ |
| O | 3,5-F₂ | H | CH₃ |
| O | 3,5-F₂ | 3,5-F₂ | CH₃ |
| O | 3,5-F₂ | 4-F | CH₃ |
| O | 3,5-F₂ | 2,6-F₂ | CH₃ |
| O | 3,5-F₂ | 2,5-F₂ | CH₃ |
| O | 3,4-F₂ | H | CH₃ |
| O | 3,4-F₂ | 3,5-F₂ | CH₃ |
| O | 3,4-F₂ | 4-F | CH₃ |
| O | 3,4-F₂ | 2,6-F₂ | CH₃ |
| O | 3,4-F₂ | 2,5-F₂ | CH₃ |
| O | 4-CF₃ | H | CH₃ |
| O | 4-CF₃ | 3,5-F₂ | CH₃ |
| O | 4-CF₃ | 4-F | CH₃ |
| O | 4-CF₃ | 2,6-F₂ | CH₃ |
| O | 4-CF₃ | 2,5-F₂ | CH₃ |

TABLE XVI

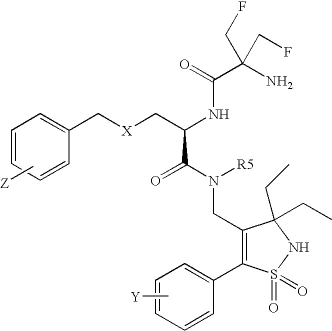

| X | Y | Z | R5 |
|---|---|---|---|
| O | 4-Cl | H | CH₂CH₃ |
| O | 4-Cl | 2-F | CH₂CH₃ |
| O | 4-Cl | 3-F | CH₂CH₃ |
| O | 4-Cl | 4-F | CH₂CH₃ |
| O | 4-Cl | 4-Cl | CH₂CH₃ |
| O | 4-Cl | 2,5-F₂ | CH₂CH₃ |
| O | 4-Cl | 2,4-F₂ | CH₂CH₃ |
| O | 4-Cl | 2-Cl | CH₂CH₃ |
| O | 4-Cl | 2,6-F₂ | CH₂CH₃ |
| O | 4-Cl | 3,5-F₂ | CH₂CH₃ |
| O | 4-Cl | 2,3-F₂ | CH₂CH₃ |
| O | 4-Cl | 3,4-F₂ | CH₂CH₃ |
| O | 4-Cl | 2,3,5-F₃ | CH₂CH₃ |
| O | 4-Cl | 2,3,6-F₃ | CH₂CH₃ |
| O | 4-Cl | 2,4,5-F₃ | CH₂CH₃ |
| O | 4-Cl | 2,6-Cl₂ | CH₂CH₃ |
| O | 4-Cl | 2-F-6-Cl | CH₂CH₃ |
| O | 4-Cl | 2-F-3-Cl | CH₂CH₃ |
| O | 4-Cl | 4-F-2-Cl | CH₂CH₃ |
| O | 4-Cl | 2-Cl-3,6-F₂ | CH₂CH₃ |
| O | 4-Cl | 3-Cl-2,6-F₂ | CH₂CH₃ |
| O | 4-Cl | 3-F-4-Cl | CH₂CH₃ |
| O | 4-Cl | 2-F-4-Cl | CH₂CH₃ |
| O | 4-Cl | 2,3-Cl₂ | CH₂CH₃ |
| O | 4-Cl | 2-CN | CH₂CH₃ |
| O | 4-F | H | CH₂CH₃ |
| O | 4-F | 2-F | CH₂CH₃ |
| O | 4-F | 3-F | CH₂CH₃ |
| O | 4-F | 4-F | CH₂CH₃ |
| O | 4-F | 4-Cl | CH₂CH₃ |
| O | 4-F | 2,5-F₂ | CH₂CH₃ |
| O | 4-F | 2,4-F₂ | CH₂CH₃ |
| O | 4-F | 2-Cl | CH₂CH₃ |
| O | 4-F | 2,6-F₂ | CH₂CH₃ |
| O | 4-F | 3,5-F₂ | CH₂CH₃ |
| O | 4-F | 2,3-F₂ | CH₂CH₃ |
| O | 4-F | 3,4-F₂ | CH₂CH₃ |
| O | 4-F | 2,3,5-F₃ | CH₂CH₃ |
| O | 4-F | 2,3,6-F₃ | CH₂CH₃ |
| O | 4-F | 2,4,5-F₃ | CH₂CH₃ |
| O | 4-F | 2,6-Cl₂ | CH₂CH₃ |
| O | 4-F | 2-F-6-Cl | CH₂CH₃ |
| O | 4-F | 2-F-3-Cl | CH₂CH₃ |
| O | 4-F | 4-F-2-Cl | CH₂CH₃ |
| O | 4-F | 2-Cl-3,6-F₂ | CH₂CH₃ |
| O | 4-F | 3-Cl-2,6-F₂ | CH₂CH₃ |
| O | 4-F | 3-F-4-Cl | CH₂CH₃ |
| O | 4-F | 2-F-4-Cl | CH₂CH₃ |
| O | 4-F | 2,3-Cl₂ | CH₂CH₃ |
| O | 4-F | 2-CN | CH₂CH₃ |
| CH₂ | 4-Cl | H | CH₂CH₃ |
| CH₂ | 4-Cl | 3,5-F₂ | CH₂CH₃ |
| CH₂ | 4-Cl | 4-F | CH₂CH₃ |
| CH₂ | 4-Cl | 2,6-F₂ | CH₂CH₃ |
| CH₂ | 4-F | H | CH₂CH₃ |
| CH₂ | 4-F | 3,5-F₂ | CH₂CH₃ |
| CH₂ | 4-F | 4-F | CH₂CH₃ |
| CH₂ | 4-F | 2,6-F₂ | CH₂CH₃ |
| O | 3-Cl | H | CH₂CH₃ |
| O | 3-Cl | 3,5-F₂ | CH₂CH₃ |
| O | 3-Cl | 4-F | CH₂CH₃ |
| O | 3-Cl | 2,6-F₂ | CH₂CH₃ |
| O | 3-Cl | 2,5-F₂ | CH₂CH₃ |
| O | 3-F | H | CH₂CH₃ |
| O | 3-F | 3,5-F₂ | CH₂CH₃ |
| O | 3-F | 4-F | CH₂CH₃ |
| O | 3-F | 2,6-F₂ | CH₂CH₃ |
| O | 3-F | 2,5-F₂ | CH₂CH₃ |
| O | 4-CN | H | CH₂CH₃ |
| O | 4-CN | 3,5-F₂ | CH₂CH₃ |
| O | 4-CN | 4-F | CH₂CH₃ |
| O | 4-CN | 2,6-F₂ | CH₂CH₃ |
| O | 4-CN | 2,5-F₂ | CH₂CH₃ |
| O | 2,5-F₂ | H | CH₂CH₃ |
| O | 2,5-F₂ | 3,5-F₂ | CH₂CH₃ |
| O | 2,5-F₂ | 4-F | CH₂CH₃ |
| O | 2,5-F₂ | 2,6-F₂ | CH₂CH₃ |
| O | 2,5-F₂ | 2,5-F₂ | CH₂CH₃ |
| O | 3,5-F₂ | H | CH₂CH₃ |
| O | 3,5-F₂ | 3,5-F₂ | CH₂CH₃ |
| O | 3,5-F₂ | 4-F | CH₂CH₃ |
| O | 3,5-F₂ | 2,6-F₂ | CH₂CH₃ |
| O | 3,5-F₂ | 2,5-F₂ | CH₂CH₃ |
| O | 3,4-F₂ | H | CH₂CH₃ |
| O | 3,4-F₂ | 3,5-F₂ | CH₂CH₃ |
| O | 3,4-F₂ | 4-F | CH₂CH₃ |
| O | 3,4-F₂ | 2,6-F₂ | CH₂CH₃ |
| O | 3,4-F₂ | 2,5-F₂ | CH₂CH₃ |
| O | 4-CF₃ | H | CH₂CH₃ |
| O | 4-CF₃ | 3,5-F₂ | CH₂CH₃ |
| O | 4-CF₃ | 4-F | CH₂CH₃ |
| O | 4-CF₃ | 2,6-F₂ | CH₂CH₃ |
| O | 4-CF₃ | 2,5-F₂ | CH₂CH₃ |
| O | 4-Cl | H | CH₃ |
| O | 4-Cl | 2-F | CH₃ |
| O | 4-Cl | 3-F | CH₃ |
| O | 4-Cl | 4-F | CH₃ |
| O | 4-Cl | 4-Cl | CH₃ |
| O | 4-Cl | 2,5-F₂ | CH₃ |
| O | 4-Cl | 2,4-F₂ | CH₃ |
| O | 4-Cl | 2-Cl | CH₃ |
| O | 4-Cl | 2,6-F₂ | CH₃ |
| O | 4-Cl | 3,5-F₂ | CH₃ |
| O | 4-Cl | 2,3-F₂ | CH₃ |
| O | 4-Cl | 3,4-F₂ | CH₃ |
| O | 4-Cl | 2,3,5-F₃ | CH₃ |
| O | 4-Cl | 2,3,6-F₃ | CH₃ |
| O | 4-Cl | 2,4,5-F₃ | CH₃ |
| O | 4-Cl | 2,6-Cl₂ | CH₃ |
| O | 4-Cl | 2-F-6-Cl | CH₃ |
| O | 4-Cl | 2-F-3-Cl | CH₃ |
| O | 4-Cl | 4-F-2-Cl | CH₃ |
| O | 4-Cl | 2-Cl-3,6-F₂ | CH₃ |
| O | 4-Cl | 3-Cl-2,6-F₂ | CH₃ |
| O | 4-Cl | 3-F-4-Cl | CH₃ |
| O | 4-Cl | 2-F-4-Cl | CH₃ |
| O | 4-Cl | 2,3-Cl₂ | CH₃ |
| O | 4-Cl | 2-CN | CH₃ |
| O | 4-F | H | CH₃ |
| O | 4-F | 2-F | CH₃ |
| O | 4-F | 3-F | CH₃ |
| O | 4-F | 4-F | CH₃ |
| O | 4-F | 4-Cl | CH₃ |
| O | 4-F | 2,5-F₂ | CH₃ |
| O | 4-F | 2,4-F₂ | CH₃ |
| O | 4-F | 2-Cl | CH₃ |
| O | 4-F | 2,6-F₂ | CH₃ |
| O | 4-F | 3,5-F₂ | CH₃ |
| O | 4-F | 2,3-F₂ | CH₃ |
| O | 4-F | 3,4-F₂ | CH₃ |
| O | 4-F | 2,3,5-F₃ | CH₃ |
| O | 4-F | 2,3,6-F₃ | CH₃ |
| O | 4-F | 2,4,5-F₃ | CH₃ |
| O | 4-F | 2,6-Cl₂ | CH₃ |
| O | 4-F | 2-F-6-Cl | CH₃ |
| O | 4-F | 2-F-3-Cl | CH₃ |
| O | 4-F | 4-F-2-Cl | CH₃ |
| O | 4-F | 2-Cl-3,6-F₂ | CH₃ |
| O | 4-F | 3-Cl-2,6-F₂ | CH₃ |
| O | 4-F | 3-F-4-Cl | CH₃ |
| O | 4-F | 2-F-4-Cl | CH₃ |
| O | 4-F | 2,3-Cl₂ | CH₃ |
| O | 4-F | 2-CN | CH₃ |
| CH₂ | 4-Cl | H | CH₃ |
| CH₂ | 4-Cl | 3,5-F₂ | CH₃ |
| CH₂ | 4-Cl | 4-F | CH₃ |
| CH₂ | 4-Cl | 2,6-F₂ | CH₃ |
| CH₂ | 4-F | H | CH₃ |
| CH₂ | 4-F | 3,5-F₂ | CH₃ |
| CH₂ | 4-F | 4-F | CH₃ |
| CH₂ | 4-F | 2,6-F₂ | CH₃ |
| O | 3-F | H | CH₃ |
| O | 3-F | 3,5-F₂ | CH₃ |
| O | 3-F | 4-F | CH₃ |
| O | 3-F | 2,6-F₂ | CH₃ |
| O | 3-F | 2,5-F₂ | CH₃ |
| O | 2,5-F₂ | H | CH₃ |
| O | 2,5-F₂ | 3,5-F₂ | CH₃ |
| O | 2,5-F₂ | 4-F | CH₃ |
| O | 2,5-F₂ | 2,6-F₂ | CH₃ |
| O | 2,5-F₂ | 2,5-F₂ | CH₃ |
| O | 3,5-F₂ | H | CH₃ |
| O | 3,5-F₂ | 3,5-F₂ | CH₃ |
| O | 3,5-F₂ | 4-F | CH₃ |
| O | 3,5-F₂ | 2,6-F₂ | CH₃ |
| O | 3,5-F₂ | 2,5-F₂ | CH₃ |
| O | 3,4-F₂ | H | CH₃ |
| O | 3,4-F₂ | 3,5-F₂ | CH₃ |
| O | 3,4-F₂ | 4-F | CH₃ |
| O | 3,4-F₂ | 2,6-F₂ | CH₃ |
| O | 3,4-F₂ | 2,5-F₂ | CH₃ |
| O | 4-CF₃ | H | CH₃ |
| O | 4-CF₃ | 3,5-F₂ | CH₃ |
| O | 4-CF₃ | 4-F | CH₃ |
| O | 4-CF₃ | 2,6-F₂ | CH₃ |
| O | 4-CF₃ | 2,5-F₂ | CH₃ |

TABLE XVII

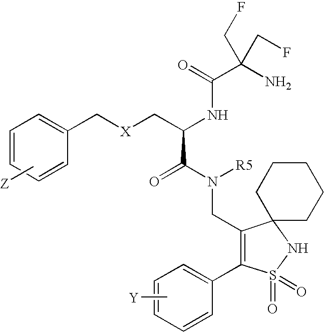

| X | Y | Z | R5 |
|---|---|---|---|
| O | 4-Cl | H | CH₂CH₃ |
| O | 4-Cl | 2-F | CH₂CH₃ |
| O | 4-Cl | 3-F | CH₂CH₃ |
| O | 4-Cl | 4-F | CH₂CH₃ |
| O | 4-Cl | 4-Cl | CH₂CH₃ |
| O | 4-Cl | 2,5-F₂ | CH₂CH₃ |
| O | 4-Cl | 2,4-F₂ | CH₂CH₃ |
| O | 4-Cl | 2-Cl | CH₂CH₃ |
| O | 4-Cl | 2,6-F₂ | CH₂CH₃ |
| O | 4-Cl | 3,5-F₂ | CH₂CH₃ |
| O | 4-Cl | 2,3-F₂ | CH₂CH₃ |
| O | 4-Cl | 3,4-F₂ | CH₂CH₃ |
| O | 4-Cl | 2,3,5-F₃ | CH₂CH₃ |
| O | 4-Cl | 2,3,6-F₃ | CH₂CH₃ |
| O | 4-Cl | 2,4,5-F₃ | CH₂CH₃ |
| O | 4-Cl | 2,6-Cl₂ | CH₂CH₃ |
| O | 4-Cl | 2-F-6-Cl | CH₂CH₃ |
| O | 4-Cl | 2-F-3-Cl | CH₂CH₃ |
| O | 4-Cl | 4-F-2-Cl | CH₂CH₃ |
| O | 4-Cl | 2-Cl-3,6-F₂ | CH₂CH₃ |
| O | 4-Cl | 3-Cl-2,6-F₂ | CH₂CH₃ |
| O | 4-Cl | 3-F-4-Cl | CH₂CH₃ |
| O | 4-Cl | 2-F-4-Cl | CH₂CH₃ |
| O | 4-Cl | 2,3-Cl₂ | CH₂CH₃ |
| O | 4-Cl | 2-CN | CH₂CH₃ |
| O | 4-F | H | CH₂CH₃ |
| O | 4-F | 2-F | CH₂CH₃ |
| O | 4-F | 3-F | CH₂CH₃ |
| O | 4-F | 4-F | CH₂CH₃ |
| O | 4-F | 4-Cl | CH₂CH₃ |
| O | 4-F | 2,5-F₂ | CH₂CH₃ |
| O | 4-F | 2,4-F₂ | CH₂CH₃ |
| O | 4-F | 2-Cl | CH₂CH₃ |
| O | 4-F | 2,6-F₂ | CH₂CH₃ |
| O | 4-F | 3,5-F₂ | CH₂CH₃ |
| O | 4-F | 2,3-F₂ | CH₂CH₃ |
| O | 4-F | 3,4-F₂ | CH₂CH₃ |
| O | 4-F | 2,3,5-F₃ | CH₂CH₃ |
| O | 4-F | 2,3,6-F₃ | CH₂CH₃ |
| O | 4-F | 2,4,5-F₃ | CH₂CH₃ |
| O | 4-F | 2,6-Cl₂ | CH₂CH₃ |
| O | 4-F | 2-F-6-Cl | CH₂CH₃ |
| O | 4-F | 2-F-3-Cl | CH₂CH₃ |
| O | 4-F | 4-F-2-Cl | CH₂CH₃ |
| O | 4-F | 2-Cl-3,6-F₂ | CH₂CH₃ |
| O | 4-F | 3-Cl-2,6-F₂ | CH₂CH₃ |
| O | 4-F | 3-F-4-Cl | CH₂CH₃ |
| O | 4-F | 2-F-4-Cl | CH₂CH₃ |
| O | 4-F | 2,3-Cl₂ | CH₂CH₃ |
| O | 4-F | 2-CN | CH₂CH₃ |
| CH₂ | 4-Cl | H | CH₂CH₃ |
| CH₂ | 4-Cl | 3,5-F₂ | CH₂CH₃ |
| CH₂ | 4-Cl | 4-F | CH₂CH₃ |
| CH₂ | 4-Cl | 2,6-F₂ | CH₂CH₃ |
| CH₂ | 4-F | H | CH₂CH₃ |
| CH₂ | 4-F | 3,5-F₂ | CH₂CH₃ |
| CH₂ | 4-F | 4-F | CH₂CH₃ |
| CH₂ | 4-F | 2,6-F₂ | CH₂CH₃ |
| O | 3-Cl | H | CH₂CH₃ |
| O | 3-Cl | 3,5-F₂ | CH₂CH₃ |
| O | 3-Cl | 4-F | CH₂CH₃ |
| O | 3-Cl | 2,6-F₂ | CH₂CH₃ |
| O | 3-Cl | 2,5-F₂ | CH₂CH₃ |
| O | 3-F | H | CH₂CH₃ |
| O | 3-F | 3,5-F₂ | CH₂CH₃ |
| O | 3-F | 4-F | CH₂CH₃ |
| O | 3-F | 2,6-F₂ | CH₂CH₃ |
| O | 3-F | 2,5-F₂ | CH₂CH₃ |
| O | 4-CN | H | CH₂CH₃ |
| O | 4-CN | 3,5-F₂ | CH₂CH₃ |
| O | 4-CN | 4-F | CH₂CH₃ |
| O | 4-CN | 2,6-F₂ | CH₂CH₃ |
| O | 4-CN | 2,5-F₂ | CH₂CH₃ |
| O | 2,5-F₂ | H | CH₂CH₃ |
| O | 2,5-F₂ | 3,5-F₂ | CH₂CH₃ |
| O | 2,5-F₂ | 4-F | CH₂CH₃ |
| O | 2,5-F₂ | 2,6-F₂ | CH₂CH₃ |
| O | 2,5-F₂ | 2,5-F₂ | CH₂CH₃ |
| O | 3,5-F₂ | H | CH₂CH₃ |
| O | 3,5-F₂ | 3,5-F₂ | CH₂CH₃ |
| O | 3,5-F₂ | 4-F | CH₂CH₃ |
| O | 3,5-F₂ | 2,6-F₂ | CH₂CH₃ |
| O | 3,5-F₂ | 2,5-F₂ | CH₂CH₃ |
| O | 3,4-F₂ | H | CH₂CH₃ |
| O | 3,4-F₂ | 3,5-F₂ | CH₂CH₃ |
| O | 3,4-F₂ | 4-F | CH₂CH₃ |
| O | 3,4-F₂ | 2,6-F₂ | CH₂CH₃ |
| O | 3,4-F₂ | 2,5-F₂ | CH₂CH₃ |
| O | 4-CF₃ | H | CH₂CH₃ |
| O | 4-CF₃ | 3,5-F₂ | CH₂CH₃ |
| O | 4-CF₃ | 4-F | CH₂CH₃ |
| O | 4-CF₃ | 2,6-F₂ | CH₂CH₃ |
| O | 4-CF₃ | 2,5-F₂ | CH₂CH₃ |
| O | 4-Cl | H | CH₃ |
| O | 4-Cl | 2-F | CH₃ |
| O | 4-Cl | 3-F | CH₃ |
| O | 4-Cl | 4-F | CH₃ |
| O | 4-Cl | 4-Cl | CH₃ |
| O | 4-Cl | 2,5-F₂ | CH₃ |
| O | 4-Cl | 2,4-F₂ | CH₃ |
| O | 4-Cl | 2-Cl | CH₃ |
| O | 4-Cl | 2,6-F₂ | CH₃ |
| O | 4-Cl | 3,5-F₂ | CH₃ |
| O | 4-Cl | 2,3-F₂ | CH₃ |
| O | 4-Cl | 3,4-F₂ | CH₃ |
| O | 4-Cl | 2,3,5-F₃ | CH₃ |
| O | 4-Cl | 2,3,6-F₃ | CH₃ |
| O | 4-Cl | 2,4,5-F₃ | CH₃ |
| O | 4-Cl | 2,6-Cl₂ | CH₃ |
| O | 4-Cl | 2-F-6-Cl | CH₃ |
| O | 4-Cl | 2-F-3-Cl | CH₃ |
| O | 4-Cl | 4-F-2-Cl | CH₃ |
| O | 4-Cl | 2-Cl-3,6-F₂ | CH₃ |
| O | 4-Cl | 3-Cl-2,6-F₂ | CH₃ |
| O | 4-Cl | 3-F-4-Cl | CH₃ |
| O | 4-Cl | 2-F-4-Cl | CH₃ |
| O | 4-Cl | 2,3-Cl₂ | CH₃ |
| O | 4-Cl | 2-CN | CH₃ |
| O | 4-F | H | CH₃ |
| O | 4-F | 2-F | CH₃ |
| O | 4-F | 3-F | CH₃ |
| O | 4-F | 4-F | CH₃ |
| O | 4-F | 4-Cl | CH₃ |
| O | 4-F | 2,5-F₂ | CH₃ |
| O | 4-F | 2,4-F₂ | CH₃ |
| O | 4-F | 2-Cl | CH₃ |
| O | 4-F | 2,6-F₂ | CH₃ |
| O | 4-F | 3,5-F₂ | CH₃ |
| O | 4-F | 2,3-F₂ | CH₃ |
| O | 4-F | 3,4-F₂ | CH₃ |
| O | 4-F | 2,3,5-F₃ | CH₃ |
| O | 4-F | 2,3,6-F₃ | CH₃ |
| O | 4-F | 2,4,5-F₃ | CH₃ |
| O | 4-F | 2,6-Cl₂ | CH₃ |
| O | 4-F | 2-F-6-Cl | CH₃ |
| O | 4-F | 2-F-3-Cl | CH₃ |
| O | 4-F | 4-F-2-Cl | CH₃ |
| O | 4-F | 2-Cl-3,6-F₂ | CH₃ |
| O | 4-F | 3-Cl-2,6-F₂ | CH₃ |
| O | 4-F | 3-F-4-Cl | CH₃ |
| O | 4-F | 2-F-4-Cl | CH₃ |
| O | 4-F | 2,3-Cl₂ | CH₃ |
| O | 4-F | 2-CN | CH₃ |
| CH₂ | 4-Cl | H | CH₃ |
| CH₂ | 4-Cl | 3,5-F₂ | CH₃ |
| CH₂ | 4-Cl | 4-F | CH₃ |
| CH₂ | 4-Cl | 2,6-F₂ | CH₃ |
| CH₂ | 4-F | H | CH₃ |
| CH₂ | 4-F | 3,5-F₂ | CH₃ |
| CH₂ | 4-F | 4-F | CH₃ |
| CH₂ | 4-F | 2,6-F₂ | CH₃ |
| O | 3-F | H | CH₃ |
| O | 3-F | 3,5-F₂ | CH₃ |
| O | 3-F | 4-F | CH₃ |
| O | 3-F | 2,6-F₂ | CH₃ |
| O | 3-F | 2,5-F₂ | CH₃ |
| O | 2,5-F₂ | H | CH₃ |
| O | 2,5-F₂ | 3,5-F₂ | CH₃ |
| O | 2,5-F₂ | 4-F | CH₃ |
| O | 2,5-F₂ | 2,6-F₂ | CH₃ |
| O | 2,5-F₂ | 2,5-F₂ | CH₃ |
| O | 3,5-F₂ | H | CH₃ |
| O | 3,5-F₂ | 3,5-F₂ | CH₃ |
| O | 3,5-F₂ | 4-F | CH₃ |
| O | 3,5-F₂ | 2,6-F₂ | CH₃ |
| O | 3,5-F₂ | 2,5-F₂ | CH₃ |
| O | 3,4-F₂ | H | CH₃ |
| O | 3,4-F₂ | 3,5-F₂ | CH₃ |
| O | 3,4-F₂ | 4-F | CH₃ |
| O | 3,4-F₂ | 2,6-F₂ | CH₃ |
| O | 3,4-F₂ | 2,5-F₂ | CH₃ |
| O | 4-CF₃ | H | CH₃ |
| O | 4-CF₃ | 3,5-F₂ | CH₃ |
| O | 4-CF₃ | 4-F | CH₃ |
| O | 4-CF₃ | 2,6-F₂ | CH₃ |
| O | 4-CF₃ | 2,5-F₂ | CH₃ |

TABLE XVIII

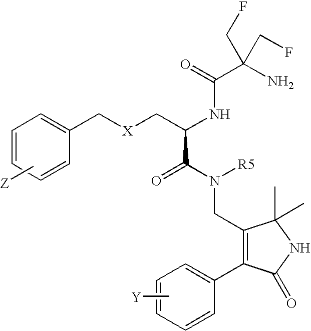

| X | Y | Z | R5 |
|---|---|---|---|
| O | 4-Cl | H | CH₂CH₃ |
| O | 4-Cl | 2-F | CH₂CH₃ |
| O | 4-Cl | 3-F | CH₂CH₃ |
| O | 4-Cl | 4-F | CH₂CH₃ |
| O | 4-Cl | 4-Cl | CH₂CH₃ |
| O | 4-Cl | 2,5-F₂ | CH₂CH₃ |
| O | 4-Cl | 2,4-F₂ | CH₂CH₃ |
| O | 4-Cl | 2-Cl | CH₂CH₃ |
| O | 4-Cl | 2,6-F₂ | CH₂CH₃ |
| O | 4-Cl | 3,5-F₂ | CH₂CH₃ |
| O | 4-Cl | 2,3-F₂ | CH₂CH₃ |
| O | 4-Cl | 3,4-F₂ | CH₂CH₃ |
| O | 4-Cl | 2,3,5-F₃ | CH₂CH₃ |
| O | 4-Cl | 2,3,6-F₃ | CH₂CH₃ |
| O | 4-Cl | 2,4,5-F₃ | CH₂CH₃ |
| O | 4-Cl | 2,6-Cl₂ | CH₂CH₃ |
| O | 4-Cl | 2-F-6-Cl | CH₂CH₃ |
| O | 4-Cl | 2-F-3-Cl | CH₂CH₃ |
| O | 4-Cl | 4-F-2-Cl | CH₂CH₃ |
| O | 4-Cl | 2-Cl-3,6-F₂ | CH₂CH₃ |
| O | 4-Cl | 3-Cl-2,6-F₂ | CH₂CH₃ |
| O | 4-Cl | 3-F-4-Cl | CH₂CH₃ |
| O | 4-Cl | 2-F-4-Cl | CH₂CH₃ |
| O | 4-Cl | 2,3-Cl₂ | CH₂CH₃ |
| O | 4-Cl | 2-CN | CH₂CH₃ |
| O | 4-F | H | CH₂CH₃ |
| O | 4-F | 2-F | CH₂CH₃ |
| O | 4-F | 3-F | CH₂CH₃ |
| O | 4-F | 4-F | CH₂CH₃ |
| O | 4-F | 4-Cl | CH₂CH₃ |
| O | 4-F | 2,5-F₂ | CH₂CH₃ |
| O | 4-F | 2,4-F₂ | CH₂CH₃ |
| O | 4-F | 2-Cl | CH₂CH₃ |
| O | 4-F | 2,6-F₂ | CH₂CH₃ |
| O | 4-F | 3,5-F₂ | CH₂CH₃ |
| O | 4-F | 2,3-F₂ | CH₂CH₃ |
| O | 4-F | 3,4-F₂ | CH₂CH₃ |
| O | 4-F | 2,3,5-F₃ | CH₂CH₃ |
| O | 4-F | 2,3,6-F₃ | CH₂CH₃ |
| O | 4-F | 2,4,5-F₃ | CH₂CH₃ |
| O | 4-F | 2,6-Cl₂ | CH₂CH₃ |
| O | 4-F | 2-F-6-Cl | CH₂CH₃ |
| O | 4-F | 2-F-3-Cl | CH₂CH₃ |
| O | 4-F | 4-F-2-Cl | CH₂CH₃ |
| O | 4-F | 2-Cl-3,6-F₂ | CH₂CH₃ |
| O | 4-F | 3-Cl-2,6-F₂ | CH₂CH₃ |
| O | 4-F | 3-F-4-Cl | CH₂CH₃ |
| O | 4-F | 2-F-4-Cl | CH₂CH₃ |
| O | 4-F | 2,3-Cl₂ | CH₂CH₃ |
| O | 4-F | 2-CN | CH₂CH₃ |
| CH₂ | 4-Cl | H | CH₂CH₃ |
| CH₂ | 4-Cl | 3,5-F₂ | CH₂CH₃ |
| CH₂ | 4-Cl | 4-F | CH₂CH₃ |
| CH₂ | 4-Cl | 2,6-F₂ | CH₂CH₃ |
| CH₂ | 4-F | H | CH₂CH₃ |
| CH₂ | 4-F | 3,5-F₂ | CH₂CH₃ |
| CH₂ | 4-F | 4-F | CH₂CH₃ |
| CH₂ | 4-F | 2,6-F₂ | CH₂CH₃ |
| O | 3-Cl | H | CH₂CH₃ |
| O | 3-Cl | 3,5-F₂ | CH₂CH₃ |
| O | 3-Cl | 4-F | CH₂CH₃ |
| O | 3-Cl | 2,6-F₂ | CH₂CH₃ |
| O | 3-Cl | 2,5-F₂ | CH₂CH₃ |
| O | 3-F | H | CH₂CH₃ |
| O | 3-F | 3,5-F₂ | CH₂CH₃ |
| O | 3-F | 4-F | CH₂CH₃ |
| O | 3-F | 2,6-F₂ | CH₂CH₃ |
| O | 3-F | 2,5-F₂ | CH₂CH₃ |
| O | 4-CN | H | CH₂CH₃ |
| O | 4-CN | 3,5-F₂ | CH₂CH₃ |
| O | 4-CN | 4-F | CH₂CH₃ |
| O | 4-CN | 2,6-F₂ | CH₂CH₃ |
| O | 4-CN | 2,5-F₂ | CH₂CH₃ |
| O | 2,5-F₂ | H | CH₂CH₃ |
| O | 2,5-F₂ | 3,5-F₂ | CH₂CH₃ |
| O | 2,5-F₂ | 4-F | CH₂CH₃ |
| O | 2,5-F₂ | 2,6-F₂ | CH₂CH₃ |
| O | 2,5-F₂ | 2,5-F₂ | CH₂CH₃ |
| O | 3,5-F₂ | H | CH₂CH₃ |
| O | 3,5-F₂ | 3,5-F₂ | CH₂CH₃ |
| O | 3,5-F₂ | 4-F | CH₂CH₃ |
| O | 3,5-F₂ | 2,6-F₂ | CH₂CH₃ |
| O | 3,5-F₂ | 2,5-F₂ | CH₂CH₃ |
| O | 3,4-F₂ | H | CH₂CH₃ |
| O | 3,4-F₂ | 3,5-F₂ | CH₂CH₃ |
| O | 3,4-F₂ | 4-F | CH₂CH₃ |
| O | 3,4-F₂ | 2,6-F₂ | CH₂CH₃ |
| O | 3,4-F₂ | 2,5-F₂ | CH₂CH₃ |
| O | 4-CF₃ | H | CH₂CH₃ |
| O | 4-CF₃ | 3,5-F₂ | CH₂CH₃ |
| O | 4-CF₃ | 4-F | CH₂CH₃ |
| O | 4-CF₃ | 2,6-F₂ | CH₂CH₃ |
| O | 4-CF₃ | 2,5-F₂ | CH₂CH₃ |
| O | 4-Cl | H | CH₃ |
| O | 4-Cl | 2-F | CH₃ |
| O | 4-Cl | 3-F | CH₃ |
| O | 4-Cl | 4-F | CH₃ |
| O | 4-Cl | 4-Cl | CH₃ |
| O | 4-Cl | 2,5-F₂ | CH₃ |
| O | 4-Cl | 2,4-F₂ | CH₃ |
| O | 4-Cl | 2-Cl | CH₃ |
| O | 4-Cl | 2,6-F₂ | CH₃ |
| O | 4-Cl | 3,5-F₂ | CH₃ |
| O | 4-Cl | 2,3-F₂ | CH₃ |
| O | 4-Cl | 3,4-F₂ | CH₃ |
| O | 4-Cl | 2,3,5-F₃ | CH₃ |
| O | 4-Cl | 2,3,6-F₃ | CH₃ |
| O | 4-Cl | 2,4,5-F₃ | CH₃ |
| O | 4-Cl | 2,6-Cl₂ | CH₃ |
| O | 4-Cl | 2-F-6-Cl | CH₃ |
| O | 4-Cl | 2-F-3-Cl | CH₃ |
| O | 4-Cl | 4-F-2-Cl | CH₃ |
| O | 4-Cl | 2-Cl-3,6-F₂ | CH₃ |
| O | 4-Cl | 3-Cl-2,6-F₂ | CH₃ |
| O | 4-Cl | 3-F-4-Cl | CH₃ |
| O | 4-Cl | 2-F-4-Cl | CH₃ |
| O | 4-Cl | 2,3-Cl₂ | CH₃ |
| O | 4-Cl | 2-CN | CH₃ |
| O | 4-F | H | CH₃ |
| O | 4-F | 2-F | CH₃ |
| O | 4-F | 3-F | CH₃ |
| O | 4-F | 4-F | CH₃ |
| O | 4-F | 4-Cl | CH₃ |
| O | 4-F | 2,5-F₂ | CH₃ |
| O | 4-F | 2,4-F₂ | CH₃ |
| O | 4-F | 2-Cl | CH₃ |
| O | 4-F | 2,6-F₂ | CH₃ |
| O | 4-F | 3,5-F₂ | CH₃ |
| O | 4-F | 2,3-F₂ | CH₃ |
| O | 4-F | 3,4-F₂ | CH₃ |
| O | 4-F | 2,3,5-F₃ | CH₃ |
| O | 4-F | 2,3,6-F₃ | CH₃ |
| O | 4-F | 2,4,5-F₃ | CH₃ |
| O | 4-F | 2,6-Cl₂ | CH₃ |
| O | 4-F | 2-F-6-Cl | CH₃ |
| O | 4-F | 2-F-3-Cl | CH₃ |
| O | 4-F | 4-F-2-Cl | CH₃ |
| O | 4-F | 2-Cl-3,6-F₂ | CH₃ |
| O | 4-F | 3-Cl-2,6-F₂ | CH₃ |
| O | 4-F | 3-F-4-Cl | CH₃ |
| O | 4-F | 2-F-4-Cl | CH₃ |
| O | 4-F | 2,3-Cl₂ | CH₃ |
| O | 4-F | 2-CN | CH₃ |
| CH₂ | 4-Cl | H | CH₃ |
| CH₂ | 4-Cl | 3,5-F₂ | CH₃ |
| CH₂ | 4-Cl | 4-F | CH₃ |
| CH₂ | 4-Cl | 2,6-F₂ | CH₃ |
| CH₂ | 4-F | H | CH₃ |
| CH₂ | 4-F | 3,5-F₂ | CH₃ |
| CH₂ | 4-F | 4-F | CH₃ |
| CH₂ | 4-F | 2,6-F₂ | CH₃ |
| O | 3-F | H | CH₃ |
| O | 3-F | 3,5-F₂ | CH₃ |
| O | 3-F | 4-F | CH₃ |
| O | 3-F | 2,6-F₂ | CH₃ |
| O | 3-F | 2,5-F₂ | CH₃ |
| O | 2,5-F₂ | H | CH₃ |
| O | 2,5-F₂ | 3,5-F₂ | CH₃ |
| O | 2,5-F₂ | 4-F | CH₃ |
| O | 2,5-F₂ | 2,6-F₂ | CH₃ |
| O | 2,5-F₂ | 2,5-F₂ | CH₃ |
| O | 3,5-F₂ | H | CH₃ |
| O | 3,5-F₂ | 3,5-F₂ | CH₃ |
| O | 3,5-F₂ | 4-F | CH₃ |
| O | 3,5-F₂ | 2,6-F₂ | CH₃ |
| O | 3,5-F₂ | 2,5-F₂ | CH₃ |
| O | 3,4-F₂ | H | CH₃ |
| O | 3,4-F₂ | 3,5-F₂ | CH₃ |
| O | 3,4-F₂ | 4-F | CH₃ |
| O | 3,4-F₂ | 2,6-F₂ | CH₃ |
| O | 3,4-F₂ | 2,5-F₂ | CH₃ |
| O | 4-CF₃ | H | CH₃ |
| O | 4-CF₃ | 3,5-F₂ | CH₃ |
| O | 4-CF₃ | 4-F | CH₃ |
| O | 4-CF₃ | 2,6-F₂ | CH₃ |
| O | 4-CF₃ | 2,5-F₂ | CH₃ |

TABLE XIX

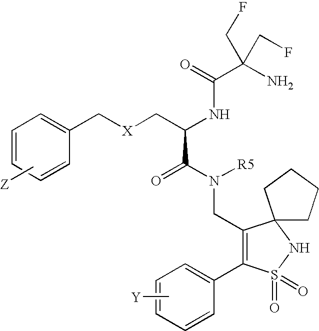

| X | Y | Z | R5 |
|---|---|---|---|
| O | 4-Cl | H | CH₂CH₃ |
| O | 4-Cl | 2-F | CH₂CH₃ |
| O | 4-Cl | 3-F | CH₂CH₃ |
| O | 4-Cl | 4-F | CH₂CH₃ |
| O | 4-Cl | 4-Cl | CH₂CH₃ |
| O | 4-Cl | 2,5-F₂ | CH₂CH₃ |
| O | 4-Cl | 2,4-F₂ | CH₂CH₃ |
| O | 4-Cl | 2-Cl | CH₂CH₃ |
| O | 4-Cl | 2,6-F₂ | CH₂CH₃ |
| O | 4-Cl | 3,5-F₂ | CH₂CH₃ |
| O | 4-Cl | 2,3-F₂ | CH₂CH₃ |
| O | 4-Cl | 3,4-F₂ | CH₂CH₃ |
| O | 4-Cl | 2,3,5-F₃ | CH₂CH₃ |
| O | 4-Cl | 2,3,6-F₃ | CH₂CH₃ |
| O | 4-Cl | 2,4,5-F₃ | CH₂CH₃ |
| O | 4-Cl | 2,6-Cl₂ | CH₂CH₃ |
| O | 4-Cl | 2-F-6-Cl | CH₂CH₃ |
| O | 4-Cl | 2-F-3-Cl | CH₂CH₃ |
| O | 4-Cl | 4-F-2-Cl | CH₂CH₃ |
| O | 4-Cl | 2-Cl-3,6-F₂ | CH₂CH₃ |
| O | 4-Cl | 3-Cl-2,6-F₂ | CH₂CH₃ |
| O | 4-Cl | 3-F-4-Cl | CH₂CH₃ |
| O | 4-Cl | 2-F-4-Cl | CH₂CH₃ |
| O | 4-Cl | 2,3-Cl₂ | CH₂CH₃ |
| O | 4-Cl | 2-CN | CH₂CH₃ |
| O | 4-F | H | CH₂CH₃ |
| O | 4-F | 2-F | CH₂CH₃ |
| O | 4-F | 3-F | CH₂CH₃ |
| O | 4-F | 4-F | CH₂CH₃ |
| O | 4-F | 4-Cl | CH₂CH₃ |
| O | 4-F | 2,5-F₂ | CH₂CH₃ |
| O | 4-F | 2,4-F₂ | CH₂CH₃ |
| O | 4-F | 2-Cl | CH₂CH₃ |
| O | 4-F | 2,6-F₂ | CH₂CH₃ |
| O | 4-F | 3,5-F₂ | CH₂CH₃ |
| O | 4-F | 2,3-F₂ | CH₂CH₃ |
| O | 4-F | 3,4-F₂ | CH₂CH₃ |
| O | 4-F | 2,3,5-F₃ | CH₂CH₃ |
| O | 4-F | 2,3,6-F₃ | CH₂CH₃ |
| O | 4-F | 2,4,5-F₃ | CH₂CH₃ |
| O | 4-F | 2,6-Cl₂ | CH₂CH₃ |
| O | 4-F | 2-F-6-Cl | CH₂CH₃ |
| O | 4-F | 2-F-3-Cl | CH₂CH₃ |
| O | 4-F | 4-F-2-Cl | CH₂CH₃ |
| O | 4-F | 2-Cl-3,6-F₂ | CH₂CH₃ |
| O | 4-F | 3-Cl-2,6-F₂ | CH₂CH₃ |
| O | 4-F | 3-F-4-Cl | CH₂CH₃ |
| O | 4-F | 2-F-4-Cl | CH₂CH₃ |
| O | 4-F | 2,3-Cl₂ | CH₂CH₃ |
| O | 4-F | 2-CN | CH₂CH₃ |
| CH₂ | 4-Cl | H | CH₂CH₃ |
| CH₂ | 4-Cl | 3,5-F₂ | CH₂CH₃ |
| CH₂ | 4-Cl | 4-F | CH₂CH₃ |
| CH₂ | 4-Cl | 2,6-F₂ | CH₂CH₃ |
| CH₂ | 4-F | H | CH₂CH₃ |
| CH₂ | 4-F | 3,5-F₂ | CH₂CH₃ |
| CH₂ | 4-F | 4-F | CH₂CH₃ |
| CH₂ | 4-F | 2,6-F₂ | CH₂CH₃ |
| O | 3-Cl | H | CH₂CH₃ |
| O | 3-Cl | 3,5-F₂ | CH₂CH₃ |
| O | 3-Cl | 4-F | CH₂CH₃ |
| O | 3-Cl | 2,6-F₂ | CH₂CH₃ |
| O | 3-Cl | 2,5-F₂ | CH₂CH₃ |
| O | 3-F | H | CH₂CH₃ |
| O | 3-F | 3,5-F₂ | CH₂CH₃ |
| O | 3-F | 4-F | CH₂CH₃ |
| O | 3-F | 2,6-F₂ | CH₂CH₃ |
| O | 3-F | 2,5-F₂ | CH₂CH₃ |
| O | 4-CN | H | CH₂CH₃ |
| O | 4-CN | 3,5-F₂ | CH₂CH₃ |
| O | 4-CN | 4-F | CH₂CH₃ |
| O | 4-CN | 2,6-F₂ | CH₂CH₃ |
| O | 4-CN | 2,5-F₂ | CH₂CH₃ |
| O | 2,5-F₂ | H | CH₂CH₃ |
| O | 2,5-F₂ | 3,5-F₂ | CH₂CH₃ |
| O | 2,5-F₂ | 4-F | CH₂CH₃ |
| O | 2,5-F₂ | 2,6-F₂ | CH₂CH₃ |
| O | 2,5-F₂ | 2,5-F₂ | CH₂CH₃ |
| O | 3,5-F₂ | H | CH₂CH₃ |
| O | 3,5-F₂ | 3,5-F₂ | CH₂CH₃ |
| O | 3,5-F₂ | 4-F | CH₂CH₃ |
| O | 3,5-F₂ | 2,6-F₂ | CH₂CH₃ |
| O | 3,5-F₂ | 2,5-F₂ | CH₂CH₃ |
| O | 3,4-F₂ | H | CH₂CH₃ |
| O | 3,4-F₂ | 3,5-F₂ | CH₂CH₃ |
| O | 3,4-F₂ | 4-F | CH₂CH₃ |
| O | 3,4-F₂ | 2,6-F₂ | CH₂CH₃ |
| O | 3,4-F₂ | 2,5-F₂ | CH₂CH₃ |
| O | 4-CF₃ | H | CH₂CH₃ |
| O | 4-CF₃ | 3,5-F₂ | CH₂CH₃ |
| O | 4-CF₃ | 4-F | CH₂CH₃ |
| O | 4-CF₃ | 2,6-F₂ | CH₂CH₃ |
| O | 4-CF₃ | 2,5-F₂ | CH₂CH₃ |
| O | 4-Cl | H | CH₃ |
| O | 4-Cl | 2-F | CH₃ |
| O | 4-Cl | 3-F | CH₃ |
| O | 4-Cl | 4-F | CH₃ |
| O | 4-Cl | 4-Cl | CH₃ |
| O | 4-Cl | 2,5-F₂ | CH₃ |
| O | 4-Cl | 2,4-F₂ | CH₃ |
| O | 4-Cl | 2-Cl | CH₃ |
| O | 4-Cl | 2,6-F₂ | CH₃ |
| O | 4-Cl | 3,5-F₂ | CH₃ |
| O | 4-Cl | 2,3-F₂ | CH₃ |
| O | 4-Cl | 3,4-F₂ | CH₃ |
| O | 4-Cl | 2,3,5-F₃ | CH₃ |
| O | 4-Cl | 2,3,6-F₃ | CH₃ |
| O | 4-Cl | 2,4,5-F₃ | CH₃ |
| O | 4-Cl | 2,6-Cl₂ | CH₃ |
| O | 4-Cl | 2-F-6-Cl | CH₃ |
| O | 4-Cl | 2-F-3-Cl | CH₃ |
| O | 4-Cl | 4-F-2-Cl | CH₃ |
| O | 4-Cl | 2-Cl-3,6-F₂ | CH₃ |
| O | 4-Cl | 3-Cl-2,6-F₂ | CH₃ |
| O | 4-Cl | 3-F-4-Cl | CH₃ |
| O | 4-Cl | 2-F-4-Cl | CH₃ |
| O | 4-Cl | 2,3-Cl₂ | CH₃ |
| O | 4-Cl | 2-CN | CH₃ |
| O | 4-F | H | CH₃ |
| O | 4-F | 2-F | CH₃ |
| O | 4-F | 3-F | CH₃ |
| O | 4-F | 4-F | CH₃ |
| O | 4-F | 4-Cl | CH₃ |
| O | 4-F | 2,5-F₂ | CH₃ |
| O | 4-F | 2,4-F₂ | CH₃ |
| O | 4-F | 2-Cl | CH₃ |
| O | 4-F | 2,6-F₂ | CH₃ |
| O | 4-F | 3,5-F₂ | CH₃ |
| O | 4-F | 2,3-F₂ | CH₃ |
| O | 4-F | 3,4-F₂ | CH₃ |
| O | 4-F | 2,3,5-F₃ | CH₃ |
| O | 4-F | 2,3,6-F₃ | CH₃ |
| O | 4-F | 2,4,5-F₃ | CH₃ |
| O | 4-F | 2,6-Cl₂ | CH₃ |
| O | 4-F | 2-F-6-Cl | CH₃ |
| O | 4-F | 2-F-3-Cl | CH₃ |
| O | 4-F | 4-F-2-Cl | CH₃ |
| O | 4-F | 2-Cl-3,6-F₂ | CH₃ |
| O | 4-F | 3-Cl-2,6-F₂ | CH₃ |
| O | 4-F | 3-F-4-Cl | CH₃ |
| O | 4-F | 2-F-4-Cl | CH₃ |
| O | 4-F | 2,3-Cl₂ | CH₃ |
| O | 4-F | 2-CN | CH₃ |
| CH₂ | 4-Cl | H | CH₃ |
| CH₂ | 4-Cl | 3,5-F₂ | CH₃ |
| CH₂ | 4-Cl | 4-F | CH₃ |
| CH₂ | 4-Cl | 2,6-F₂ | CH₃ |
| CH₂ | 4-F | H | CH₃ |
| CH₂ | 4-F | 3,5-F₂ | CH₃ |
| CH₂ | 4-F | 4-F | CH₃ |
| CH₂ | 4-F | 2,6-F₂ | CH₃ |
| O | 3-F | H | CH₃ |
| O | 3-F | 3,5-F₂ | CH₃ |
| O | 3-F | 4-F | CH₃ |
| O | 3-F | 2,6-F₂ | CH₃ |
| O | 3-F | 2,5-F₂ | CH₃ |
| O | 2,5-F₂ | H | CH₃ |
| O | 2,5-F₂ | 3,5-F₂ | CH₃ |
| O | 2,5-F₂ | 4-F | CH₃ |
| O | 2,5-F₂ | 2,6-F₂ | CH₃ |
| O | 2,5-F₂ | 2,5-F₂ | CH₃ |
| O | 3,5-F₂ | H | CH₃ |
| O | 3,5-F₂ | 3,5-F₂ | CH₃ |
| O | 3,5-F₂ | 4-F | CH₃ |
| O | 3,5-F₂ | 2,6-F₂ | CH₃ |
| O | 3,5-F₂ | 2,5-F₂ | CH₃ |
| O | 3,4-F₂ | H | CH₃ |
| O | 3,4-F₂ | 3,5-F₂ | CH₃ |
| O | 3,4-F₂ | 4-F | CH₃ |
| O | 3,4-F₂ | 2,6-F₂ | CH₃ |
| O | 3,4-F₂ | 2,5-F₂ | CH₃ |
| O | 4-CF₃ | H | CH₃ |
| O | 4-CF₃ | 3,5-F₂ | CH₃ |
| O | 4-CF₃ | 4-F | CH₃ |
| O | 4-CF₃ | 2,6-F₂ | CH₃ |
| O | 4-CF₃ | 2,5-F₂ | CH₃ |

TABLE XX

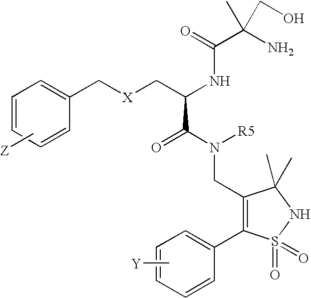

| X | Y | Z | R5 |
|---|---|---|---|
| O | 4-Cl | H | CH₂CH₃ |
| O | 4-Cl | 2-F | CH₂CH₃ |
| O | 4-Cl | 3-F | CH₂CH₃ |
| O | 4-Cl | 4-F | CH₂CH₃ |
| O | 4-Cl | 4-Cl | CH₂CH₃ |
| O | 4-Cl | 2,5-F₂ | CH₂CH₃ |
| O | 4-Cl | 2,4-F₂ | CH₂CH₃ |
| O | 4-Cl | 2-Cl | CH₂CH₃ |
| O | 4-Cl | 2,6-F₂ | CH₂CH₃ |
| O | 4-Cl | 3,5-F₂ | CH₂CH₃ |
| O | 4-Cl | 2,3-F₂ | CH₂CH₃ |
| O | 4-Cl | 3,4-F₂ | CH₂CH₃ |
| O | 4-Cl | 2,3,5-F₃ | CH₂CH₃ |
| O | 4-Cl | 2,3,6-F₃ | CH₂CH₃ |
| O | 4-Cl | 2,4,5-F₃ | CH₂CH₃ |
| O | 4-Cl | 2,6-Cl₂ | CH₂CH₃ |
| O | 4-Cl | 2-F-6-Cl | CH₂CH₃ |
| O | 4-Cl | 2-F-3-Cl | CH₂CH₃ |
| O | 4-Cl | 4-F-2-Cl | CH₂CH₃ |
| O | 4-Cl | 2-Cl-3,6-F₂ | CH₂CH₃ |
| O | 4-Cl | 3-Cl-2,6-F₂ | CH₂CH₃ |
| O | 4-Cl | 3-F-4-Cl | CH₂CH₃ |
| O | 4-Cl | 2-F-4-Cl | CH₂CH₃ |
| O | 4-Cl | 2,3-Cl₂ | CH₂CH₃ |
| O | 4-Cl | 2-CN | CH₂CH₃ |
| O | 4-F | H | CH₂CH₃ |
| O | 4-F | 2-F | CH₂CH₃ |
| O | 4-F | 3-F | CH₂CH₃ |
| O | 4-F | 4-F | CH₂CH₃ |
| O | 4-F | 4-Cl | CH₂CH₃ |
| O | 4-F | 2,5-F₂ | CH₂CH₃ |
| O | 4-F | 2,4-F₂ | CH₂CH₃ |
| O | 4-F | 2-Cl | CH₂CH₃ |
| O | 4-F | 2,6-F₂ | CH₂CH₃ |
| O | 4-F | 3,5-F₂ | CH₂CH₃ |
| O | 4-F | 2,3-F₂ | CH₂CH₃ |
| O | 4-F | 3,4-F₂ | CH₂CH₃ |
| O | 4-F | 2,3,5-F₃ | CH₂CH₃ |
| O | 4-F | 2,3,6-F₃ | CH₂CH₃ |
| O | 4-F | 2,4,5-F₃ | CH₂CH₃ |
| O | 4-F | 2,6-Cl₂ | CH₂CH₃ |
| O | 4-F | 2-F-6-Cl | CH₂CH₃ |
| O | 4-F | 2-F-3-Cl | CH₂CH₃ |
| O | 4-F | 4-F-2-Cl | CH₂CH₃ |
| O | 4-F | 2-Cl-3,6-F₂ | CH₂CH₃ |
| O | 4-F | 3-Cl-2,6-F₂ | CH₂CH₃ |
| O | 4-F | 3-F-4-Cl | CH₂CH₃ |
| O | 4-F | 2-F-4-Cl | CH₂CH₃ |
| O | 4-F | 2,3-Cl₂ | CH₂CH₃ |
| O | 4-F | 2-CN | CH₂CH₃ |
| CH₂ | 4-Cl | H | CH₂CH₃ |
| CH₂ | 4-Cl | 3,5-F₂ | CH₂CH₃ |
| CH₂ | 4-Cl | 4-F | CH₂CH₃ |
| CH₂ | 4-Cl | 2,6-F₂ | CH₂CH₃ |
| CH₂ | 4-F | H | CH₂CH₃ |
| CH₂ | 4-F | 3,5-F₂ | CH₂CH₃ |
| CH₂ | 4-F | 4-F | CH₂CH₃ |
| CH₂ | 4-F | 2,6-F₂ | CH₂CH₃ |
| O | 3-Cl | H | CH₂CH₃ |
| O | 3-Cl | 3,5-F₂ | CH₂CH₃ |
| O | 3-Cl | 4-F | CH₂CH₃ |
| O | 3-Cl | 2,6-F₂ | CH₂CH₃ |
| O | 3-Cl | 2,5-F₂ | CH₂CH₃ |
| O | 3-F | H | CH₂CH₃ |
| O | 3-F | 3,5-F₂ | CH₂CH₃ |
| O | 3-F | 4-F | CH₂CH₃ |
| O | 3-F | 2,6-F₂ | CH₂CH₃ |
| O | 3-F | 2,5-F₂ | CH₂CH₃ |
| O | 4-CN | H | CH₂CH₃ |
| O | 4-CN | 3,5-F₂ | CH₂CH₃ |
| O | 4-CN | 4-F | CH₂CH₃ |
| O | 4-CN | 2,6-F₂ | CH₂CH₃ |
| O | 4-CN | 2,5-F₂ | CH₂CH₃ |
| O | 2,5-F₂ | H | CH₂CH₃ |
| O | 2,5-F₂ | 3,5-F₂ | CH₂CH₃ |
| O | 2,5-F₂ | 4-F | CH₂CH₃ |
| O | 2,5-F₂ | 2,6-F₂ | CH₂CH₃ |
| O | 2,5-F₂ | 2,5-F₂ | CH₂CH₃ |
| O | 3,5-F₂ | H | CH₂CH₃ |
| O | 3,5-F₂ | 3,5-F₂ | CH₂CH₃ |
| O | 3,5-F₂ | 4-F | CH₂CH₃ |
| O | 3,5-F₂ | 2,6-F₂ | CH₂CH₃ |
| O | 3,5-F₂ | 2,5-F₂ | CH₂CH₃ |
| O | 3,4-F₂ | H | CH₂CH₃ |
| O | 3,4-F₂ | 3,5-F₂ | CH₂CH₃ |
| O | 3,4-F₂ | 4-F | CH₂CH₃ |
| O | 3,4-F₂ | 2,6-F₂ | CH₂CH₃ |
| O | 3,4-F₂ | 2,5-F₂ | CH₂CH₃ |
| O | 4-CF₃ | H | CH₂CH₃ |
| O | 4-CF₃ | 3,5-F₂ | CH₂CH₃ |
| O | 4-CF₃ | 4-F | CH₂CH₃ |
| O | 4-CF₃ | 2,6-F₂ | CH₂CH₃ |
| O | 4-CF₃ | 2,5-F₂ | CH₂CH₃ |
| O | 4-Cl | H | CH₃ |
| O | 4-Cl | 2-F | CH₃ |
| O | 4-Cl | 3-F | CH₃ |
| O | 4-Cl | 4-F | CH₃ |
| O | 4-Cl | 4-Cl | CH₃ |
| O | 4-Cl | 2,5-F₂ | CH₃ |
| O | 4-Cl | 2,4-F₂ | CH₃ |
| O | 4-Cl | 2-Cl | CH₃ |
| O | 4-Cl | 2,6-F₂ | CH₃ |
| O | 4-Cl | 3,5-F₂ | CH₃ |
| O | 4-Cl | 2,3-F₂ | CH₃ |
| O | 4-Cl | 3,4-F₂ | CH₃ |
| O | 4-Cl | 2,3,5-F₃ | CH₃ |
| O | 4-Cl | 2,3,6-F₃ | CH₃ |
| O | 4-Cl | 2,4,5-F₃ | CH₃ |
| O | 4-Cl | 2,6-Cl₂ | CH₃ |
| O | 4-Cl | 2-F-6-Cl | CH₃ |
| O | 4-Cl | 2-F-3-Cl | CH₃ |
| O | 4-Cl | 4-F-2-Cl | CH₃ |
| O | 4-Cl | 2-Cl-3,6-F₂ | CH₃ |
| O | 4-Cl | 3-Cl-2,6-F₂ | CH₃ |
| O | 4-Cl | 3-F-4-Cl | CH₃ |
| O | 4-Cl | 2-F-4-Cl | CH₃ |
| O | 4-Cl | 2,3-Cl₂ | CH₃ |
| O | 4-Cl | 2-CN | CH₃ |
| O | 4-F | H | CH₃ |
| O | 4-F | 2-F | CH₃ |
| O | 4-F | 3-F | CH₃ |
| O | 4-F | 4-F | CH₃ |
| O | 4-F | 4-Cl | CH₃ |
| O | 4-F | 2,5-F₂ | CH₃ |
| O | 4-F | 2,4-F₂ | CH₃ |
| O | 4-F | 2-Cl | CH₃ |
| O | 4-F | 2,6-F₂ | CH₃ |
| O | 4-F | 3,5-F₂ | CH₃ |
| O | 4-F | 2,3-F₂ | CH₃ |
| O | 4-F | 3,4-F₂ | CH₃ |
| O | 4-F | 2,3,5-F₃ | CH₃ |
| O | 4-F | 2,3,6-F₃ | CH₃ |
| O | 4-F | 2,4,5-F₃ | CH₃ |
| O | 4-F | 2,6-Cl₂ | CH₃ |
| O | 4-F | 2-F-6-Cl | CH₃ |
| O | 4-F | 2-F-3-Cl | CH₃ |
| O | 4-F | 4-F-2-Cl | CH₃ |
| O | 4-F | 2-Cl-3,6-F₂ | CH₃ |
| O | 4-F | 3-Cl-2,6-F₂ | CH₃ |
| O | 4-F | 3-F-4-Cl | CH₃ |
| O | 4-F | 2-F-4-Cl | CH₃ |
| O | 4-F | 2,3-Cl₂ | CH₃ |
| O | 4-F | 2-CN | CH₃ |
| CH₂ | 4-Cl | H | CH₃ |
| CH₂ | 4-Cl | 3,5-F₂ | CH₃ |
| CH₂ | 4-Cl | 4-F | CH₃ |
| CH₂ | 4-Cl | 2,6-F₂ | CH₃ |
| CH₂ | 4-F | H | CH₃ |
| CH₂ | 4-F | 3,5-F₂ | CH₃ |
| CH₂ | 4-F | 4-F | CH₃ |
| CH₂ | 4-F | 2,6-F₂ | CH₃ |
| O | 3-F | H | CH₃ |
| O | 3-F | 3,5-F₂ | CH₃ |
| O | 3-F | 4-F | CH₃ |
| O | 3-F | 2,6-F₂ | CH₃ |
| O | 3-F | 2,5-F₂ | CH₃ |
| O | 2,5-F₂ | H | CH₃ |
| O | 2,5-F₂ | 3,5-F₂ | CH₃ |
| O | 2,5-F₂ | 4-F | CH₃ |
| O | 2,5-F₂ | 2,6-F₂ | CH₃ |
| O | 2,5-F₂ | 2,5-F₂ | CH₃ |
| O | 3,5-F₂ | H | CH₃ |
| O | 3,5-F₂ | 3,5-F₂ | CH₃ |
| O | 3,5-F₂ | 4-F | CH₃ |
| O | 3,5-F₂ | 2,6-F₂ | CH₃ |
| O | 3,5-F₂ | 2,5-F₂ | CH₃ |
| O | 3,4-F₂ | H | CH₃ |
| O | 3,4-F₂ | 3,5-F₂ | CH₃ |
| O | 3,4-F₂ | 4-F | CH₃ |
| O | 3,4-F₂ | 2,6-F₂ | CH₃ |
| O | 3,4-F₂ | 2,5-F₂ | CH₃ |
| O | 4-CF₃ | H | CH₃ |
| O | 4-CF₃ | 3,5-F₂ | CH₃ |
| O | 4-CF₃ | 4-F | CH₃ |
| O | 4-CF₃ | 2,6-F₂ | CH₃ |
| O | 4-CF₃ | 2,5-F₂ | CH₃ |

TABLE XXI

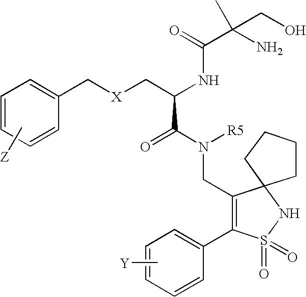

| X | Y | Z | R5 |
|---|---|---|---|
| O | 4-Cl | H | CH₂CH₃ |
| O | 4-Cl | 2-F | CH₂CH₃ |
| O | 4-Cl | 3-F | CH₂CH₃ |
| O | 4-Cl | 4-F | CH₂CH₃ |
| O | 4-Cl | 4-Cl | CH₂CH₃ |
| O | 4-Cl | 2,5-F₂ | CH₂CH₃ |
| O | 4-Cl | 2,4-F₂ | CH₂CH₃ |
| O | 4-Cl | 2-Cl | CH₂CH₃ |
| O | 4-Cl | 2,6-F₂ | CH₂CH₃ |
| O | 4-Cl | 3,5-F₂ | CH₂CH₃ |
| O | 4-Cl | 2,3-F₂ | CH₂CH₃ |
| O | 4-Cl | 3,4-F₂ | CH₂CH₃ |
| O | 4-Cl | 2,3,5-F₃ | CH₂CH₃ |
| O | 4-Cl | 2,3,6-F₃ | CH₂CH₃ |
| O | 4-Cl | 2,4,5-F₃ | CH₂CH₃ |
| O | 4-Cl | 2,6-Cl₂ | CH₂CH₃ |
| O | 4-Cl | 2-F-6-Cl | CH₂CH₃ |
| O | 4-Cl | 2-F-3-Cl | CH₂CH₃ |
| O | 4-Cl | 4-F-2-Cl | CH₂CH₃ |
| O | 4-Cl | 2-Cl-3,6-F₂ | CH₂CH₃ |
| O | 4-Cl | 3-Cl-2,6-F₂ | CH₂CH₃ |
| O | 4-Cl | 3-F-4-Cl | CH₂CH₃ |
| O | 4-Cl | 2-F-4-Cl | CH₂CH₃ |
| O | 4-Cl | 2,3-Cl₂ | CH₂CH₃ |
| O | 4-Cl | 2-CN | CH₂CH₃ |
| O | 4-F | H | CH₂CH₃ |
| O | 4-F | 2-F | CH₂CH₃ |
| O | 4-F | 3-F | CH₂CH₃ |
| O | 4-F | 4-F | CH₂CH₃ |
| O | 4-F | 4-Cl | CH₂CH₃ |
| O | 4-F | 2,5-F₂ | CH₂CH₃ |
| O | 4-F | 2,4-F₂ | CH₂CH₃ |
| O | 4-F | 2-Cl | CH₂CH₃ |
| O | 4-F | 2,6-F₂ | CH₂CH₃ |
| O | 4-F | 3,5-F₂ | CH₂CH₃ |
| O | 4-F | 2,3-F₂ | CH₂CH₃ |
| O | 4-F | 3,4-F₂ | CH₂CH₃ |
| O | 4-F | 2,3,5-F₂ | CH₂CH₃ |
| O | 4-F | 2,3,6-F₃ | CH₂CH₃ |
| O | 4-F | 2,4,5-F₃ | CH₂CH₃ |
| O | 4-F | 2,6-Cl₂ | CH₂CH₃ |
| O | 4-F | 2-F-6-Cl | CH₂CH₃ |
| O | 4-F | 2-F-3-Cl | CH₂CH₃ |
| O | 4-F | 4-F-2-Cl | CH₂CH₃ |
| O | 4-F | 2-Cl-3,6-F₂ | CH₂CH₃ |
| O | 4-F | 3-Cl-2,6-F₂ | CH₂CH₃ |
| O | 4-F | 3-F-4-Cl | CH₂CH₃ |
| O | 4-F | 2-F-4-Cl | CH₂CH₃ |
| O | 4-F | 2,3-Cl₂ | CH₂CH₃ |
| O | 4-F | 2-CN | CH₂CH₃ |
| CH₂ | 4-Cl | H | CH₂CH₃ |
| CH₂ | 4-Cl | 3,5-F₂ | CH₂CH₃ |
| CH₂ | 4-Cl | 4-F | CH₂CH₃ |
| CH₂ | 4-Cl | 2,6-F₂ | CH₂CH₃ |
| CH₂ | 4-F | H | CH₂CH₃ |
| CH₂ | 4-F | 3,5-F₂ | CH₂CH₃ |
| CH₂ | 4-F | 4-F | CH₂CH₃ |
| CH₂ | 4-F | 2,6-F₂ | CH₂CH₃ |
| O | 3-Cl | H | CH₂CH₃ |
| O | 3-Cl | 3,5-F₂ | CH₂CH₃ |
| O | 3-Cl | 4-F | CH₂CH₃ |
| O | 3-Cl | 2,6-F₂ | CH₂CH₃ |
| O | 3-Cl | 2,5-F₂ | CH₂CH₃ |
| O | 3-F | H | CH₂CH₃ |
| O | 3-F | 3,5-F₂ | CH₂CH₃ |
| O | 3-F | 4-F | CH₂CH₃ |
| O | 3-F | 2,6-F₂ | CH₂CH₃ |
| O | 3-F | 2,5-F₂ | CH₂CH₃ |
| O | 4-CN | H | CH₂CH₃ |
| O | 4-CN | 3,5-F₂ | CH₂CH₃ |
| O | 4-CN | 4-F | CH₂CH₃ |
| O | 4-CN | 2,6-F₂ | CH₂CH₃ |
| O | 4-CN | 2,5-F₂ | CH₂CH₃ |
| O | 2,5-F₂ | H | CH₂CH₃ |
| O | 2,5-F₂ | 3,5-F₂ | CH₂CH₃ |
| O | 2,5-F₂ | 4-F | CH₂CH₃ |
| O | 2,5-F₂ | 2,6-F₂ | CH₂CH₃ |
| O | 2,5-F₂ | 2,5-F₂ | CH₂CH₃ |
| O | 3,5-F₂ | H | CH₂CH₃ |
| O | 3,5-F₂ | 3,5-F₂ | CH₂CH₃ |
| O | 3,5-F₂ | 4-F | CH₂CH₃ |
| O | 3,5-F₂ | 2,6-F₂ | CH₂CH₃ |
| O | 3,5-F₂ | 2,5-F₂ | CH₂CH₃ |
| O | 3,4-F₂ | H | CH₂CH₃ |
| O | 3,4-F₂ | 3,5-F₂ | CH₂CH₃ |
| O | 3,4-F₂ | 4-F | CH₂CH₃ |
| O | 3,4-F₂ | 2,6-F₂ | CH₂CH₃ |
| O | 3,4-F₂ | 2,5-F₂ | CH₂CH₃ |
| O | 4-CF₃ | H | CH₂CH₃ |
| O | 4-CF₃ | 3,5-F₂ | CH₂CH₃ |
| O | 4-CF₃ | 4-F | CH₂CH₃ |
| O | 4-CF₃ | 2,6-F₂ | CH₂CH₃ |
| O | 4-CF₃ | 2,5-F₂ | CH₂CH₃ |
| O | 4-Cl | H | CH₃ |
| O | 4-Cl | 2-F | CH₃ |
| O | 4-Cl | 3-F | CH₃ |
| O | 4-Cl | 4-F | CH₃ |
| O | 4-Cl | 4-Cl | CH₃ |
| O | 4-Cl | 2,5-F₂ | CH₃ |
| O | 4-Cl | 2,4-F₂ | CH₃ |
| O | 4-Cl | 2-Cl | CH₃ |
| O | 4-Cl | 2,6-F₂ | CH₃ |
| O | 4-Cl | 3,5-F₂ | CH₃ |
| O | 4-Cl | 2,3-F₂ | CH₃ |
| O | 4-Cl | 3,4-F₂ | CH₃ |
| O | 4-Cl | 2,3,5-F₃ | CH₃ |
| O | 4-Cl | 2,3,6-F₂ | CH₃ |
| O | 4-Cl | 2,4,5-F₃ | CH₃ |
| O | 4-Cl | 2,6-Cl₂ | CH₃ |
| O | 4-Cl | 2-F-6-Cl | CH₃ |
| O | 4-Cl | 2-F-3-Cl | CH₃ |
| O | 4-Cl | 4-F-2-Cl | CH₃ |
| O | 4-Cl | 2-Cl-3,6-F₂ | CH₃ |
| O | 4-Cl | 3-Cl-2,6-F₂ | CH₃ |
| O | 4-Cl | 3-F-4-Cl | CH₃ |
| O | 4-Cl | 2-F-4-Cl | CH₃ |
| O | 4-Cl | 2,3-Cl₂ | CH₃ |
| O | 4-Cl | 2-CN | CH₃ |
| O | 4-F | H | CH₃ |
| O | 4-F | 2-F | CH₃ |
| O | 4-F | 3-F | CH₃ |
| O | 4-F | 4-F | CH₃ |
| O | 4-F | 4-Cl | CH₃ |
| O | 4-F | 2,5-F₂ | CH₃ |
| O | 4-F | 2,4-F₂ | CH₃ |
| O | 4-F | 2-Cl | CH₃ |
| O | 4-F | 2,6-F₂ | CH₃ |
| O | 4-F | 3,5-F₂ | CH₃ |
| O | 4-F | 2,3-F₂ | CH₃ |
| O | 4-F | 3,4-F₂ | CH₃ |
| O | 4-F | 2,3,5-F₃ | CH₃ |
| O | 4-F | 2,3,6-F₃ | CH₃ |
| O | 4-F | 2,4,5-F₃ | CH₃ |
| O | 4-F | 2,6-Cl₂ | CH₃ |
| O | 4-F | 2-F-6-Cl | CH₃ |
| O | 4-F | 2-F-3-Cl | CH₃ |
| O | 4-F | 4-F-2-Cl | CH₃ |
| O | 4-F | 2-Cl-3,6-F₂ | CH₃ |
| O | 4-F | 3-Cl-2,6-F₂ | CH₃ |
| O | 4-F | 3-F-4-Cl | CH₃ |
| O | 4-F | 2-F-4-Cl | CH₃ |
| O | 4-F | 2,3-Cl₂ | CH₃ |
| O | 4-F | 2-CN | CH₃ |
| CH₂ | 4-Cl | H | CH₃ |
| CH₂ | 4-Cl | 3,5-F₂ | CH₃ |
| CH₂ | 4-Cl | 4-F | CH₃ |
| CH₂ | 4-Cl | 2,6-F₂ | CH₃ |
| CH₂ | 4-F | H | CH₃ |
| CH₂ | 4-F | 3,5-F₂ | CH₃ |
| CH₂ | 4-F | 4-F | CH₃ |
| CH₂ | 4-F | 2,6-F₂ | CH₃ |
| O | 3-F | H | CH₃ |
| O | 3-F | 3,5-F₂ | CH₃ |
| O | 3-F | 4-F | CH₃ |
| O | 3-F | 2,6-F₂ | CH₃ |
| O | 3-F | 2,5-F₂ | CH₃ |
| O | 2,5-F₂ | H | CH₃ |
| O | 2,5-F₂ | 3,5-F₂ | CH₃ |
| O | 2,5-F₂ | 4-F | CH₃ |
| O | 2,5-F₂ | 2,6-F₂ | CH₃ |
| O | 2,5-F₂ | 2,5-F₂ | CH₃ |
| O | 3,5-F₂ | H | CH₃ |
| O | 3,5-F₂ | 3,5-F₂ | CH₃ |
| O | 3,5-F₂ | 4-F | CH₃ |
| O | 3,5-F₂ | 2,6-F₂ | CH₃ |
| O | 3,5-F₂ | 2,5-F₂ | CH₃ |
| O | 3,4-F₂ | H | CH₃ |
| O | 3,4-F₂ | 3,5-F₂ | CH₃ |
| O | 3,4-F₂ | 4-F | CH₃ |
| O | 3,4-F₂ | 2,6-F₂ | CH₃ |
| O | 3,4-F₂ | 2,5-F₂ | CH₃ |
| O | 4-CF₃ | H | CH₃ |
| O | 4-CF₃ | 3,5-F₂ | CH₃ |
| O | 4-CF₃ | 4-F | CH₃ |
| O | 4-CF₃ | 2,6-F₂ | CH₃ |
| O | 4-CF₃ | 2,5-F₂ | CH₃ |

The compounds of the present invention may be prepared by a number of routes, many of which are known to those of skill in the art. The particular order of steps to be employed in the synthesis of compounds of formula I is dependent upon the compound to be synthesized, the starting material employed, and the relative lability of the various substituted moieties.

During any of the following synthetic sequences it may be necessary or desirable to protect sensitive or reactive groups on any of the molecules concerned. This may be achieved by employing conventional protecting groups as described, supra.

The compounds used in the method of the present invention may have one or more asymmetric centers. As a consequence of these chiral centers, the compounds of the present invention occur as racemates, mixtures of enantiomers and as individual enantiomers, as well as diastereomers and mixtures of diastereomers. All asymmetric forms, individual isomers and combinations thereof, are within the scope of the present invention.

The terms “R” and “S” are used herein as commonly used in organic chemistry to denote specific configuration of a chiral center. The term “R” (rectus) refers to that configuration of a chiral center with a clockwise relationship of group priorities (highest to second lowest) when viewed along the bond toward the lowest priority group. The term “S” (sinister) refers to that configuration of a chiral center with a counterclockwise relationship of group priorities (highest to second lowest) when viewed along the bond toward the lowest priority group. The priority of groups is based upon their atomic number (in order of decreasing atomic number). A partial list of priorities and a discussion of stereochemistry is contained in Nomenclature of Organic Compounds: Principles and Practice, (J. H. Fletcher, et al., eds., 1974) at pages 103-120.

In addition to the (R)-(S) system, the older D-L system is also used in this document to denote absolute configuration, especially with reference to amino acids. In this system, a Fischer projection formula is oriented so that the number 1 carbon of the main chain is at the top. The prefix “D” is used to represent the absolute configuration of the isomer in which the functional (determining) group is on the right side of the carbon atom at the chiral center and “L”, that of the isomer in which it is on the left.

In order to preferentially prepare one optical isomer over its enantiomer, a number of routes are available. As an example, a mixture of enantiomers may be prepared, and then the two enantiomers may be separated. A commonly employed method for the resolution of the racemic mixture (or mixture of enantiomers) into the individual enantiomers is to first convert the enantiomers to diastereomers by way of forming a salt with an optically active acid or base. These diastereomers may then be separated using differential solubility, fractional crystallization, chromatography, or the like. Further details regarding resolution of enantiomeric mixtures may be found in J. Jacques, et al., Enantiomers, Racemates, and Resolutions, (1991).

Representative starting material for this synthesis is a compound of formula Va, which may be reacted with an ethinylamine of formula VI, with R6 and R7 as defined in Formula I, by methods known in the art to yield a compound of formula VII. Alternatively, a compound of formula Vb may be coupled with a compound of formula VI using activating agents for N-acylation reactions known in the art, like HOBT, DCC, EDC, oxalyl chloride, TBTU or other coupling reagents known to the skilled artisan, to result in a compound of formula VII. Preferred for the practice of the present invention is TBTU. Intermediates of formula Vb and VI are commercially available or can be prepared by methods known in the art. Intermediates of formula Va may be prepared from commercial compounds by standard methods as described in Tetrahedron Lett. 25 (1984), 4553-4556.

A compound of formula VII may be hydrated by standard methods to yield a compound of formula VIII and subsequently cyclized by treatment with a deprotonating agent, such as sodium hydride, optionally in the presence of an alkylating agent to yield a compound of formula IX. Treatment of the resulting compound with a bromination reagent, such as N-bromosuccinimide, results in a compound of formula X. Reaction with an amine generates compounds of formula XI. Representative reactions are provided in Scheme A below. An example of formula IX where Q is SO₂, R8 is hydrogen and R9 is 4-chlorophenyl is described in Pestic. Sci. 39 (1993), 185-192. 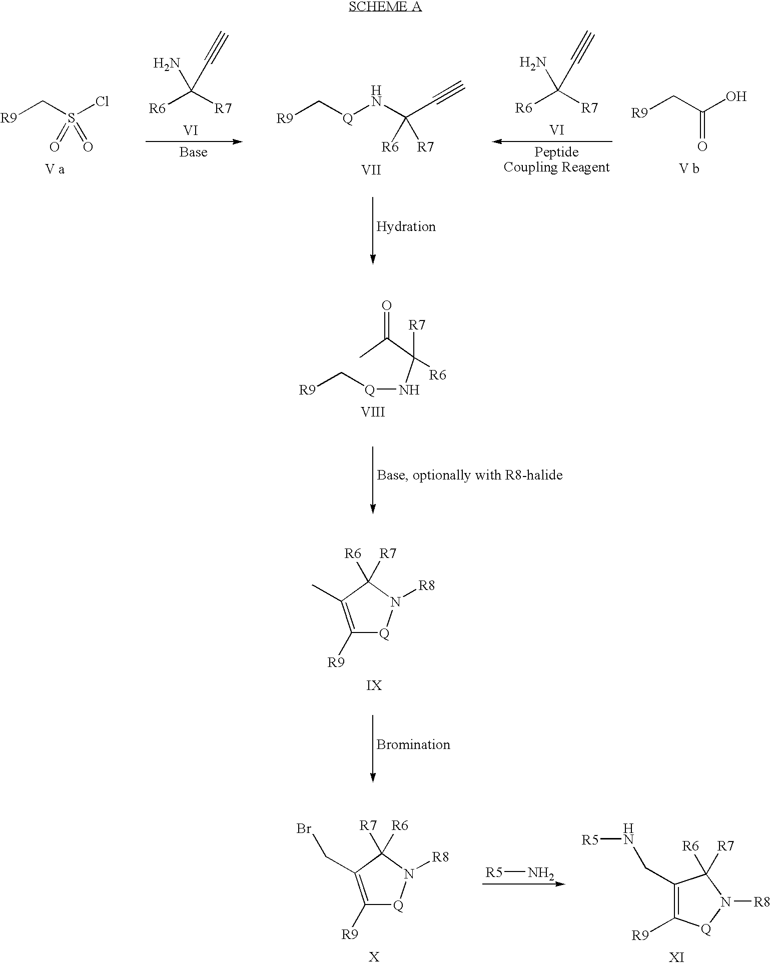
Scheme B shows an alternative synthesis for acetyl intermediates of Formula VIII:

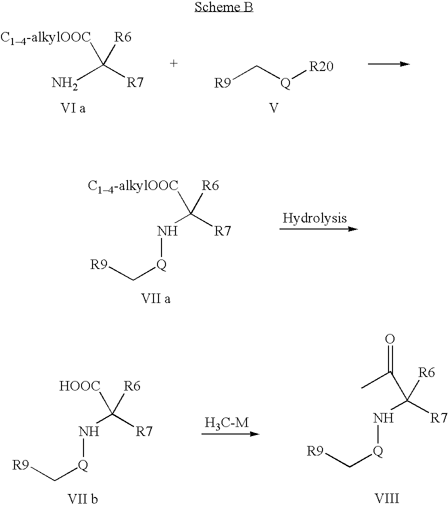

Esters of aminoacids of Formula VI a, preferably methyl or ethyl esters, are coupled with derivatives of carboxylic acids or sulfonic acids of Formula V (with R20 meaning OH or Cl, respectively) by methods described in Scheme A to give intermediates of Formula VIIa. The esters are hydrolized by standard methods to give carboxylic acids of Formula VIIb. These are treated with organometallic methyl compounds to prepare the acetyl intermediates of Formula VIII. Preferred organometallic reagents are methyl Grignard reagents (M=MgCl, MgBr, or MgI) or methyl lithium (M=Li), more preferred is methyl lithium. Examples for this reaction are known from the literature, e.g. J. Org. Chem. 58 (1993), 4758; J. Org. Chem. 62 (1997), 6862; Tetrahedron Lett. 35 (1994), 3745. In a preferred method a solution of the carboxylic acid in a solvent like THF or DME is treated with an excess of methyl lithium in diethylether at a temperature below −60° C. followed by warming to room temperature.

Compounds of Formula I in which m=2 may be prepared as shown in Scheme C below.

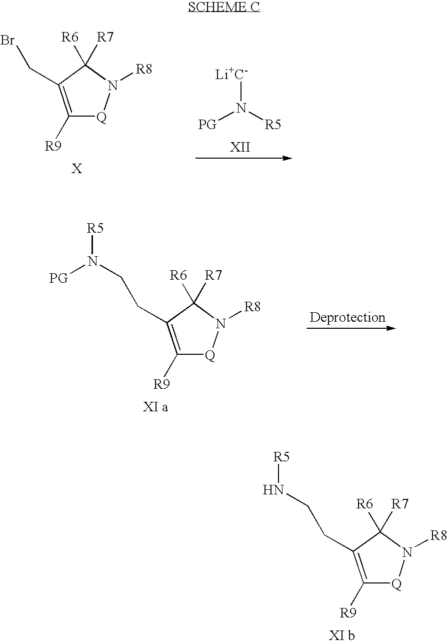

A compound of formula XII is obtained by treatment of a protected methylamine with a deprotonating agent like butyllithium as described for example in Tetrahedron Lett. 35(24), 1994, 4067-70. The substituent “PG” means a protecting group, which is known to the artisan, and all other substituents are as defined by Formula I, herein. One preferred protecting group is the BOC group or another N-protecting group known in the art and stable under the reaction conditions. A compound of formula X is treated with a compound of formula XII to yield a compound of formula XIa.

It is to be understood that the bromine group on the compound of formula X may in fact be any suitable leaving group, as defined herein.

The term “leaving group” refers to a group of atoms that is displaced from a carbon atom by the attack of a nucleophile in a nucleophilic substitution reaction. Suitable leaving groups include bromo, chloro, and iodo, benzenesulfonyloxy, methanesulfonyloxy, and toluenesulfonyloxy. The term “leaving group” includes activating groups as defined above.

A second portion of the overall synthesis of compounds of formula I is provided in Scheme D below.

Representative starting material for this synthesis is a compound of formula XIIIa, which may be a chemically-protected derivative of the amino acid serine. By chemically-protected it is meant that both the amino- and carboxy-functional groups have been suitably protected in order to facilitate further reactions with this molecule. Such protection reactions are known to those of skill in the art, and may be applied to other suitable starting materials. Intermediates of formula XIIIa are commercially available, or may be prepared by standard syntheses of amino acids. Such syntheses are well known to persons of ordinary skill in the art and are described, for example, in Chemistry and Biochemistry of Amino Acids, (G. C. Chapman ed., 1985). The protected amino group may be specifically deprotected, e.g. if PG is a Boc group, using trifluoroacetic acid and methylene chloride, to allow for further reactions with this amino functional group. This deprotection reaction results in a compound of formula XIIIb.

A compound of formula XIIIb may then be N-acylated with an amino-protected compound of formula XIV for instance HOOC—C₁-C₆alkylNHR10 or HOOC— (substituted C₁-C₆alkyl)NHR10 or HOOC-(unsubstituted or substituted C₃-C₈ cycloalkyl)NHR10, wherein R10 is an amino protecting group (PG), to produce a compound of formula XIIIc.

Compounds of formula XIV are commercially available, or are readily prepared from suitable available starting materials. The protected carboxy group on the compound of formula XIIIc is then selectively deprotected, typically using lithium hydroxide, to generate a compound of formula XIII. A compound of formula XIII is then coupled with a compound of formula XI and subsequently deprotected to generate a compound of formula Ia.

Representative reactions are provided below in Scheme D.

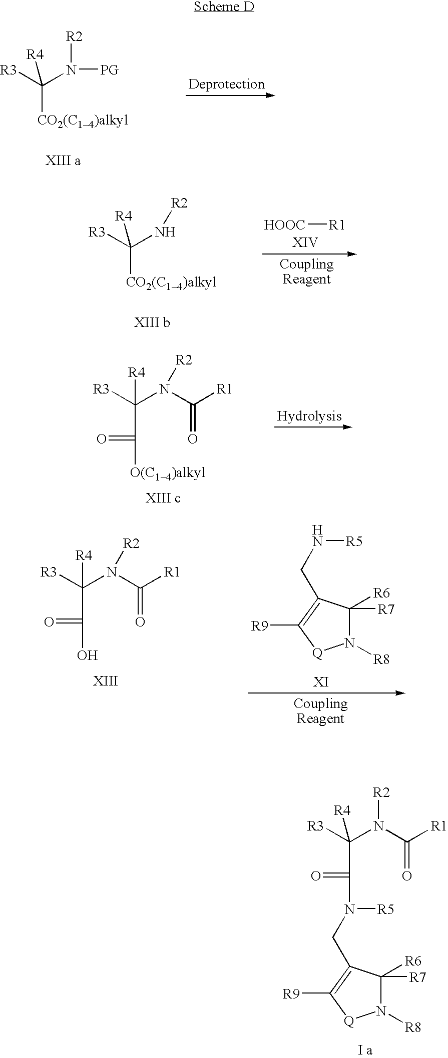

An alternative synthesis for compounds of formula Ia is shshown in Scheme E below:

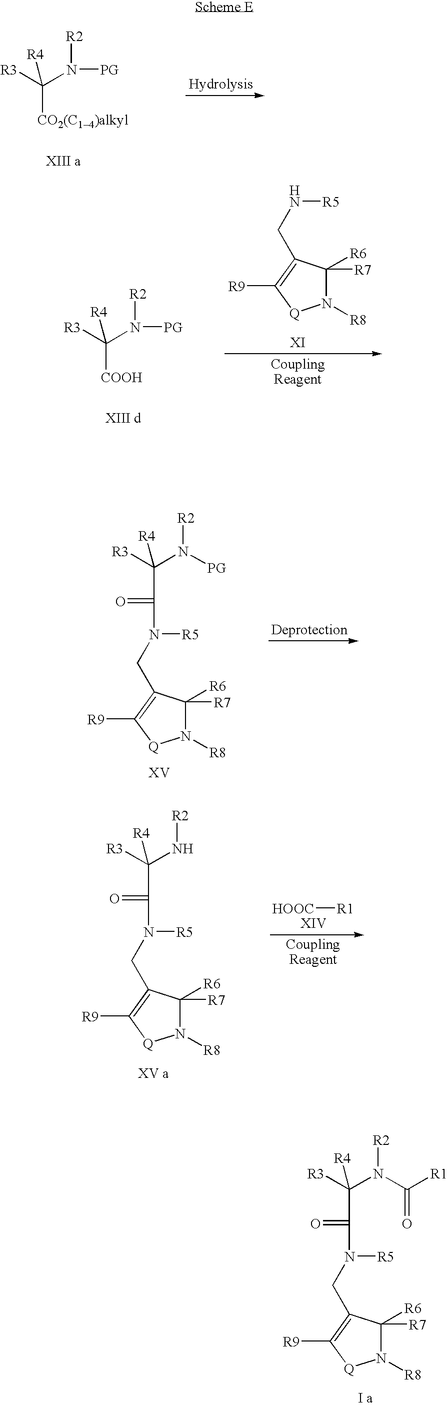

A compound of formula XIIIa, as defined for Scheme D, is selectively deprotected, typically using lithium hydroxide, to generate a compound of formula XIIId, which may then be employed to N-acylate a compound of formula XI, generating a compound of formula XV. Subsequent deprotection results in a compound of formula XVa. A compound of formula XVa is then coupled with a compound of formula XIV, as defined for Scheme D, and subsequently deprotected to generate a compound of formula I.

Suitable activating agents for the N-acylation reactions in Scheme D and Scheme E are known in the art and include DCC, HOBT, EDC, and oxalyl chloride. Preferred for the practice of the present invention are HOBT or TBTU.

Compounds of formula XIII in which the starting material XIIIa is optionally substituted 2-Nboc-amino-5-arylpentanoic acid methyl ester, optionally substituted 2-Nboc-amino-4-arylbutanoic acid methyl ester or 2-Nboc-amino-3-(3-indolyl)-propionic acid methyl ester may also be prepared by the routes described in Scheme D and Scheme E.

Compounds of formula XIb may also be employed in the reactions described in Scheme D and Scheme E.

R1, R2, R3, R4, R5, R6, R7, R8, R9 and Q in Schemes A through E are as defined for Formula I.

The preferred reaction temperature range employed in these reactions is between −40 and 150° C., and the most preferred range is between 10 and 40° C. These reactions may be conveniently carried out in situ, without isolation of the particular compound after its preparation.

The compounds of the present invention can be useful for modulating growth hormone secretion and as research tools.

Compounds of formula I possess growth hormone secretagogue activity. Growth hormone secretagogue activity can be determined using a typical assay which may employ pituitary cells established in culture, followed by a challenge with the various compounds of formula I, and the levels of growth hormone determined accordingly. Growth hormone levels may be calculated using various radioimmunoassay techniques known to those of skill in the art. One example of such an assay is detailed herein.

Thus compounds of formula I find use in the treatment of physiological conditions which are modulated or ameliorated by an increase in endogenous growth hormone. In particular the compounds of formula I are useful in the treatment of conditions or diseases which cause or are mediated by growth hormone deficiencies and maladies associated with ageing in humans. The compounds of formula I are hence useful in the treatment of osteoporosis, physiological short stature including growth hormone deficient children and short stature associated with chronic illness, growth retardation associated with the Prader-Willi syndrome, intrauterine growth retardation, pulmonary dysfunction and ventilator dependency, insulin resistance, cachexia and protein loss due to chronic illness such as cancer or AIDS, as well as congestive heart failure. The compounds of formula I also hence find use in improving muscle strength and mobility, metabolic homeostasis, renal homeostasis especially in the elderly, accelerating the recovery of patients having undergone trauma especially major surgery, improving a negative energy balance in a patient, accelerating bone fracture repair, preventing catabolic side effects associated with therapy, the attenuation of protein catabolic responses following major surgery, the acceleration of wound healing and the treatment of immunosupressed patients. In this connection, compounds of formula I also find use in the manufacture of a medicament for the treatment of the human or animal body by therapy, in particular the therapeutic treatment of conditions or diseases which cause or are mediated by growth hormone deficiencies maladies associated with ageing in humans. In particular compounds of formula I also find use in the manufacture of a medicament for any of the specific uses indicated above.

The compounds of formula I also find use in a method of increasing endogenous levels of growth hormone in mammals and in particular humans and farm or companion animals. Thus the compounds of formula I find use in a method of promoting growth, in particular, increasing lean muscle mass, in an animal, in particular an animal farmed for food including cow, sheep, pig and chicken. The compounds also find particular use in the treatment of disorders of ageing in companion animals.

The invention further encompasses methods employing the pharmaceutically acceptable salts of the compounds defined by formula I. Although generally neutral, a compound of this invention can possess a sufficiently acidic, a sufficiently basic, or both functional groups, and accordingly react with any of a number of inorganic bases, and inorganic and organic acids, to form a pharmaceutically acceptable salt.

The term “pharmaceutically acceptable salt” as used herein refers to salts of the compounds of formula I which are substantially non-toxic to living organisms. Typical pharmaceutically acceptable salts include those salts prepared by reaction of the compounds of the present invention with a pharmaceutically acceptable mineral or organic acid or an inorganic base. Such salts are known as acid addition and base addition salts.

Acids commonly employed to form acid addition salts are inorganic acids such as hydrochloric acid, hydrobromic acid, hydroiodic acid, sulfuric acid, phosphoric acid, and the like, and organic acids such as p-toluenesulfonic, methanesulfonic acid, oxalic acid, p-bromophenylsulfonic acid, carbonic acid, succinic acid, citric acid, benzoic acid, acetic acid, and the like. Examples of such pharmaceutically acceptable salts are the sulfate, pyrosulfate, bisulfate, sulfite, bisulfite, phosphate, monohydrogenphosphate, dihydrogenphosphate, metaphosphate, pyrophosphate, chloride, bromide, iodide, acetate, propionate, decanoate, caprylate, acrylate, formate, isobutyrate, caproate, heptanoate, propiolate, oxalate, malonate, succinate, suberate, sebacate, fumarate, maleate, butyne-1,4-dioate, hexyne-1,6-dioate, benzoate, chlorobenzoate, methylbenzoate, dinitrobenzoate, hydroxybenzoate, methoxybenzoate, phthalate, sulfonate, xylenesulfonate, phenylacetate, phenylpropionate, phenylbutyrate, citrate, lactate, γ-hydroxybutyrate, glycollate, tartrate, methanesulfonate, propanesulfonate, naphthalene-1-sulfonate, naphthalene-2-sulfonate, mandelate, mesylate, and the like. Preferred pharmaceutically acceptable acid addition salts are those formed with mineral acids such as hydrochloric acid and hydrobromic acid, and those formed with organic acids such as maleic acid and methanesulfonic acid.

Salts of amine groups may also comprise quaternary ammonium salts in which the amino nitrogen carries a suitable organic group such as an alkyl, alkenyl, alkynyl, or aralkyl moiety.

Base addition salts include those derived from inorganic bases, such as ammonium or alkali or alkaline earth metal hydroxides, carbonates, bicarbonates, and the like. Such bases useful in preparing the salts of this invention thus include sodium hydroxide, potassium hydroxide, ammonium hydroxide, potassium carbonate, sodium carbonate, sodium bicarbonate, potassium bicarbonate, calcium hydroxide, calcium carbonate, and the like. The potassium and sodium salt forms are particularly preferred.

It should be recognized that the particular counterion forming a part of any salt of this invention is not of a critical nature, so long as the salt as a whole is pharmacologically acceptable and as long as the counterion does not contribute undesired qualities to the salt as a whole.

This invention further encompasses methods employing pharmaceutically acceptable solvates of the compounds of Formula I. Many of the formula I compounds can combine with solvents such as water, methanol, and ethanol to form pharmaceutically acceptable solvates such as the corresponding hydrate, methanolate, and ethanolate.

This invention also encompasses methods employing the pharmaceutically acceptable prodrugs of the compounds of formula I. A prodrug is a drug which has been chemically modified and may be biologically inactive at its site of action, but which may be degraded or modified by one or more enzymatic or other in vivo processes to the parent bioactive form. This prodrug should have a different pharmacokinetic profile than the parent, enabling easier absorption across the mucosal epithelium, better salt formation or solubility, or improved systemic stability (an increase in plasma half-life, for example).

Typically, such chemical modifications include:

1) ester or amide derivatives which may be cleaved by esterases or lipases;

2) peptides which may be recognized by specific or nonspecific proteases; or

3) derivatives that accumulate at a site of action through membrane selection of a prodrug form or a modified prodrug form; or any combination of 1 to 3, supra. Conventional procedures for the selection and preparation of suitable prodrug derivatives are described, for example, in H, Bundgaard, Design of Prodrugs, (1985).

As used herein, the term “effective amount” means an amount of compound of the instant invention which is capable of inhibiting, alleviating, ameliorating, treating, or preventing further symptoms in mammals, including humans, which may be due to decreased levels of endogenous growth hormone.

By “pharmaceutically acceptable formulation” it is meant that the carrier, diluent, excipients and salt must be compatible with the active ingredient (a compound of formula I) of the formulation, and not be deleterious to the recipient thereof. Pharmaceutical formulations can be prepared by procedures known in the art. For example, the compounds of this invention can be formulated with common excipients, diluents, or carriers, and formed into tablets, capsules, and the like. Examples of excipients, diluents, and carriers that are suitable for such formulations include the following: fillers and extenders such as starch, sugars, mannitol, and silicic derivatives; binding agents such as carboxymethyl cellulose and other cellulose derivatives, alginates, gelatin, and polyvinyl pyrrolidone; moisturizing agents such as glycerol; disintegrating agents such as agar agar, calcium carbonate, and sodium bicarbonate; agents for retarding dissolution such as paraffin; resorption accelerators such as quaternary ammonium compounds; surface active agents such as cetyl alcohol, glycerol monostearate; adsorptive carriers such as kaolin and bentonite; and lubricants such as talc, calcium and magnesium stearate and solid polyethylene glycols. Final pharmaceutical forms may be: pills, tablets, powders, lozenges, syrups, aerosols, saches, cachets, elixirs, suspensions, emulsions, ointments, suppositories, sterile injectable solutions, or sterile packaged powders, and the like, depending on the type of excipient used.

Additionally, the compounds of this invention are well suited to formulation as sustained release dosage forms. The formulations can also be so constituted that they release the active ingredient only or preferably in a particular part of the intestinal tract, possibly over a period of time. Such formulations would involve coatings, envelopes, or protective matrices which may be made from polymeric substances or waxes.

The particular dosage of a compound required to treat, inhibit, or prevent the symptoms and/or disease of congestive heart failure in a mammal, including humans, according to this invention will depend upon the particular disease, symptoms, and severity. Dosage, routes of administration, and frequency of dosing is best decided by the attending physician. Generally, accepted and effective doses will be from 15 mg to 1000 mg, and more typically from 15 mg to 80 mg. Such dosages will be administered to a patient in need of treatment from one to three times each day or as often as needed for efficacy.

In addition, the growth hormone secretagogue compounds as disclosed herein may be administered to a patient in need of treatment in combination with other growth hormone secretagogues known in the art, and/or with a suitable bone anti-resorptive agent or agents for the prevention or treatment of osteoporosis and/or loss of muscle strength. Said suitable bone anti-resorptive agents include selective estrogen receptor modulators, bisphophonates, calcitonin, and hormone replacement therapeutic agents. Additionally, PTH may be administered in combination with said growth hormone secretagogues. Said combination therapy may be administered concomitantly or sequentially.

The compositions are preferably formulated in a unit dosage form, each dosage containing from about 0.01 to about 500 mg, more usually about 0.5 to about 200 mg, of the active ingredient. However, it will be understood that the therapeutic dosage administered will be determined by the physician in the light of the relevant circumstances including the condition to be treated, the choice of compound to be administered and the chosen route of administration, and therefore the above dosage ranges are not intended to limit the scope of the invention in any way. The compounds can be administered by a variety of routes including the oral, rectal, transdermal, subcutaneous, topical, intravenous, intramuscular or intranasal routes. For all indications, a typical daily dose will contain from about 0.01 mg/kg to about 20 mg/kg of the active compound of this invention. Preferred daily doses will be about 0.1 to about 10 mg/kg, ideally about 0.1 to about 5 mg/kg. However, for topical administration a typical dosage is about 1 to about 500 mg compound per cm² of an affected tissue. Preferably, the applied amount of compound will range from about 30 to about 300 mg/cm², more preferably, from about 50 to about 200 mg/cm², and, most preferably, from about 60 to about 100 mg/cm².

Suitable dosing ranges of compounds of formula I include 0.01 mg/kg/day to 60 mg/kg/day. Representative pharmaceutical formulations containing compounds of formula I-IV are provided below.

The formulations which follow are given for purposes of illustration and are not intended to be limiting in any way. The total active ingredients in such formulations comprises from 0.1% to 99.9% by weight of the formulation. The term “active ingredient” means a compound of formula I, including but not limited to compounds of formulas II, III, and IV.

### Formulation 1

Hard gelatin capsules containing the following ingredients are prepared:

| | Quantity |
|---|---|
| Ingredient | (mg/capsule) |
| Active Ingredient | 30.0 |
| Starch | 305.0 |
| Magnesium stearate | 5.0 |

The above ingredients are mixed and filled into hard gelatin capsules in 340 mg quantities.

### Formulation 2

A tablet formula is prepared using the ingredients below:

| | Quantity |
|---|---|
| Ingredient | (mg/tablet) |
| Active Ingredient | 25.0 |
| Cellulose, microcrystalline | 200.0 |
| Colloidal silicon dioxide | 10.0 |
| Stearic acid | 5.0 |

The components are blended and compressed to form tablets, each weighing 240 mg.

### Formulation 3

A dry powder inhaler formulation is prepared containing the following components:

| Ingredient | Weight % |
|---|---|
| Active Ingredient | 5 |
| Lactose | 95 |

The active mixture is mixed with the lactose and the mixture is added to a dry powder inhaling appliance.

### Formulation 4

Tablets, each containing 30 mg of active ingredient, are prepared as follows:

| | Quantity | |
|---|---|---|
| Ingredient | (mg/tablet) | |
| Active Ingredient | 30.0 | mg |
| Starch | 45.0 | mg |
| Microcrystalline cellulose | 35.0 | mg |
| Polyvinylpyrrolidone | 4.0 | mg |
| (as 10% solution in water) | | |
| Sodium carboxymethyl starch | 4.5 | mg |
| Magnesium stearate | 0.5 | mg |
| Talc | 1.0 | mg |
| Total | 120 | mg |

The active ingredient, starch and cellulose are passed through a No. 20 mesh U.S. sieve and mixed thoroughly. The solution of polyvinylpyrrolidone is mixed with the resultant powders, which are then passed through a 16 mesh U.S. sieve. The granules so produced are dried at 50-60° C. and passed through a 16 mesh U.S. sieve. The sodium carboxymethyl starch, magnesium stearate, and talc, previously passed through a No. 30 mesh U.S. sieve, are then added to the granules which, after mixing, are compressed on a tablet machine to yield tablets each weighing 120 mg.

### Formulation 5

Capsules, each containing 40 mg of medicament are made as follows:

| | Quantity | |
|---|---|---|
| Ingredient | (mg/capsule) | |
| Active Ingredient | 40.0 | mg |
| Starch | 109.0 | mg |
| Magnesium stearate | 1.0 | mg |
| Total | 150.0 | mg |

The active ingredient, cellulose, starch, and magnesium stearate are blended, passed through a No. 20 mesh U.S. sieve, and filled into hard gelatin capsules in 150 mg quantities.

### Formulation 6

Suppositories, each containing 25 mg of active ingredient are made as follows:

| Ingredient | Amount | |
|---|---|---|
| Active Ingredient | 25 | mg |
| Saturated fatty acid glycerides to | 2,000 | mg |

The active ingredient is passed through a No. 60 mesh U.S. sieve and suspended in the saturated fatty acid glycerides previously melted using the minimum heat necessary. The mixture is then poured into a suppository mold of nominal 2.0 g capacity and allowed to cool.

### Formulation 7

Suspensions, each containing 50 mg of medicament per 5.0 mL dose are made as follows:

| Ingredient | Amount | |
|---|---|---|
| Active Ingredient | 50.0 | mg |
| Xanthan gum | 4.0 | mg |
| Sodium carboxymethyl cellulose (11%) | 50.0 | mg |
| Microcrystalline cellulose (89%) | | |
| Sucrose | 1.75 | g |
| Sodium benzoate | 10.0 | mg |
| Flavor and Color | q.v. | |
| Purified water to | 5.0 | mL |

The medicament, sucrose and xanthan gum are blended, passed through a No. 10 mesh U.S. sieve, and then mixed with a previously made solution of the microcrystalline cellulose and sodium carboxymethyl cellulose in water. The sodium benzoate, flavor, and color are diluted with some of the water and added with stirring. Sufficient water is then added to produce the required volume.

### Formulation 8

Capsules, each containing 15 mg of medicament, are made as follows:

| | Quantity | |
|---|---|---|
| Ingredient | (mg/capsule) | |
| Active Ingredient | 15.0 | mg |
| Starch | 407.0 | mg |
| Magnesium stearate | 3.0 | mg |
| Total | 425.0 | mg |

The active ingredient, cellulose, starch, and magnesium stearate are blended, passed through a No. 20 mesh U.S. sieve, and filled into hard gelatin capsules in 425 mg quantities.

### Formulation 9

An intravenous formulation may be prepared as follows:

| Ingredient | Quantity | |
|---|---|---|
| Active Ingredient | 250.0 | mg |
| Isotonic saline | 1000 | mL |

### Formulation 10

A topical formulation may be prepared as follows:

| Ingredient | Quantity | |
|---|---|---|
| Active Ingredient | 1-10 | g |
| Emulsifying Wax | 30 | g |
| Liquid Paraffin | 20 | g |
| White Soft Paraffin | to 100 | g |

The white soft paraffin is heated until molten. The liquid paraffin and emulsifying wax are incorporated and stirred until dissolved. The active ingredient is added and stirring is continued until dispersed. The mixture is then cooled until solid.

### Formulation 11

Sublingual or buccal tablets, each containing 10 mg of active ingredient, may be prepared as follows:

| | Quantity | |
|---|---|---|
| Ingredient | Per Tablet | |
| Active Ingredient | 10.0 | mg |
| Glycerol | 210.5 | mg |
| Water | 143.0 | mg |
| Sodium Citrate | 4.5 | mg |
| Polyvinyl Alcohol | 26.5 | mg |
| Polyvinylpyrrolidone | 15.5 | mg |
| Total | 410.0 | mg |

The glycerol, water, sodium citrate, polyvinyl alcohol, and polyvinylpyrrolidone are admixed together by continuous stirring and maintaining the temperature at about 90° C. When the polymers have gone into solution, the solution is cooled to about 50-55° C. and the medicament is slowly admixed. The homogenous mixture is poured into forms made of an inert material to produce a drug-containing diffusion matrix having a thickness of about 2-4 mm. This diffusion matrix is then cut to form individual tablets having the appropriate size.

Another formulation employed in the methods of the present invention employs transdermal delivery devices or patches. Such transdermal patches may be used to provide continuous or discontinuous infusion of the compounds of the present invention in controlled amounts. The construction and use of transdermal patches for the delivery of pharmaceutical agents is well known in the art. See, for example, U.S. Pat. No. 5,023,252, the disclosure of which is herein incorporated by reference. Such patches may be constructed for continuous, pulsatile, or on demand delivery of pharmaceutical agents.

Frequently, it will be desirable or necessary to introduce the pharmaceutical composition to the brain, either directly or indirectly. Direct techniques usually involve placement of a drug delivery catheter into the host's ventricular system to bypass the blood-brain barrier. One such implantable delivery system, used for the transport of biological factors to specific anatomical regions of the body, is described in U.S. Pat. No. 5,011,472, the disclosure of which is herein incorporated by reference.

Indirect techniques, which are generally preferred, usually involve formulating the compositions to provide for drug latentiation by the conversion of hydrophilic drugs into lipid-soluble drugs or prodrugs. Latentiation is generally achieved through blocking of the hydroxy, carbonyl, sulfate, and primary amine groups present on the drug to render the drug more lipid soluble and amenable to transportation across the blood-brain barrier. Alternatively, the delivery of hydrophilic drugs may be enhanced by intra-arterial infusion of hypertonic solutions which can transiently open the blood-brain barrier.

The following Examples and Preparations are illustrative of the processes employed in the synthesis of the compounds of the present invention. As would be understood by persons skilled in the art, other synthetic schemes may be employed to prepare the compounds of the instant invention.

### Intermediate 1

### N-(5-(4-Chlorophenyl)-3,3-dimethyl-1,1-dioxo-2,3-dihydroisothiazol-4-yl)methyl-N-ethylamine

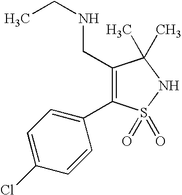

To a solution of sodium (4-chlorophenyl)methanesulfonate, 8.9 g (39.0 mmol) in 20 mL of phosphorus oxychloride at 0° C., was added 11.6 g of phosphorus pentachloride. The reaction mixture was slowly warmed to ambient temperature, stirred 48 h and concentrated to dryness.

To a solution of 1,1-dimethylpropargylamine, 3.23 g (39.0 mmol, as described in J. Am. Chem. Soc. 75 (1954), 1653) in 50 mL of dichloromethane at 0° C. was added 6.41 mL (42.9 mmol) of 1,8-diazabicyclo(5.4.0)undec-7-ene. After stirring for 10 min, 8.8 g (39.0 mmol) of the above residue in 70 mL of dichloromethane was added. The reaction mixture was stirred for 2 h at 0° C. and was concentrated to dryness and partitioned between ethyl acetate and water. The mixture was acidified to pH=2.0 with 1 N HCl and was extracted with ethyl acetate. The combined organics were washed with brine, dried over sodium sulfate, filtered and concentrated to dryness. The resulting residue was chromatographed over silica gel using 5% methanol/chloroform as eluant to yield 6.15 g (58%) of the desired product, shown below, as a white solid. ¹H-NMR is consistent with the desired product, shown below; MS (ion spray) 270.3 (M−1); Anal. Calc'd for C₁₂H₁₄ClNO₂S: C, 53.04; H, 5.19; N, 5.15. Found: C, 52.54; H, 5.19; N, 4.93. C-(4-Chlorophenyl)-N-(1,1-dimethyl-prop-2-ynyl)methanesulfonamide:

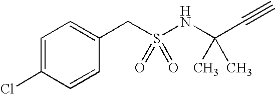

To a solution of C-(4-chlorophenyl)-N-(1,1-dimethyl-prop-2-ynyl)methanesulfonamide, 5.88 g (22.0 mmol) in 40 mL of ethylene glycol was added 0.3 g of mercury oxide (yellow), 4 mL of water and 6 drops concentrated sulfuric acid. The mixture was heated at 170° C. for 80 min then was cooled to ambient temperature, poured into water and extracted with ethyl acetate. The combined organics were washed with brine, dried over sodium sulfate, filtered and concentrated to dryness. The resulting residue was chromatographed on silica gel using chloroform as eluant to yield 4.31 g (68%) of the desired product, shown below, as a tan solid. ¹H-NMR is consistent with structure; MS (ion spray) 288.0 (M−1); Anal. Calc'd for C₁₂H₁₆ClNO₃S: C, 49.74; H, 5.56; N, 4.83. Found: C, 49.59; H, 5.50; N, 4.73. C-(4-Chlorophenyl)-N-(1,1-dimethyl-2-oxo-propyl)-methanesulfonamide:

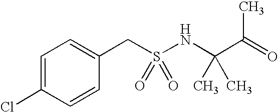

To a solution of C-(4-chlorophenyl)-N-(1,1-dimethyl-2-oxo-propyl)-methanesulfonamide, 4.2 g (15.0 mmol) in 60 mL of dimethylformamide was added 1.3 g (31.5 mmol) of sodium hydride. The reaction mixture was heated at 90° C. for 24 h, then cooled to ambient temperature and concentrated to dryness. The residue was partitioned between ethyl acetate and water and was acidified to pH=3.0 with 1 N HCl. The mixture was extracted with ethyl acetate. The combined organics were washed with brine, dried over sodium sulfate, filtered and concentrated to dryness. The residue was chromatographed over silica using chloroform as eluant to yield 3.27 g (80%) of the desired product, shown below, as a tan solid. ¹H-NMR is consistent with structure; MS (ion spray) 270.3 (M−1); Anal. Calc'd for C₁₂H₃₄ClNO₂S: C, 53.04; H. 5.19; N, 5.15. Found: C, 52.72; H, 5.18; N, 4.98. 5-(4-Chlorophenyl)-3,3,4-trimethyl-2,3-dihydro-isothiazole-1,1-dioxide:

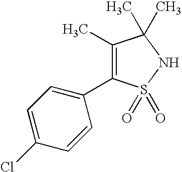

To a solution of 5-(4-chlorophenyl)-3,3,4-trimethyl-1,1-dioxo-2,3-dihydro-isothiazole, 1.5 g (5.5 mmol) in 150 mL of carbon tetrachloride was added 1.5 g (8.25 mmol) of N-bromosuccinimide and 0.13 g of 2,2′-azobis(2-methyl-propionitrile). The mixture was heated to reflux for 4 h and then cooled to ambient temperature. Chloroform was added and the solution was washed with water, washed with brine, dried over sodium sulfate, filtered and concentrated to dryness. To a solution of the residue in 75 mL of absolute ethanol was added 3.6 mL (55.0 mmol) of ethylamine (70% solution in water). The reaction mixture was stirred 24 h at ambient temperature then concentrated to dryness. The residue was purified by chromatography on silica gel with methanol/chloroform as eluant to yield 0.21 g (12%) of the desired title amine as a tan oil. ¹H-NMR is consistent with structure; MS (ion spray) 313.0 (M−1); Anal. Calc'd for C₁₄H₁₉ClN₂O₂S.0.1CHCl₃: C, 51.83; H, 5.89; N, 8.57. Found: C, 51.58; H, 6.38; N, 8.04.

### Intermediate 2

### 2-(R)-2-Amino-3-benzyloxy-propionic acid N-[5-(4-chlorophenyl)-3,3-dimethyl-1,1-dioxo-2,3-dihydroisothiazol-4-ylmethyl]-N-ethylamide

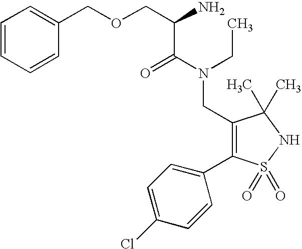

To a suspension of the amine of Intermediate 1, 189 mg (0.60 mmol), in 2.9 mL of IPAC were added water (1.7 ml), DCC (136 mg, 0.66 mmol), HOBt (89 mg, 0.66 mmol), IPAC (0.6 mL) and the O-benzyl-D-serine of Intermediate 6e (177 mg, 0.60 mmol). The mixture was allowed to stir for 14 h, then was filtered rinsing with IPAC. The aqueous phase was separated. The organic layer was washed with citric acid 0.1 M and saturated NaHCO₃, dried over Na₂SO₄ and evaporated to yield 195 mg (55%) of the desired product, shown below, as a white-off solid. ¹H-NMR is consistent with structure; MS (ion spray) 492.0 (M-Boc+1).

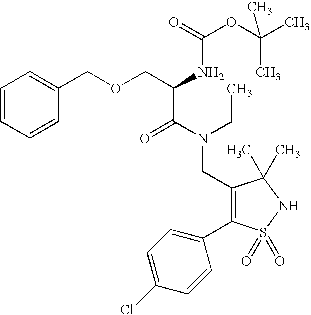

To a solution of the tert-butylcarbamate, 190 mg (0.32 mmol) in 1.5 mL of dichloromethane was added 1.5 mL of trifluoroacetic acid. The mixture was stirred at room temperature for 1 h, then poured into diethyl ether (300 mL) and stirred for 2 h. The white precipitate formed was filtered and dried to afford a white solid, which was dissolved in dichloromethane and washed with saturated NaHCO₃ to yield 135 mg (86%) of the desired title compound as a white solid. ¹H-NMR is consistent with structure; MS (ion spray) 492.0 (M+1).

### Intermediate 3

### N-(5-(4-Chlorophenyl)-3,3-dimethyl-1,1-dioxo-2,3-dihydroisothiazol-4-yl)methyl-N-(2-fluoroethyl)amine

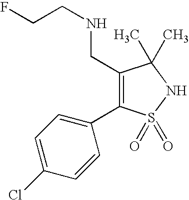

A solution of 2-fluoroethylamine hydrochloride (709 mg, 7.1 mmol; prepared as described in J. Med Chem. 9 (1966), 892-911) in 10 mL of absolute MeOH was treated with triethylamine (1.60 mL, 1.15 g, 11.4 mmol) at room temperature. Solid 4-bromomethyl-5-(4-chlorophenyl)-3,3-dimethyl-1,1-dioxo-2,3-dihydro-isothiazole (1.0 g, 2.85 mmol; as described for Intermediate 1) was added. The resulting mixture was stirred for 16 h at ambient temperature and concentrated to dryness under reduced pressure. The residue was purified by column chromatography on silica gel using dichloromethane/methanol as eluent to yield the title intermediate. Yield: 880 mg (93%); MS (IS): 333.1 [M+H]⁺. ¹H-NMR is consistent with the structure depicted above.

### Intermediate 4

### N-(3-(4-Chlorophenyl)-2,2-dioxo-2-thia-1-aza-spiro[4.4]non-3-en-4-ylmethyl)-N-(2-fluoroethyl)amine

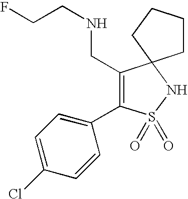

The title compound, as shown above, was prepared as follows:

1-Amino-1-cyclopentanecarboxylic acid (5.00 g, 38.8 mmol) was dissolved in methanol (100 mL) and then thionyl chloride (9.25 g, 77.7 mmol) was added dropwise with stirring. The resulting mixture was stirred overnight at room temperature and then concentrated in vacuo which left a white solid. The solid was tritruated in ethyl ether, filtered, and dried to give 6.78 g (97%) of methyl 1-amino-1-cyclopentanecarboxylate hydrochloride as a white solid. ¹H NMR was consistent with product. ESMS (M+1) 144.2

The amino-ester hydrochloride (2.50 g, 14.0 mmol) was combined with triethylamine (9.0 mL, 64.7 mmol), and 4-dimethylaminopyridine (cat. 50 mg), in dichloromethane (75 mL) at room temperature. Then (4-chlorophenyl)methanesulfonyl chloride (as described above) (3.00 g, 13.4 mmol) was added and the resulting mixture stirred overnight at room temperature. Water was then added and the pH of the aqueous phase adjusted to 2.5 with aqueous hydrochloric acid. The mixture was then extracted with dichloromethane and the combined extracts were dried over sodium sulfate and concentrated in vacuo. The resulting residue was chromatographed over silica (chloroform/methanol) to give 2.00 g (45%) of the desired sulfonamide, shown below, as a light yellow solid. ¹H NMR was consistent with product. ESMS: (M−1)⁻330.1, 331.2. Anal. Calcd. for C₁₄H₁₈NO₄SCl: C, 50.68; H, 5.47; N, 4.22.Found: C, 50.14; H, 5.50; N, 4.21. Methyl 1-(4-Chlorophenylmethanesulfonylamino)-1-cyclopentanecarboxylate:

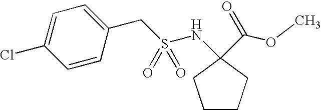

The ester from above (1.90 g, 5.74 mmol) was combined with 2 N aqueous sodium hydroxide (40 mL), tetrahydrofuran (5 mL), and ethanol (5 mL) and the mixture stirred at room temperature until hydrolysis was complete. Aqueous hydrochloric acid (5 N) was added until the aqueous mixture reached pH 2.0 and the aqueous phase was then extracted with ethyl acetate. The combined extracts were dried over sodium sulfate and the solution concentrated in vacuo. The resulting solid was triturated in diethyl ether, filtered and dried to give 1.75 g (97 t) of the desired acid, shown below, as a white solid.

¹H NMR was consistent with product. ESMS: (M+1)⁺316.0, 317.1. Anal. Calcd. for C₁₃H₁₆NO₄SCl: C, 49.13; H, 5.08; N, 4.41. Found: C, 49.16; H, 5.01; N, 4.20. 1-(4-Chlorophenylmethanesulfonylamino)-1-cyclopentanecarboxylic acid:

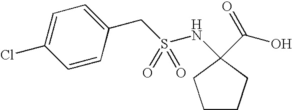

The acid from above (2.90 g, 9.2 mmol) was dissolved in anhydrous dimethoxyethane (75 mL) and the mixture cooled to −60° C. (dry ice/acetone bath) under nitrogen. Then methyl lithium (32.7 mL, 1.4 M in ethyl ether) was added via syringe and the resulting mixture stirred for 4.5 hours while slowly warming to near 0° C. The reaction was then quenched into a stirred mixture of ice/1N aqueous hydrochloric acid and the aqueous mixture extracted with ethyl acetate. The combined extracts were concentrated and the resulting residue chromatographed over silica (chloroform/methanol) which allowed for isolation of 2.30 g (79%) of the desired ketone, shown below, as a white solid. ¹H NMR was consistent with product. ESMS: (M+1)⁺316.1. Anal. Calcd. for C₁₄H₁₈NO₃SCl: C, 53.24; H, 5.74; N, 4.43. Found: C, 52.50; H, 5.48; N, 4.29. N-(1-Acetylcyclopentyl)-C-(4-chlorophenyl)methanesulfonamide:

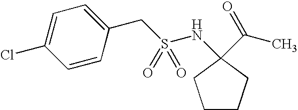

The ketone (2.50 g, 7.94 mmol) was dissolved in N,N-dimethylformamide (40 mL) and then sodium hydride (60%, 0.70 g, 17.4 mmol) was added and the resulting mixture heated at 100° C. overnight. The solvent was then removed in vacuo and the resulting residue taken up in dilute aqueous hydrochloric acid. The aqueous mixture was extracted with ethyl acetate and the combined extracts were concentrated to leave a residue. This residue was chromatographed over silica (chloroform/methanol) which allowed for isolation of the desired product, shown below, 2.00 g (84%) as a white solid. ESMS: (M+1)⁺298.4. ¹H NMR was consistent with product. Anal. Calcd. for C₁₄H₁₆NO₂SCl: C, 56.46; H, 5.41; N, 4.70. Found: C, 56.17; H, 5.32; N, 4.69. 3-(4-Chlorophenyl)-4-methyl-2,2-dioxo-2-thia-1-aza-spiro[4.4]non-3-ene:

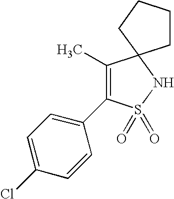

The product from above (1.80 g, 6.1 mmol) was slurried in carbon tetrachloride (50 mL) and N-bromosuccinimide (1.62 g, 9.1 mmol) and 2,2′-azobis(2-methylpropionitrile) (0.05 g, cat.) were added. This mixture was heated at reflux for 4 hours after which time the reaction was cooled to ambient temperature and diluted with dichloromethane. The organic mixture was washed with water (2×40 mL) and dried over sodium sulfate. Column chromatography on silica gel with dichloromethane as eluent yielded the bromide as slightly yellow crystals. Yield: 1.36 g (59%). MS (IS): 357.9 [M−H]⁻. ¹H-NMR is consistent with the structure shown below. 4-Bromomethyl-3-(4-chlorophenyl)-2,2-dioxo-2-thia-1-aza-spiro[4.4]non-3-ene:

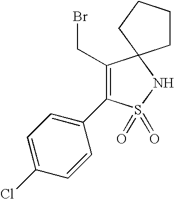

A solution of 2-fluoroethylamine hydrochloride (1.08 g, 10.9 mmol; prepared as described in J. Med Chem. 9 (1966), 892-911) in 30 mL of absolute MeOH was treated with triethylamine (2.50 mL, 1.76 g, 17.4 mmol) at 0° C. Solid 4-bromomethyl-3-(4-chlorophenyl)-2,2-dioxo-2-thia-1-aza-spiro[4.4]non-3-ene (1.64 g, 4.35 mmol) was added. The resulting mixture was stirred for 16 h at ambient temperature and concentrated to dryness under reduced pressure. The residue was purified by column chromatography on silica gel using dichloromethane/methanol as eluent to yield the title intermediate. Yield: 1.00 g (64%); MS (IS): 359.1 [M+H]⁺.

### Intermediate 5

### N-(5-(4-Chlorophenyl)-3,3-dimethyl-1,1-dioxo-2,3-dihydroisothiazol-4-yl)methyl-N-methylamine

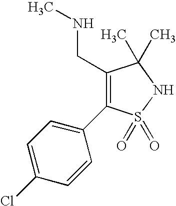

The title compound was prepared from (4-chlorophenyl)methanesulfonyl chloride and 1,1-dimethylpropargylamine according to the procedure described for Intermediate 1 replacing ethylamine by methylamine in the last step. ¹H-NMR (DMSO-d₆) δ 7.72 (bs, 1H), 7.53 (m, 4H), 3.33 (s, 2H), 2.16 (s, 3H), 1.45 (s, 6H).

### Intermediates 6a-6e

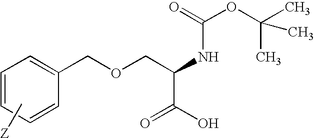

### INTERMEDIATE 6a: (R)-2-tert-Butoxycarbonylamino-3-(4-fluorobenzyloxy)propionic acid (Z=4-F)

N-tert-Butoxycarbonyl-D-serine (0.5 g, 2.4 mmol) was dissolved in dry DMF (12 ml), potassium tert-butanolate (0.56 g, 5 mmol) in 4 ml dry DMF was added and the mixture was stirred for 30 min at 0° C. 4-Fluorobenzyl chloride (0.293 ml, 2.45 mmol) was added and the solution was stirred for 4 hours at room temperature. Water (50 ml) was added and the mixture was extracted with tert-butylmethylether. The aqueous layer was acidified with citric acid to pH 3 and extracted with ethylacetate. This organic layer was dried (NaSO₄) and evaporated and the residue purified by chromatography on silica (eluent CH₂Cl₂/EtOH 97.5:2.5) to yield the product as a colourless oil. Yield: 318 mg (42%); ¹H-NMR (CDCl₃) δ 8.85 (bs, 1H), 7.26 (m, 2H), 6.99 (m, 2H), 5.45 (d, 1H), 4.49 (s, 2H), 3.90 (d, 1H), 3.70 (d, 1H), 1.43 (s, 9H); MS (IS): 312.2 [M+H]⁺

The following Intermediates were prepared by the same procedure from N-tert-butoxycarbonyl-D-serine and the corresponding benzyl halide:

### INTERMEDIATE 6b: (R)-2-tert-Butoxycarbonylamino-3-(3,5-difluorobenzyloxy)propionic acid (Z=3,5-F₂)

Prepared from N-tert-butoxycarbonylamino-D-serine and 3,5-difluorobenzyl chloride:

Yield: 208 mg (12%); MS: 330.1 [M−H]⁻

### INTERMEDIATE 6c: (R)-2-tert-Butoxycarbonylamino-3-(2,6-difluorobenzyloxy)propionic acid (Z=2,6-F₂)

Prepared from N-tert-butoxycarbonylamino-D-serine and 2,6-difluorobenzyl chloride:

Yield: 318 mg (18%); MS (IS): 330.1 [M−H]⁻

### INTERMEDIATE 6d: (R)-2-tert-Butoxycarbonylamino-3-(2,4-difluorobenzyloxy)propionic acid (Z=2,4-F₂)

MS (IS): 332.3 [M+H]⁺

### INTERMEDIATE 6e: (R)-2-tert-Butoxycarbonylamino-3-benzyloxypropionic acid (Z=H)

The compound was prepared according to a procedure described in Tetrahedron 53 (1997), 10983.

### Intermediate 7

### N-(5-(4-Chlorophenyl)-3,3-dimethyl-1,1-dioxo-2,3-dihydroisothiazol-4-yl)methyl-O-methyl-hydroxylamine

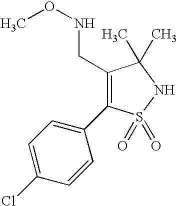

O-Methylhydroxylamine hydrochloride (360 mg, 4.30 mmol) and NEt₃ (0.84 mL, 614 mg, 6.02 mmol) were aded to a solution of 4-bromomethyl-5-(4-chlorophenyl)-3,3-dimethyl-1,1-dioxo-2,3-dihydro-isothiazole (150 mg, 0.43 mmol; prepared as described for Intermediate 1) in DMF. The mixture was stirred at room temperature for 2 d then treated with saturated aqueous NaHCO₃ solution and extracted with ethyl acetate. The combined organic extracts were washed with water and brine. Concentration under reduced pressure and purification by column chromatography on silica gel using hexanes/ethyl acetate as eluent afforded 58 mg (43%) of the title compound. MS (IS): 317.1 [M+H]⁺. ¹H-NMR is consistent with the structure shown above.

### Intermediate 8

### 2-tert-Butoxycarbonylamino-3-fluoro-2-fluoromethyl-propionic Acid

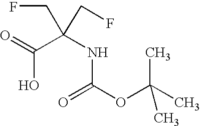

To a suspension of methyl 2-amino-3-fluoro-2-fluoromethyl-propionate hydrochloride (250 mg, 1.32 mmol; prepared as described in Synthesis, 1994, pp 701-702) in 10 mL of acetonitrile, was added Me₄NOH 5H₂O (400 mg, 2.20 mmol). The mixture was stirred at room temperature under argon for 30 min, and then di-tert-butyl dicarbonate (432 mg, 1.98 mmol) was added. The mixture was stirred for 48 h, the solvent evaporated and the residue portioned between water and ether. The aqueous layer was washed with ether, acidified with solid citric acid to pH 3-4 and extracted with ethyl acetate. The combined organic layers were washed with water, brine, dried over Na₂SO₄ and evaporated to yield 294 mg (92%) of the desired product as a white solid. ¹H-NMR is consistent with structure; MS (ion spray) 238.1 (M−1).

### Intermediate 9

### (R)-2-tert-Butoxycarbonylamino-5-phenylpentanoic acid

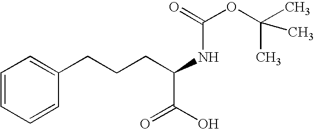 The compound was prepared according to a procedure described in WO 97/36873.

### Intermediates 10a-10f

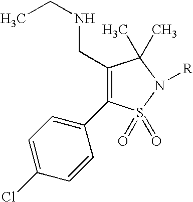

### INTERMEDIATE 10a: N-(5-(4-Chlorophenyl)-3,3-dimethyl-1,1-dioxo-2-(2-fluoroethyl)-2,3-dihydroisothiazol-4-yl)methyl-N-ethylamine (R═CH₂CH₂F)

A suspension of sodium hydride, 795 mg (33.1 mmol) in 50 mL of dimethyl formamide was treated with 5-(4-chlorophenyl)-3,3,4-trimethyl-2,3-dihydro-1,1-dioxo-isothiazole (3.0 g, 11.0 mmol; prepared as described for Intermediate 1) in several portions with stirring at ambient temperature under argon. Stirring was continued for 30 min, before 1-bromo-2-fluoroethane 2.80 g (22.1 mmol) was added. The resulting mixture was stirred for 16 h at 110° C. and then cooled to room temperature. Saturated aqueous ammonium chloride solution was added and the resulting mixture extracted with ethyl acetate. The separated organic layer was washed with saturated aqueous sodium bicarbonate solution and brine and concentrated under reduced pressure. The residue was purified by column chromatography on silica gel using hexanes/ethyl acetate as eluent to yield the desired product shown below. Yield: 3.29 g (94%); MS (IS): 318.0 [M+H]⁺; 335.1 [M+NH₄]⁺. ¹H-NMR is consistent with the structure. 5-(4-Chlorophenyl)-2-(2-fluoroethyl)-3,3,4-trimethyl-1,1-dioxo-2,3-dihydro-isothiazole:

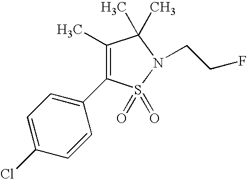

To a solution of 5-(4-Chlorophenyl)-2-(2-fluoroethyl)-3,3,4-trimethyl-1,1-dioxo-2,3-dihydro-isothiazole, 1.75 g (5.5 mmol) in 150 mL of carbon tetrachloride was added 1.47 g (8.25 mmol) of N-bromosuccinimide and 0.13 g of 2,2′-azobis(2-methylpropionitrile). The mixture was heated at reflux for 4 h then cooled to ambient temperature. Chloroform was added and the solution was washed with water and brine, successively, dried over sodium sulfate, filtered and concentrated to dryness.

To a solution of the residue in 155 mL of absolute methanol was added 11.0 mL (22.0 mmol) of ethylamine (2 M solution in methanol). The reaction mixture was stirred for 24 h at ambient temperature and concentrated to dryness. The residue was purified by chromatography on silica gel with methanol/dichloromethane as eluant to yield the desired title amine. Yield: 1.19 g (60%); MS (IS): 361.1 [M+H]⁺; 721.2 [2M+H]⁺. ¹H-NMR is consistent with the structure.

### INTERMEDIATE 10b: N-(5-(4-Chlorophenyl)-3,3-dimethyl-1,1-dioxo-2-(2-methoxyethyl)-2,3-dihydroisothiazol-4-yl)methyl-N-ethylamine (R═CH₂CH₂OCH₃)

The amine from Intermediate 1 was Boc-protected and then alkylated with 2-bromoethyl-methylether followed by deprotection using trifluoroacetic acid in dichloromethane; MS (IS): 373.1 [M+H]⁺.

### INTERMEDIATE 10c: 2-[5-(4′-Chlorophenyl)-3,3-dimethyl-1,1-dioxo-4-(ethylaminomethyl)-2,3-dihydroisothiazol-2-yl]-acetamide (R═CH₂CONH₂)

The compound was prepared in the same manner as Intermediate 10b by alkylation with iodoacetamide; MS (IS): 372.1 [M+H]⁺.

### INTERMEDIATE 10d: 2-[5-(4-Chlorophenyl)-3,3-dimethyl-1,1-dioxo-4-(ethylaminomethyl)-2,3-dihydroisothiazol-2-yl]-N,N-dimethylacetamide (R═CH₂CON(CH₃)₂)

The compound was prepared in the same manner as Intermediate 10b by alkylation with N,N-dimethyl chloroacetamide; MS (IS): 400.1 [M+H]⁺.

### INTERMEDIATE 10e: N-(5-(4-Chlorophenyl)-2-cyclopropylmethyl-3,3-dimethyl-1,1-dioxo-2,3-dihydroisothiazol-4-yl)methyl-N-ethylamine (R═CH₂C₃H₅)

The compound was prepared in the same manner as Intermediate 10b by alkylation with cyclopropylmethyl bromide; MS (IS): 369.1 [M+H]⁺.

### INTERMEDIATE 10f: N-(5-(4-Chlorophenyl)-3,3-dimethyl-1,1-dioxo-2-(4,4,4-trifluorobutyl)-2,3-dihydroisothiazol-4-yl)methyl-N-ethylamine (R═CH₂CH₂CH₂CF₃)

The amine from Intermediate 1 was Boc-protected and then alkylated with 4,4,4-trifluorobutan-1-ol employing CMMP as activating reagent according to a procedure from the literature (Tetrahedron Lett. 37 (1996), 2459-2462); MS (IS): 425.0 [M+H]⁺.

### Intermediate 11

### N-(3-(4-Chlorophenyl)-1-cyclopropylmethyl-2,2-dioxo-2-thia-1-aza-spiro[4.4]non-3-en-4-ylmethyl)-N-ethylamine

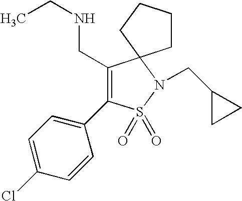

The compound was prepared from 4-bromomethyl-3-(4-chlorophenyl)-2,2-dioxo-2-thia-1-aza-spiro[4.4]non-3-ene (prepared as described during the synthesis of Intermediate 4) with ethylamine followed by alkylation with cyclopropylmethyl bromide in the same manner as described for Intermediate 10e; MS (IS): 395.1 [M+H]⁺.

### Intermediate 12

### N-(5-(4-Chlorophenyl)-3,3-bis(fluoromethyl)-1,1-dioxo-2,3-dihydroisothiazol-4-yl)methyl-N-ethylamine

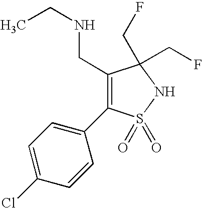

Methyl 2-amino-3-fluoro-2-fluoromethyl-propionate hydrochloride, 189 mg (1.0 mmol, as described in Synthesis 1994, 701-702) is combined with triethylamine (0.6 mL, 4.6 mmol), and 4-dimethylaminopyridine (cat. 5 mg), in dichloromethane (3.3 mL) at room temperature. Then (4-chlorophenyl)methanesulfonyl chloride, 225 mg (1.0 mmol, as described for Intermediate 1) is added and the resulting mixture stirred at room temperature until the reaction is complete. Water is then added and the pH of the aqueous phase adjusted to 2.5 with aqueous hydrochloric acid. The mixture is extracted with dichloromethane and the combined extracts are dried over sodium sulfate and concentrated in vacuo. The resulting residue is chromatographed over silica gel (chloroform/methanol) to give the desired product, shown below. Methyl 2-(4-chlorophenyl)methanesulfonylamino)-3-fluoro-2-fluoromethyl-propionate:

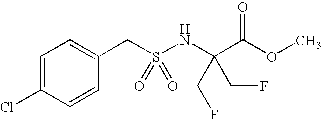

The ester from above (342 mg, 1.0 mmol) is combined with 2 N aqueous sodium hydroxide (7 mL), tetrahydrofuran (0.9 mL), and ethanol (0.9 mL) and the mixture stirred at room temperature until hydrolysis is complete. Aqueous hydrochloric acid (5 N) is added until the aqueous mixture has reached pH 2.0 and the aqueous phase is then extracted with ethyl acetate. The combined extracts are dried over sodium sulfate and the solution concentrated in vacuo. The resulting solid is triturated with diethyl ether, filtered and dried to give the desired acid, shown below. 2-(4-Chlorophenyl)methanesulfonylamino)-3-fluoro-2-fluoromethyl-propionic acid:

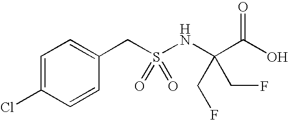

The acid from above (328 mg, 1.0 mmol) is dissolved in anhydrous dimethoxyethane (8 mL) and the mixture cooled to 60° C. (dry ice/acetone bath) under nitrogen. Then methyl lithium (3.6 mL, 1.4 M in ethyl ether) is added via syringe and the resulting mixture stirred until the reaction is complete. The reaction is then quenched by pouring into a stirred mixture of ice/1N aqueous hydrochloric acid and the aqueous mixture extracted with ethyl acetate. The combined extracts are concentrated and the resulting residue chromatographed over silica (chloroform/methanol) to give the desired ketone, shown below. 3-(4-Chlorophenyl)methanesulfonylamino)-4-fluoro-3-fluoromethyl-butan-2-one:

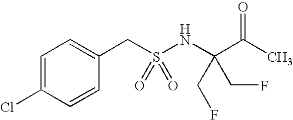

The ketone (328 mg, 1.0 mmol) is dissolved in dimethylformamide (5 mL) and then sodium hydride (60%, 88 mg, 2.2 mmol) is added and the resulting mixture heated at 100° C. until the reaction is complete. The solvent is removed in vacuo and the resulting residue taken up in dilute aqueous hydrochloric acid. The aqueous mixture is extracted with ethyl acetate and the combined extracts are concentrated to leave a residue which is chromatographed over silica (chloroform/methanol) to give the desired product, shown below. 5-(4-Chlorophenyl)-3,3bis(fluoromethyl)-4-methyl-2,3-dihydro-1,1-dioxo-isothiazole:

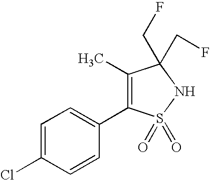

The product from above (308 mg, 1.0 mmol) is slurried in carbon tetrachloride (8.2 mL), N-bromosuccinimide (265 mg, 1.5 mmol), and 2,2′-azobis(2-methyl-propionitrile) (10 mg, cat.) are added. This mixture is heated at reflux until the reaction is complete, then is cooled to ambient temperature and diluted with dichloromethane. The organic mixture is washed with water (2×10 mL) and dried over sodium sulfate. Concentration leaves a residue which is taken up in ethanol (6.6 mL) followed by the addition of ethylamine (70%, 0.66 mL). This mixture is allowed to stir at room temperature until the reaction is complete, then concentrated and the residue chromatographed over silica (chloroform/methanol) to give the desired title amine.

### EXAMPLE 1

### 2-(R)-2-(2-Amino-3-fluoro-2-fluoromethyl-propionylamino)-3-benzyloxy-propionic acid N-[5-(4-chlorophenyl)-3,3-dimethyl-1,1-dioxo-2,3-dihydroisothiazol-4-ylmethyl)-N-ethylamide Hydrochloride

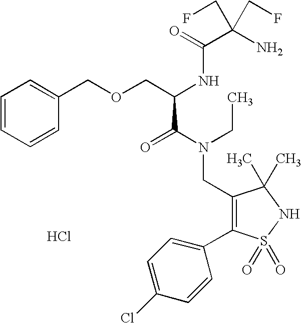

To a suspension of the sulfonamide of Intermediate 2 (260 mg, 0.53 mmol), in 3.9 mL of IPAC were added water (3.8 mL), DCC (120 mg, 0.58 mmol), HOBt (78 mg, 0.58 mmol) and 2-tert-butoxycarbonylamino-3-fluoro-2-fluoromethyl-propionic acid (Intermediate 8; 126 mg, 0.53 mmol). The mixture was allowed to stir for 2 h, and then was filtered rinsing with IPAC. The aqueous phase was separated, and the organic layer was washed with citric acid 0.1 M and saturated NaHCO₃, dried over Na₂SO₄ and evaporated to dryness. The residue was chromatographed over silica with ethanol/dichloromethane 2:98 as eluant to yield 210 mg (56%) of the desired product, shown below, as a white-off solid. ¹H-NMR is consistent with structure; MS (ion spray) 613.1 (M-Boc+1):

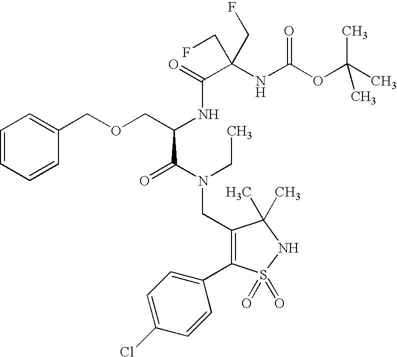

To the tert-butoxycarbamate (200 mg, 0.28 mmol) was added a solution of HCl 10% in ethanol (1.7 mL). The mixture was stirred at room temperature for 4 h, then poured into diethyl ether (300 mL) and stirred for 2 h. The white precipitate formed was filtered and dried to yield 120 mg (66%) of the desired title product, as a white solid. ¹H-NMR is consistent with structure; MS (ion spray) 613.2 (M+1).

### EXAMPLE 2

### 2-(R)-2-(2-(R,S)-2-Amino-4,4,4-trifluorobutanoylamino)-3-benzyloxy-propionic acid N-[5-(4-chlorophenyl)-3,3-dimethyl-1,1-dioxo-2,3-dihydroisothiazol-4-ylmethyl]-N-ethylamide Hydrochloride

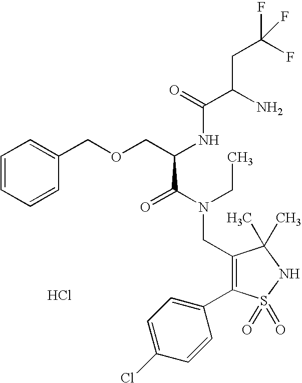

Commercial 2-amino-4,4,4-trifluorobutanoic acid was protected as described for Intermediate 8 and the title compound prepared from the resulting tert-butylcarbamate and Intermediate 2 in the same manner as described for Example 1: MS (ion spray): 631.2 (M+1); yield of the last coupling step 38%.

### EXAMPLE 3

### 2-(R)-2-((1-Amino-cyclopropancarbonyl)amino)-3-benzyloxy-propionic acid N-(5-(4-chlorophenyl)-3,3-dimethyl-1,1-dioxo-2,3-dihydroisothiazol-4-ylmethyl]-N-ethylamide Hydrochloride

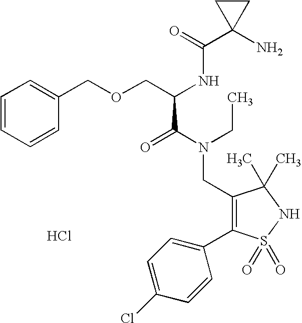

The title compound was prepared from commercial 1-(tert-butoxycarbonylamino)cyclopropanecarboxylic acid and Intermediate 2 as described for Example 1; MS (IS): 575.2 [M+H]⁺.

### EXAMPLE 4

### 2-(R)-2-((2-(R)-2-Amino-3-hydroxy-2-methylpropionyl)amino)-3-benzyloxy-propionic acid N-[5-(4-chlorophenyl)-3,3-dimethyl-1,1-dioxo-2,3-dihydroisothiazol-4-ylmethyl]-N-ethylamide Hydrochloride

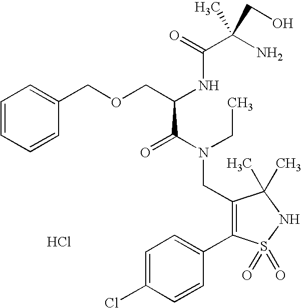

The title compound was prepared according to the methods described in Example 1 from 2-(R)-2-(tert-butoxycarbonylamino)-3-hydroxy-2-methylpropionic acid and Intermediate 2; MS (IS): 593.4 [M+H]⁺.

### EXAMPLE 5

### 2-(R)-2-((2-(S)-2-Amino-3-hydroxy-2-methylpropionyl)amino)-3-benzyloxy-propionic acid N-[5-(4-chlorophenyl)-3,3-dimethyl-1,1-dioxo-2,3-dihydroisothiazol-4-ylmethyl]-N-ethylamide Hydrochloride

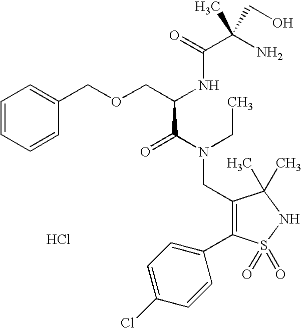

The title compound was prepared according to the methods described in Example 1 from 2-(S)-2-(tert-butoxycarbonylamino)-3-hydroxy-2-methylpropionic acid and Intermediate 2; MS (IS): 595.1 [M+H]⁺.

### EXAMPLE 6

### 2-(R)-2-(2-Amino-2-methylpropionylamino)-3-benzyloxy-propionic acid N-[5-(4-chlorophenyl)-3,3-dimethyl-1,1-dioxo-2,3-dihydroisothiazol-4-ylmethyl]-N-(2-fluoroethyl)amide Hydrochloride

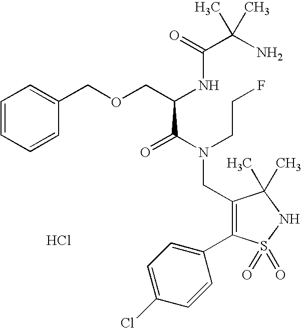

To a solution of Intermediate 6e, 384 mg (1.3 mmol) in 25 mL of dichloromethane was added 183 μL (1.3 mmol) of triethylamine and 418 mg (1.3 mmol) of TBTU. The mixture was stirred for 30 min at ambient temperature, 433 mg (1.3 mmol) of Intermediate 3 was added and the mixture stirred at ambient temperature overnight. The mixture was washed with citric acid (10% in water), saturated sodium bicarbonate solution, and brine. The organic phase was dried over sodium sulfate and evaporated to dryness. The residue was purified by chromatography on silica gel with methanol/dichloromethane as eluent to yield the desired product. Yield: 563 mg (71%);. MS (IS): 510.1 [M-COOC(CH₃)₃]⁺; 631.1 [M+Na]⁺; 2-(R)-2-(tert.-butoxycarbonylamino)-3-benzyloxy-propionic acid N-[5-(4-chlorophenyl)-3,3-dimethyl-1,1-dioxo-2,3-dihydroisothiazol-4-ylmethyl]-N-(2-fluoroethyl)amide, shown below:

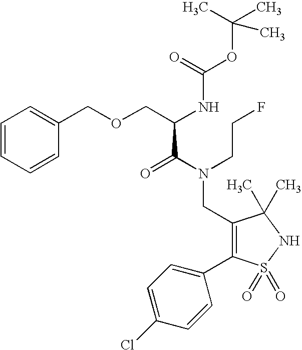

The tert-butyl carbamate from above, 563 mg (0.92 mmol) was dissolved in 20 mL 2-propanol and treated with 20 mL hydrochloric acid (5-6 N in 2-propanol) at ambient temperature. The resulting mixture was stirred overnight and evaporated to dryness. The residue was taken up in ethyl acetate, washed with saturated aqueous sodium bicarbonate solution and brine. The organics were dried with magnesium sulfate and evaporated to dryness to yield the desired product, shown below. Yield: 460 mg (98%);. MS (IS): 511.1 [M+H]⁺. 2-(R)-2-amino-3-benzyloxy-propionic acid N-[5-(4-chlorophenyl)-3,3-dimethyl-1,1-dioxo-2,3-dihydroisothiazol-4-ylmethyl]-N-(2-fluoroethyl)amide:

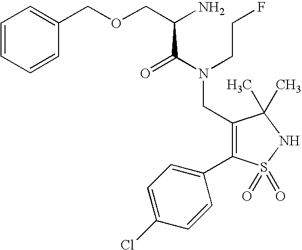

To a solution of the commercially available N-Boc-protected amino isobutyric acid, 142 mg (0.7 mmol) in 15 mL of dichloromethane were added 98 μL (0.7 mmol) of triethylamine and 225 mg (0.7 mmol) of TBTU. The mixture was stirred for 30 min at ambient temperature, 357 mg (0.7 mmol) of 2-(R)-2-amino-3-benzyloxy-propionic acid N-[5-(4-chlorophenyl)-3,3-dimethyl-1,1-dioxo-2,3-dihydroisothiazol-4-ylmethyl]-N-(2-fluoroethyl)amide was added and the mixture stirred at ambient temperature overnight. The mixture was washed with citric acid (10% in water), saturated sodium bicarbonate solution, and brine. The organic phase was dried with sodium sulfate and evaporated to dryness. The residue was purified by chromatography on silica gel with methanol/dichloromethane as eluant to yield the desired Boc-protected title compound. Yield: 389 mg (80%);. MS (IS): 693.2 [M−H]⁻.

348 mg (0.5 mmol) of the intermediate above were stirred with a mixture of 15 mL 2-propanol and 15 mL hydrochloric acid (5-6 N in 2-propanol) at ambient temperature overnight. It was evaporated to dryness, and the residue was taken up in ethyl acetate, and washed with saturated sodium bicarbonate solution and brine. The organics were dried with magnesium sulfate and evaporated to dryness. The residue was purified by chromatography on silica gel using methanol/dichloromethane as eluent to yield the amine of the desired title compound which was converted to the hydrochloride salt by addition of HCl in ether followed by evaporation. Yield: 208 mg (65%);. MS (IS): 595.2 [M+H]⁺.

### EXAMPLE 7

### 2-(R)-2-(2-Amino-2-methylpropionylamino)-3-benzyloxy-propionic acid N-(3-(4-chlorophenyl)-2,2-dioxo-2-thia-1-azaspiro[4.4]non-3-ene-4-ylmethyl)-N-(2-fluoroethyl)amide Hydrochloride

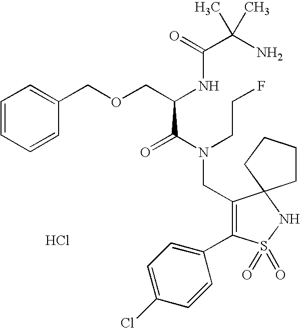 The title compound was prepared from Intermediate 4 as described for Example 6. MS (IS): 621.2 [M+H]⁺.

### EXAMPLE 8

### 2-(R)-2-(2-Amino-2-methylpropionylamino)-3-benzyloxy-propionic acid N-[5-(4-chlorophenyl)-3,3-dimethyl-1,1-dioxo-2,3-dihydroisothiazol-4-ylmethyl]-O-methyl-hydroxylamide Hydrochloride

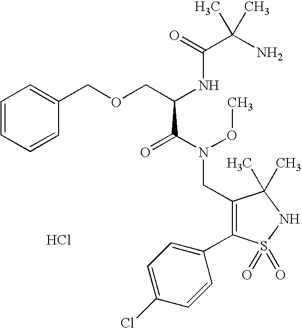 The title compound was prepared from Intermediates 6e and 7 as described for Example 1. MS (IS): 579.1 [M+H]⁺.

### EXAMPLE 9

### 2-(R)-2-(2-Amino-2-methylpropionylamino)-5-phenylpentanoic acid N-[5-(4-chlorophenyl)-3,3-dimethyl-1,1-dioxo-2,3-dihydroisothiazol-4-ylmethyl]-O-methyl-hydroxylamide Hydrochloride

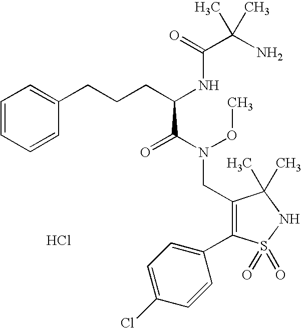 The title compound was prepared from Intermediates 7 and 9 as described for Example 1. MS (IS): 577.2 [M+H]⁺.

### EXAMPLE 10

### 2-(R)-2-(2-Amino-2-methylpropionylamino)-3-benzyloxy-propionic acid N-(3-(4-chlorophenyl)-1-cyclopropylmethyl-2,2-dioxo-2-thia-1-azaspiro[4.4]non-3-ene-4-ylmethyl)-N-ethylamide Hydrochloride

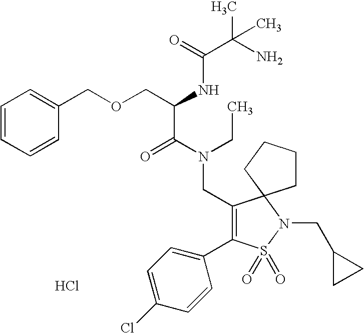 The title compound was prepared from Intermediate 6e and Intermediate 11 in the same manner as described for Example 6. MS (IS): 657.2 [M+H]⁺.

The following Examples 11-16 were prepared in the same manner as described for Example 6 from Intermediate 6e and the corresponding sulfonamide Intermediates 10:

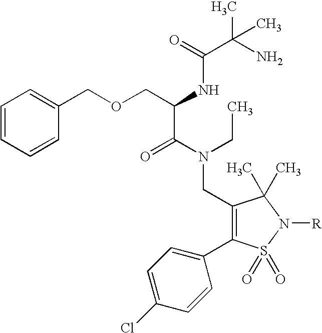

### EXAMPLE 11

### 2-(R)-2-(2-Amino-2-methylpropionylamino)-3-benzyloxy-propionic acid N-[5-(4-chlorophenyl)-2-cyclopropylmethyl-3,3-dimethyl-1,1-dioxo-2,3-dihydroisothiazol-4-ylmethyl]-N-ethylamide Hydrochloride (R═CH₂C₃H₅)

Prepared from Intermediate 10e. MS (IS): 631.2 [M+H]⁺.

### EXAMPLE 12

### 2-(R)-2-(2-Amino-2-methylpropionylamino)-3-benzyloxy-propionic acid N-[5-(4-chlorophenyl)-3,3-dimethyl-1,1-dioxo-2-(2-methoxyethyl)-2,3-dihydroisothiazol-4-ylmethyl]-N-ethylamide Hydrochloride (R═CH₂CH₂OCH₅)

Prepared from Intermediate lob. MS (IS): 635.2 [M+H]⁺.

### EXAMPLE 13

### 2-(R)-2-(2-Amino-2-methylpropionylamino)-3-benzyloxy-propionic acid N-[2-carbamoylmethyl-5-(4-chlorophenyl)-3,3-dimethyl-1,1-dioxo-2,3-dihydroisothiazol-4-ylmethyl]-N-ethylamide Hydrochloride (R═CH₂CONH₂)

Prepared from Intermediate 10c. MS (IS): 634.2 [M+H]⁺.

### EXAMPLE 14

### 2-(R)-2-(2-Amino-2-methylpropionylamino)-3-benzyloxy-propionic acid N-[5-(4-chlorophenyl)-3,3-dimethyl-2-(N′,N′-dimethylcarbamoyl)methyl-1,1-dioxo-2,3-dihydroisothiazol-4-ylmethyl]-N-ethylamide Hydrochloride (R═CH₂CON(CH₃)₂)

Prepared from Intermediate 10d. MS (IS): 662.2 [M+H]⁺.

### EXAMPLE 15

### 2-(R)-2-(2-Amino-2-methylpropionylamino)-3-benzyloxy-propionic acid N-[5-(4-chlorophenyl)-3,3-dimethyl-1,1-dioxo-2-(2-fluoroethyl)-2,3-dihydroisothiazol-4-ylmethyl]-N-ethylamide Hydrochloride (R═CH₂CH₂F)

Prepared from Intermediate 10a. MS (IS): 623.2 [M+H]⁺.

### EXAMPLE 16

### 2-(R)-2-(2-Amino-2-methylpropionylamino)-3-benzyloxy-propionic acid N-[5-(4-chlorophenyl)-3,3-dimethyl-1,1-dioxo-2-(4,4,4-trifluorobutyl)-2,3-dihydroisothiazol-4-ylmethyl]-N-ethylamide (R═CH₂CH₂CH₂CF₃)

Prepared from Intermediate 10f. MS (IS): 687.1 [M+H]⁺.

### EXAMPLE 17

### 2-(R)-2-(2-Amino-2-methylpropionylamino)-3-(3,5-difluorophenyl)methoxypropionic acid N-(3-(4-chlorophenyl)-2,2-dioxo-2-thia-1-azaspiro[4.4]non-3-ene-4-ylmethyl)-N-(2-fluoroethyl)amide Hydrochloride

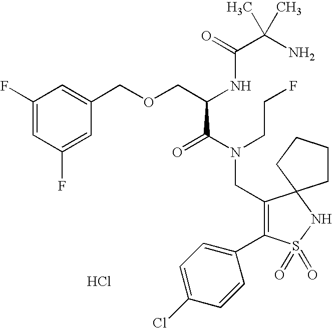

The title compound was prepared from Intermediates 4 and 6b as described for Example 7. MS (IS): 657.1 [M+H]⁺.

### EXAMPLE 18

### 2-(R)-2-((1-Amino-cyclopropancarbonyl)amino)-3-(4-fluorophenylmethoxy)-propionic acid N-[5-(4-chlorophenyl)-3,3-dimethyl-1,1-dioxo-2,3-dihydroisothiazol-4-ylmethyl]-N-ethylamide Hydrochloride

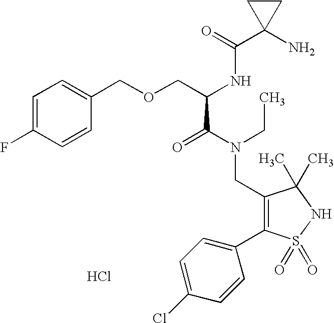

The title compound was prepared according to the methods described in Example 6 from Intermediates 1 and 6a followed by coupling with commercial 1-(tert-butoxycarbonylamino)cyclopropanecarboxylic acid. Yield: 20 mg (35 W); MS (IS): 593.0 [M+H]⁺

### EXAMPLE 19

### 2-(R)-2-((1-Amino-cyclopropancarbonyl)amino)-3-(2,4-difluorophenylmethoxy)-propionic acid N-[5-(4-chlorophenyl)-3,3-dimethyl-1,1-dioxo-2,3-dihydroisothiazol-4-ylmethyl]-N-methylamide Hydrochloride

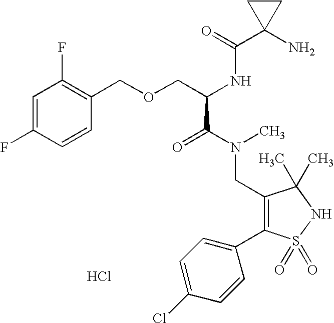

The title compound was prepared in the same manner as described for Example 18 from Intermediates 5 and 6d. Yield: 19 mg (73%); MS (IS): 598.0 [M+H]⁺.

### EXAMPLE 20

### 2-(R)-2-(2-Amino-3-fluoro-2-fluoromethyl-propionylamino)-3-(2,6-difluorophenylmethoxy)-propionic acid N-[5-(4-chlorophenyl)-3,3-dimethyl-1,1-dioxo-2,3-dihydroisothiazol-4-ylmethyl]-N-ethylamide Hydrochloride

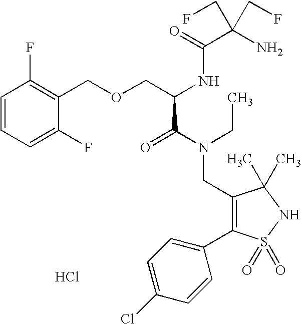

The title compound was prepared by coupling between Intermediates 1 and 6c followed by the second coupling step with Intermediate 8 in the same manner as described for Example 1 (MS, ion spray, 649.2 (M+1); overall yield for the last coupling and deprotection sequence 25%.

### EXAMPLE 21

### 2-(R)-2-(2-Amino-2-methylpropionylamino)-3-benzyloxy propionic acid N-[5-(4-chlorophenyl)-3,3-bis(fluoromethyl)-1,1-dioxo-2,3-dihydroisothiazol-4-ylmethyl]-N-ethylamide Hydrochloride

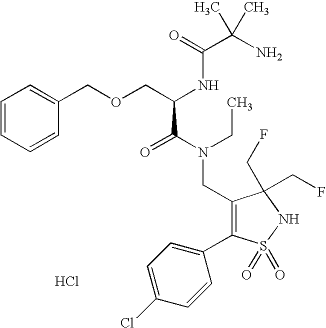

To a suspension of Intermediate 12 (351 mg, 1.0 mmol) in 4.8 mL of IPAC are added water (2.8 mL), DCC (227 mg, 1.1 mmol), HOBt (143 mg, 1.1 mmol), IPAC (1 mL) and Intermediate 6e (245 mg, 1.0 mmol). The mixture is allowed to stir for 14 h, and it is filtered and rinsed with IPAC. The aqueous phase is separated, and the organic layer is washed with citric acid 0.1 M and saturated NaHCO₃, dried over Na₂SO₄ and evaporated to give the desired product, shown below. 2-(R)-2-(tert.-Butoxycarbonylamino)-3-benzyloxy-propionic acid N-[5-(4-chlorophenyl)-3,3-bis(fluoromethyl)-1,1-dioxo-2,3-dihydroisothiazol-4-ylmethyl]-N-ethylamide:

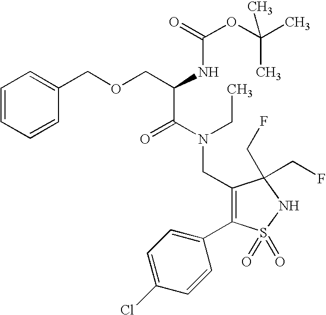

To a solution of the above tert-butyl carbamate (628 mg, 1.0 mmol) in 4.7 mL of dichloromethane is added 4.7 mL of trifluoroacetic acid. The mixture is stirred at room temperature until the reaction is complete, then poured into diethyl ether (500 mL) and stirred for 2 h. The white precipitate formed is filtered and dried to afford a white solid which is dissolved in dichloromethane and washed with saturated NaHCO₃ to give the desired amine, shown below. 2-(R)-2-Amino-3-benzyloxy-propionic acid N-[5-(4-chlorophenyl)-3,3-bis(fluoromethyl)-1,1-dioxo-2,3-dihydroisothiazol-4-ylmethyl]-N-ethylamide:

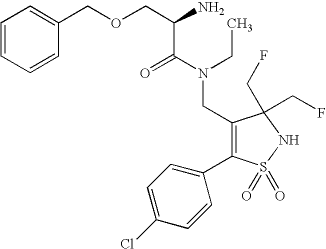

To a suspension of the above amine (528 mg, 1.0 mmol) in 7.3 mL of IPAC are added water (7.6 mL), DCC (226 mg, 1.1 mmol), HOBt (147 mg, 1.1 mmol), and 2-tert-Butoxycarbonylamino-2-methyl-propionic acid (222 mg, 1.1 mmol). The mixture is allowed to stir until the reaction is complete, and then is filtered rinsing with IPAC. The aqueous phase is separated, and the organic layer is washed with citric acid (0.1 M) and saturated NaHCO₃, dried over Na₂SO₄ and evaporated to dryness. The residue is chromatographed over silica with ethanol/dichloromethan to give the desired product, shown below. 2-(R)-2-(2-tert-Butoxycarbonyl-2-methylpropionylamino)-3-benzyloxy propionic acid N-[5-(4-chlorophenyl)-3,3-bis(fluoromethyl)-1,1-dioxo-2,3-dihydroisothiazol-4-ylmethyl]-N-ethylamide.

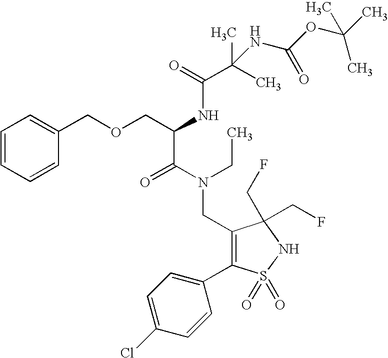

To the above carbamate (713 mg, 1.0 mmol) is added a solution of HCl (10%) in ethanol. The mixture is stirred at room temperature until the reaction is complete, then poured into diethyl ether (500 mL) and stirred for 2 h. The white precipitated formed is filtered and dried to give the desired title product.

### Pituitary Cell Culture Assay for Growth Hormone (GH) Secretion

Fifteen 250 g male Sprague-Dawley rats are used for each assay. The animals are killed by decapitation and anterior pituitaries are removed and placed into ice cold culture medium. The pituitaries are sectioned in small pieces and enzymatically digested using trypsin (Difco) to weaken connective tissue. Pituitary cells are dispersed by mechanical agitation, collected, pooled and then seeded into 96-well plates (50,000 cells/well). After 5 days of culture, the cells formed as monolayer (70-80% confluent). Cells are then washed with medium (without phenol red) and incubated for 90 min at 37° C. Afterwards the cells are challenged to secrete GH by the addition of GH secretagogues to the medium. After 45 min at room temperature, the medium is removed, filtered and stored frozen until radioimmunoassays for rat GH were performed. Doses of secretagogue are added in triplicates. Compounds disclosed herein are active in the assay as described. The compounds cause a stimulation of GH secretion resulting in at least 20% increase of the basal level of GH with and EC50<500 nM. Preferred compounds caused a 50% increase with an EC50<50 nM, and more preferred compounds a 50% increase with an EC50<10 nM. Both EC50 and efficacy values were calculated by the 4-parameter logistic equation. Such values were pooled and represented as mean+/−standard error, when appropriate.

1. A compound of the Formula I 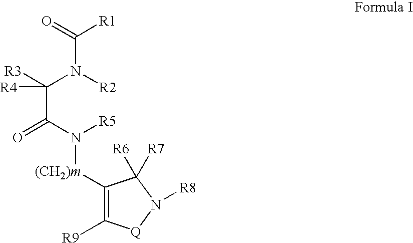
wherein:
R1 is NHR10, (substituted or unsubstituted C₁-C₆alkyl)NHR10 or (unsubstituted or substituted C₃-C₈ cycloalkyl)NHR10;
R10 is hydrogen, C₁-C₆alkyl, C₁-C₆alkyl(OH), C₁-C₆alkylidenyl(OH)R11, or an amino protecting group;
R11 is C₁-C₆alkyl, C₂-C₆alkenyl, C₁-C₆alkyl(O)C₁-C₆alkyl, C(O)O—C₁-C₆alkyl, aryl, or C₁-C₆alkylaryl;
R2 is hydrogen, C₁-C₆alkyl, aryl, or C₁-C₆alkylaryl;
R3 is unsubstituted or substituted aryl, unsubstituted or substituted C₁-C₆alkylaryl, unsubstituted or substituted C₁-C₆alkyl(O)—C₁-C₆alkylaryl, unsubstituted or substituted C₃-C₈ cycloalkyl, unsubstituted or substituted (C₁-C₆ alkyl) C₃-C₈ cycloalkyl, indolinyl;
R4 is hydrogen, C₁-C₆alkyl, aryl, C₁-C₆alkylaryl, or C₂-C₆alkenyl;
R5 is hydrogen, aryl, C₁-C₆alkylaryl, hydroxy, C₁-C₆alkoxy, unsubstituted or substituted C₁-C₆alkyl;
R6 and R7 are independently hydrogen, unsubstituted or substituted C₁-C₆alkyl, unsubstituted or substituted C₂-C₆alkenyl, or R6 and R7 together with the carbon atom to which they are attached form a carbocyclic ring of up to 8 atoms which is optionally partly unsaturated or a substituted C₃-C₈ cycloalkyl group which is optionally partly unsaturated;
R8 is hydrogen, unsubstituted or substituted C₁-C₆alkyl, unsubstituted or substituted aryl, unsubstituted or substituted (C₁-C₆alkyl)C₃-C₈cycloalkyl, or unsubstituted or substituted C₁-C₆alkylaryl;
R9 is hydrogen, C₁-C₆alkyl, C₂-C₆alkenyl, C₂-C₆alkynyl, C₃-C₈cycloalkyl, C₃-C₈cycloalkenyl, cyano, unsubstituted or substituted aryl, unsubstituted or substituted —O-aryl, unsubstituted or substituted —N-aryl, unsubstituted or substituted —S-aryl, -aryl-aryl(K1)(K2), —O-aryl-aryl(K1)(K2), —N-aryl-aryl(K1)(K2), —S-aryl-aryl(K1)(K2), —O—C₁-C₆alkyl, or Cl —C₆alkylaryl, wherein K1 is halo or —CF₃, and K2 is hydrogen, halo or —CF₃ or K1 and K2 together form a methylenedioxy group;
Q is —S(O)₂— or —C(O)—; and
m is a number selected from 1 or 2;
provided that R1 is (substituted C₁-C₆alkyl)NHR10 or (unsubstituted or substituted C₃-C₈ cycloalkyl)NHR10; or
R5 is hydroxy, C₁-C₆alkoxy, or substituted C₁-C₆alkyl; or
R6 and R7 are independently unsubstituted or substituted C₁-C₆alkyl or unsubstituted or substituted C₂-C₆alkenyl with the proviso that at least one group is substituted; or
R6 is hydrogen and R7 is substituted C₁-C₆alkyl or substituted C₂-C₆alkenyl; or
R6 and R7 together with the carbon atom to which they are attached may form a substituted C₃-C₈ cycloalkyl group which is optionally partly unsaturated; or
R8 is substituted C₁-C₆alkyl, substituted aryl, unsubstituted or substituted (C₁-C₆alkyl)C₃-C₈cycloalkyl or substituted C₁-C₆alkylaryl;
or a pharmaceutically acceptable salt or solvate thereof.

2. A compound according to claim 1 having Formula I 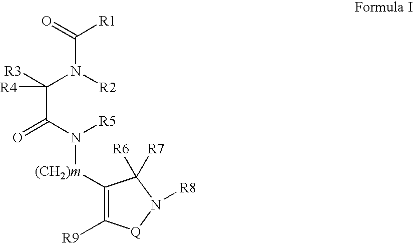
wherein:
R1 is NHR10 or C₁-C₆alkylNHR10;
R10 is hydrogen, C₁-C₆alkyl, C₁-C₆alkyl(0H), C₁-C₆alkylidenyl(OH)R11, or an amino protecting group;
R11 is C₁-C₆alkyl, C₂-C₆alkenyl, C₁-C₆alkyl(O)C₁-C₆alkyl, C(O)O—C₁-C₆alkyl, aryl, or C₁-C₆alkylaryl;
R2 is hydrogen, C₁-C₆alkyl, aryl, or C₁-C₆alkylaryl;
R3 is unsubstituted or substituted aryl, unsubstituted or substituted C₁-C₆alkylaryl, unsubstituted or substituted C₁-C₆alkyl(O)—C₁-C₆alkylaryl, unsubstituted or substituted C₃-C₈ cycloalkyl, unsubstituted or substituted (C₁-C₆ alkyl) C₃-C₈ cycloalkyl, indolinyl;
R4 is hydrogen, C₁-C₆alkyl, aryl, C₁-C₆alkylaryl, or C₂-C₆alkenyl;
R5 is hydroxy, C₁-C₆alkoxy, or substituted C₁-C₆alkyl;
R6 and R7 are independently hydrogen, C₁-C₆alkyl, C₂-C₆alkenyl, or R6 and R7 together with the carbon atom to which they are attached form a carbocyclic ring of up to 8 atoms which is optionally partly unsaturated;
R8 is hydrogen, C₁-C₆alkyl, aryl, or C₁-C₆alkylaryl;
R9 is hydrogen, C₁-C₆alkyl, C₂-C₆alkenyl, C₂-C₆alkynyl, C₃-C₈cycloalkyl, C₃-C₈cycloalkenyl, cyano, unsubstituted or substituted aryl, unsubstituted or substituted —O-aryl, unsubstituted or substituted —N-aryl, unsubstituted or substituted —S-aryl, -aryl-aryl(K1)(K2), —O-aryl-aryl(K1)(K2), —N-aryl-aryl(K1)(K2), —S-aryl-aryl(K1)(K2), —O—C₁-C₆alkyl, or C₁-C₆alkylaryl, wherein K1 is halo or —CF₃, and K2 is hydrogen, halo or —CF₃ or K1 and K2 together form a methylenedioxy group;
Q is —S(O)₂— or —C(O)—;
m is a number selected from 1 or 2;
or a pharmaceutically acceptable salt or solvate thereof.

3. A compound according to claim 1 having Formula I 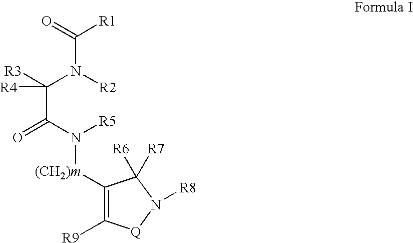
wherein:
R1 is NHR10 or C₁-C₆alkylNHR10;
R10 is hydrogen, C₁-C₆alkyl, C₁-C₆alkyl(OH), C₁-C₆alkylidenyl(OH)R11, or an amino protecting group;
R11 is C₁-C₆alkyl, C₂-C₆alkenyl, C₁-C₆alkyl(O)C₁-C₆alkyl, C(O)O—C₁-C₆alkyl, aryl, or C₁-C₆alkylaryl;
R2 is hydrogen, C₁-C₆alkyl, aryl, or C₁-C₆alkylaryl;
R3 is unsubstituted or substituted aryl, unsubstituted or substituted C₁-C₆alkylaryl, unsubstituted or substituted C₁-C₆alkyl(O)—C₁-C₆alkylaryl, unsubstituted or substituted C₃-C₈ cycloalkyl, unsubstituted or substituted (C₁-C₆ alkyl) C₃-C₈ cycloalkyl, indolinyl;
R4 is hydrogen, C₁-C₆alkyl, aryl, C₁-C₆alkylaryl, or C₂-C₆alkenyl;
R5 is hydrogen, aryl, C₁-C₆alkylaryl, hydroxy, C₁-C₆alkoxy, unsubstituted or substituted C₁-C₆alkyl;
R6 and R7 are independently hydrogen, C₁-C₆alkyl, C₂-C₆alkenyl, or R6 and R7 together with the carbon atom to which they are attached form a carbocyclic ring of up to 8 atoms which is optionally partly unsaturated;
R8 is substituted C₁-C₆alkyl, substituted aryl, unsubstituted or substituted (C₁-C₆alkyl)C₃-C₈cycloalkyl or substituted C₁-C₆alkylaryl;
R9 is hydrogen, C₁-C₆alkyl, C₂-C₆alkenyl, C₂-C₆alkynyl, C₃-C₈cycloalkyl, C₃-C₈cycloalkenyl, cyano, unsubstituted or substituted aryl, unsubstituted or substituted —O-aryl, unsubstituted or substituted —N-aryl, unsubstituted or substituted —S-aryl, -aryl-aryl(K1)(K2), —O-aryl-aryl(K 1)(K2), —N-aryl-aryl(K 1)(K2), —S-aryl-aryl(K 1)(K2), —O—C₁-C₆alkyl, or Cl —C₆alkylaryl, wherein K1 is halo or —CF₃, and K2 is hydrogen, halo or —CF₃ or K1 and K2 together form a methylenedioxy group;
Q is —S(O)₂— or —C(O)—;
m is a number selected from 1 or 2;
or a pharmaceutically acceptable salt or solvate thereof.

4. A compound according to claim 1 having Formula I 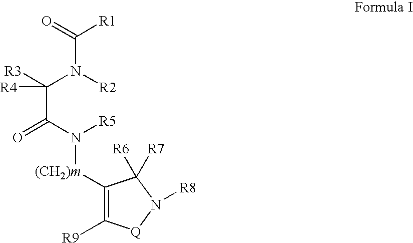
wherein:
R1 is NHR10 or C₁-C₆alkylNHR10;
R10 is hydrogen, C₁-C₆alkyl, C₁-C₆alkyl(OH), C₁-C₆alkylidenyl(OH)R11, or an amino protecting group;
R11 is C₁-C₆alkyl, C₂-C₆alkenyl, C₁-C₆alkyl(O)C₁-C₆alkyl, C(O)O—C₁-C₆alkyl, aryl, or C₁-C₆alkylaryl;
R2 is hydrogen, C₁-C₆alkyl, aryl, or C₁-C₆alkylaryl;
R3 is unsubstituted or substituted aryl, unsubstituted or substituted C₁-C₆alkylaryl, unsubstituted or substituted C₁-C₆alkyl(O)—C₁-C₆alkylaryl, unsubstituted or substituted C₃-C₈ cycloalkyl, unsubstituted or substituted (C₁-C₆ alkyl) C₃-C₈ cycloalkyl, indolinyl;
R4 is hydrogen, C₁-C₆alkyl, aryl, C₁-C₆alkylaryl, or C₂-C₆alkenyl;
R5 is hydrogen, aryl, C₁-C₆alkylaryl, hydroxy, C₁-C₆alkoxy, unsubstituted or substituted C₁-C₆alkyl;
R6 and R7 are independently unsubstituted or substituted C₁-C₆alkyl or unsubstituted or substituted C₂-C₆alkenyl with the proviso that at least one group is substituted; or
R6 is hydrogen and R7 is substituted C₁-C₆alkyl or substituted C₂-C₆alkenyl; or
or R6 and R7 together with the carbon atom to which they are attached may form a substituted C₃-C₈ cycloalkyl group which is optionally partly unsaturated;
R8 is hydrogen, unsubstituted or substituted C₁-C₆alkyl, unsubstituted or substituted aryl, unsubstituted or substituted (C₁-C₆alkyl)C₃-C₈cycloalkyl or unsubstituted or substituted C₁-C₆alkylaryl;
R9 is hydrogen, C₁-C₆alkyl, C₂-C₆alkenyl, C₂-C₆alkynyl, C₃-C₈cycloalkyl, C₃-C₈cycloalkenyl, cyano, unsubstituted or substituted aryl, unsubstituted or substituted —O-aryl, unsubstituted or substituted —N-aryl, unsubstituted or substituted —S-aryl, -aryl-aryl(K1)(K2), -O-aryl-aryl(K1)(K2), —N-aryl-aryl(K1)(K2), —S-aryl-aryl(K1)(K2), —O—C₁-C₆alkyl, or C₁-C₆alkylaryl, wherein K1 is halo or —CF₃, and K2 is hydrogen, halo or —CF₃ or K1 and K2 together form a methylenedioxy group;
Q is —S(O)₂— or —C(O)—;
m is a number selected from 1 or 2;
or a pharmaceutically acceptable salt or solvate thereof.

5. A compound according to claim 1 having Formula I 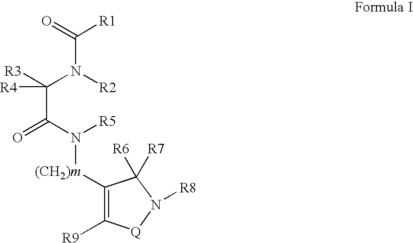
wherein:
R1 is (substituted C₁-C₆alkyl)NHR10 or (unsubstituted or substituted C₃-C₈ cycloalkyl)NHR10;
R10 is hydrogen, C₁-C₆alkyl, C₁-C₆alkyl(OH), C₁-C₆alkylidenyl(OH)R11, or an amino protecting group;
R11 is C₁-C₆alkyl, C₂-C₆alkenyl, C₁-C₆alkyl(O)C₁-C₆alkyl, C(O)O—C₁-C₆alkyl, aryl, or C₁-C₆alkylaryl;
R2 is hydrogen, C₁-C₆alkyl, aryl, or C₁-C₆alkylaryl;
R3 is unsubstituted or substituted aryl, unsubstituted or substituted C₁-C₆alkylaryl, unsubstituted or substituted C₁-C₆alkyl(O)—C₁-C₆alkylaryl, unsubstituted or substituted C₃-C₈ cycloalkyl, unsubstituted or substituted (C₁-C₆ alkyl) C₃-C₈ cycloalkyl, indolinyl;
R4 is hydrogen, C₁-C₆alkyl, aryl, C₁-C₆alkylaryl, or C₂-C₆alkenyl;
R5 is hydrogen, aryl, C₁-C₆alkylaryl, hydroxy, C₁-C₆alkoxy, unsubstituted or substituted C₁-C₆alkyl;
R6 and R7 are independently hydrogen, unsubstituted or substituted C₁-C₆alkyl, unsubstituted or substituted C₂-C₆alkenyl, or R6 and R7 together with the carbon atom to which they are attached form a carbocyclic ring of up to 8 atoms which is optionally partly unsaturated or a substituted C₃-C₈ cycloalkyl group which is optionally partly unsaturated;
R8 is hydrogen, unsubstituted or substituted C₁-C₆alkyl, unsubstituted or substituted aryl, unsubstituted or substituted (C₁-C₆alkyl)C₃-C₈cycloalkyl or unsubstituted or substituted C₁-C₆alkylaryl;
R9 is hydrogen, C₁-C₆alkyl, C₂-C₆alkenyl, C₂-C₆alkynyl, C₃-C₈cycloalkyl, C₃-C₆cycloalkenyl, cyano, unsubstituted or substituted aryl, unsubstituted or substituted —O-aryl, unsubstituted or substituted —N-aryl, unsubstituted or substituted —S-aryl, -aryl-aryl(K1)(K2), —O-aryl-aryl(K1)(K2), —N-aryl-aryl(K1)(K2), —S-aryl-aryl(K1)(K2), —O—C₁-C₆alkyl, or Cl —C₆alkylaryl, wherein K1 is halo or —CF₃, and K2 is hydrogen, halo or —CF₃ or K1 and K2 together form a methylenedioxy group;
Q is —S(O)₂— or —C(O)—;
m is a number selected from 1 or 2;
or a pharmaceutically acceptable salt or solvate thereof.

6. A compound according to claim 2 wherein R1 is 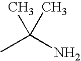
or a pharmaceutically acceptable salt or solvate thereof.

7. A compound according to claim 6 wherein R6 and R7 are each C₁-C₃ alkyl or form a five or six membered carbocyclic ring; or a pharmaceutically acceptable salt or solvate thereof.

8. A compound according to claim 7 wherein R5 is hydroxy, C₁-C₆alkoxy, C₁-C₆alkyl which is substituted by hydroxy or C₁-C₆alkyl which is substituted by one, two, or three halo atoms, or a pharmaceutically acceptable salt or solvate thereof.

9. A compound according to claim 8 wherein R8 is hydrogen, methyl, ethyl or benzyl, or a pharmaceutically acceptable salt or solvate thereof.

10. A compound according to claim 3 wherein R1 is 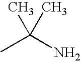
or a pharmaceutically acceptable salt or solvate thereof.

11. A compound according to claim 10 wherein R6 and R7 are each C₁-C₃ alkyl or form a five or six membered carbocyclic ring, or a pharmaceutically acceptable salt or solvate thereof.

12. A compound according to claim 11 wherein R5 is hydrogen, C₁-C₆alkyl, C₁-C₆alkoxy, C₁-C₆alkyl which is substituted by hydroxy or C₁-C₆alkyl which is substituted by one, two, or three halo atoms, or a pharmaceutically acceptable salt or solvate thereof.

13. A compound according to claim 12 wherein R8 is C₁-C₆alkyl which is substituted by hydroxy or C₁-C₆alkyl which is substituted by one, two, or three halo atoms, phenyl substituted by one, two, or three halo atoms or benzyl substituted by one, two, or three halo atoms, or a pharmaceutically acceptable salt or solvate thereof.

14. A compound according to claim 4 wherein R1 is 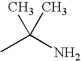
or a pharmaceutically acceptable salt or solvate thereof.

15. A compound according to claim 11 or 14 wherein R6 and R7 are independently C₁-C₆alkyl or C₂-C₆alkenyl, in which one or both groups are substituted by one, two, or three halo atoms; or R6 is hydrogen and R7 is C₁-C₆alkyl, C₂-C₆alkenyl which is substituted by one, two, or three halo atoms; or R6 and R7 together with the carbon atom to which they are attached may form a C3-C8cycloalkyl group which is optionally partly unsaturated and which is substituted by one, two, or three halo atoms, or a pharmaceutically acceptable salt or solvate thereof.

16. A compound according to claim 15 wherein R5 is hydrogen, C₁-C₆alkyl, C₁-C₆alkoxy, C₁-C₆alkyl which is substituted by hydroxy or C₁-C₆alkyl which is substituted by one, two, or three halo atoms, or a pharmaceutically acceptable salt or solvate thereof.

17. A compound according to claim 16 wherein R8 is hydrogen, C₁-C₆alkyl, (C₁-C₆alkyl)C₃-C₈cycloalkyl, benzyl, 1-phenylethyl, C₁-C₆alkyl which is substituted by hydroxy, methoxy, CONH₂, or CON(CH₃)₂, or C₁-C₆alkyl which is substituted by one, two, or three halo atoms, phenyl substituted by one, two, or three halo atoms or benzyl substituted by one, two, or three halo atoms, or a pharmaceutically acceptable salt or solvate thereof.

18. A compound according to claim 5 wherein R1 is selected from —C(CH₃)(CH₂OH)NH₂, —C(CH₂F)₂NH₂, —C(CH₂F)(CH₂CH₂F)NH₂, —C(CF₃)(CH₃)NH₂, —C(CH₂CH₂F)₂NH₂, —C(CH₂CH₃)(CH₂CF₃)NH₂, 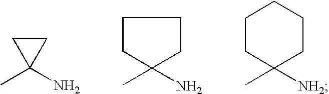
or a pharmaceutically acceptable salt or solvate thereof.

19. A compound according to claim 18 wherein R6 and R7 are each C₁-C₃ alkyl or form a five or six membered carbocyclic ring; or R6 and R7 are independently C₁-C₆alkyl or C₂-C₆alkenyl, in which one or both groups are substituted by one, two, or three halo atoms; or R6 is hydrogen and R7 is C₁-C₆alkyl, C₂-C₆alkenyl which is substituted by one, two, or three halo atoms; or R6 and R7 together with the carbon atom to which they are attached may form a C3-C8cycloalkyl group which is optionally partly unsaturated and which is substituted by one, two, or three halo atoms; or a pharmaceutically acceptable salt or solvate thereof.

20. A compound according to claim 19 wherein R5 is hydrogen, C₁-C₆alkyl, C₁-C₆alkoxy, C₁-C₆alkyl which is substituted by hydroxy or C₁-C₆alkyl which is substituted by one, two, or three halo atoms, or a pharmaceutically acceptable salt or solvate thereof.

21. A compound according to claim 20 wherein R8 is hydrogen, C₁-C₆alkyl, benzyl, C₁-C₆alkyl which is substituted by hydroxy, C₁-C₆alkyl which is substituted by one, two, or three halo atoms, phenyl substituted by one, two, or three halo atoms or benzyl substituted by one, two, or three halo atoms, or a pharmaceutically acceptable salt or solvate thereof.

22. A compound according to any one of claims claim 1 having Formula II 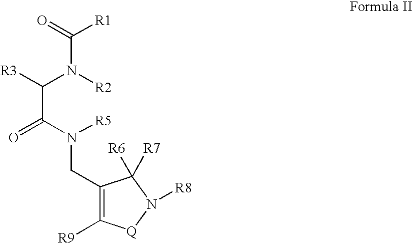
wherein
R1, R2, R3, R5, R6, R7, R8, R9 and Q are as defined in claim 1 or a pharmaceutically acceptable salt or solvate thereof.

23. A compound according to claim 1 wherein R3 is selected from unsubstituted or substituted aryl, unsubstituted or substituted C₁-C₆alkylaryl, unsubstituted or substituted C₁-C₆alkyl(O)—C₁-C₆alkylaryl, unsubstituted or substituted (C₁-C₆ alkyl) C₃-C₈ cycloalkyl; or a pharmaceutically acceptable salt or solvate thereof.

24. A compound according to claim 23 wherein the unsubstituted or substituted aryl group, unsubstituted or substituted C₁-C₆alkylaryl or unsubstituted or substituted C₁-C₆alkyl(O)—C₁-C₆alkylaryl group contains an aryl moiety selected from phenyl, thiazolyl, pyridyl, naphthyl, thienyl, oxazolyl, isoxazolyl and indolyl optionally substituted by from one to three groups independently selected from C₁-C₆ alkyl, —OC₁-C₆ alkyl, —OCF₃, amide, aryl, aryloxy, SO₂(C₁₋₆ alkyl), SO₂CF₃, NHamide, carboxamide, sulfonamide, NHsulfonamide, imide, hydroxy, carboxy, nitro, halo, tri(chloro or fluoro)methyl, and cyano; or a pharmaceutically acceptable salt or solvate thereof.

25. A compound according to claim 1 wherein R3 is an unsubstituted or substituted aryl group, an unsubstituted or substituted C₁-C₆ alkylaryl group or an unsubstituted or substituted C₁-C₆alkyl(O)—C₁-C₆alkyl aryl group wherein:
the C₁-C₆alkyl moiety within the unsubstituted or substituted C₁-C₆ alkylaryl group is methyl, ethyl or propyl;
the C₁-C₆alkyl(O)—C₁-C₆alkyl moiety within the unsubstituted or substituted C₁-C₆alkyl(O)—C₁-C₆alkyl aryl group is a moiety of formula —CH₂OCH₂—;
the unsubstituted or substituted aryl moiety is phenyl, thiazolyl, pyridyl, naphthyl, thienyl, oxazolyl, isoxazolyl and indolyl which is unsubstituted or substituted by from one to three groups independently selected from halo (preferably chloro or fluoro), methyl, methoxy, cyano, SO₂Me, trifluoromethyl, and trifluoromethoxy. Most preferably the unsubstituted aryl moiety is phenyl, naphthyl, thiazolyl or indolyl and the substituted aryl moiety in said groups is 2-fluorophenyl, 3-fluorophenyl, 4-fluorophenyl, 2,3-difluorophenyl, 2,4-difluorophenyl, 2,5-difluorophenyl, 2,6-difluorophenyl, 3,4-difluorophenyl, 3,5-difluorophenyl, 2,4,6-trifluorophenyl, 2,3,4-trifluorophenyl, 2,4,5-trifluorophenyl, 2,3,6-trifluorophenyl, 2,3,5-trifluorophenyl, 2-chlorophenyl, 3-chlorophenyl, 4-chlorophenyl, 2,6-dichlorophenyl, 2,3-dichlorophenyl, 2,4-dichlorophenyl, 2,5-dichlorophenyl, 2-chloro-4-fluorophenyl, 2-methylphenyl, 2,6-difluoro-3-methylphenyl, 3,6-difluoro-2-chlorophenyl, 2-fluoro-6-chlorophenyl, 2-fluoro-3-chlorophenyl, 2-fluoro-4-chlorophenyl, 2,6-difluoro-3-chlorophenyl, 4-trifluoromethylphenyl, 3-trifluoromethylphenyl, 2-trifluoromethylphenyl, 2-fluoro-5-trifluoromethylphenyl, 2-fluoro-3-trifluoromethylphenyl, 2-fluoro-6-trifluoromethylphenyl, 2-chloro-3-trifluoromethylphenyl, 4-trifluoromethoxyphenyl, 3trifluoromethoxyphenyl, 2-trifluoromethoxyphenyl, 2-cyanophenyl, 3-cyanophenyl, 4-cyanophenyl, 4-methanesulphonylphenyl, and 2-methyl thiazolyl.;
or a pharmaceutically acceptable salt or solvate thereof.

26. A compound according to claim 1 wherein R3 is selected from the group consisting of unsubstituted or substituted aryl, C₁-C₆alkylaryl, C₁-C₆alkyl(O)—C₁-C₆alkylaryl, C₃-C₈ cycloalkyl, (C₁-C₆ alkyl) C₃-C₈ cycloalkyl, indolyl, indolinyl, (C₁-C₆ alkyl) indolyl.

27. A compound according to claim 1 wherein R4 is hydrogen or methyl, or a pharmaceutically acceptable salt or solvate thereof.

28. A compound according to claim 1 wherein R9 is selected from the group consisting of unsubstituted or substituted thienyl, unsubstituted or substituted naphthyl, unsubstituted or substituted phenoxy and unsubstituted or substituted phenyl; wherein the substituents when present are each independently selected from the group consisting of halo, methyl, ethyl, propyl, t-butyl, trifluoromethyl, trifluoromethoxy, methoxy, ethoxy, cyano, methylsulphonyl, phenyl, phenoxy, thienyl, pyridyl, thiazolyl, oxazolyl, nitro, CONH₂, furanyl, benzothiophenyl and benzofuranyl; or a pharmaceutically acceptable salt or solvate thereof.

29. A compound of according to claim 28 wherein R9 is selected from phenyl, 4-methylsulphonylphenyl, 3-methylsulphonylphenyl, 4-fluorophenyl, 2-fluorophenyl, 3-fluorophenyl, 3-chlorophenyl, 2-chlorophenyl, 4-chlorophenyl, 4-t-butylphenyl, 4-trifluoromethylphenyl, 3-trifluoromethylphenyl, 4-nitrophenyl, 3-nitrophenyl, 4-bromophenyl, 3-bromophenyl, 2-bromophenyl, 4-methylphenyl, 3-methylphenyl, 4-phenylphenyl, 3-phenylphenyl, 4-phenoxyphenyl, 3-phenoxyphenyl, 4-cyanophenyl, 3-cyanophenyl, 4-carbamoylphenyl, 4-methoxyphenyl, 3-methoxyphenyl, thienyl, thiazolyl, pyridyl, phenoxy, 4-chlorophenoxy, 2,3-dichlorophenyl, 3,4-dichlorophenyl, naphthyl, oxazolyl, 2,4-difluorophenyl, 3,4-difluorophenyl, 3,5-difluorophenyl, 2,3-difluorophenyl, 2,6-difluorophenyl, 2,5-difluorophenyl, 2-fluoro-3-chlorophenyl, 4-ethylphenyl, 4-ethoxyphenyl 3,4,5-trifluorophenyl, 3-fluoro-4-chlorophenyl and 4-carbamoylphenyl;
or a pharmaceutically acceptable salt or solvate thereof.

30. A pharmaceutical formulation comprising one or more compounds according to claim 1 or a pharmaceutically acceptable salt or solvate thereof, and one or more pharmaceutically acceptable diluents or carriers therefor.

31. A pharmaceutical formulation according to claim 30 wherein the formulation further comprises one or more growth hormone secretagogue compounds and/or a bone-antiresorptive agent.

32. A process for producing a compound of Formula I as defined in claim 1 comprising coupling a compound of Formula XI or XIb 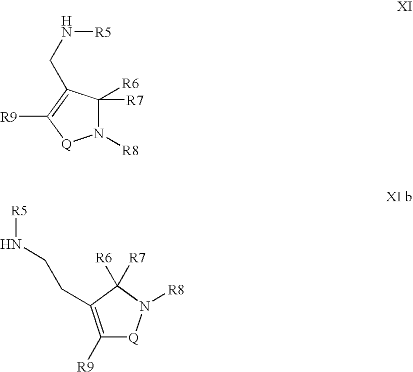
with a compound of formula XIII 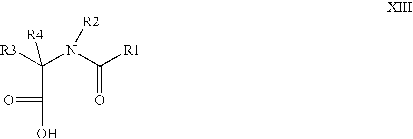
wherein R1, R2, R3, R4, R5, R6, R7, R8, R9 and Q are as defined in claim 1.

33. A process for producing a compound of Formula I as defined in claim 1 comprising deprotecting a compound of Formula 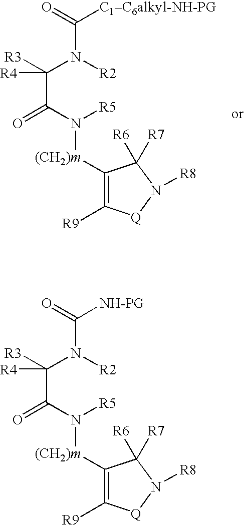
wherein R2, R3, R4, R5, R6, R7, R8, R9, m and Q are as defined in claim 1, and PG is an amino protecting group.

34. A process for producing a compound of Formula I as defined in claim 1 comprising coupling a compound of Formula 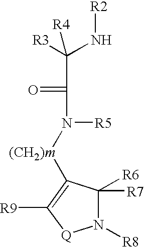
with a compound of formula XIV
HOOC—R1 XIV
wherein R1, R2, R3, R4, R5, R6, R7, R8, R9 and Q are as defined in 1.

35-36. (canceled)

37. A method comprising administering an effective amount of a compound of claim 1 or a pharmaceutically acceptable salt or solvate thereof for the treatment of a physiological condition which is modulated or ameliorated by an increase in endogenous growth hormone to an animal in need of said treatment.

38. A method-comprising administering an effective amount of a compound of claim 1 or a pharmaceutically acceptable salt or solvate thereof for treating a condition selected from osteoporosis, physiological short stature caused by growth hormone deficiency, short stature associated with chronic illness, growth retardation associated with the Prader-Willi syndrome, intrauterine growth retardation, pulmonary dysfunction and ventricular dependency, insulin resistance, cachexia and protein loss due to cancer or AIDS to an animal in need of said treatment.

39. A compound selected from the group consisting of
2-(R)-2-(2-Amino-2-methylpropionylamino)-3-benzyloxy-propionic acid N-[5-(4-chlorophenyl)-2-cyclopropylmethyl-3,3-dimethyl-1,1-dioxo-2,3-dihydroisothiazol-4-ylmethyl]-N-ethylamide;
2-(R)-2-(2-Amino-2-methylpropionylamino)-3-benzyloxy-propionic acid N-[5-(4-chlorophenyl)-3,3-dimethyl-1,1-dioxo-2-(2-methoxyethyl)-2,3-dihydroisothiazol-4-ylmethyl]-N-ethylamide;
2-(R)-2-(2-Amino-2-methylpropionylamino)-3-benzyloxy-propionic acid N-[5-(4-chlorophenyl)-3,3-dimethyl-1,1-dioxo-2-(2-fluoroethyl)-2,3-dihydroisothiazol-4-ylmethyl]-N-ethylamide;
2-(R)-2-(2-Amino-2-methylpropionylamino)-3-benzyloxy-propionic acid N-[5-(4-chlorophenyl)-3,3-dimethyl-1,1-dioxo-2-(4,4,4-trifluorobutyl)-2,3-dihydroisothiazol-4-ylmethyl]-N-ethylamide;
2-(R)-2-(2-Amino-2-methylpropionylamino)-3-benzyloxy-propionic acid N-[2-carbamoylmethyl-5-(4-chlorophenyl)-3,3-dimethyl-1,1-dioxo-2,3-dihydroisothiazol-4-ylmethyl]-N-ethylamide; and
2-(R)-2-(2-Amino-2-methylpropionylamino)-3-benzyloxy-propionic acid N-[5-(4-chlorophenyl)-3,3-dimethyl-2-(N′,N′-dimethylcarbamoyl)methyl-1,1-dioxo-2,3-dihydroisothiazol-4-ylmethyl]-N-ethylamide;
or a pharmaceutically acceptable salt or solvate thereof.

40. A compound of the formula 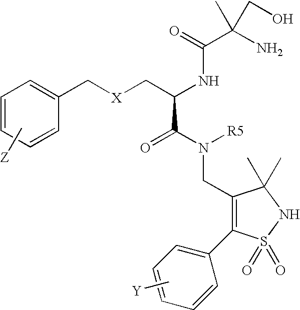
wherein
X is O, Y is 4-Cl, Z is H and R5 is CH₂CH₃; or a pharmaceutically acceptable salt or solvate thereof.

41. A method comprising administering an effect amount of a compound of claim 39 or a pharmaceutically acceptable salt or solvate thereof for the treatment of a physiological condition which is modulated or ameliorated by an increase in endogenous growth hormone-to an animal in need of said treatment.

42. A method comprising administering an effective amount of a compound of claim 39 or a pharmaceutically acceptable salt or solvate thereof for treating a condition selected from osteoporosis, physiological short stature caused by growth hormone deficiency, short stature associated with chronic illness, growth retardation associated with the Prader-Willi syndrome, intrauterine growth retardation, pulmonary dysfunction and ventricular dependency, insulin resistance, cachexia and protein loss due to cancer or AIDS to an animal in need of said treatment.

43. A method comprising administering an effect amount of a compound of claim 40 or a pharmaceutically acceptable salt or solvate thereof for the treatment of a physiological condition which is modulated or ameliorated by an increase in endogenous growth hormone-to an animal in need of said treatment.

44. A method comprising administering an effective amount of a compound of claim 40 or a pharmaceutically acceptable salt or solvate thereof for treating a condition selected from osteoporosis, physiological short stature caused by growth hormone deficiency, short stature associated with chronic illness, growth retardation associated with the Prader-Willi syndrome, intrauterine growth retardation, pulmonary dysfunction and ventricular dependency, insulin resistance, cachexia and protein loss due to cancer or AIDS to an animal in need of said treatment.

45. A compound according to claim 2 having Formula II 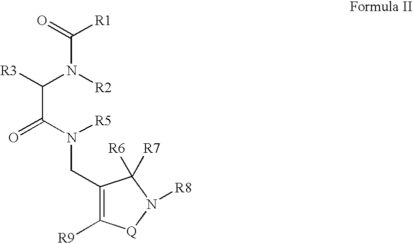
wherein
R1, R2, R3, R5, R6, R7, R8, R9 and Q are as defined in claim 2 or a pharmaceutically acceptable salt or solvate thereof.

46. A compound according to claim 45 wherein R3 is selected from unsubstituted or substituted aryl, unsubstituted or substituted C₁-C₆alkylaryl, unsubstituted or substituted C₁-C₆alkyl(O)—C₁-C₆alkylaryl, unsubstituted or substituted (C₁-C₆ alkyl) C₃-C₈ cycloalkyl; wherein the unsubstituted or substituted aryl group, unsubstituted or substituted C₁-C₆alkylaryl or unsubstituted or substituted C₁-C₆alkyl(O)—C₁-C₆alkylaryl group contains an aryl moiety selected from phenyl, thiazolyl, pyridyl, naphthyl, thienyl, oxazolyl, isoxazolyl and indolyl optionally substituted by from one to three groups independently selected from C₁-C₆ alkyl, —OC₁-C₆ alkyl, —OCF₃, amide, aryl, aryloxy, SO₂(C₁₋₆ alkyl), SO₂CF₃, NHamide, carboxamide, sulfonamide, NHsulfonamide, imide, hydroxy, carboxy, nitro, halo, tri(chloro or fluoro)methyl, and cyano; or a pharmaceutically acceptable salt or solvate thereof.

47. A compound according to claim 45 wherein R3 is an unsubstituted or substituted aryl group, an unsubstituted or substituted C₁-C₆ alkylaryl group or an unsubstituted or substituted C₁-C₆alkyl(O)—C₁-C₆alkyl aryl group wherein:
the C₁-C₆alkyl moiety within the unsubstituted or substituted C₁-C₆ alkylaryl group is methyl, ethyl or propyl;
the C₁-C₆alkyl(O)—C₁-C₆alkyl moiety within the unsubstituted or substituted C—C₆alkyl(O)—C₁-C₆alkyl aryl group is a moiety of formula —CH₂OCH₂—;
the unsubstituted or substituted aryl moiety is phenyl, thiazolyl, pyridyl, naphthyl, thienyl, oxazolyl, isoxazolyl and indolyl which is unsubstituted or substituted by from one to three groups independently selected from halo (preferably chloro or fluoro), methyl, methoxy, cyano, SO₂Me, trifluoromethyl, and trifluoromethoxy. Most preferably the unsubstituted aryl moiety is phenyl, naphthyl, thiazolyl or indolyl and the substituted aryl moiety in said groups is 2-fluorophenyl, 3-fluorophenyl, 4-fluorophenyl, 2,3-difluorophenyl, 2,4-difluorophenyl, 2,5-difluorophenyl, 2,6-difluorophenyl, 3,4-difluorophenyl, 3,5-difluorophenyl, 2,4,6-trifluorophenyl, 2,3,4-trifluorophenyl, 2,4,5-trifluorophenyl, 2,3,6-trifluorophenyl, 2,3,5-trifluorophenyl, 2-chlorophenyl, 3-chlorophenyl, 4-chlorophenyl, 2,6-dichlorophenyl, 2,3-dichlorophenyl, 2,4-dichlorophenyl, 2,5-dichlorophenyl, 2-chloro-4-fluorophenyl, 2-methylphenyl, 2,6-difluoro-3-methylphenyl, 3,6-difluoro-2-chlorophenyl, 2-fluoro-6-chlorophenyl, 2-fluoro-3-chlorophenyl, 2-fluoro-4-chlorophenyl, 2,6-difluoro-3-chlorophenyl, 4-trifluoromethylphenyl, 3-trifluoromethylphenyl, 2-trifluoromethylphenyl, 2-fluoro-5-trifluoromethylphenyl, 2-fluoro-3-trifluoromethylphenyl, 2-fluoro-6-trifluoromethylphenyl, 2-chloro-3-trifluoromethylphenyl, 4-trifluoromethoxyphenyl, 3trifluoromethoxyphenyl, 2-trifluoromethoxyphenyl, 2-cyanophenyl, 3-cyanophenyl, 4-cyanophenyl, 4-methanesulphonylphenyl, and 2-methyl thiazolyl;
or a pharmaceutically acceptable salt or solvate thereof.

48. A compound according to claim 45 wherein R3 is selected from the group consisting of unsubstituted or substituted aryl, C₁-C₆alkylaryl, C₁-C₆alkyl(O)—C₁-C₆alkylaryl, C₃-C₈ cycloalkyl, (C₁-C₆ alkyl) C₃-C₈ cycloalkyl, indolyl, indolinyl, (C₁-C₆ alkyl) indolyl.

49. A compound according to claim 45 wherein R9 is selected from the group consisting of unsubstituted or substituted thienyl, unsubstituted or substituted naphthyl, unsubstituted or substituted phenoxy and unsubstituted or substituted phenyl; wherein the substituents when present are each independently selected from the group consisting of halo, methyl, ethyl, propyl, t-butyl, trifluoromethyl, trifluoromethoxy, methoxy, ethoxy, cyano, methylsulphonyl, phenyl, phenoxy, thienyl, pyridyl, thiazolyl, oxazolyl, nitro, CONH₂, furanyl, benzothiophenyl and benzofuranyl; or a pharmaceutically acceptable salt or solvate thereof.

50. A compound of according to claim 49 wherein R9 is selected from phenyl, 4-methylsulphonylphenyl, 3-methylsulphonylphenyl, 4-fluorophenyl, 2-fluorophenyl, 3-fluorophenyl, 3-chlorophenyl, 2-chlorophenyl, 4-chlorophenyl, 4-t-butylphenyl, 4-trifluoromethylphenyl, 3-trifluoromethylphenyl, 4-nitrophenyl, 3-nitrophenyl, 4-bromophenyl, 3-bromophenyl, 2-bromophenyl, 4-methylphenyl, 3-methylphenyl, 4-phenylphenyl, 3-phenylphenyl, 4-phenoxyphenyl, 3-phenoxyphenyl, 4-cyanophenyl, 3-cyanophenyl, 4-carbamoylphenyl, 4-methoxyphenyl, 3-methoxyphenyl, thienyl, thiazolyl, pyridyl, phenoxy, 4-chlorophenoxy, 2,3-dichlorophenyl, 3,4-dichlorophenyl, naphthyl, oxazolyl, 2,4-difluorophenyl, 3,4-difluorophenyl, 3,5-difluorophenyl, 2,3-difluorophenyl, 2,6-difluorophenyl, 2,5-difluorophenyl, 2-fluoro-3-chlorophenyl, 4-ethylphenyl, 4-ethoxyphenyl 3,4,5-trifluorophenyl, 3-fluoro-4-chlorophenyl and 4-carbamoylphenyl;
or a pharmaceutically acceptable salt or solvate thereof.

51. A compound according to claim 3 having Formula II 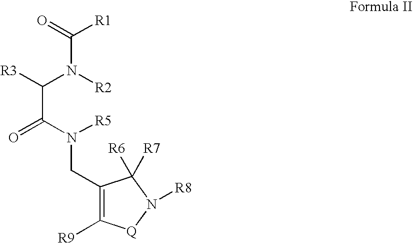
wherein
R1, R2, R3, R5, R6, R7, R8, R9 and Q are as defined in claim 3 or a pharmaceutically acceptable salt or solvate thereof.

52. A compound according to claim 51 wherein R3 is selected from unsubstituted or substituted aryl, unsubstituted or substituted C₁-C₆alkylaryl, unsubstituted or substituted C₁-C₆alkyl(O)—C₁-C₆alkylaryl, unsubstituted or substituted (C₁-C₆ alkyl) C₃-C₈ cycloalkyl; wherein the unsubstituted or substituted aryl group, unsubstituted or substituted C₁-C₆alkylaryl or unsubstituted or substituted C₁-C₆alkyl(O)—C₁-C₆alkylaryl group contains an aryl moiety selected from phenyl, thiazolyl, pyridyl, naphthyl, thienyl, oxazolyl, isoxazolyl and indolyl optionally substituted by from one to three groups independently selected from C₁-C₆ alkyl, —OC₁-C₆ alkyl, —OCF₃, amide, aryl, aryloxy, SO₂(C₁₋₆ alkyl), SO₂CF₃, NHamide, carboxamide, sulfonamide, NHsulfonamide, imide, hydroxy, carboxy, nitro, halo, tri(chloro or fluoro)methyl, and cyano; or a pharmaceutically acceptable salt or solvate thereof.

53. A compound according to claim 51 wherein R3 is an unsubstituted or substituted aryl group, an unsubstituted or substituted C₁-C₆ alkylaryl group or an unsubstituted or substituted C₁-C₆alkyl(O)—C₁-C₆alkyl aryl group wherein:
the C₁-C₆alkyl moiety within the unsubstituted or substituted C₁-C₆ alkylaryl group is methyl, ethyl or propyl;
the C₁-C₆alkyl(O)—C₁-C₆alkyl moiety within the unsubstituted or substituted C₁-C₆alkyl(O)—C₁-C₆alkyl aryl group is a moiety of formula —CH₂OCH₂—;
the unsubstituted or substituted aryl moiety is phenyl, thiazolyl, pyridyl, naphthyl, thienyl, oxazolyl, isoxazolyl and indolyl which is unsubstituted or substituted by from one to three groups independently selected from halo (preferably chloro or fluoro), methyl, methoxy, cyano, SO₂Me, trifluoromethyl, and trifluoromethoxy. Most preferably the unsubstituted aryl moiety is phenyl, naphthyl, thiazolyl or indolyl and the substituted aryl moiety in said groups is 2-fluorophenyl, 3-fluorophenyl, 4-fluorophenyl, 2,3-difluorophenyl, 2,4-difluorophenyl, 2,5-difluorophenyl, 2,6-difluorophenyl, 3,4-difluorophenyl, 3,5-difluorophenyl, 2,4,6-trifluorophenyl, 2,3,4-trifluorophenyl, 2,4,5-trifluorophenyl, 2,3,6-trifluorophenyl, 2,3,5-trifluorophenyl, 2-chlorophenyl, 3-chlorophenyl, 4-chlorophenyl, 2,6-dichlorophenyl, 2,3-dichlorophenyl, 2,4-dichlorophenyl, 2,5-dichlorophenyl, 2-chloro-4-fluorophenyl, 2-methylphenyl, 2,6-difluoro-3-methylphenyl, 3,6-difluoro-2-chlorophenyl, 2-fluoro-6-chlorophenyl, 2-fluoro-3-chlorophenyl, 2-fluoro-4-chlorophenyl, 2,6-difluoro-3-chlorophenyl, 4-trifluoromethylphenyl, 3-trifluoromethylphenyl, 2-trifluoromethylphenyl, 2-fluoro-5-trifluoromethylphenyl, 2-fluoro-3-trifluoromethylphenyl, 2-fluoro-6-trifluoromethylphenyl, 2-chloro-3-trifluoromethylphenyl, 4-trifluoromethoxyphenyl, 3-trifluoromethoxyphenyl, 2-trifluoromethoxyphenyl, 2-cyanophenyl, 3-cyanophenyl, 4-cyanophenyl, 4-methanesulphonylphenyl, and 2-methyl thiazolyl;
or a pharmaceutically acceptable salt or solvate thereof.

54. A compound according to claim 51 wherein R3 is selected from the group consisting of unsubstituted or substituted aryl, C₁-C₆alkylaryl, C₁-C₆alkyl(O)—C₁-C₆alkylaryl, C₃-C₈ cycloalkyl, (C₁-C₆ alkyl) C₃-C₈ cycloalkyl, indolyl, indolinyl, (C₁-C₆ alkyl) indolyl.

55. A compound according to claim 51 wherein R9 is selected from the group consisting of unsubstituted or substituted thienyl, unsubstituted or substituted naphthyl, unsubstituted or substituted phenoxy and unsubstituted or substituted phenyl; wherein the substituents when present are each independently selected from the group consisting of halo, methyl, ethyl, propyl, t-butyl, trifluoromethyl, trifluoromethoxy, methoxy, ethoxy, cyano, methylsulphonyl, phenyl, phenoxy, thienyl, pyridyl, thiazolyl, oxazolyl, nitro, CONH₂, furanyl, benzothiophenyl and benzofuranyl; or a pharmaceutically acceptable salt or solvate thereof.

56. A compound of according to claim 55 wherein R9 is selected from phenyl, 4-methylsulphonylphenyl, 3-methylsulphonylphenyl, 4-fluorophenyl, 2-fluorophenyl, 3-fluorophenyl, 3-chlorophenyl, 2-chlorophenyl, 4-chlorophenyl, 4-t-butylphenyl, 4-trifluoromethylphenyl, 3-trifluoromethylphenyl, 4-nitrophenyl, 3-nitrophenyl, 4-bromophenyl, 3-bromophenyl, 2-bromophenyl, 4-methylphenyl, 3-methylphenyl, 4-phenylphenyl, 3-phenylphenyl, 4-phenoxyphenyl, 3-phenoxyphenyl, 4-cyanophenyl, 3-cyanophenyl, 4-carbamoylphenyl, 4-methoxyphenyl, 3-methoxyphenyl, thienyl, thiazolyl, pyridyl, phenoxy, 4-chlorophenoxy, 2,3-dichlorophenyl, 3,4-dichlorophenyl, naphthyl, oxazolyl, 2,4-difluorophenyl, 3,4-difluorophenyl, 3,5-difluorophenyl, 2,3-difluorophenyl, 2,6-difluorophenyl, 2,5-difluorophenyl, 2-fluoro-3-chlorophenyl, 4-ethylphenyl, 4-ethoxyphenyl 3,4,5-trifluorophenyl, 3-fluoro-4-chlorophenyl and 4-carbamoylphenyl;
or a pharmaceutically acceptable salt or solvate thereof.

57. A compound according to claim 4 having Formula II 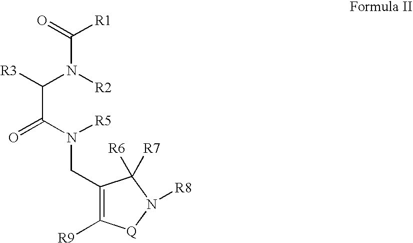
wherein
R1, R2, R3, R5, R6, R7, R8, R9 and Q are as defined in claim 4 or a pharmaceutically acceptable salt or solvate thereof.

58. A compound according to claim 57 wherein R3 is selected from unsubstituted or substituted aryl, unsubstituted or substituted C₁-C₆alkylaryl, unsubstituted or substituted C₁-C₆alkyl(O)—C₁-C₆alkylaryl, unsubstituted or substituted (C₁-C₆ alkyl) C₃-C₈ cycloalkyl; wherein the unsubstituted or substituted aryl group, unsubstituted or substituted C₁-C₆alkylaryl or unsubstituted or substituted C₁-C₆alkyl(O)—C₁-C₆alkylaryl group contains an aryl moiety selected from phenyl, thiazolyl, pyridyl, naphthyl, thienyl, oxazolyl, isoxazolyl and indolyl optionally substituted by from one to three groups independently selected from C₁-C₆ alkyl, —OC₁-C₆ alkyl, —OCF₃, amide, aryl, aryloxy, SO₂(C₁₋₆ alkyl), SO₂CF₃, NHamide, carboxamide, sulfonamide, NHsulfonamide, imide, hydroxy, carboxy, nitro, halo, tri(chloro or fluoro)methyl, and cyano; or a pharmaceutically acceptable salt or solvate thereof.

59. A compound according to claim 57 wherein R3 is an unsubstituted or substituted aryl group, an unsubstituted or substituted C₁-C₆ alkylaryl group or an unsubstituted or substituted C₁-C₆alkyl(O)—C₁-C₆alkyl aryl group wherein:
the C₁-C₆alkyl moiety within the unsubstituted or substituted C₁-C₆ alkylaryl group is methyl, ethyl or propyl;
the C₁-C₆alkyl(O)—C₁-C₆alkyl moiety within the unsubstituted or substituted C₁-C₆alkyl(O)—C₁-C₆alkyl aryl group is a moiety of formula —CH₂OCH₂—;
the unsubstituted or substituted aryl moiety is phenyl, thiazolyl, pyridyl, naphthyl, thienyl, oxazolyl, isoxazolyl and indolyl which is unsubstituted or substituted by from one to three groups independently selected from halo (preferably chloro or fluoro), methyl, methoxy, cyano, SO₂Me, trifluoromethyl, and trifluoromethoxy. Most preferably the unsubstituted aryl moiety is phenyl, naphthyl, thiazolyl or indolyl and the substituted aryl moiety in said groups is 2-fluorophenyl, 3-fluorophenyl, 4-fluorophenyl, 2,3-difluorophenyl, 2,4-difluorophenyl, 2,5-difluorophenyl, 2,6-difluorophenyl, 3,4-difluorophenyl, 3,5-difluorophenyl, 2,4,6-trifluorophenyl, 2,3,4-trifluorophenyl, 2,4,5-trifluorophenyl, 2,3,6-trifluorophenyl, 2,3,5-trifluorophenyl, 2-chlorophenyl, 3-chlorophenyl, 4-chlorophenyl, 2,6-dichlorophenyl, 2,3-dichlorophenyl, 2,4-dichlorophenyl, 2,5-dichlorophenyl, 2-chloro-4-fluorophenyl, 2-methylphenyl, 2,6-difluoro-3-methylphenyl, 3,6-difluoro-2-chlorophenyl, 2-fluoro-6-chlorophenyl, 2-fluoro-3-chlorophenyl, 2-fluoro-4-chlorophenyl, 2,6-difluoro-3-chlorophenyl, 4-trifluoromethylphenyl, 3-trifluoromethylphenyl, 2-trifluoromethylphenyl, 2-fluoro-5-trifluoromethylphenyl, 2-fluoro-3-trifluoromethylphenyl, 2-fluoro-6-trifluoromethylphenyl, 2-chloro-3-trifluoromethylphenyl, 4-trifluoromethoxyphenyl, 3-trifluoromethoxyphenyl, 2-trifluoromethoxyphenyl, 2-cyanophenyl, 3-cyanophenyl, 4-cyanophenyl, 4-methanesulphonylphenyl, and 2-methyl thiazolyl;
or a pharmaceutically acceptable salt or solvate thereof.

60. A compound according to claim 57 wherein R3 is selected from the group consisting of unsubstituted or substituted aryl, C₁-C₆alkylaryl, C₁-C₆alkyl(O)—C₁-C₆alkylaryl, C₃-C₈ cycloalkyl, (C₁-C₆ alkyl) C₃-C₈ cycloalkyl, indolyl, indolinyl, (C₁-C₆ alkyl) indolyl.

61. A compound according to claim 57 wherein R9 is selected from the group consisting of unsubstituted or substituted thienyl, unsubstituted or substituted naphthyl, unsubstituted or substituted phenoxy and unsubstituted or substituted phenyl; wherein the substituents when present are each independently selected from the group consisting of halo, methyl, ethyl, propyl, t-butyl, trifluoromethyl, trifluoromethoxy, methoxy, ethoxy, cyano, methylsulphonyl, phenyl, phenoxy, thienyl, pyridyl, thiazolyl, oxazolyl, nitro, CONH₂, furanyl, benzothiophenyl and benzofuranyl; or a pharmaceutically acceptable salt or solvate thereof.

62. A compound of according to claim 61 wherein R9 is selected from phenyl, 4-methylsulphonylphenyl, 3-methylsulphonylphenyl, 4-fluorophenyl, 2-fluorophenyl, 3-fluorophenyl, 3-chlorophenyl, 2-chlorophenyl, 4-chlorophenyl, 4-t-butylphenyl, 4-trifluoromethylphenyl, 3-trifluoromethylphenyl, 4-nitrophenyl, 3-nitrophenyl, 4-bromophenyl, 3-bromophenyl, 2-bromophenyl, 4-methylphenyl, 3-methylphenyl, 4-phenylphenyl, 3-phenylphenyl, 4-phenoxyphenyl, 3-phenoxyphenyl, 4-cyanophenyl, 3-cyanophenyl, 4-carbamoylphenyl, 4-methoxyphenyl, 3-methoxyphenyl, thienyl, thiazolyl, pyridyl, phenoxy, 4-chlorophenoxy, 2,3-dichlorophenyl, 3,4-dichlorophenyl, naphthyl, oxazolyl, 2,4-difluorophenyl, 3,4-difluorophenyl, 3,5-difluorophenyl, 2,3-difluorophenyl, 2,6-difluorophenyl, 2,5-difluorophenyl, 2-fluoro-3-chlorophenyl, 4-ethylphenyl, 4-ethoxyphenyl 3,4,5-trifluorophenyl, 3-fluoro-4-chlorophenyl and 4-carbamoylphenyl;
or a pharmaceutically acceptable salt or solvate thereof.

63. A compound according to claim 5 having Formula II 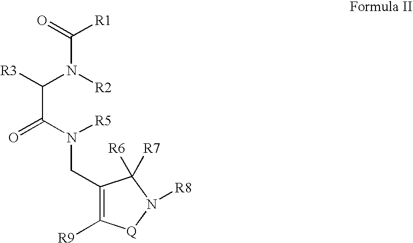
wherein
R1, R2, R3, R5, R6, R7, R8, R9 and Q are as defined in claim 5 or a pharmaceutically acceptable salt or solvate thereof.

64. A compound according to claim 63 wherein R3 is selected from unsubstituted or substituted aryl, unsubstituted or substituted C₁-C₆alkylaryl, unsubstituted or substituted C₁-C₆alkyl(O)—C₁-C₆alkylaryl, unsubstituted or substituted (C₁-C₆ alkyl) C₃-C₈ cycloalkyl; wherein the unsubstituted or substituted aryl group, unsubstituted or substituted C₁-C₆alkylaryl or unsubstituted or substituted C₁-C₆alkyl(O)—C₁-C₆alkylaryl group contains an aryl moiety selected from phenyl, thiazolyl, pyridyl, naphthyl, thienyl, oxazolyl, isoxazolyl and indolyl optionally substituted by from one to three groups independently selected from C₁-C₆ alkyl, —OC₁-C₆ alkyl, —OCF₃, amide, aryl, aryloxy, SO₂(C₁₋₆ alkyl), SO₂CF₃, NHamide, carboxamide, sulfonamide, NHsulfonamide, imide, hydroxy, carboxy, nitro, halo, tri(chloro or fluoro)methyl, and cyano; or a pharmaceutically acceptable salt or solvate thereof.

65. A compound according to claim 63 wherein R3 is an unsubstituted or substituted aryl group, an unsubstituted or substituted C₁-C₆ alkylaryl group or an unsubstituted or substituted C₁-C₆alkyl(O)—C₁-C₆alkyl aryl group wherein:
the C₁-C₆alkyl moiety within the unsubstituted or substituted C₁-C₆ alkylaryl group is methyl, ethyl or propyl;
the C₁-C₆alkyl(O)—C₁-C₆alkyl moiety within the unsubstituted or substituted C₁-C₆alkyl(O)—C₁-C₆alkyl aryl group is a moiety of formula —CH₂OCH₂—;
the unsubstituted or substituted aryl moiety is phenyl, thiazolyl, pyridyl, naphthyl, thienyl, oxazolyl, isoxazolyl and indolyl which is unsubstituted or substituted by from one to three groups independently selected from halo (preferably chloro or fluoro), methyl, methoxy, cyano, SO₂Me, trifluoromethyl, and trifluoromethoxy. Most preferably the unsubstituted aryl moiety is phenyl, naphthyl, thiazolyl or indolyl and the substituted aryl moiety in said groups is 2-fluorophenyl, 3-fluorophenyl, 4-fluorophenyl, 2,3-difluorophenyl, 2,4-difluorophenyl, 2,5-difluorophenyl, 2,6-difluorophenyl, 3,4-difluorophenyl, 3,5-difluorophenyl, 2,4,6-trifluorophenyl, 2,3,4-trifluorophenyl, 2,4,5-trifluorophenyl, 2,3,6-trifluorophenyl, 2,3,5-trifluorophenyl, 2-chlorophenyl, 3-chlorophenyl, 4-chlorophenyl, 2,6-dichlorophenyl, 2,3-dichlorophenyl, 2,4-dichlorophenyl, 2,5-dichlorophenyl, 2-chloro-4-fluorophenyl, 2-methylphenyl, 2,6-difluoro-3-methylphenyl, 3,6-difluoro-2-chlorophenyl, 2-fluoro-6-chlorophenyl, 2-fluoro-3-chlorophenyl, 2-fluoro-4-chlorophenyl, 2,6-difluoro-3-chlorophenyl, 4-trifluoromethylphenyl, 3-trifluoromethylphenyl, 2-trifluoromethylphenyl, 2-fluoro-5-trifluoromethylphenyl, 2-fluoro-3-trifluoromethylphenyl, 2-fluoro-6-trifluoromethylphenyl, 2-chloro-3-trifluoromethylphenyl, 4-trifluoromethoxyphenyl, 3-trifluoromethoxyphenyl, 2-trifluoromethoxyphenyl, 2-cyanophenyl, 3-cyanophenyl, 4-cyanophenyl, 4-methanesulphonylphenyl, and 2-methyl thiazolyl;
or a pharmaceutically acceptable salt or solvate thereof.

66. A compound according to claim 63 wherein R3 is selected from the group consisting of unsubstituted or substituted aryl, C₁-C₆alkylaryl, C₁-C₆alkyl(O)—C₁-C₆alkylaryl, C₃-C₈ cycloalkyl, (C₁-C₆ alkyl) C₃-C₈ cycloalkyl, indolyl, indolinyl, (C₁-C₆ alkyl) indolyl.

67. A compound according to claim 63 wherein R9 is selected from the group consisting of unsubstituted or substituted thienyl, unsubstituted or substituted naphthyl, unsubstituted or substituted phenoxy and unsubstituted or substituted phenyl; wherein the substituents when present are each independently selected from the group consisting of halo, methyl, ethyl, propyl, t-butyl, trifluoromethyl, trifluoromethoxy, methoxy, ethoxy, cyano, methylsulphonyl, phenyl, phenoxy, thienyl, pyridyl, thiazolyl, oxazolyl, nitro, CONH₂, furanyl, benzothiophenyl and benzofuranyl; or a pharmaceutically acceptable salt or solvate thereof.

68. A compound of according to claim 63 wherein R9 is selected from phenyl, 4-methylsulphonylphenyl, 3-methylsulphonylphenyl, 4-fluorophenyl, 2-fluorophenyl, 3-fluorophenyl, 3-chlorophenyl, 2-chlorophenyl, 4-chlorophenyl, 4-t-butylphenyl, 4-trifluoromethylphenyl, 3-trifluoromethylphenyl, 4-nitrophenyl, 3-nitrophenyl, 4-bromophenyl, 3-bromophenyl, 2-bromophenyl, 4-methylphenyl, 3-methylphenyl, 4-phenylphenyl, 3-phenylphenyl, 4-phenoxyphenyl, 3-phenoxyphenyl, 4-cyanophenyl, 3-cyanophenyl, 4-carbamoylphenyl, 4-methoxyphenyl, 3-methoxyphenyl, thienyl, thiazolyl, pyridyl, phenoxy, 4-chlorophenoxy, 2,3-dichlorophenyl, 3,4-dichlorophenyl, naphthyl, oxazolyl, 2,4-difluorophenyl, 3,4-difluorophenyl, 3,5-difluorophenyl, 2,3-difluorophenyl, 2,6-difluorophenyl, 2,5-difluorophenyl, 2-fluoro-3-chlorophenyl, 4-ethylphenyl, 4-ethoxyphenyl 3,4,5-trifluorophenyl, 3-fluoro-4-chlorophenyl and 4-carbamoylphenyl;
or a pharmaceutically acceptable salt or solvate thereof.

69. A compound according to claim 21 having Formula II 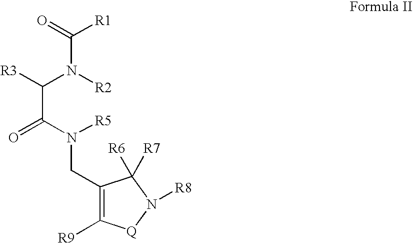
wherein
R1, R2, R3, R5, R6, R7, R8, R9 and Q are as defined in claim 21 or a pharmaceutically acceptable salt or solvate thereof.

70. A compound according to claim 69 wherein R3 is selected from unsubstituted or substituted aryl, unsubstituted or substituted C₁-C₆alkylaryl, unsubstituted or substituted C₁-C₆alkyl(O)—C₁-C₆alkylaryl, unsubstituted or substituted (C₁-C₆ alkyl) C₃-C₈ cycloalkyl; wherein the unsubstituted or substituted aryl group, unsubstituted or substituted C₁-C₆alkylaryl or unsubstituted or substituted C₁-C₆alkyl(O)—C₁-C₆alkylaryl group contains an aryl moiety selected from phenyl, thiazolyl, pyridyl, naphthyl, thienyl, oxazolyl, isoxazolyl and indolyl optionally substituted by from one to three groups independently selected from C₁-C₆ alkyl, —OC₁-C₆ alkyl, —OCF₃, amide, aryl, aryloxy, SO₂(C₁₋₆ alkyl), SO₂CF₃, NHamide, carboxamide, sulfonamide, NHsulfonamide, imide, hydroxy, carboxy, nitro, halo, tri(chloro or fluoro)methyl, and cyano; or a pharmaceutically acceptable salt or solvate thereof.

71. A compound according to claim 69 wherein R3 is an unsubstituted or substituted aryl group, an unsubstituted or substituted C₁-C₆ alkylaryl group or an unsubstituted or substituted C₁-C₆alkyl(O)—C₁-C₆alkyl aryl group wherein:
the C₁-C₆alkyl moiety within the unsubstituted or substituted C₁-C₆ alkylaryl group is methyl, ethyl or propyl;
the C₁-C₆alkyl(O)—C₁-C₆alkyl moiety within the unsubstituted or substituted C₁-C₆alkyl(O)—C₁-C₆alkyl aryl group is a moiety of formula —CH₂OCH₂—;
the unsubstituted or substituted aryl moiety is phenyl, thiazolyl, pyridyl, naphthyl, thienyl, oxazolyl, isoxazolyl and indolyl which is unsubstituted or substituted by from one to three groups independently selected from halo (preferably chloro or fluoro), methyl, methoxy, cyano, SO₂Me, trifluoromethyl, and trifluoromethoxy. Most preferably the unsubstituted aryl moiety is phenyl, naphthyl, thiazolyl or indolyl and the substituted aryl moiety in said groups is 2-fluorophenyl, 3-fluorophenyl, 4-fluorophenyl, 2,3-difluorophenyl, 2,4-difluorophenyl, 2,5-difluorophenyl, 2,6-difluorophenyl, 3,4-difluorophenyl, 3,5-difluorophenyl, 2,4,6-trifluorophenyl, 2,3,4-trifluorophenyl, 2,4,5-trifluorophenyl, 2,3,6-trifluorophenyl, 2,3,5-trifluorophenyl, 2-chlorophenyl, 3-chlorophenyl, 4-chlorophenyl, 2,6-dichlorophenyl, 2,3-dichlorophenyl, 2,4-dichlorophenyl, 2,5-dichlorophenyl, 2-chloro-4-fluorophenyl, 2-methylphenyl, 2,6-difluoro-3-methylphenyl, 3,6-difluoro-2-chlorophenyl, 2-fluoro-6-chlorophenyl, 2-fluoro-3-chlorophenyl, 2-fluoro-4-chlorophenyl, 2,6-difluoro-3-chlorophenyl, 4-trifluoromethylphenyl, 3-trifluoromethylphenyl, 2-trifluoromethylphenyl, 2-fluoro-5-trifluoromethylphenyl, 2-fluoro-3-trifluoromethylphenyl, 2-fluoro-6-trifluoromethylphenyl, 2-chloro-3-trifluoromethylphenyl, 4-trifluoromethoxyphenyl, 3-trifluoromethoxyphenyl, 2-trifluoromethoxyphenyl, 2-cyanophenyl, 3-cyanophenyl, 4-cyanophenyl, 4-methanesulphonylphenyl, and 2-methyl thiazolyl;
or a pharmaceutically acceptable salt or solvate thereof.

72. A compound according to claim 69 wherein R3 is selected from the group consisting of unsubstituted or substituted aryl, C₁-C₆alkylaryl, C₁-C₆alkyl(O)—C₁-C₆alkylaryl, C₃-C₈ cycloalkyl, (C₁-C₆ alkyl) C₃-C₈ cycloalkyl, indolyl, indolinyl, (C₁-C₆ alkyl) indolyl.

73. A compound according to claim 69 wherein R9 is selected from the group consisting of unsubstituted or substituted thienyl, unsubstituted or substituted naphthyl, unsubstituted or substituted phenoxy and unsubstituted or substituted phenyl; wherein the substituents when present are each independently selected from the group consisting of halo, methyl, ethyl, propyl, t-butyl, trifluoromethyl, trifluoromethoxy, methoxy, ethoxy, cyano, methylsulphonyl, phenyl, phenoxy, thienyl, pyridyl, thiazolyl, oxazolyl, nitro, CONH₂, furanyl, benzothiophenyl and benzofuranyl; or a pharmaceutically acceptable salt or solvate thereof.

74. A compound of according to claim 69 wherein R9 is selected from phenyl, 4-methylsulphonylphenyl, 3-methylsulphonylphenyl, 4-fluorophenyl, 2-fluorophenyl, 3-fluorophenyl, 3-chlorophenyl, 2-chlorophenyl, 4-chlorophenyl, 4-t-butylphenyl, 4-trifluoromethylphenyl, 3-trifluoromethylphenyl, 4-nitrophenyl, 3-nitrophenyl, 4-bromophenyl, 3-bromophenyl, 2-bromophenyl, 4-methylphenyl, 3-methylphenyl, 4-phenylphenyl, 3-phenylphenyl, 4-phenoxyphenyl, 3-phenoxyphenyl, 4-cyanophenyl, 3-cyanophenyl, 4-carbamoylphenyl, 4-methoxyphenyl, 3-methoxyphenyl, thienyl, thiazolyl, pyridyl, phenoxy, 4-chlorophenoxy, 2,3-dichlorophenyl, 3,4-dichlorophenyl, naphthyl, oxazolyl, 2,4-difluorophenyl, 3,4-difluorophenyl, 3,5-difluorophenyl, 2,3-difluorophenyl, 2,6-difluorophenyl, 2,5-difluorophenyl, 2-fluoro-3-chlorophenyl, 4-ethylphenyl, 4-ethoxyphenyl 3,4,5-trifluorophenyl, 3-fluoro-4-chlorophenyl and 4-carbamoylphenyl;
or a pharmaceutically acceptable salt or solvate thereof.
